(19) United States
(12) Patent Application Publication
(10) Pub. No.: US 2012/0129100 A1
(43) Pub. Date: May 24, 2012

(54) ACTINIC RAY-SENSITIVE OR RADIATION-SENSITIVE RESIN COMPOSITION, AND RESIST FILM AND PATTERN FORMING METHOD USING THE SAME

(75) Inventors: Akinori Shibuya, Shizuoka (JP); Shuhei Yamaguchi, Shizuoka (JP); Shohei Kataoka, Shizuoka (JP); Michihiro Shirakawa, Shizuoka (JP); Takayuki Kato, Shizuoka (JP); Naohiro Tango, Shizuoka (JP)

(73) Assignee: FUJIFILM CORPORATION, Tokyo (JP)

(21) Appl. No.: 13/388,404

(22) PCT Filed: Aug. 17, 2010
(86) PCT No.: PCT/JP2010/064128

§ 371 Date: Feb. 1, 2012

(30) Foreign Application Priority Data
Aug. 28, 2009 (JP) 2009-199037
Jun. 22, 2010 (JP) 2010-142061

(51) Int. Cl. G03F 7/20; C07D 409/12; C07D 409/14; G03F 7/027

(52) U.S. Cl. 430/281.1; 430/325; 544/379; 546/213; 544/373; 546/202; 546/187; 546/280.4; 548/466

(57) ABSTRACT

An actinic ray-sensitive or radiation-sensitive resin composition including: (PA) a compound having a proton acceptor functional group and undergoing decomposition upon irradiation with an actinic ray or radiation to generate a compound reduced in or deprived of proton acceptor property or changed to be acidic from being proton acceptor-functioning, wherein a molar extinction coefficient ε of the compound (PA) at a wavelength of 193 nm as measured in acetonitrile solvent is 55,000 or less, and a pattern forming method using the composition are provided.

### TECHNICAL FIELD

The present invention relates to an actinic ray-sensitive or radiation-sensitive resin composition for use in the production process of a semiconductor device such as IC, in the production of a liquid crystal device or a circuit board such as thermal head and further in other photofabrication processes, and a pattern forming method using the composition. More specifically, the present invention relates to an actinic ray-sensitive or radiation-sensitive resin composition suitable when using a far ultraviolet ray at a wavelength of 250 nm or less, an electron beam or the like as the light source, and a resist film and a pattern forming method each using the composition.

### BACKGROUND ART

A chemical amplification resist produces an acid in the exposed area upon irradiation with radiation such as far ultraviolet light and through a reaction using the acid as a catalyst, causes a change in the developer solubility of the area irradiated with radiation and that of the non-irradiated area, thereby forming a pattern on a substrate.

In the case of using a KrF excimer laser as the exposure light source, a good pattern with high sensitivity and high resolution is formed because a resin exhibiting small absorption mainly in the 248-nm region and having poly(hydroxystyrene) as the basic structure is used as the main component, and this is a good system as compared with the conventional naphthoquinone diazide/novolak resin system.

On the other hand, in the case where a light source at a shorter wavelength, for example, an ArF excimer laser (193 nm) is used as the exposure light source, even the above-described chemical amplification system is not sufficient because the compound having an aromatic group inherently exhibits large absorption in the 193-nm region.

Therefore, various resists for an ArF excimer laser, containing an alicyclic hydrocarbon structure, have been developed. However, in view of overall performance as a resist, it is in fact very difficult to find out an appropriate combination of a resin, a photo-acid generator, additives, a solvent and the like used.

In JP-A-2006-330098 (the term “JP-A” as used herein means an “unexamined published Japanese patent application”) and Japanese Patent 3,577,743, it is proposed to solve the problem (PED) related to the length of time from exposure to post-exposure bake (PEB) and satisfy the pattern profile or suppression of the line edge roughness by using a specific compound capable of decomposing upon irradiation with an actinic ray or radiation.

In the latest-generation pattern formation with a line width of 45 nm or less, where an immersion process is applied, the above-described related arts are not necessarily sufficient, and more improvements are demanded in terms of line width roughness (LWR) and depth-of-focus (DOF).

### SUMMARY OF INVENTION

Considering those problems in the background art, an object of the present invention is to provide an actinic ray-sensitive or radiation-sensitive resin composition improved in LWR and DOF and suitable also for an immersion process with a line width of 45 nm or less, and a resist film and a pattern forming method each using the composition.

The above-described object can be attained by the following techniques.

That is, the present invention includes the following configurations.

(1) An actinic ray-sensitive or radiation-sensitive resin composition, comprising:

(PA) a compound having a proton acceptor functional group and undergoing decomposition upon irradiation with an actinic ray or radiation to generate a compound reduced in or deprived of proton acceptor property or changed to be acidic from being proton acceptor-functioning,

wherein a molar extinction coefficient ε of the compound (PA) at a wavelength of 193 nm as measured in acetonitrile solvent is 55,000 or less.

(2) The actinic ray-sensitive or radiation-sensitive resin composition as described in (1) above, further comprising:

(B1) a resin capable of increasing a solubility of the resin (B1) in an alkali developer by an action of an acid,

wherein the resin (B1) contains a resin having a repeating unit represented by the following formula (V), and

the compound (PA) is a compound capable of decomposing upon irradiation with an actinic ray or radiation to generate a compound represented by the following formula (PA-1):

Q-A_{PA1}-(X)ₙ—R (PA-1)

wherein Q represents —SO₃H, —CO₂H or —W₁—NH—W₂—Rf;

each of X, W₁ and W₂ independently represents —SO₂— or —CO—;

Rf represents an alkyl group which may be substituted with a halogen atom, a cycloalkyl group which may be substituted with a halogen atom, or an aryl group which may be substituted with a halogen atom;

A_{PA1} represents a single bond or a divalent linking group;

n represents 0 or 1; and

R represents a monovalent organic group having a proton acceptor functional group:

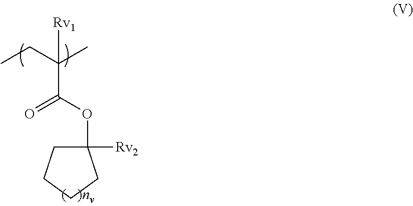

wherein each of Rv₁ and Rv₂ independently represents an alkyl group having a carbon number of 1 to 10; and

nᵥ represents an integer of 1 to 6.

(3) The actinic ray-sensitive or radiation-sensitive resin composition as described in (1) or (2) above, wherein the compound (PA) is represented by the following formula (II) or (III):

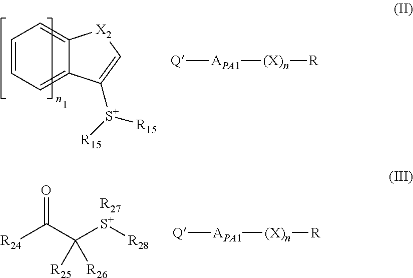

wherein each R₁₅ independently represents an alkyl group or a cycloalkyl group, two R₁₅'s may combine with each other to form a ring;

X₂ represents any one of —CR₂₁═CR₂₂—, —NR₂₃—, —S— and —O—;

each of R₂₁ to R₂₃ independently represents a hydrogen atom, an alkyl group, a cycloalkyl group, an alkoxy group or an aryl group;

R₂₄ represents an aryl group;

each of R₂₅ and R₂₆ independently represents a hydrogen atom, an alkyl group or a cycloalkyl group, R₂₅ and R₂₆ may combine with each other to form a ring;

each of R₂₇ and R₂₈ independently represents a hydrogen atom, an alkyl group, a cycloalkyl group, an allyl group or a vinyl group, R₂₇ and R₂₈ may combine with each other to form a ring;

n₁ represents an integer of 0 to 3;

Q′ represents —SO₃—, —CO₂— or —W₁—N⁻—W₂—Rf; and

X, W₁, W₂, Rf, A_{PA1}, R and n have the same meanings as X, W₁, W₂, Rf, A_{PA1}, R and n in formula (PA-1).

(4) The actinic ray-sensitive or radiation-sensitive resin composition as described in (3) above,

wherein Q′ in formula (II) or (III) is —W₁—N⁻—W₂—Rf,

wherein each of W₁ and W₂ independently represents —SO₂— or —CO—; and

Rf represents an alkyl group which may be substituted with a halogen atom, a cycloalkyl group which may be substituted with a halogen atom, or an aryl group which may be substituted with a halogen atom.

(5) The actinic ray-sensitive or radiation-sensitive resin composition as described in any one of (1) to (4) above, further comprising:

(C) a compound capable of generating an acid upon irradiation with an actinic ray or radiation.

(6) The actinic ray-sensitive or radiation-sensitive resin composition as described in any one of (1) to (5) above,

wherein the resin (B1) has a lactone group substituted with a cyano group.

(7) The actinic ray-sensitive or radiation-sensitive resin composition as described in any one of (1) to (6) above,

wherein the resin (B1) contains a repeating unit having a lactone structure represented by the following formula (III):

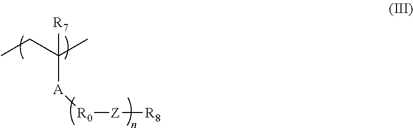

wherein A represents an ester bond (a group represented by —COO—) or an amide bond (a group represented by —CONH—);

R₀ represents an alkylene group, a cycloalkylene group or a combination thereof, and when a plurality of R₀'s are present, the plurality of R₀'s are the same or different;

Z represents an ether bond, an ester bond, an amide bond, a group represented by —O—C(═O)—N(R)— or —N(R)—C(═O)—O—, or a group represented by —N(R)—C(═O)—N(R)—, and when a plurality of Z's are present, the plurality of Z's are the same or different, in which R represents a hydrogen atom, an alkyl group, a cycloalkyl group or an aryl group;

R₈ represents a monovalent organic group having a lactone structure;

n represents an integer of 1 to 5; and

R₇ represents a hydrogen atom, a halogen atom or an alkyl group.

(8) The actinic ray-sensitive or radiation-sensitive resin composition as described in any one of (1) to (7) above, further comprising:

a hydrophobic resin.

(9) A resist film, which is formed from the actinic ray-sensitive or radiation-sensitive resin composition as described in any one of (1) to (8) above.

(10) A pattern forming method, comprising:

forming a resist film by using the actinic ray-sensitive or radiation-sensitive resin composition as described in any one of (1) to (8) above; and

exposing and developing the resist film.

(11) The pattern forming method as described in (10) above,

wherein exposure in the exposing is immersion exposure.

The present invention preferably further includes the following configurations.

(12) The actinic ray-sensitive or radiation-sensitive resin composition as described in any one of (1) to (8) above,

wherein the molar extinction coefficient ε of the compound (PA) at a wavelength of 193 nm as measured in acetonitrile solvent is from 6,500 to 55,000.

(13) The actinic ray-sensitive or radiation-sensitive resin composition as described in any one of (1) to (8) and (12) above, which is used for ArF excimer laser exposure.

(14) The actinic ray-sensitive or radiation-sensitive resin composition as described in any one of (1) to (8), (12) and (13) above, which is used for immersion exposure.

### DESCRIPTION OF EMBODIMENTS

The present invention is described in detail below.

In the present invention, when a group (atomic group) is denoted without specifying whether substituted or unsubstituted, the group includes both a group having no substituent and a group having a substituent. For example, “an alkyl group” includes not only an alkyl group having no substituent (unsubstituted alkyl group) but also an alkyl group having a substituent (substituted alkyl group).

In the present invention, the term “actinic ray” or “radiation” indicates, for example, a bright line spectrum of mercury lamp, a far ultraviolet ray typified by excimer laser, an extreme-ultraviolet ray (EUV light), an X-ray or an electron beam. Also, in the present invention, the “light” means an actinic ray or radiation.

Furthermore, in the present invention, unless otherwise indicated, the “exposure” includes not only exposure with a mercury lamp, a far ultraviolet ray typified by excimer laser, an X-ray, EUV light or the like but also lithography with a particle beam such as electron beam and ion beam.

### [1] Proton Acceptor Compound (PA)

The composition of the present invention contains (PA) a compound having a proton acceptor functional group and undergoing decomposition upon irradiation with an actinic ray or radiation to generate a compound reduced in or deprived of the proton acceptor property or changed to be acidic from being proton acceptor-functioning (hereinafter sometimes referred to as a “compound (PA)”).

As regards the compound (PA) for use in the present invention, the molar extinction coefficient ε at a wavelength of 193 nm as measured in an acetonitrile solvent is 0.8 or less in terms of the relative ratio to triphenylsulfonium nonafluorobutanesulfonate.

The proton acceptor functional group is a functional group having a group or electron capable of electrostatically interacting with a proton and indicates, for example, a functional group having a macrocyclic structure such as cyclic polyether, or a functional group containing a nitrogen atom having an unshared electron pair not contributing to π-conjugation. The nitrogen atom having an unshared electron pair not contributing to π-conjugation is, for example, a nitrogen atom having a partial structure represented by the following formulae:

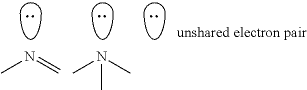

Preferred examples of the proton acceptor functional group include a crown ether residue, an aza-crown ether residue, a primary to tertiary amine residue, a pyrrole residue, a pyridine residue, an imidazole residue, a pyrazine residue, a piperidine residue and a piperazine residue.

In the case where the proton acceptor functional group contains a nitrogen atom, from the standpoint of bringing out the proton acceptor property, it is preferred that all atoms adjacent to nitrogen atom contained in the functional group are a carbon atom or a hydrogen atom. Also, from the standpoint of bringing out the proton acceptor property, an electron-withdrawing functional group (e.g., carbonyl group, sulfonyl group, cyano group, halogen atom) is preferably not bonded directly to nitrogen atom.

The compound (PA) decomposes upon irradiation with an actinic ray or radiation to generate a compound reduced in or deprived of the proton acceptor property or changed to be acidic from being proton acceptor-functioning.

Similarly to the conventional basic compound described later, in the unexposed area, the compound (PA) acts to prevent the acid (proton) generated in the exposed area from diffusing even to the unexposed area and exerting its action. Also, in the exposed area, the compound (PA) generates, as described above, a compound reduced in or deprived of the proton acceptor property or changed to be acidic from being proton acceptor-functioning and therefore, does not inhibit the action of the acid in the exposed area.

The expression “reduced in the proton acceptor property” as used herein means that when a noncovalent complex as a proton adduct is produced from the proton acceptor functional group-containing compound (PA) and a proton, the equilibrium constant in the chemical equilibrium decreases.

The proton acceptor property can be confirmed by measuring the pH.

The compound (PA) is preferably an ionic compound. The proton acceptor functional group may be contained in either the anion moiety or the cation moiety but is preferably contained in the anion moiety.

The molar extinction coefficient ε (unit: l/mol/cm) of the compound (PA) at a wavelength of 193 nm is 55,000 or less, preferably from 6,500 to 55,000, more preferably from 13,000 to 47,000, still more preferably from 15,000 to 32,500. When the molar extinction coefficient is 55,000 or less, the transmittance of the composition film is increased to allow for transmission of light to the substrate interface of the resist film and this enables elevating the rectangularity of the pattern and obtaining the objective performance in terms of LWR and DOF, and when the molar extinction coefficient is 6,500 or more, the light absorption efficiency can be suitably ensured and the sensitivity can be maintained.

The relative molar extinction coefficient at a wavelength of 193 nm is subject to the effect of an aromatic ring and therefore, the absorbance can be adjusted to the range above by designing the structure of the compound (PA), for example, by controlling the number of aromatic rings. Specific examples thereof include the later-described compound represented by formula (II) or (III).

The compound (PA) decomposes upon irradiation with an actinic ray or radiation to generate, for example, a compound represented by the following formula (PA-1). The compound represented by formula (PA-1) is a compound having an acidic group together with a proton acceptor functional group and thereby being reduced in or deprived of the proton acceptor property or changed to be acidic from being proton acceptor-functioning as compared with the compound (PA).

Q-A_{PA1}-(X)ₙ—R (PA-1)

In formula (PA-1), Q represents —SO₃H, —CO₂H or —W₁—NH—W₂—Rf.

Each of X, W₁ and W₂ independently represents —SO₂— or —CO—, and Rf represents an alkyl group which may be substituted with a halogen atom, a cycloalkyl group which may be substituted with a halogen atom, or an aryl group which may be substituted with a halogen atom.

A_{PA1} represents a single bond or a divalent linking group,

n represents 0 or 1.

R represents a monovalent organic group having a proton acceptor functional group.

Formula (PA-1) is described in detail below.

The divalent linking group in A_{PA1} is preferably a divalent organic group having a carbon number of 2 to 12, and examples thereof include an alkylene group and a phenylene group. An alkylene group having at least one fluorine atom is preferred, and the carbon number thereof is preferably from 2 to 6, more preferably from 2 to 4. The alkylene chain may contain a linking group such as oxygen atom and sulfur atom. The alkylene group is preferably an alkylene group where from 30 to 100% by number of the hydrogen atom is replaced by a fluorine atom, more preferably an alkylene group where the carbon atom bonded to the Q site has a fluorine atom, still more preferably a perfluoroalkylene group, yet still more preferably a perfluoroethylene group, a perfluoropropylene group or a perfluorobutylene group.

The monovalent organic group having a proton acceptor functional group of R is preferably a monovalent organic group having a carbon number of 4 to 30, and examples thereof include an alkyl group, a cycloalkyl group, an aryl group, an aralkyl group and an alkenyl group, where an arbitrary atom is substituted with a proton acceptor functional group. These groups may further have a substituent other than a proton acceptor functional group.

The proton acceptor functional group in R is as described above, and examples thereof include a crown ether residue, an aza-crown ether residue, a primary to tertiary amine residue, a pyrrole residue, a pyridine residue, an imidazole residue, a pyrazine residue, a piperidine residue and a piperazine residue.

The alkyl group in R may have a substituent other than a proton acceptor functional group and is preferably a linear or branched alkyl group having a carbon number of 1 to 20, and the alkyl chain may contain an oxygen atom, a sulfur atom or a nitrogen atom.

Here, the alkyl group having a proton acceptor functional group and further having a substituent other than that includes particularly a group where a proton acceptor functional group and a cycloalkyl group are substituted on a linear or branched alkyl group (for example, an adamantylmethyl group, an adamantylethyl group, a cyclohexylethyl group and a camphor residue each having a proton acceptor functional group).

The cycloalkyl group in R may have a substituent other than a proton acceptor functional group and is preferably a cycloalkyl group having a carbon number of 3 to 20, and the cycloalkyl group may contain an oxygen atom in the ring.

The aryl group in R may have a substituent other than a proton acceptor functional group and is preferably an aryl group having a carbon number of 6 to 14.

The aralkyl group in R may have a substituent other than a proton acceptor functional group and is preferably an aralkyl group having a carbon number of 7 to 20.

The alkenyl group in R may have a substituent other than a proton acceptor functional group, and examples thereof include a group having a double bond at an arbitrary position of the alkyl group described for R.

Examples of the substituent which the groups for R may further have in addition to a proton acceptor functional group include a halogen atom, a hydroxyl group, a nitro group, a cyano group, a carboxy group, a carbonyl group, an alkyl group (preferably having a carbon number of 1 to 10), a cycloalkyl group (preferably having a carbon number of 3 to 10), an aryl group (preferably having a carbon number of 6 to 14), an alkoxy group (preferably having a carbon number of 1 to 10), an aryloxy group (preferably having a carbon number of 6 to 14), an acyl group (preferably having a carbon number of 2 to 20), an acyloxy group (preferably having a carbon number of 2 to 10), an alkoxycarbonyl group (preferably having a carbon number of 2 to 20), an aminoacyl group (preferably having a carbon number of 2 to 20), an alkylthio group (preferably having a carbon number of 1 to 10), and an arylthio group (preferably having a carbon number of 6 to 14). As for the cyclic structure in the aryl group, cycloalkyl group and the like and the aminoacyl group, examples of the substituent further include an alkyl group (preferably having a carbon number of 1 to 20).

Q preferably represents —SO₃H or —W₁—NH—W₂—Rf and in view of LWR performance, more preferably represents —W₁—NH—W₂—Rf. Preferably, at least either one of W₁ and W₂ is —SO₂—; and more preferably, both W₁ and W₂ are —SO₂—.

Rf represents an alkyl group which may be substituted with a halogen atom, a cycloalkyl group which may be substituted with a halogen atom, or an aryl group which may be substituted with a halogen atom. Specific examples thereof include the alkyl, cycloalkyl and aryl groups exemplified as the monovalent organic group in R, and a group formed by substituting a halogen atom on these groups (provided that the group does not have a proton acceptor functional group). The halogen atom is preferably a fluorine atom, a chlorine atom or a bromine atom, more preferably a fluorine atom. Rf is preferably an alkyl group having a carbon number of 1 to 6, which may have a fluorine atom, more preferably a perfluoroalkyl group having a carbon number of 1 to 3.

These groups (A_{PA1}, X, W₁, W₂ and Rf) may further have a substituent, and examples of the substituent which these groups may further have include a halogen atom, a hydroxyl group, a nitro group, a cyano group, a carboxy group, a carbonyl group, an alkyl group (preferably having a carbon number of 1 to 10), a cycloalkyl group (preferably having a carbon number of 3 to 10), an aryl group (preferably having a carbon number of 6 to 14), an alkoxy group (preferably having a carbon number of 1 to 10), an aryloxy group (preferably having a carbon number of 6 to 14), an acyl group (preferably having a carbon number of 2 to 20), an acyloxy group (preferably having a carbon number of 2 to 10), an alkoxycarbonyl group (preferably having a carbon number of 2 to 20), an aminoacyl group (preferably having a carbon number of 2 to 20), an alkylthio group (preferably having a carbon number of 1 to 10), and an arylthio group (preferably having a carbon number of 6 to 14). As for the cyclic structure in the aryl group, cycloalkyl group and the like and the aminoacyl group, examples of the substituent further include an alkyl group (preferably having a carbon number of 1 to 20).

Out of the compounds represented by formula (PA-1), the compound where the Q site is a sulfonic acid can be synthesized by using a general sulfonamidation reaction. For example, the compound may be obtained by a method of selectively reacting one sulfonyl halide moiety of a bis-sulfonyl halide compound with an amine compound to form a sulfonamide bond and then hydrolyzing the other sulfonyl halide moiety, or a method of ring-opening a cyclic sulfonic anhydride through a reaction with an amine compound.

The compound (PA) is preferably a compound represented by the following formula (II) or (III):

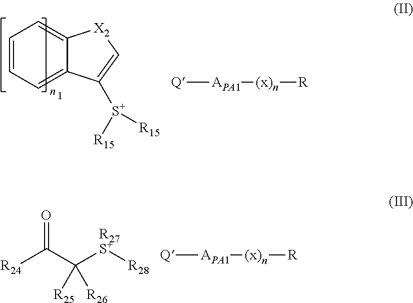

wherein each R₁₅ independently represents an alkyl group or a cycloalkyl group, two R₁₅'s may combine with each other to form a ring,

X₂ represents any of —CR₂₁═CR₂₂—, —NR₂₃—, —S— and —O—, each of R₂₁ to R₂₃ independently represents a hydrogen atom, an alkyl group, a cycloalkyl group, an alkoxy group or an aryl group,

R₂₄ represents an aryl group,

each of R₂₅ and R₂₆ independently represents a hydrogen atom, an alkyl group or a cycloalkyl group, R₂₅ and R₂₆ may combine with each other to form a ring,

each of R₂₇ and R₂₈ independently represents a hydrogen atom, an alkyl group, a cycloalkyl group, an allyl group or a vinyl group, R₂₇ and R₂₈ may combine with each other to form a ring,

n₁ represents an integer of 0 to 3,

Q′ represents —SO₃—, —CO₂— or —W₁—N⁻—W₂—Rf, and

X, W₁, W₂, Rf, A_{PA1}, R and n have the same meanings as those in formula (PA-1).

Examples of the alkyl group, cycloalkyl group and aryl group in R₁₅ and R₂₁ to R₂₈ are the same as those for R in formula (PA-1) (provided that the group does not have a proton acceptor functional group). The alkoxy group of R₂₁ to R₂₃ is preferably an alkoxy group having a carbon number of 1 to 10.

Each of these groups may further have a substituent, and examples of the substituent which each of these groups may have are the same as those of the substituent which the group and the like of R in formula (PA-1) may have.

The ring which may be formed by combining two R₁₅′ is a ring structure formed together with —S⁺ in formula (II) and is preferably a 5-membered ring containing one sulfur atom or a condensed ring containing the ring. In the case of a condensed ring, the condensed ring is preferably a ring containing one sulfur atom and 18 or less carbon atoms, more preferably a ring structure represented by the following formulae (IV-1) to (IV-3). In the formulae, * represents a bond. R represents an arbitrary substituent, and examples thereof are the same as those of the substituent which the group and the like of R in formula (PA-1) may have, n represents an integer of 0 to 4, and n2 represents an integer of 0 to 3.

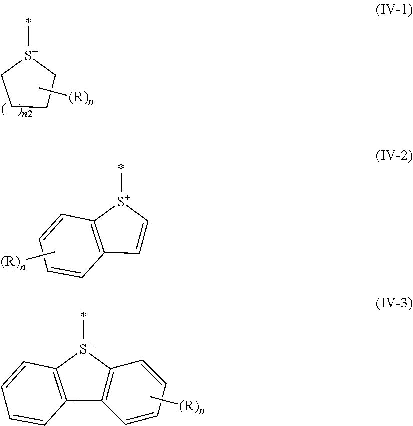

Out of the compounds represented by formulae (II) and (III), preferred cation structures include the following cation structures (ZI-1) to (ZI-5).

The cation structure (ZI-1) is a structure represented by the following formula (ZI-1):

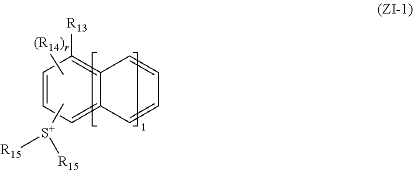

In formula (ZI-1), R₁₃ represents a hydrogen atom, a fluorine atom, a hydroxyl group, an alkyl group, a cycloalkyl group, an alkoxy group, an alkoxycarbonyl group, or a group having a cycloalkyl group. These groups may have a substituent. R₁₃ is preferably a hydrogen atom, a fluorine atom, a hydroxyl group, an alkyl group, a cycloalkyl group, an alkoxy group or an alkoxycarbonyl group and in view of LWR performance, is preferably a group having a cycloalkyl group.

R₁₄ represents, when a plurality of R₁₄'s are present, each independently represents, a hydroxyl group, an alkyl group, a cycloalkyl group, an alkoxy group, an alkoxycarbonyl group, an alkylcarbonyl group, an alkylsulfonyl group, a cycloalkylsulfonyl group, or a group having a cycloalkyl group. These groups may have a substituent. R₁₄ is preferably an alkyl group, a cycloalkyl group, an alkoxy group, an alkylsulfonyl group or a cycloalkylsulfonyl group.

Each R₁₅ independently represents an alkyl group or a cycloalkyl group. Two R₁₅'s may combine with each other to form a ring.

l represents an integer of 0 to 2.

r represents an integer of 0 to 10.

In formula (ZI-1), the alkyl group as R₁₃, R₁₄ and R₁₅ is a linear or branched alkyl group preferably having a carbon number of 1 to 10, and examples thereof include a methyl group, an ethyl group, an n-propyl group, an i-propyl group, an n-butyl group, a 2-methylpropyl group, a 1-methylpropyl group, a tert-butyl group, an n-pentyl group, a neopentyl group, an n-hexyl group, an n-heptyl group, an n-octyl group, a 2-ethylhexyl group, an n-nonyl group and an n-decyl group. Among these alkyl groups, a methyl group, an ethyl group, an n-butyl group and a tert-butyl group are preferred.

The cycloalkyl group of R₁₃, R₁₄ and R₁₅ may be monocyclic or polycyclic and is preferably a cycloalkyl group having a carbon number of 3 to 12, and examples thereof include cyclopropyl, cyclobutyl, cyclopentyl, cyclohexyl, cycloheptyl, cyclooctyl, cyclododecanyl, cyclopentenyl, cyclohexenyl, cyclooctadienyl, bicycloheptyl(norbornyl) and adamantyl. Among these, cyclopropyl, cyclopentyl, cyclohexyl, cycloheptyl and cyclooctyl are preferred.

The alkoxy group of R₁₃ and R₁₄ may be linear, branched or cyclic (that is, a cycloalkyloxy group; may be either monocyclic or polycyclic) and is preferably an alkoxy group having a carbon number of 1 to 10, and examples thereof include a linear or branched alkyloxy group such as methoxy group, ethoxy group, n-propoxy group, i-propoxy group, n-butoxy group, 2-methylpropoxy group, 1-methylpropoxy group, tert-butoxy group, n-pentyloxy group, neopentyloxy group, n-hexyloxy group, n-heptyloxy group, n-octyloxy group, 2-ethylhexyloxy group, n-nonyloxy group and n-decyloxy group; and an alkyloxy group having a cycloalkyl group, such as cyclopentylmethyloxy group, cyclohexylmethyloxy group, cycloheptylmethyloxy group, cycloheptyloxy group, cyclooctyloxy group, cyclohexylethyloxy group and norbornylmethyloxy group. Among these alkoxy groups, an alkoxy group having a carbon number of 7 or more, such as n-heptyloxy group, cyclohexylmethyloxy group, n-octyloxy group, cyclohexylethyloxy group, 2-ethylhexyloxy group, n-nonyloxy group and n-decyloxy group, is more preferred, and an alkoxy group having a cycloalkyl group, such as cyclohexylmethyloxy group and cyclohexylethyloxy group, is still more preferred.

The alkoxycarbonyl group of R₁₃ is preferably a linear or branched alkoxycarbonyl group having a carbon number of 2 to 11 and includes, for example, an alkoxycarbonyl group where the alkyl group in R₁₃, R₁₄ and R₁₅ is substituted on a carbonyl group, and examples thereof a methoxycarbonyl group, an ethoxycarbonyl group, an n-propoxycarbonyl group, an i-propoxycarbonyl group, an n-butoxycarbonyl group, a 2-methylpropoxycarbonyl group, a 1-methylpropoxycarbonyl group, a tert-butoxycarbonyl group, an n-pentyloxycarbonyl group, a neopentyloxycarbonyl group, an n-hexyloxycarbonyl group, an n-heptyloxycarbonyl group, an n-octyloxycarbonyl group, a 2-ethylhexyloxycarbonyl group, an n-nonyloxycarbonyl group and an n-decyloxycarbonyl group. Among these alkoxycarbonyl groups, a methoxycarbonyl group, an ethoxycarbonyl group and an n-butoxycarbonyl group are preferred. The group having a cycloalkyl group of R₁₃ and R₁₄ includes a group having a monocyclic or polycyclic cycloalkyl group (preferably a cycloalkyl group having a carbon number of 3 to 20), and examples thereof include a monocyclic or polycyclic cycloalkyloxy group and a monocyclic or polycyclic cycloalkyl group-containing alkoxy group. These groups may further have a substituent.

The monocyclic or polycyclic cycloalkyloxy group of R₁₃ and R₁₄ is preferably a cycloalkyloxy group having a total carbon number of 7 or more, more preferably a total carbon number of 7 to 15, and preferably has a monocyclic cycloalkyl group. The monocyclic cycloalkyloxy group having a total carbon number of 7 or more indicates a monocyclic cycloalkyloxy group that is a cycloalkyloxy group (e.g., cyclopropyloxy, cyclobutyloxy, cyclopentyloxy, cyclohexyloxy, cyclobutyloxy, cyclooctyloxy, cyclododecanyloxy) having an arbitrary substituent such as alkyl group (e.g., methyl group, ethyl group, propyl group, butyl group, pentyl group, hexyl group, heptyl group, octyl group, dodecyl group, 2-ethylhexyl group, isopropyl group, sec-butyl group, tert-butyl group, isoamyl group), hydroxyl group, halogen atom (e.g., fluorine, chlorine, bromine, iodine), nitro group, cyano group, amido group, sulfonamide group, alkoxy group (e.g., methoxy group, ethoxy group, hydroxyethoxy group, propoxy group, hydroxypropoxy group, butoxy group), alkoxycarbonyl group (e.g., methoxycarbonyl group, ethoxycarbonyl group), acyl group (e.g., formyl group, acetyl group, benzoyl group), acyloxy group (e.g., acetoxy group, butyryloxy group) and carboxy group, where the total carbon number inclusive of the carbon number of an arbitrary substituent on the cycloalkyl group is 7 or more.

Examples of the polycyclic cycloalkyloxy group having a total carbon number of 7 or more include a norbornyloxy group, a tricyclodecanyloxy group, a tetracyclodecanyloxy group and an adamantyloxy group.

The alkoxy group having a monocyclic or polycyclic cycloalkyl group of R₁₃ and R₁₄ preferably has a total carbon number of 7 or more, more preferably a total carbon number of 7 to 15, and is preferably alkoxy group having a monocyclic cycloalkyl skeleton. The alkoxy group having a total carbon number of 7 or more and having a monocyclic cycloalkyl group indicates an alkoxy group (e.g., methoxy, ethoxy, propoxy, butoxy, pentyloxy, hexyloxy, heptoxy, octyloxy, dodecyloxy, 2-ethylhexyloxy, isopropoxy, sec-butoxy, tert-butoxy, isoamyloxy) substituted with the above-described monocyclic cycloalkyl group which may have a substituent, where the total carbon number inclusive of the carbon number of the substituent is 7 or more. Examples thereof include a cyclohexylmethoxy group, a cyclopentylethoxy group and a cyclohexylethoxy group, with a cyclohexylmethoxy group being preferred.

Examples of the alkoxy group having a total carbon number of 7 or more and having a polycyclic cycloalkyl group include a norbornylmethoxy group, a norbornylethoxy group, a tricyclodecanylmethoxy group, a tricyclodecanylethoxy group, a tetracyclodecanylmethoxy group, a tetracyclodecanylethoxy group, an adamantylmethoxy group and an adamantylethoxy group, with a norbornylmethoxy group and a norbornylethoxy group being preferred.

Specific examples of the alkyl group in the alkylcarbonyl group of R₁₄ are the same as those of the alkyl group of R₁₃ to R₁₅ above.

The alkylsulfonyl group and cycloalkylsulfonyl group of R₁₄ are preferably a linear, branched or cyclic alkylsulfonyl or cycloalkylsulfonyl group having a carbon number of 1 to 10 and include, for example, those where the alkyl group in R₁₃, R₁₄ and R₁₅ is substituted on a sulfonyl group. Examples thereof include a methanesulfonyl group, an ethanesulfonyl group, an n-propanesulfonyl group, an n-butanesulfonyl group, a tert-butanesulfonyl group, an n-pentanesulfonyl group, a neopentanesulfonyl group, an n-hexanesulfonyl group, an n-heptanesulfonyl group, an n-octanesulfonyl group, a 2-ethylhexanesulfonyl group, an n-nonanesulfonyl group, an n-decanesulfonyl group, a cyclopentanesulfonyl group and a cyclohexanesulfonyl group. Among these alkylsulfonyl groups and cycloalkylsulfonyl groups, a methanesulfonyl group, an ethanesulfonyl group, an n-propanesulfonyl group, an n-butanesulfonyl group, a cyclopentanesulfonyl group and a cyclohexanesulfonyl group are preferred.

l is preferably 0 or 1, more preferably 1, and r is preferably a number of 0 to 2.

Each of the groups for R₁₃, R₁₄ and R₁₅ may further have a substituent. Examples of the substituent which each group may have include an alkyl group such as methyl group, ethyl group, propyl group, butyl group, pentyl group, hexyl group, heptyl group, octyl group, dodecyl group, 2-ethylhexyl group, isopropyl group, sec-butyl group, tert-butyl group and isoamyl group, a cycloalkyl group (may be monocyclic or polycyclic; preferably having a carbon number of 3 to 20, more preferably from 5 to 8), a hydroxyl group, a halogen atom (fluorine, chlorine, bromine, iodine), a nitro group, a cyano group, an amido group, a sulfonamido group, an alkoxy group, an alkoxyalkyl group, an alkoxycarbonyl group, an alkoxycarbonyloxy group, an acyl group such as formyl group, acetyl group and benzoyl group, an acyloxy group such as acetoxy group and butyryloxy group, and a carboxyl group.

Examples of the alkoxy group include a linear, branched or cyclic alkoxy group having a carbon number of 1 to 20, such as methoxy group, ethoxy group, n-propoxy group, i-propoxy group, n-butoxy group, 2-methylpropoxy group, 1-methylpropoxy group, tert-butoxy group, cyclopentyloxy group and cyclohexyloxy group.

Examples of the alkoxyalkyl group include a linear, branched or cyclic alkoxyalkyl group having a carbon number of 2 to 21, such as methoxymethyl group, ethoxymethyl group, 1-methoxyethyl group, 2-methoxyethyl group, 1-ethoxyethyl group and 2-ethoxyethyl group.

Examples of the alkoxycarbonyl group include a linear, branched or cyclic alkoxycarbonyl group having a carbon number of 2 to 21, such as methoxycarbonyl group, ethoxycarbonyl group, n-propoxycarbonyl group, i-propoxycarbonyl group, n-butoxycarbonyl group, 2-methylpropoxycarbonyl group, 1-methylpropoxycarbonyl group, tert-butoxycarbonyl group, cyclopentyloxycarbonyl group and cyclohexyloxycarbonyl group.

Examples of the alkoxycarbonyloxy group include a linear, branched or cyclic alkoxycarbonyloxy group having a carbon number of 2 to 21, such as methoxycarbonyloxy group, ethoxycarbonyloxy group, n-propoxycarbonyloxy group, i-propoxycarbonyloxy group, n-butoxycarbonyloxy group, tert-butoxycarbonyloxy group, cyclopentyloxycarbonyl group and cyclohexyloxycarbonyl group.

As for the ring structure which may be formed by combining two R₁₅'s with each other, a group capable of forming a 5- or 6-membered ring together with the sulfur atom in formula (ZI-1) is preferred, and a group capable of forming a 5-membered ring (that is, a tetrahydrothiophene ring) is more preferred. Examples of the substituent on the divalent group include a hydroxyl group, a carboxyl group, a cyano group, a nitro group, an alkyl group, a cycloalkyl group, an alkoxy group, an alkoxyalkyl group, an alkoxycarbonyl group and an alkoxycarbonyloxy group. In formula (ZI-1), R₁₅ is preferably, for example, a methyl group, an ethyl group, or a divalent group of combining two R₁₅'s to form a tetrahydrothiophene ring structure together with the sulfur atom.

Each of the alkyl group, cycloalkyl group, alkoxy group and alkoxycarbonyl group of R₁₃ and the alkyl group, cycloalkyl group, alkoxy group, alkylsulfonyl group and cycloalkylsulfonyl group of R₁₄ may be substituted, as described above, and the substituent is preferably a hydroxyl group, an alkoxy group, an alkoxycarbonyl group or a halogen atom (particularly a fluorine atom).

Each of R₁₃ and R₁₄ is preferably a linear or branched alkoxy group, more preferably an n-heptyloxy group, a cyclohexylmethyloxy group, an n-octyloxy group, a cyclohexylethyloxy group, a 2-ethylhexyloxy group, an n-nonyloxy group or an n-decyloxy group, still more preferably an alkoxy group having an alicyclic alkyl group, such as cyclohexylmethyloxy group and cyclohexylethyloxy group.

Each of R₁₃ and R₁₄ is also preferably a group having a cycloalkyl group, and examples thereof include an alkoxy group substituted with a cycloalkyl group, an alkyl group substituted with a cycloalkyl group, and a cycloalkyl group itself. Examples of the cycloalkyl group in these groups are the same as those of the cycloalkyl group as R₁₃ and R₁₄. The group having a cycloalkyl group is preferably a group having a total carbon number of 7 or more, more preferably a group having a total carbon number of 7 to 15. More preferably, R₁₃ is an alkoxy group substituted with a cycloalkyl group and is a group having a total carbon number of 7 or more. These groups may further have a substituent, and examples of the substituent which these groups may have are the same as those in R₁₃, R₁₄ and R₁₅.

Specific preferred examples of the cation structure represented by formula (ZI-1) are illustrated below.

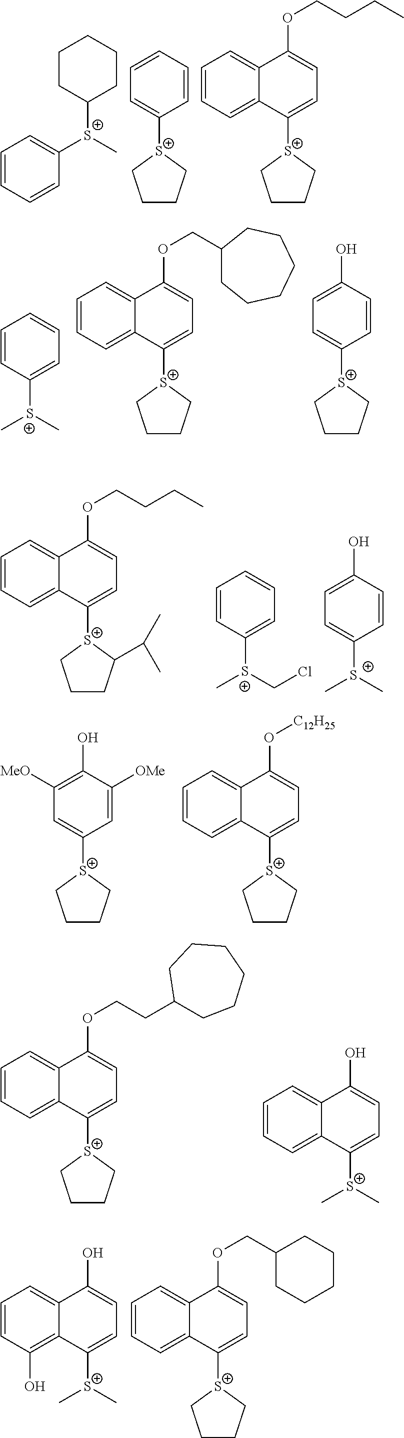

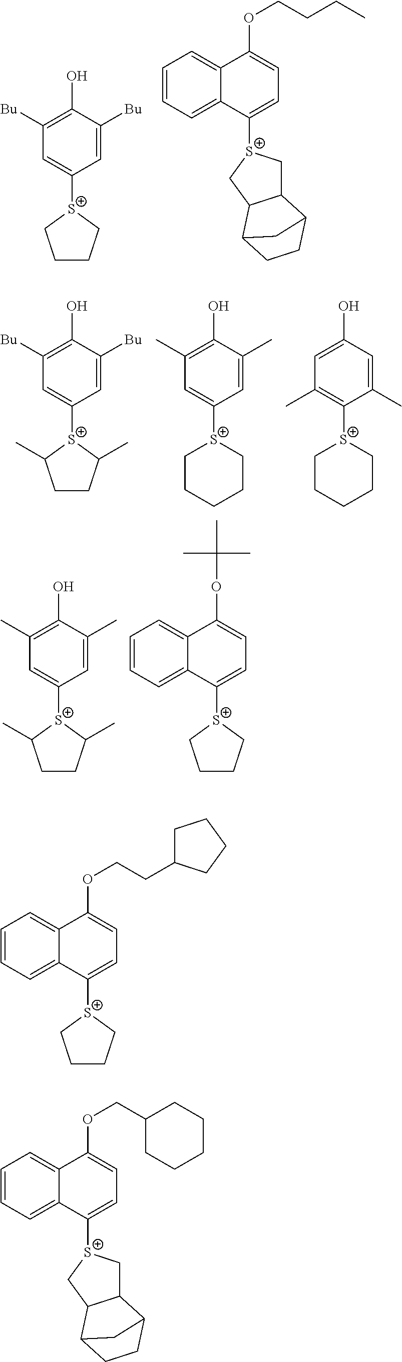

The cation structure (ZI-2) is a structure represented by the following formula (ZI-2):

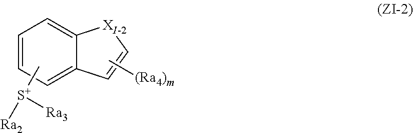

In formula (ZI-2), X_{I-2} represents an oxygen atom, a sulfur atom or an —NRa₁-group, and Ra₁ represents a hydrogen atom, an alkyl group, a cycloalkyl group, an aryl group or an acyl group.

Each of Ra₂ and Ra₃ independently represents an alkyl group, a cycloalkyl group or an alkenyl group, and Ra₂ and Ra₃ may combine with each other to form a ring.

Ra₄ represents, when a plurality of Ra₄'s are present, each independently represents, an organic group.

m represents an integer of 0 to 3.

Each of the group represented by —S⁺(Ra₂)(Ra₃) and m Ra₄'s may be substituted on an arbitrary position of the carbon atom constituting the X_{I-2}-containing 5-membered ring and the 6-membered ring in formula (ZI-2).

The alkyl group of Ra₁ to Ra₃ is preferably a linear or branched alkyl group having a carbon number of 1 to 20, and examples thereof include a methyl group, an ethyl group, a propyl group, an isopropyl group, an n-butyl group, an isobutyl group, a sec-butyl group, a pentyl group, a neopentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group and an eicosyl group.

The cycloalkyl group of Ra₁ to Ra₃ is preferably a cycloalkyl group having a carbon number of 3 to 20, and examples thereof include a cyclopropyl group, a cyclobutyl group, a cyclopentyl group, a cyclohexyl group, a cyclooctyl group, an adamantyl group, a norbornyl group, an isoboronyl group, a camphanyl group, a dicyclopentyl group, an α-pinel group, a tricyclodecanyl group, a tetracyclododecyl group and an androstanyl group.

The aryl group of Ra₁ to Ra₃ is preferably an aryl group having a carbon number of 6 to 10, and examples thereof include a phenyl group and a naphthyl group.

The acyl group of Ra₁ is preferably an acyl group having a carbon number of 2 to 20, and examples thereof include a formyl group, an acetyl group, a propanoyl group, a butanoyl group, a pivaloyl group and a benzoyl group.

The alkenyl group of Ra₂ and Ra₃ is preferably an alkenyl group having a carbon number of 2 to 15, and examples thereof include a vinyl group, an allyl group, a butenyl group and a cyclohexenyl group.

As for the ring structure which may be formed by combining Ra₂ and Ra₃ with each other, a group capable of forming a 5- or 6-membered ring together with the sulfur atom in formula (ZI-2) is preferred, and a group capable of forming a 5-membered ring (that is, a tetrahydrothiophene ring) is more preferred. The ring structure may contain an oxygen atom, and specific examples of the ring structure are the same as those of the ring which may be formed by combining R₁₅'s with each other in formula (ZI-1).

Examples of the organic group of Ra₄ include an alkyl group (preferably having a carbon number of 1 to 20), a cycloalkyl group (preferably having a carbon number of 3 to 20), an aryl group (preferably having a carbon number of 6 to 10), an alkoxy group (preferably having a carbon number of 1 to 20), an acyl group (preferably having a carbon number of 2 to 20), an acyloxy group (preferably having a carbon number of 2 to 20), a fluorine atom, a chlorine atom, a bromine atom, an iodine atom, a hydroxyl group, a carboxyl group, a nitro group, a cyano group, an alkoxycarbonyl group, an alkylsulfonyl group, an arylsulfonyl group, an arylcarbonyl group, an alkylcarbonyl group and an alkenylcarbonyl group.

Ra₁ is preferably an alkyl group, more preferably an alkyl group having a carbon number of 1 to 4.

More preferably, Ra₂ and Ra₃ are combined with each other to constitute a 5- or 6-membered ring.

Each of the groups for Ra₁ to Ra₄ may further have a substituent, and examples of the further substituent which each group may have are the same as those of the further substituent which each of the groups for R₁₃ to R₁₅ in formula (ZI-1) may have.

Specific preferred examples of the cation in the compound represented by formula (ZI-2) are illustrated below.

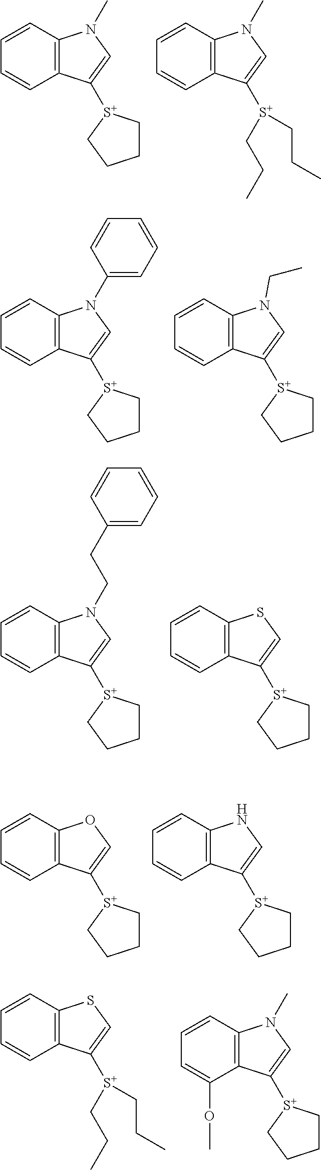

The cation structure (ZI-3) is a structure represented by the following formula (ZI-3):

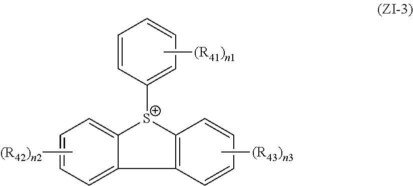

In formula (ZI-3), each of R₄₁ to R₄₃ independently represents an alkyl group, an alkoxy group or a hydroxy group.

In R₄₁ to R₄₃, the alkyl group is preferably a lower alkyl group having a carbon number of 1 to 5, more preferably a linear or branched alkyl group, still more preferably a methyl group, an ethyl group, a propyl group, an n-butyl group or a tert-butyl group.

The alkoxy group is preferably an alkoxy group having a carbon number of 1 to 5, more preferably a linear or branched alkoxy group, still more preferably a methoxy group or an ethoxy group.

Each of n1 to n3 is independently an integer of 0 to 2, preferably 0 or 1, and more preferably, all are 0.

Incidentally, when each of n1 to n3 is 2, each R₄₁, R₄₂ or R₄₃ may be the same as or different from every other R₄₁, R₄₂ or R₄₃.

Each of the groups in R₄₁ to R₄₃ may further have a substituent, and examples of the further substituent which each group may have are the same as those of the further substituent which each of the groups for R₁₃ to R₁₅ in formula (ZI-1) may have.

Specific preferred examples of the cation structure in the compound represented by formula (ZI-3) are illustrated below.

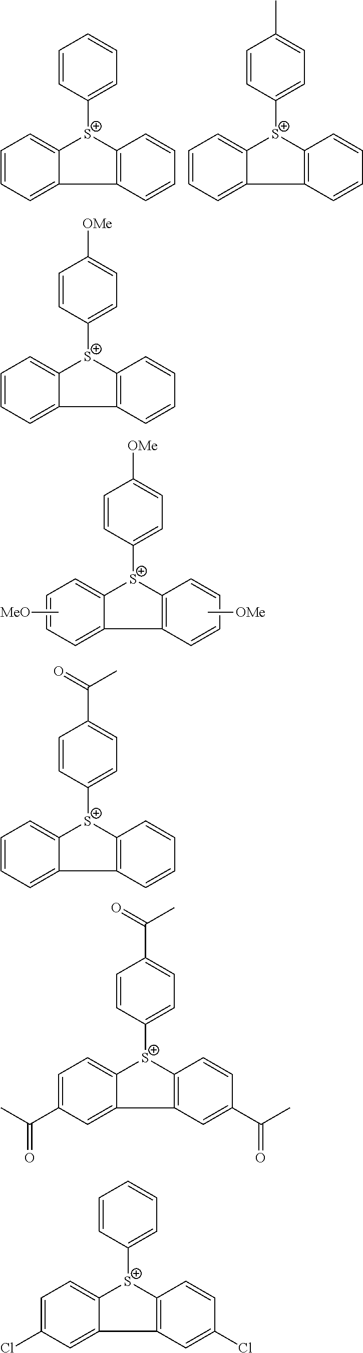

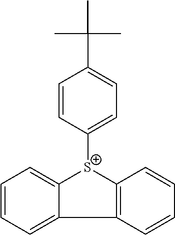

The cation structure (ZI-4) is a structure represented by the following formula (ZI-4):

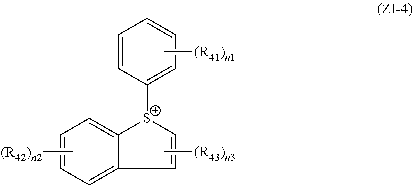

In formula (ZI-4), each of R₄₁ to R₄₃ is independently an alkyl group, an acetyl group, an alkoxy group, a carboxy group, a hydroxyl group or a hydroxyalkyl group.

The alkyl group and alkoxy group as R₄₁ to R₄₃ are the same as those of R₄₁ to R₄₃ in formula (ZI-3).

The hydroxyalkyl group is preferably a group formed by replacing one hydrogen atom or a plurality of hydrogen atoms in the alkyl group above with a hydroxy group, and examples thereof include a hydroxymethyl group, a hydroxyethyl group and a hydroxypropyl group.

n1 is an integer of 0 to 3, preferably 1 or 2, more preferably 1.

n2 is an integer of 0 to 3, preferably 0 or 1, more preferably 0.

n3 is an integer of 0 to 2, preferably 0 or 1, more preferably 1.

However, it is not allowed that n1, n2 and n3 are 0 at the same time.

Each of the groups in R₄₁ to R₄₃ may further have a substituent, and examples of the further substituent which each group may have are the same as those of the further substituent which each of the groups for R₁₃ to R₁₅ in formula (ZI-1) may have.

Specific preferred examples of the cation in the compound represented by formula (ZI-4) are illustrated below.

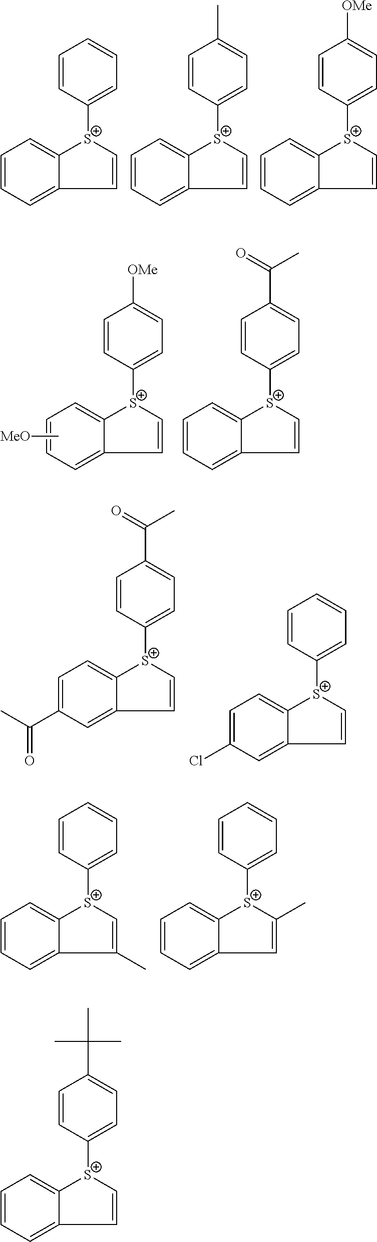

The cation structure (ZI-5) is a structure represented by the following formula (ZI-5):

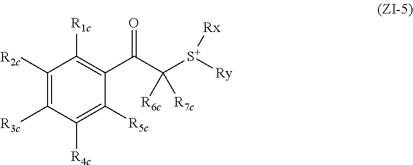

In formula (ZI-5), each of R_{1c} to R_{5c} independently represents a hydrogen atom, an alkyl group, a cycloalkyl group, an alkoxy group, an aryloxy group, an alkylthio group, an arylthio group or a halogen atom. Any two or more members out of R_{1c} to R_{5c} may combine to form a ring structure.

Each of R_{6c} and R_{7c} independently represents a hydrogen atom, an alkyl group or a cycloalkyl group. R_{6c} and R_{7c} may combine to form a ring structure.

Each of Rₓ and R_{y} independently represents an alkyl group, a cycloalkyl group, an allyl group or a vinyl group. Rₓ and R_{y} may combine with each other to form a ring.

Any two or more members out of R_{1c} to R_{5c}, a pair of R_{6c} and R_{7c}, and a pair of Rₓ and R_{y} may combine to form a ring structure, respectively. This ring structure may contain an oxygen atom, a sulfur atom, an ester bond or an amido bond. Examples of the group formed by combining any two or more members out of R_{1c} to R_{5c}, a pair of R_{6c} and R_{7c}, and a pair of Rₓ and R_{y} include a butylene group and a pentylene group.

The alkyl group as R_{1c} to R_{7c} may be either linear or branched and is, for example, an alkyl group having a carbon number of 1 to 20, preferably a linear or branched alkyl group having a carbon number of 1 to 12 (e.g., methyl, ethyl, linear or branched propyl, linear or branched butyl, linear or branched pentyl). The cycloalkyl group is, for example, a cycloalkyl group having a carbon number of 3 to 8 (e.g., cyclopentyl, cyclohexyl).

In the case where R_{6c} and R_{7c} are combined to form a ring, the group formed by combining R_{6c} and R_{7c} is preferably an alkylene group having a carbon number of 2 to 10, and examples thereof include an ethylene group, a propylene group, a butylene group, a pentylene group and a hexylene group. Also, the ring formed by combining R_{6c} and R_{7c} may contain a heteroatom such as oxygen atom in the ring.

The alkoxy group as R_{1c} to R_{5c} may be linear, branched or cyclic and is, for example, an alkoxy group having a carbon number of 1 to 10, preferably a linear or branched alkoxy group having a carbon number of 1 to 5 (e.g., methoxy, ethoxy, linear or branched propoxy, linear or branched butoxy, linear or branched pentoxy), or a cyclic alkoxy group having a carbon number of 3 to 8 (e.g., cyclopentyloxy, cyclohexyloxy).

The aryloxy group as R_{1c} to R_{5c} is, for example, an aryloxy group having a carbon number of 6 to 14, preferably an aryloxy group having a carbon number of 6 to 10 (e.g., phenoxy, naphthalenoxy).

The alkylthio group as R_{1c} to R_{5c} may be linear, branched or cyclic and is, for example, an alkylthio group having a carbon number of 1 to 10, preferably a linear or branched alkylthio group having a carbon number of 1 to 5 (e.g., methylthio, ethylthio, linear or branched propylthio, linear or branched butylthio, linear or branched pentylthio), or a cyclic alkylthio group having a carbon number of 3 to 8 (e.g., cyclopentylthio, cyclohexylthio).

The arylthio group as R_{1c} to R_{5c} is, for example, an arylthio group having a carbon number of 6 to 14, preferably an arylthio group having a carbon number of 6 to 10 (e.g., phenylthio, naphthalenethio).

A compound where any one of R_{1c} to R_{5c} is a linear or branched alkyl group, a cycloalkyl group or a linear, branched or cyclic alkoxy group is preferred, and a compound where the sum of carbon numbers of R_{1c} to R_{5c} is from 2 to 15 is more preferred. Thanks to such a compound, the solvent solubility is more enhanced and production of particles during storage can be suppressed.

Examples of the alkyl group and cycloalkyl group as Rₓ and R_{y} are the same as those of the alkyl group and cycloalkyl group in R_{1c} to R_{7c}. Among these, a 2-oxoalkyl group, a 2-oxocycloalkyl group and an alkoxycarbonylmethyl group are preferred.

Examples of the 2-oxoalkyl group and 2-oxocycloalkyl group include a group having C═O at the 2-position of the alkyl group or cycloalkyl group as R_{1c} to R_{7c}.

The allyl group is not particularly limited but is preferably an unsubstituted allyl group or an allyl group substituted with a monocyclic or polycyclic cycloalkyl group.

The vinyl group is not particularly limited but is preferably an unsubstituted vinyl group or a vinyl group substituted with a monocyclic or polycyclic cycloalkyl group.

Examples of the alkoxy group in the alkoxycarbonylalkyl group are the same as those of the alkoxy group in R_{1c} to R_{5c}.

The ring which may be formed by combining Rₓ and R_{y} is a ring containing —S⁺ in formula (ZI-5) and is preferably a ring having a carbon number of 3 to 10, more preferably a ring having a carbon number of 4 to 6. Specifically, the ring structure represented by formula (IV-1) exemplified as Y₂ of formula (II) is preferred. As for the ring, a group capable of forming a 5- or 6-membered ring together with the sulfur atom in formula (ZI-5) is preferred, and a group capable of forming a 5-membered ring (that is, a tetrahydrothiophene ring) is more preferred. The ring may contain an oxygen atom. Specific examples of the ring are the same as those of the ring which may be formed by combining R₁₅'s with each other in formula (ZI-1).

Each of the groups for R_{1c} to R_{7c}, Rₓ and R_{y} may further have a substituent, and examples of the further substituent which each group may have are the same as those of the further substituent which each of the groups for R₁₃ to R₁₅ in formula (ZI-1) may have.

Specific preferred examples of the cation in the compound represented by formula (ZI-5) are illustrated below.

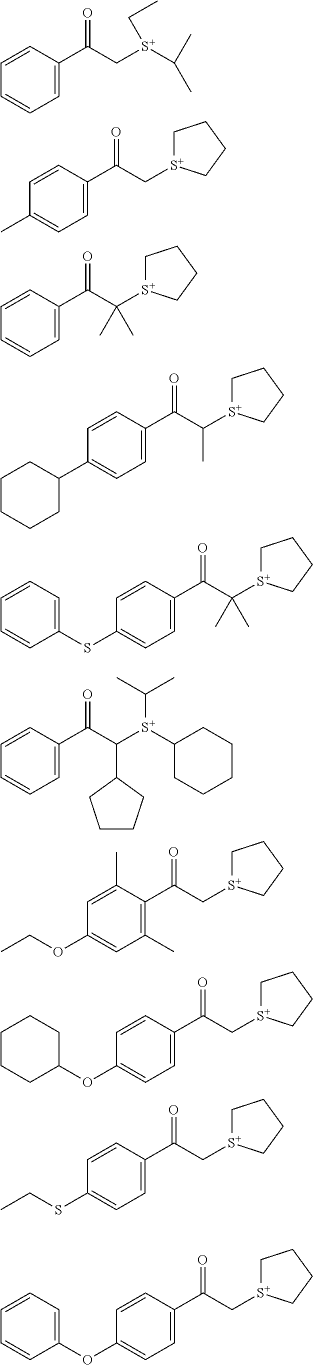

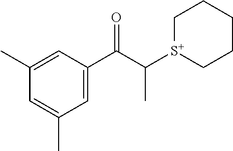

Among the cation structures represented by formulae (ZI-1) to (ZI-5), structures (ZI-1), (ZI-2) and (ZI-5) are preferred, and (ZI-1) and (ZI-5) are more preferred.

The molar extinction coefficient ε of the compound (PA) at a wavelength of 193 nm as measured in an acetonitrile solvent is 55,000 or less, and its relative ratio to triphenylsulfonium nonafluorobutanesulfonate is preferably 0.8 or less, more preferably from 0.1 to 0.8, more preferably from 0.2 to 0.6, still more preferably from 0.3 to 0.5.

With respect to representative specific examples of the compound having a cation structure represented by formulae (ZI-1) to (ZI-5), the molar extinction coefficient ε at a wavelength of 193 nm, in terms of the relative ratio to triphenylsulfonium nonafluorobutanesulfonate, are shown below. The molar extinction coefficient ε is calculated in accordance with the Lambert-Beer law, where the UV spectrum of a measurement solution prepared by dissolving the compound (PA) in an acetonitrile solvent is measured using a 1 cm-square cell and the molar extinction coefficient is calculated from the absorbance (A) at 193 nm and the concentration (c) of the measurement solvent. Incidentally, in order to compare the characteristics of cation structures, the anion structure is uniformly configured by nonafluorobutanesulfonate.

TABLE 1

| | |
|---|---|
| 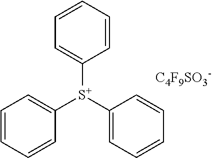 | 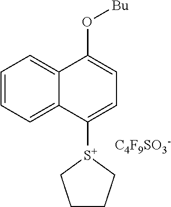 |
| Relative molar extinction | Relative molar extinction |
| coefficient: 1.00 | coefficient: 0.38 |
| 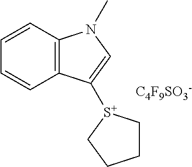 | 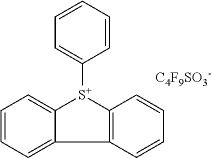 |
| Relative molar extinction | Relative molar extinction |
| coefficient: 0.13 | coefficient: 0.65 |
| 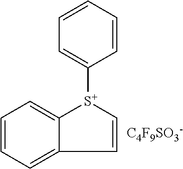 | 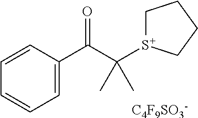 |
| Relative molar extinction | Relative molar extinction |
| coefficient: 0.70 | coefficient: 0.23 |
| 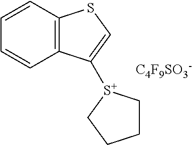 | 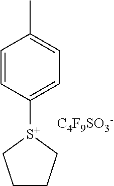 |
| Relative molar extinction | Relative molar extinction |
| coefficient: 0.21 | coefficient: 0.30 |

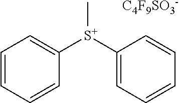

Relative molar extinction
coefficient: 0.74

Specific examples of the compound (PA) for use in the present invention are illustrated below, but the present invention is not limited thereto.

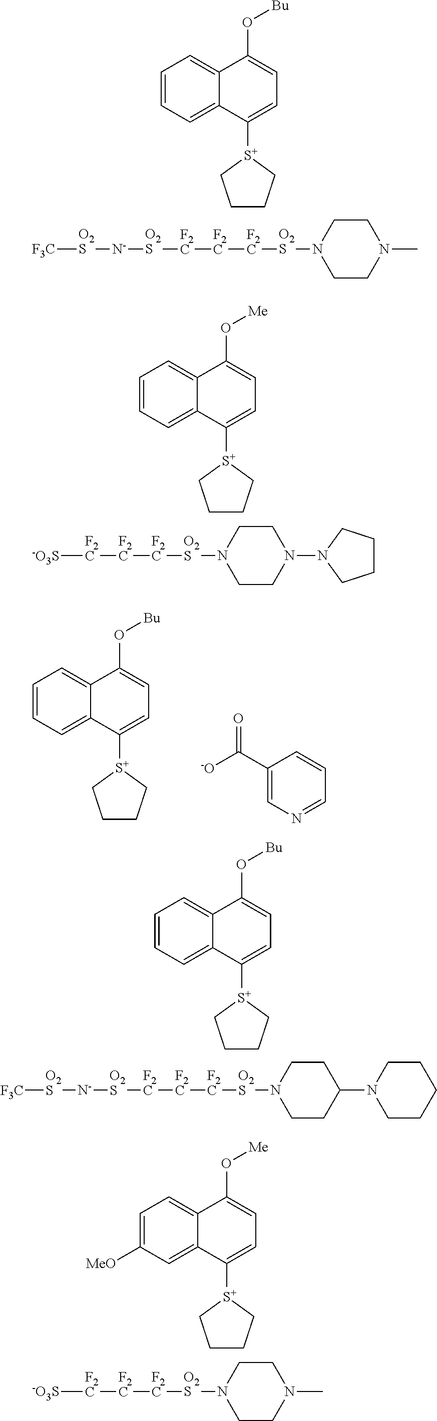

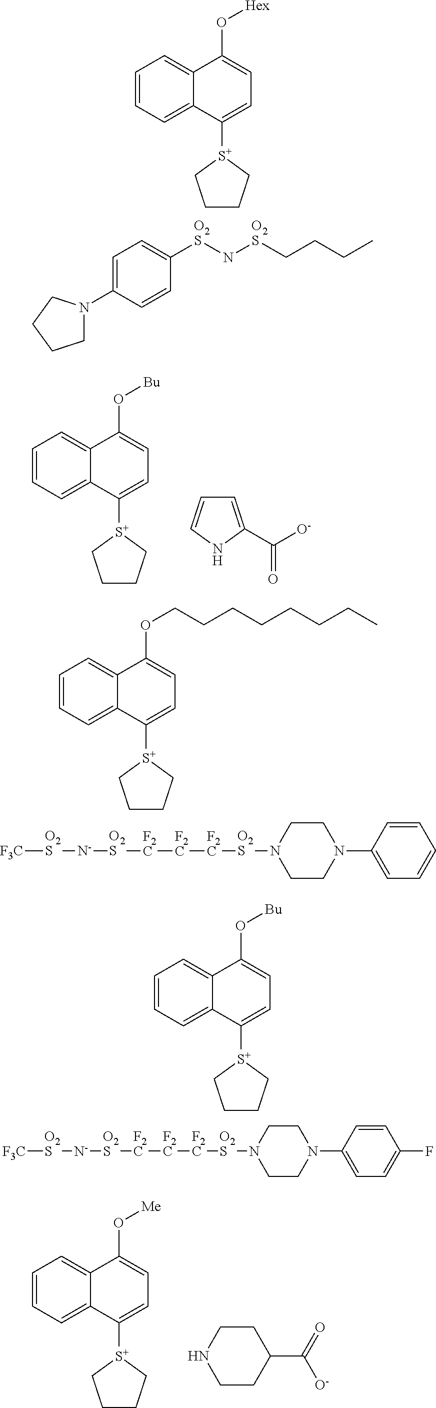

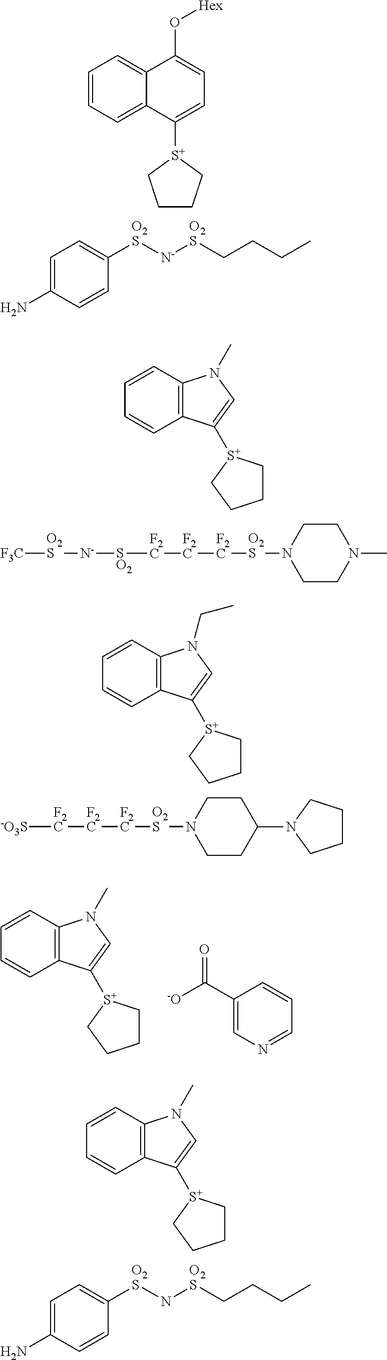

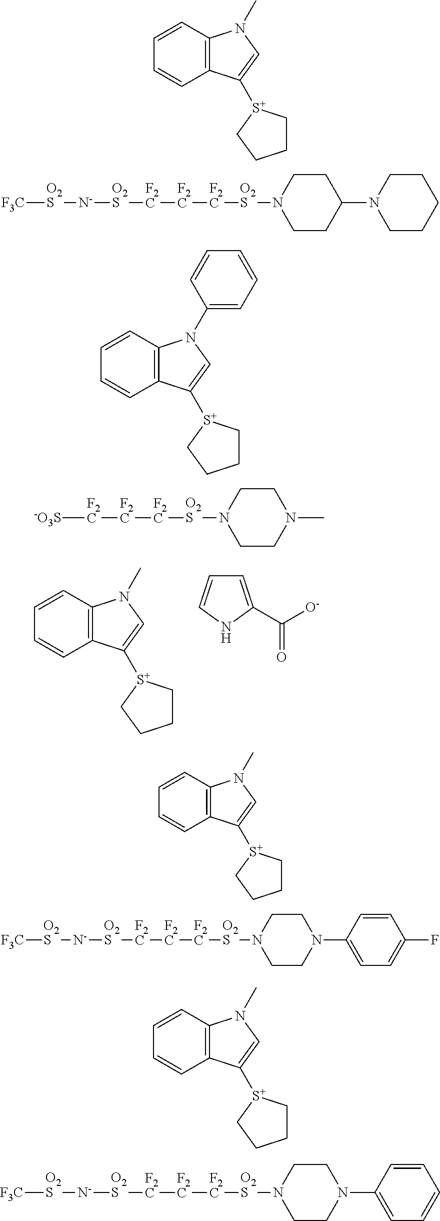

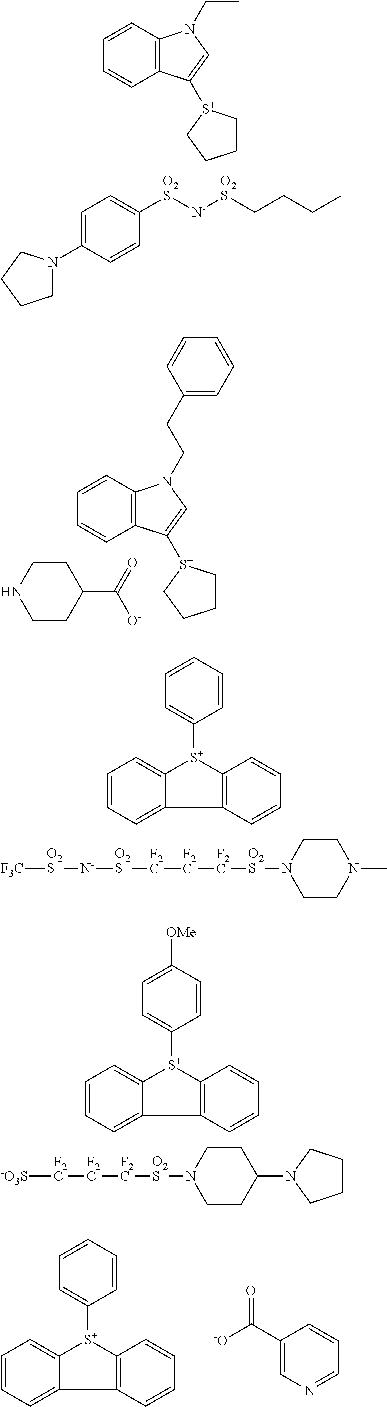

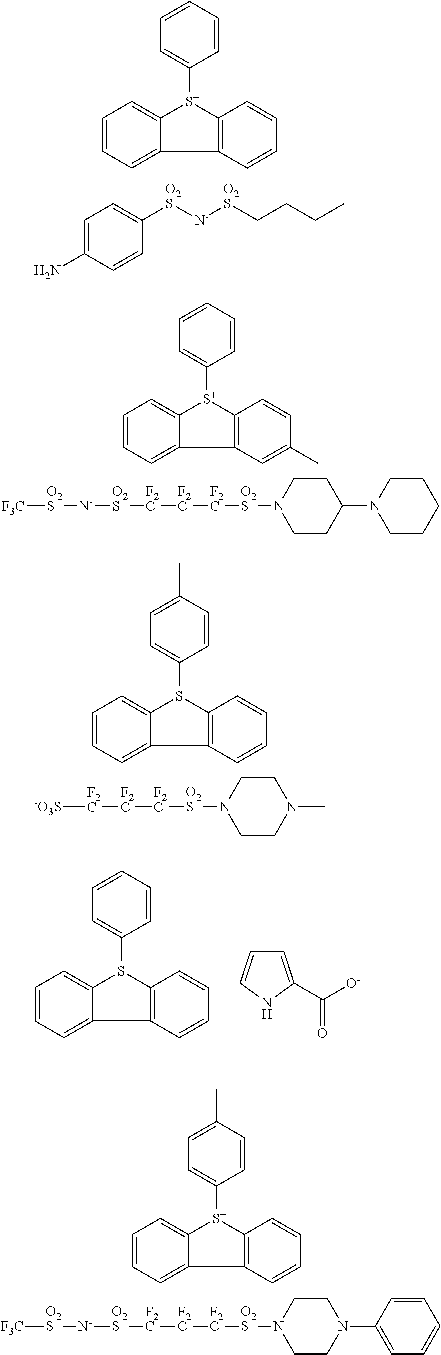

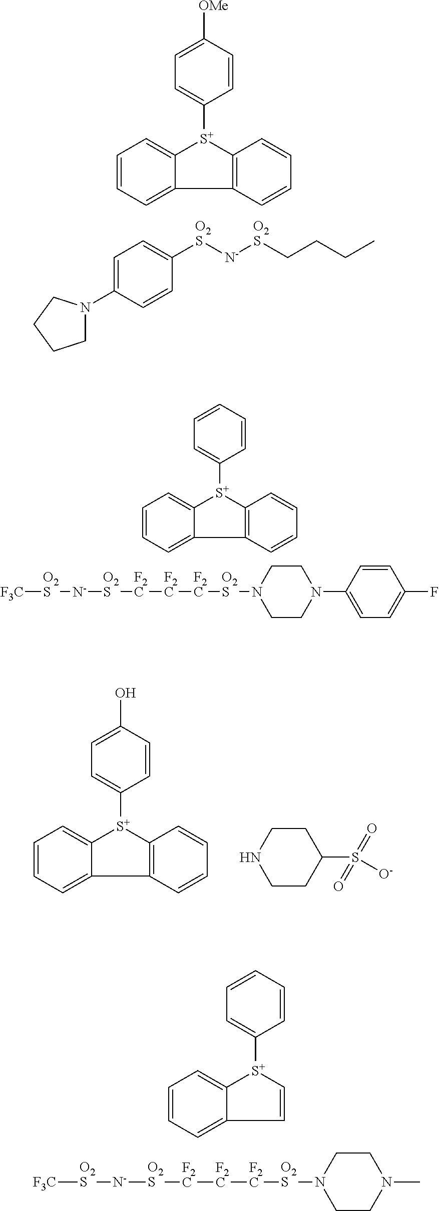

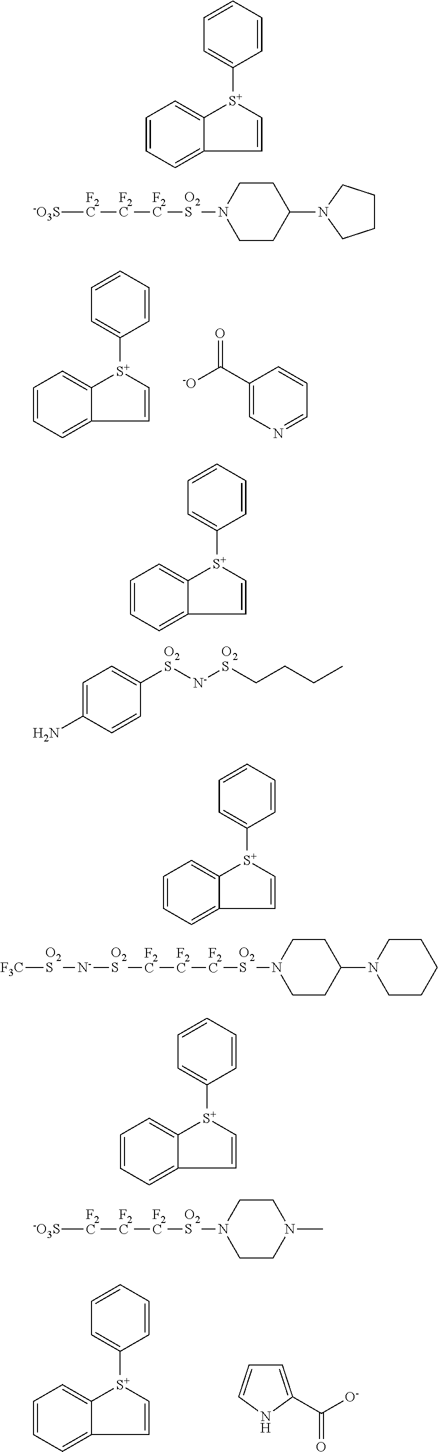

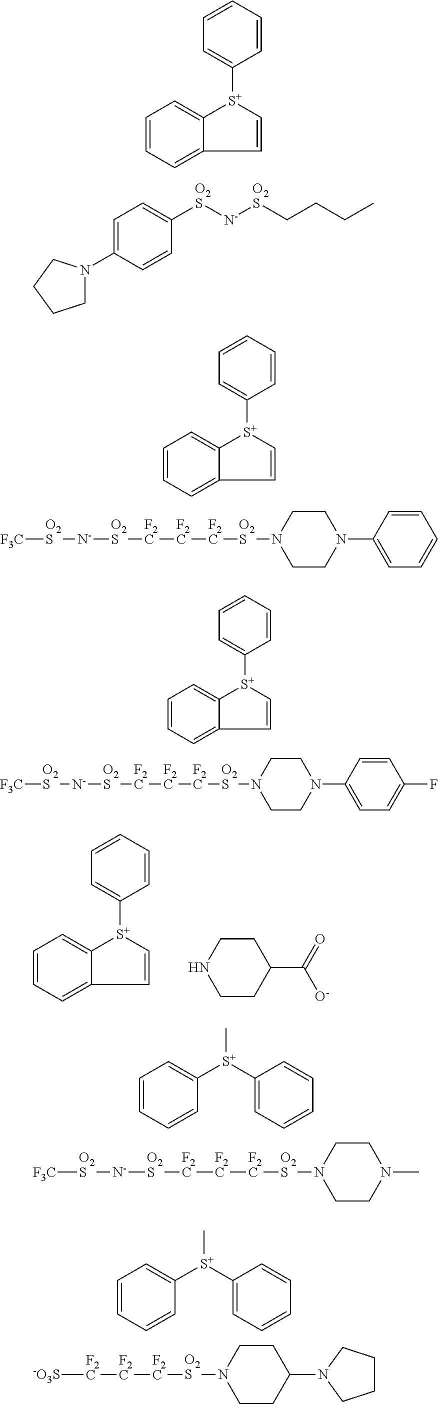

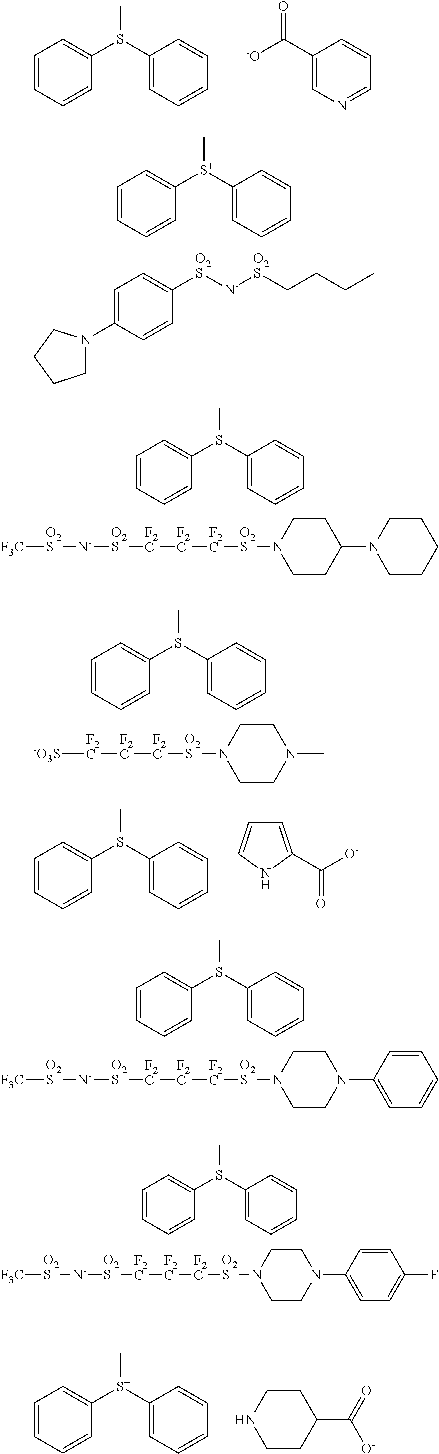

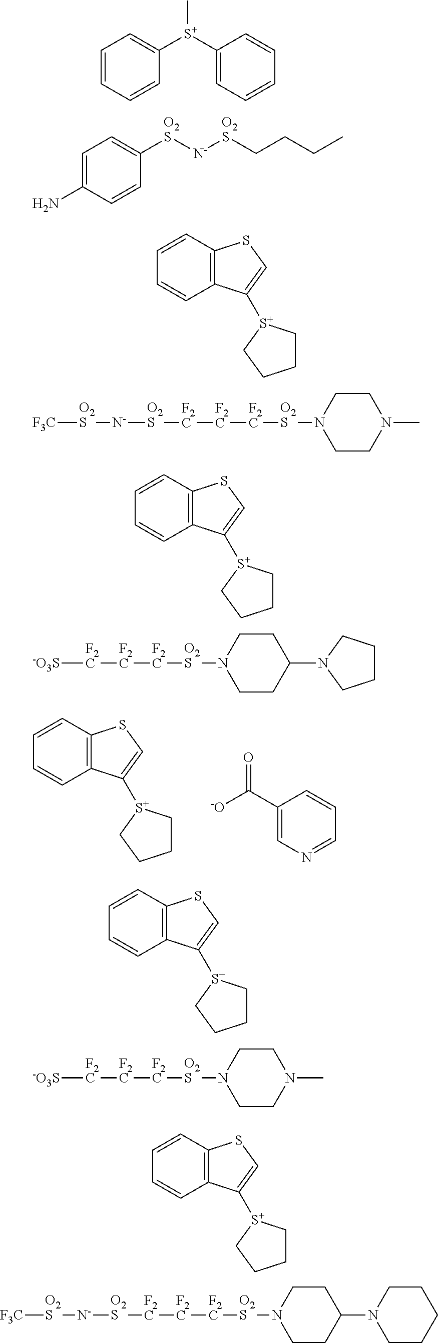

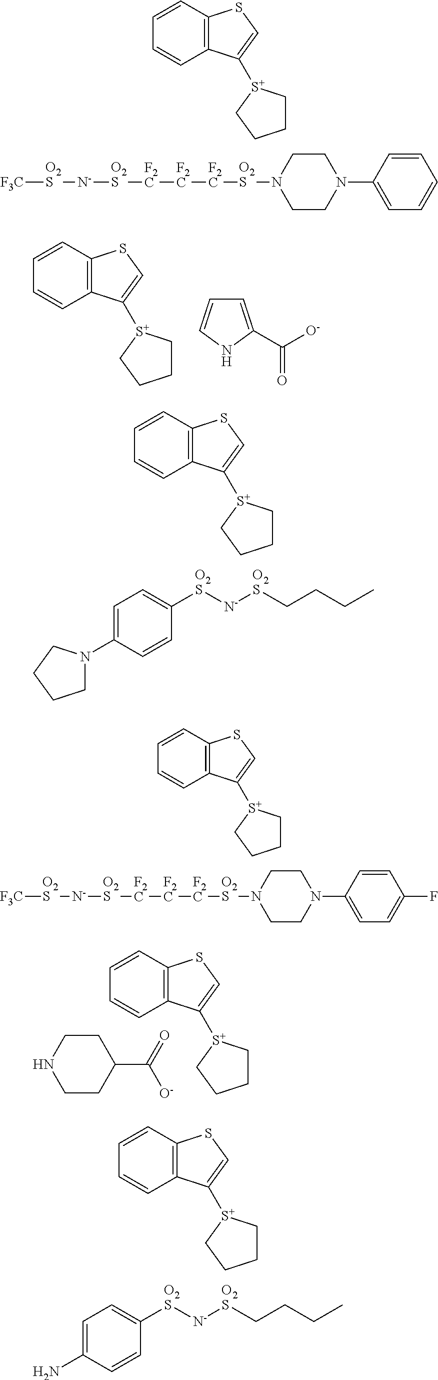

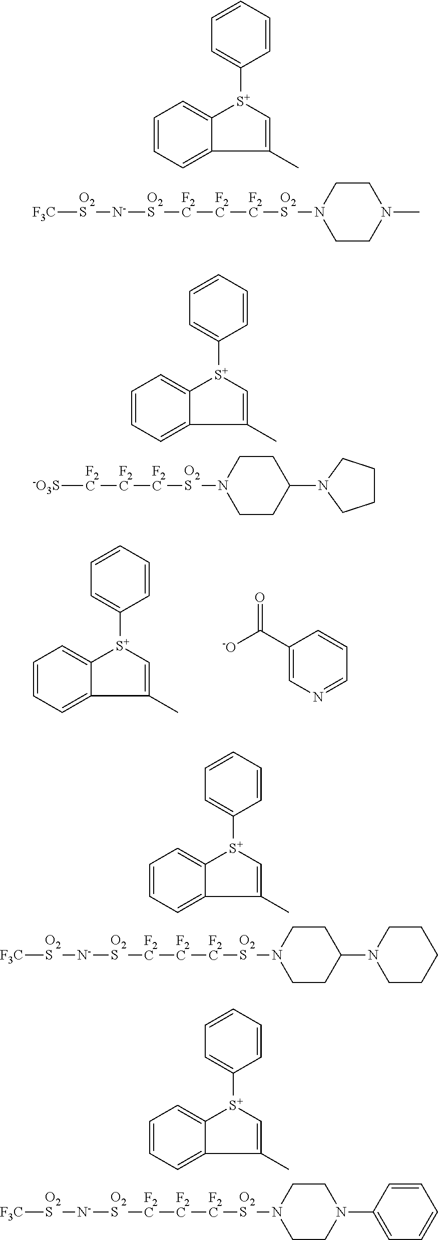

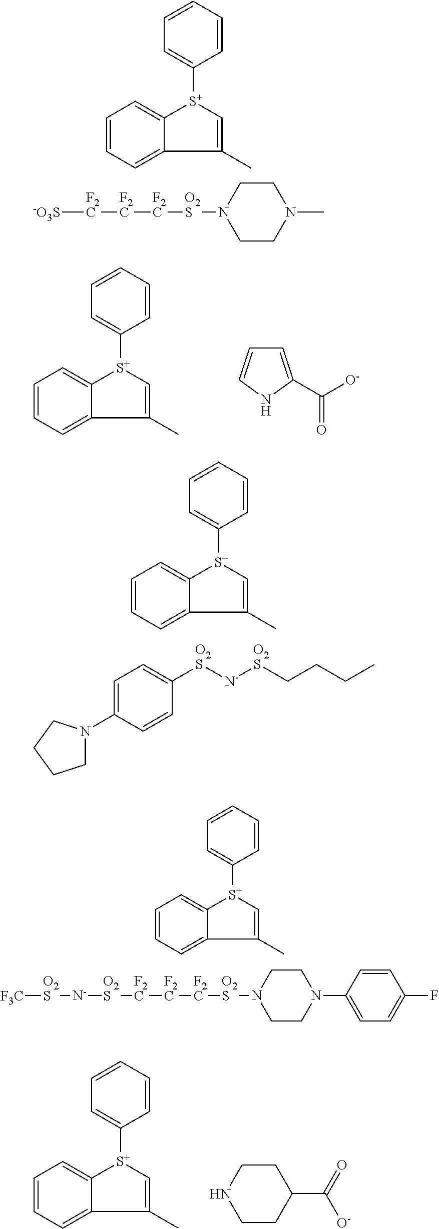

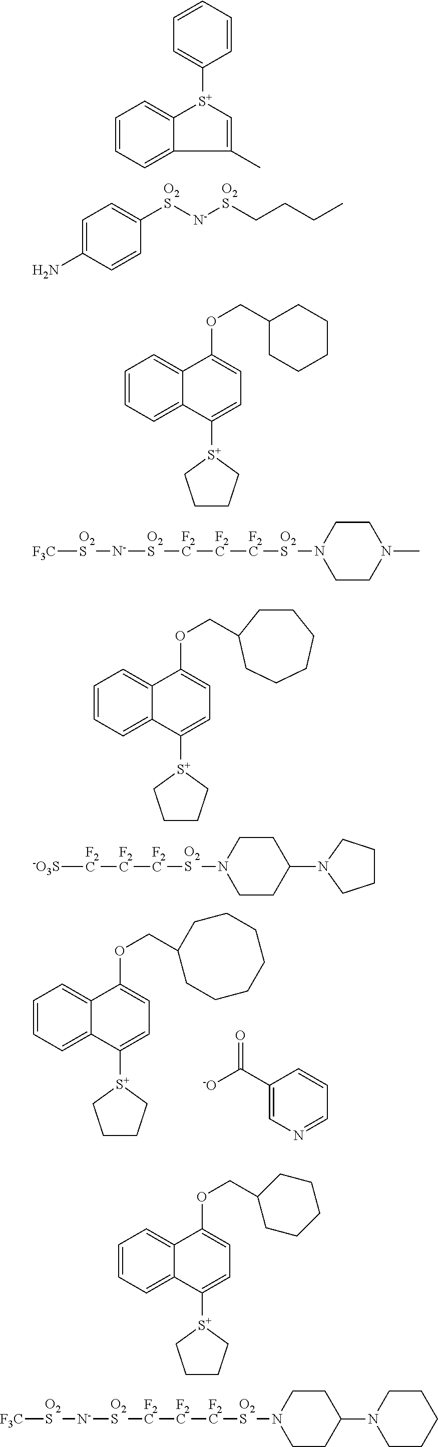

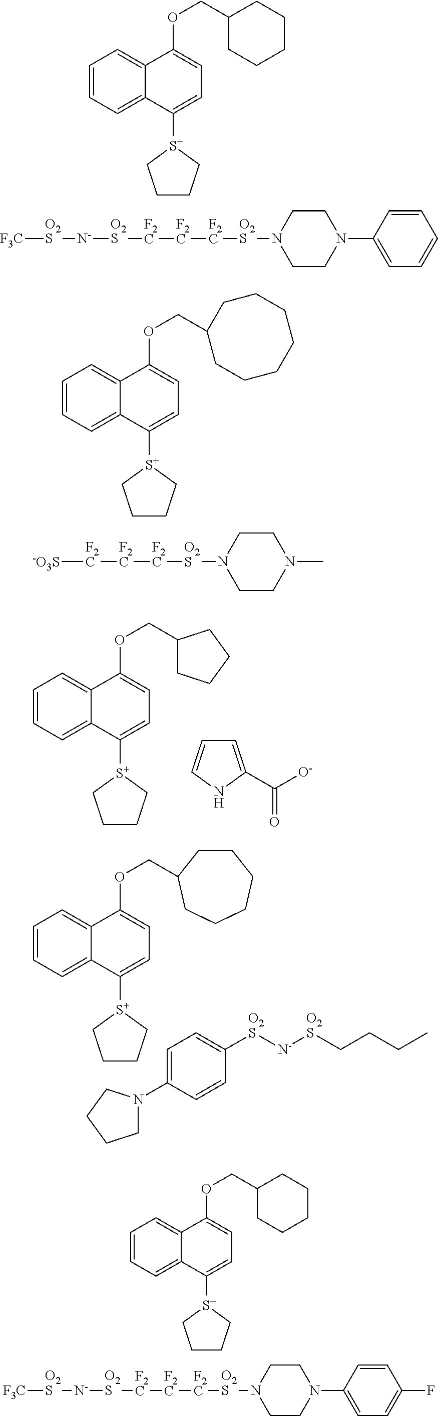

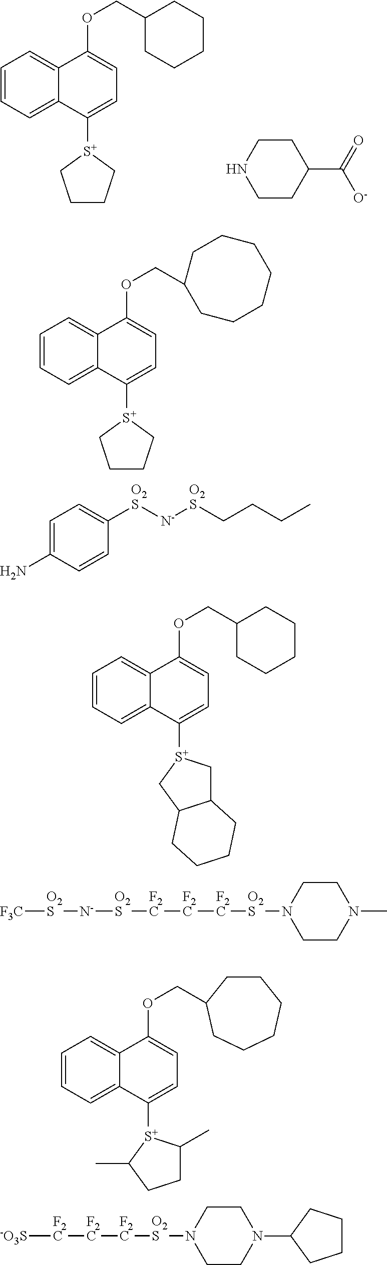

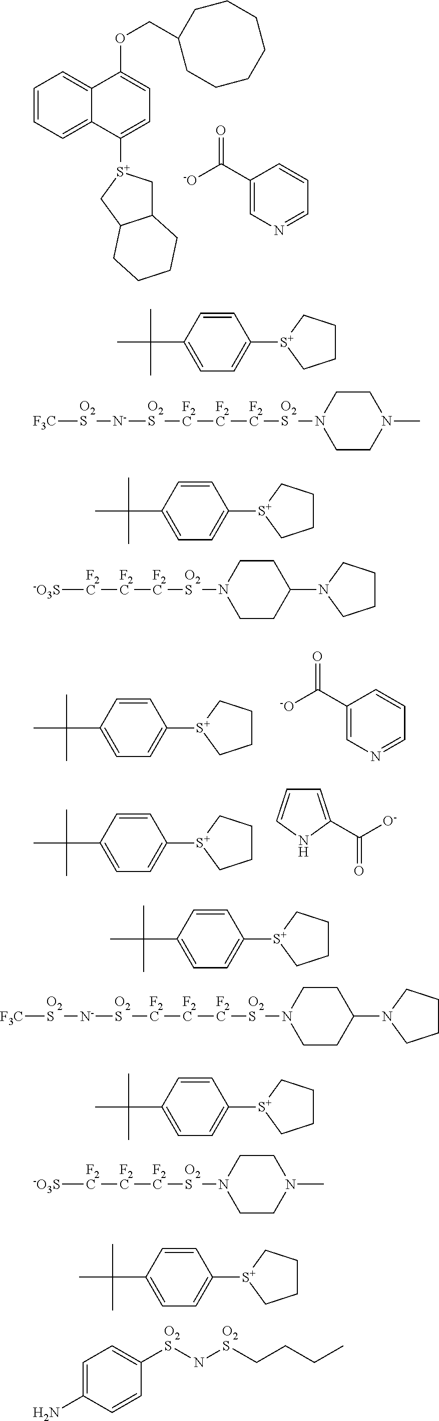

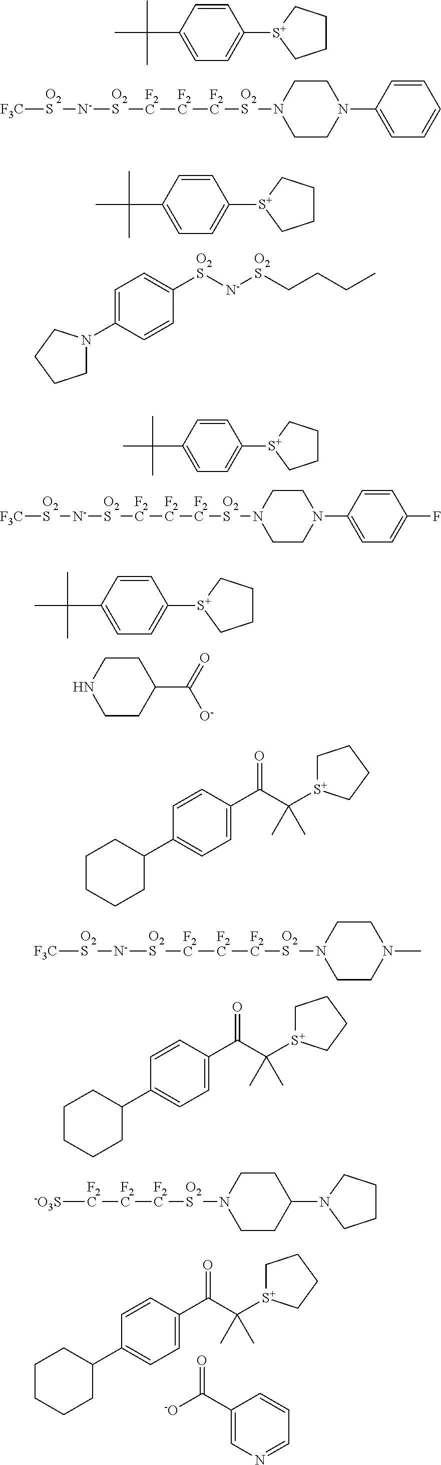

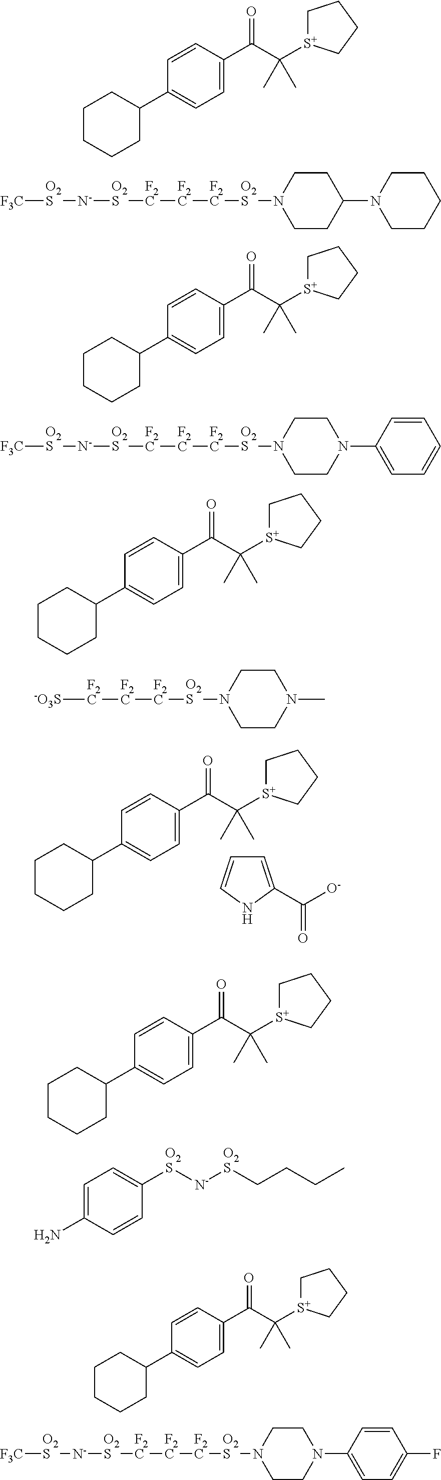

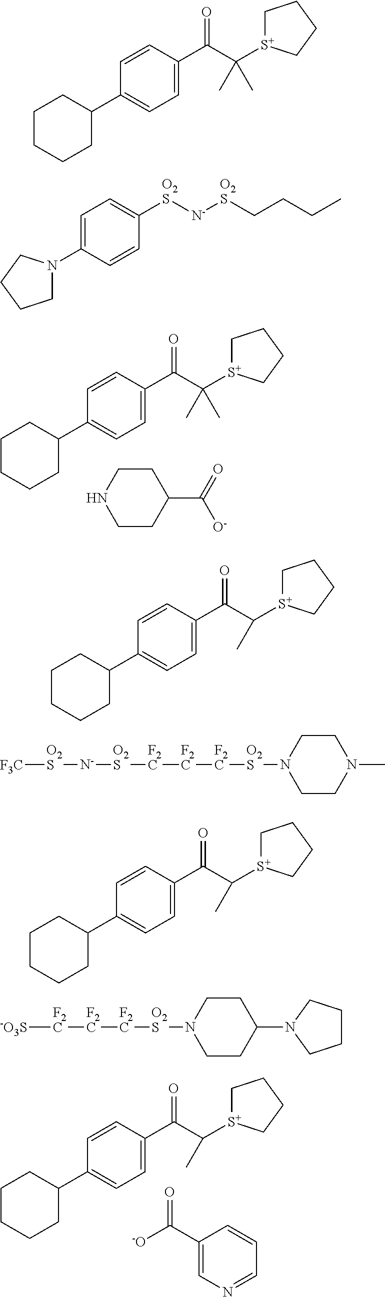

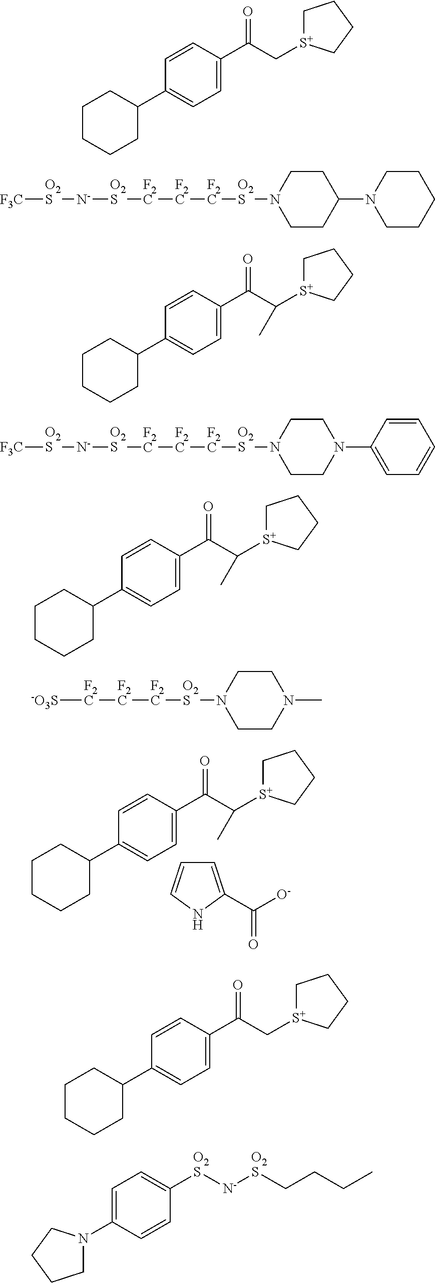

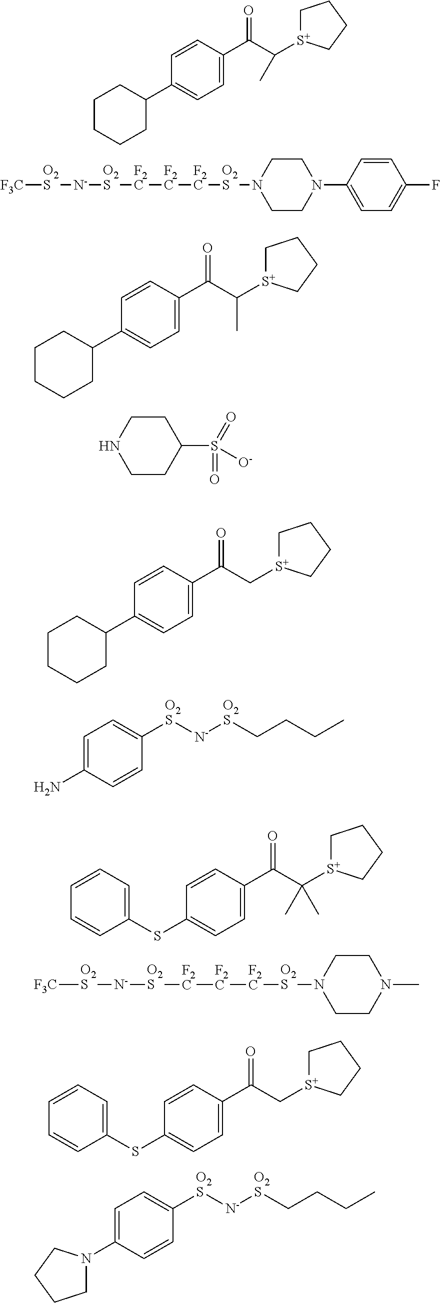

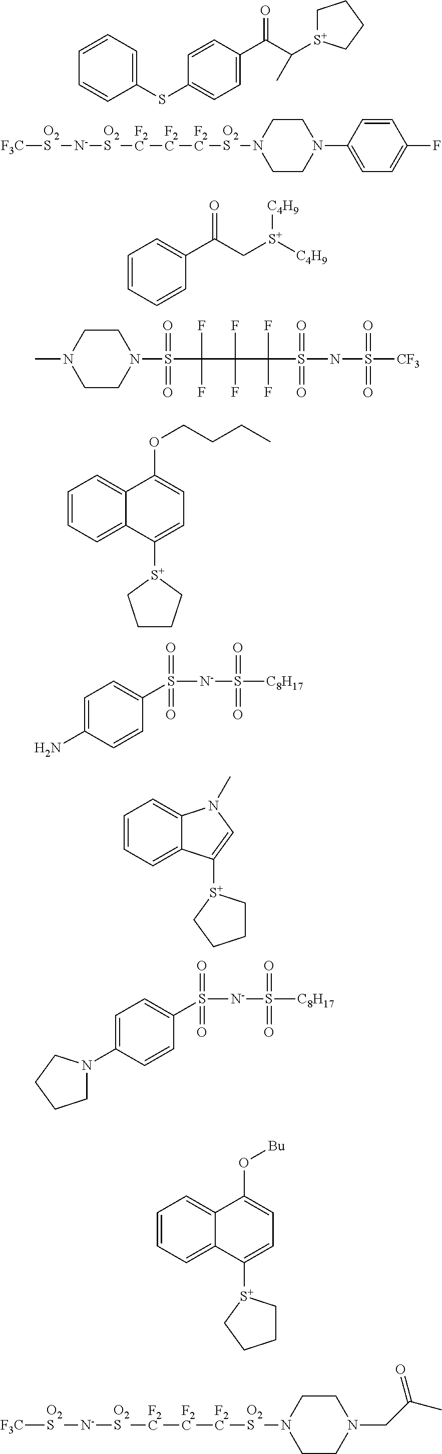

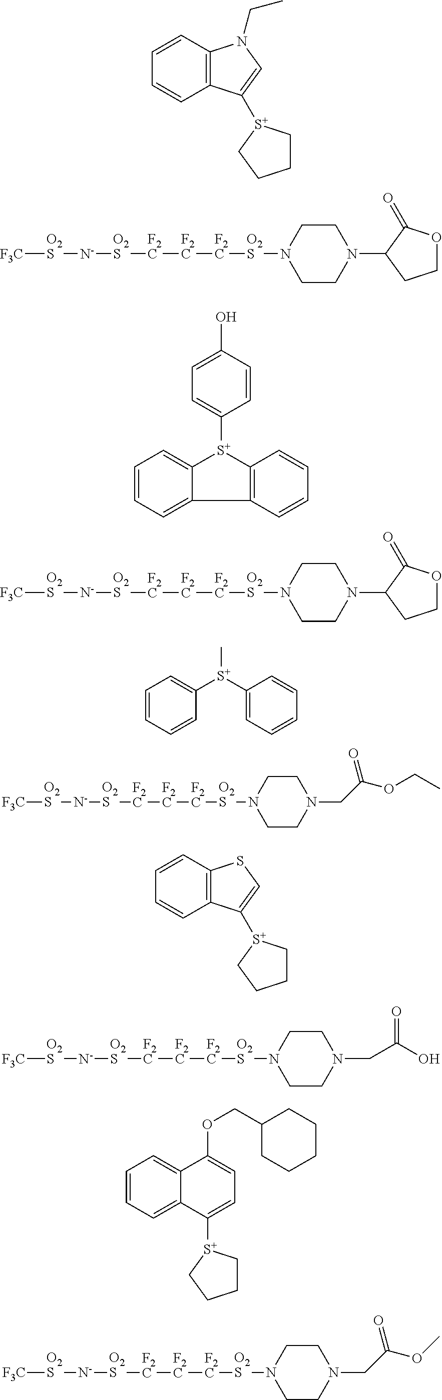

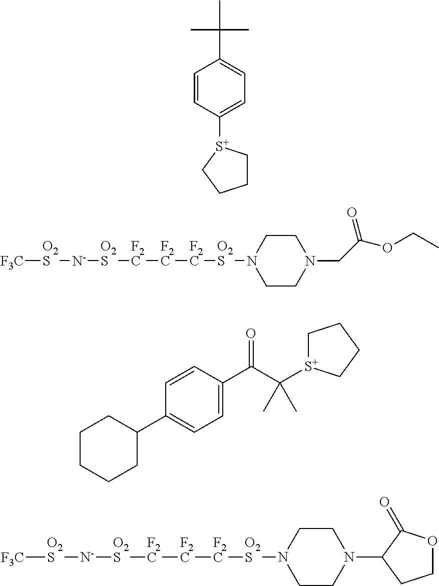

In the present invention, other than the compound capable of generating a compound represented by formula (PA-1), a compound having a proton acceptor functional group and undergoing decomposition upon irradiation with an actinic ray or radiation to generate a compound reduced in or deprived of the proton acceptor property or changed to be acidic from being proton acceptor-functioning (sometimes referred to as a proton acceptor compound or a compound (PA′)) can be appropriately selected. As the compound (PA′), for example, a compound that is an ionic compound and has a proton acceptor site in the cation moiety may used. More specifically, examples thereof include a compound represented by the following formula (7):

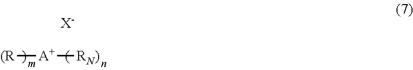

wherein A represents a sulfur atom or an iodine atom,

m represents 1 or 2, n represents 1 or 2, provided that when A is a sulfur atom, m+n=3 and when A is an iodine atom, m+n=2,

R represents an aryl group,

R_{N} represents an aryl group substituted with a proton acceptor functional group, and

X⁻ represents a counter anion.

Specific examples of X⁻ are the same as those of Z⁻ in formula (ZI) that is an acid generator described later.

Specific preferred examples of the aryl group of R and R_{N} include a phenyl group.

Specific examples of the proton acceptor functional group contained in R_{N} are the same as those of the proton acceptor functional group described above in formula (PA-1).

In the composition of the present invention, the content of the compound (PA) in the entire composition is preferably from 0.1 to 10 mass %, more preferably from 1 to 8 mass %, based on the entire solid content of the composition, (In this specification, mass ratio is equal to weight ratio.)

In the case where the composition of the present invention contains the compound (PA′), the content thereof is, in terms of the weight ratio of compound (PA)/compound (PA′), preferably from 9/1 to 5/5, more preferably from 8/2 to 6/4.

As for the compounds (PA) and (PA′), a commercial product may be used, or the compound may be synthesized by a known method.

The components suitably used in the actinic ray-sensitive or radiation-sensitive resin composition of the present invention, except for the compounds (PA) and (PA′), are described below.

As described above, the compound (PA) decomposes upon exposure to an actinic ray or radiation to generate an acid in the exposed area, but this compound is characterized in that the proton acceptor functional group present within the molecule traps the proton of the acid and the compound is thereby neutralized. That is, an acid is substantially not generated from the compound (PA) even in the exposed area (the compound does not contribute to the chemical amplification action). Accordingly, in view of sensitivity, the actinic ray-sensitive or radiation-sensitive resin composition of the present invention is preferably a chemical amplification resist composition containing (C) an acid generator. Also, the composition is preferably a positive resist composition containing (B1) a resin capable of increasing the solubility in an alkali developer by the action of an acid.

[2] (B1) Resin capable of increasing the solubility in an alkali developer by the action of an acid

The actinic ray-sensitive or radiation-sensitive resin composition of the present invention contains (B1) a resin capable of increasing the solubility in an alkali developer by the action of an acid.

The resin (acid-decomposable resin) capable of increasing the solubility in an alkali developer by the action of an acid has a group that decomposes by the action of an acid to produce an alkali-soluble group (hereinafter sometimes referred to as an “acid-decomposable group”), on either one or both of the main chain and the side chain of the resin.

The resin (B1) is preferably insoluble or sparingly soluble in an alkali developer.

The acid-decomposable group preferably has a structure where an alkali-soluble group is protected by a group capable of decomposing and leaving by the action of an acid.

Examples of the alkali-soluble group include a phenolic hydroxyl group, a carboxyl group, a fluorinated alcohol group, a sulfonic acid group, a sulfonamide group, a sulfonylimide group, an (alkylsulfonyl)(alkylcarbonyl)methylene group, an (alkylsulfonyl)(alkylcarbonyl)imide group, a bis(alkylcarbonyl)methylene group, a bis(alkylcarbonyl)imide group, a bis(alkylsulfonyl)methylene group, a bis(alkylsulfonyl)imide group, a tris(alkylcarbonyl)methylene group and a tris(alkylsulfonyl)methylene group.

Preferred alkali-soluble groups include a carboxyl group, a fluorinated alcohol group (preferably hexafluoroisopropanol) and a sulfonic acid group.

The group preferred as the acid-decomposable group is a group where a group capable of leaving by the action of an acid is substituted for a hydrogen atom of the alkali-soluble group above.

Examples of the group capable of leaving by the action of an acid include —C(R₃₆)(R₃₇)(R₃₈) and —C(R₀₁)(R₀₂)(OR₃₉).

In the formulae, each of R₃₆ to R₃₉ independently represents an alkyl group, a cycloalkyl group, an aryl group, an aralkyl group or an alkenyl group, and R₃₆ and R₃₇ may combine with each other to form a ring.

Each of R₀₁ and R₀₂ independently represents a hydrogen atom, an alkyl group, a cycloalkyl group, an aryl group, an aralkyl group or an alkenyl group. R₀₁ and R₀₂ may combine with each other to form a ring.

The alkyl group of R₃₆ to R₃₉, R₀₁ and R₀₂ is preferably an alkyl group having a carbon number of 1 to 8, and examples thereof include a methyl group, an ethyl group, a propyl group, an n-butyl group, a sec-butyl group, a hexyl group and an octyl group.

The cycloalkyl group of R₃₆ to R₃₉, R₀₁ and R₀₂ may be either monocyclic or polycyclic. The monocyclic cycloalkyl group is preferably a cycloalkyl group having a carbon number of 3 to 8, and examples thereof include a cyclopropyl group, a cyclobutyl group, a cyclopentyl group, a cyclohexyl group and a cyclooctyl group. The polycyclic cycloalkyl group is preferably a cycloalkyl group having a carbon number of 6 to 20, and examples thereof include an adamantyl, a norbornyl group, an isoboronyl group, a camphanyl group, a dicyclopentyl group, an α-pinel group, a tricyclodecanyl group, a tetracyclododecyl group and an androstanyl group. Incidentally, a part of carbon atoms in the cycloalkyl group may be substituted with a heteroatom such as oxygen atom.

The aryl group of R₃₆ to R₃₉, R₀₁ and R₀₂ is preferably an aryl group having a carbon number of 6 to 10, and examples thereof include a phenyl group, a naphthyl group and an anthryl group.

The aralkyl group of R₃₆ to R₃₉, R₀₁ and R₀₂ is preferably an aralkyl group having a carbon number of 7 to 12, and examples thereof include a benzyl group, a phenethyl group and a naphthylmethyl group.

The alkenyl group of R₃₆ to R₃₉, R₀₁ and R₀₂ is preferably an alkenyl group having a carbon number of 2 to 8, and examples thereof include a vinyl group, an allyl group, a butenyl group and a cyclohexenyl group.

The acid-decomposable group is preferably a cumyl ester group, an enol ester group, an acetal ester group, a tertiary alkyl ester group or the like, more preferably a tertiary alkyl ester group.

The acid-decomposable group-containing repeating unit which can be contained in the resin (B1) is preferably a repeating unit represented by the following formula (AI):

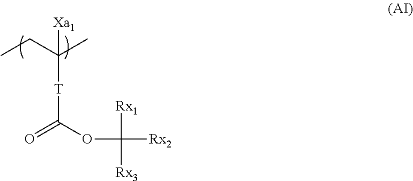

In formula (AI), Xa₁ represents a hydrogen atom, a methyl group which may have a substituent, or a group represented by —CH₂—R₉. R₉ represents a hydroxyl group or a monovalent organic group. Examples of the monovalent organic group include an alkyl group having a carbon number of 5 or less and an acyl group having a carbon number of 5 or less. Of these, an alkyl group having a carbon number of 3 or less is preferred, and a methyl group is more preferred. Xa₁ is preferably a hydrogen atom, a methyl group, a trifluoromethyl group or a hydroxymethyl group.

T represents a single bond or a divalent linking group.

Each of Rx₁ to Rx₃ independently represents an alkyl group (linear or branched) or a cycloalkyl group (monocyclic or polycyclic).

Two members out of Rx₁ to Rx₃ may combine to form a cycloalkyl group (monocyclic or polycyclic).

Examples of the divalent linking group of T include an alkylene group, a —COO-Rt- group, a —O-Rt- group, and a group formed by combining two or more of these groups. Among these, an alkylene group, a —COO-Rt- group and a —O-Rt- group are preferred. In the formulae, Rt represents an alkylene group or a cycloalkylene group. The total carbon number of the divalent linking group of T is preferably from 1 to 20, more preferably from 1 to 15, still more preferably from 2 to 10.

T is preferably a single bond or a —COO-Rt- group. Rt is preferably an alkylene group having a carbon number of 1 to 5, more preferably a —CH₂— group, a —(CH₂)₂— group or a —(CH₂)₃— group.

The alkyl group of Rx₁ to Rx₃ is preferably an alkyl group having a carbon number of 1 to 4, such as methyl group, ethyl group, n-propyl group, isopropyl group, n-butyl group, isobutyl group and tert-butyl group.

The cycloalkyl group of Rx₁ to Rx₃ is preferably a monocyclic cycloalkyl group such as cyclopentyl group and cyclohexyl group, or a polycyclic cycloalkyl group such as norbornyl group, tetracyclodecanyl group, tetracyclododecanyl group and adamantyl group.

The cycloalkyl group formed by combining two members out of Rx₁ to Rx₃ is preferably a monocyclic cycloalkyl group such as cyclopentyl group and cyclohexyl group, or a polycyclic cycloalkyl group such as norbornyl group, tetracyclodecanyl group, tetracyclododecanyl group and adamantyl group. Above all, a monocyclic cycloalkyl group having a carbon number of 5 to 6 is preferred.

An embodiment where Rx₁ is a methyl group or an ethyl group and Rx₂ and Rx₃ are combined to form the above-described cycloalkyl group is preferred.

Each of the groups above may have a substituent, and examples of the substituent include an alkyl group (having a carbon number of 1 to 4), a halogen atom, a hydroxyl group, an alkoxy group (having a carbon number of 1 to 4), a carboxyl group and an alkoxycarbonyl group (having a carbon number of 2 to 6). The carbon number is preferably 8 or less.

The content in total of the acid-decomposable group-containing repeating units is preferably from 20 to 70 mol %, more preferably from 30 to 50 mol %, based on all repeating units in the resin.

Specific preferred examples of the repeating unit having an acid-decomposable group are illustrated below, but the present invention is not limited thereto.

In specific examples, each of Rₓ and Xa₁ represents a hydrogen atom, CH₃, CF₃ or CH₂OH, and each of Rxa and Rxb represents an alkyl group having a carbon number of 1 to 4. Z represents a substituent containing a polar group, and when a plurality of Z's are present, each is independent from every others, p represents 0 or a positive integer. Specific examples of Z are the same as those of R₁₀ in formula (II-1) described later, p represents 0 or a positive integer.

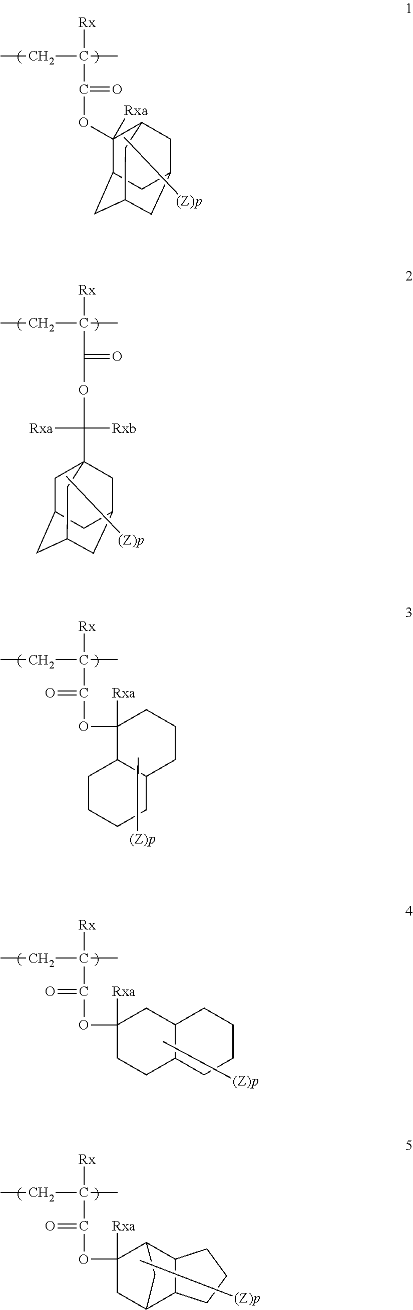

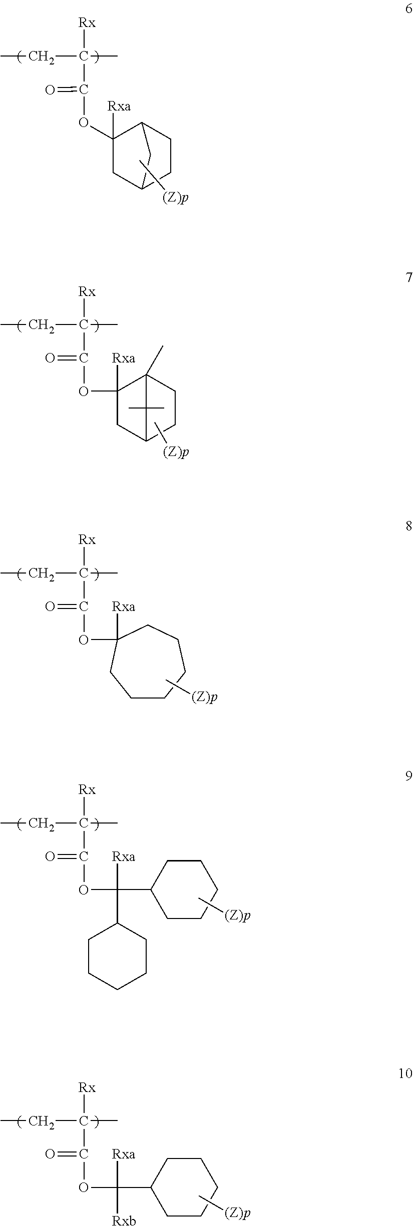

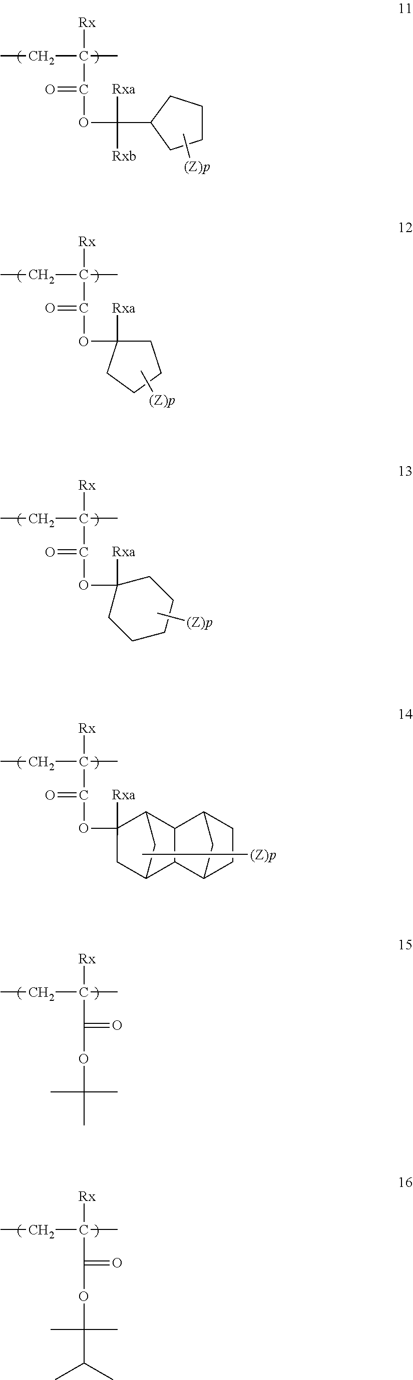

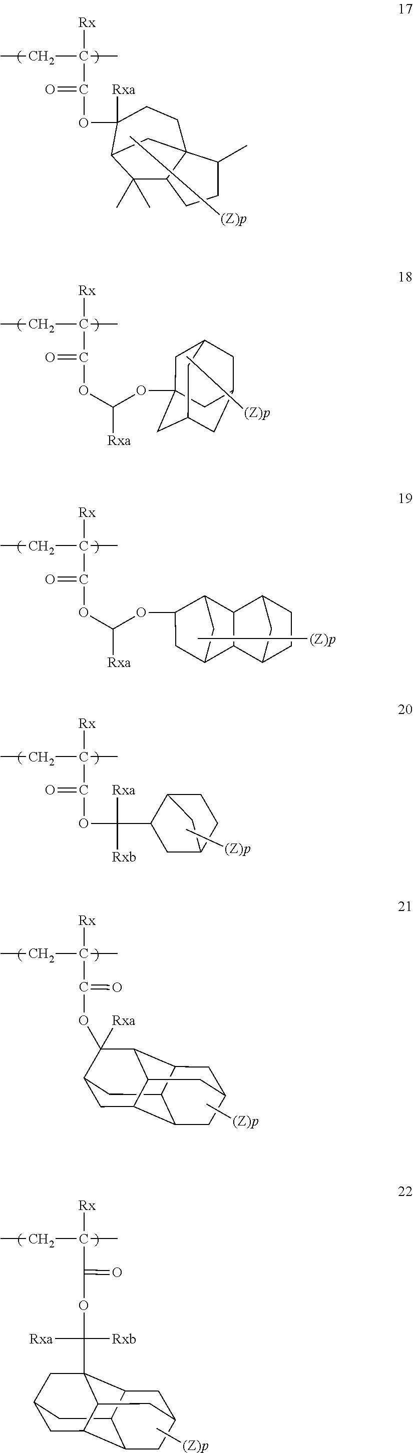

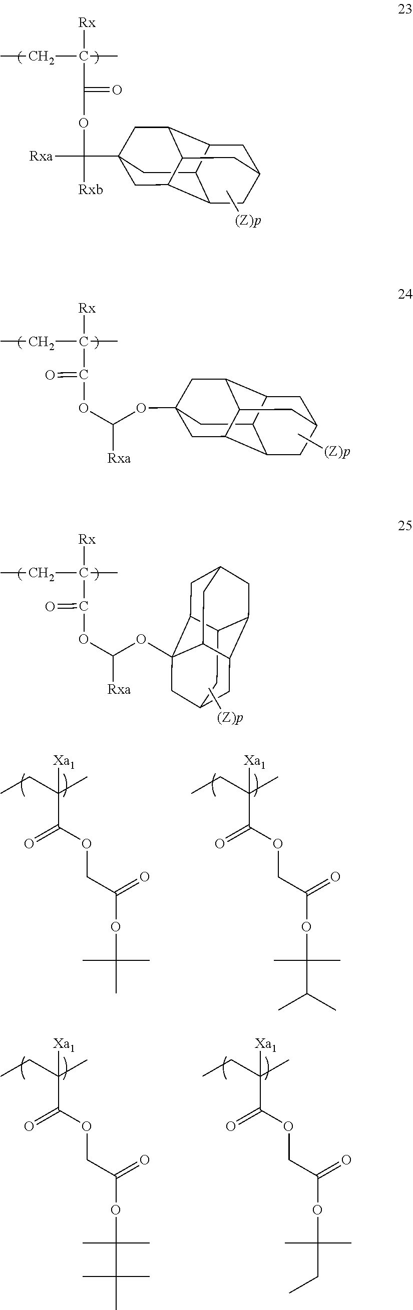

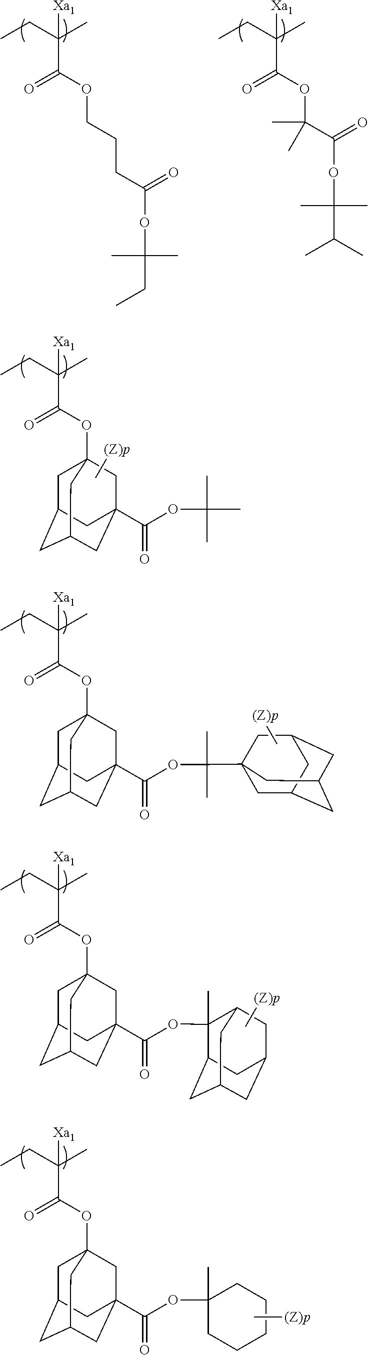

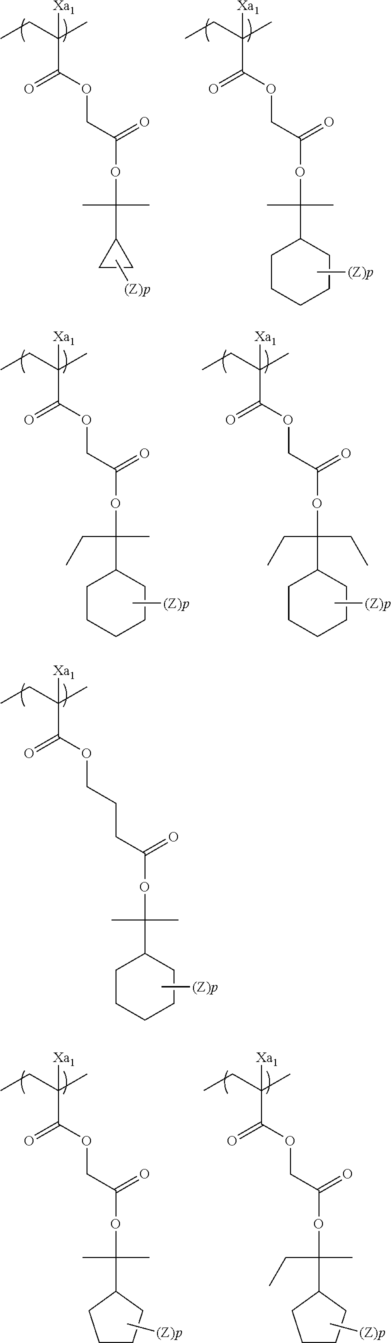

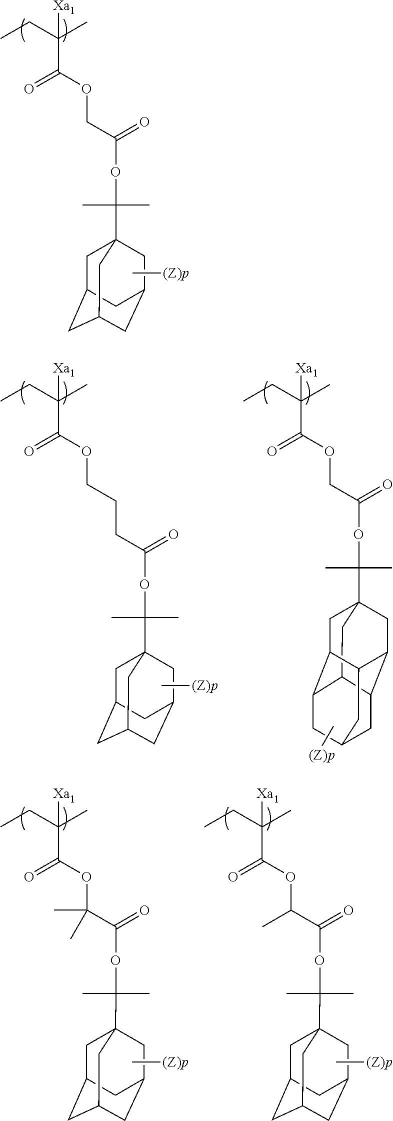

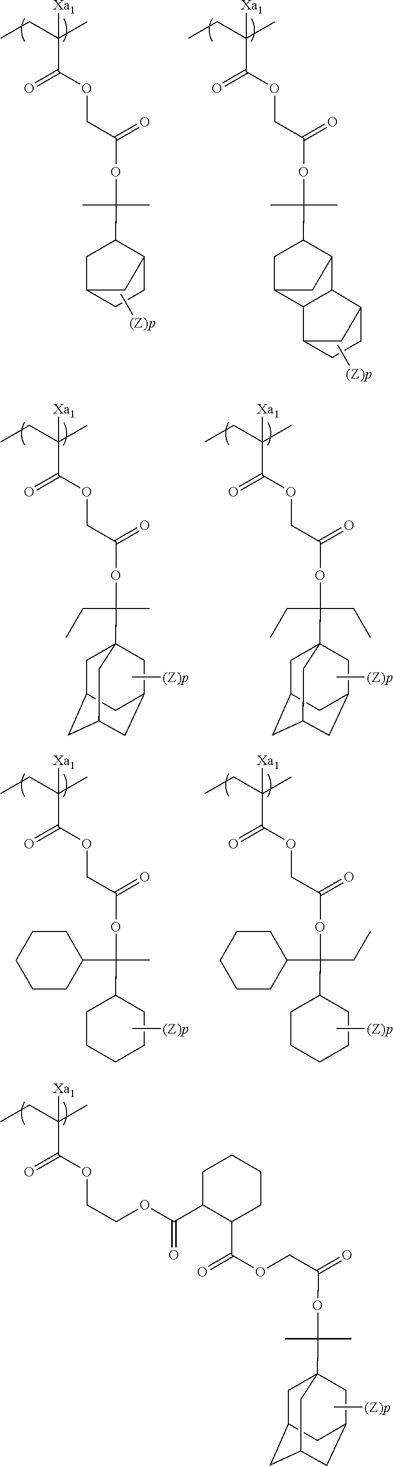

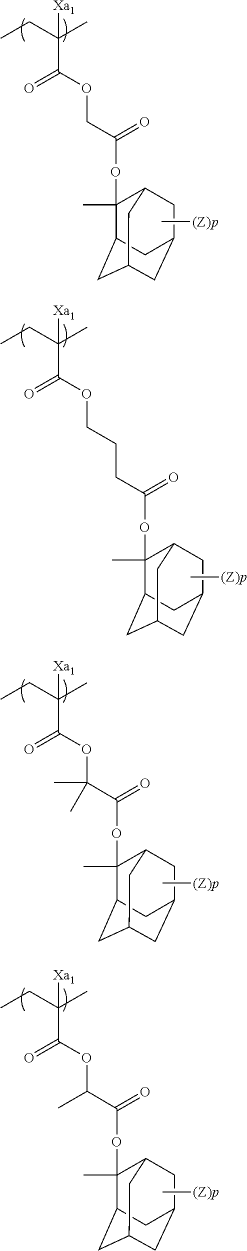

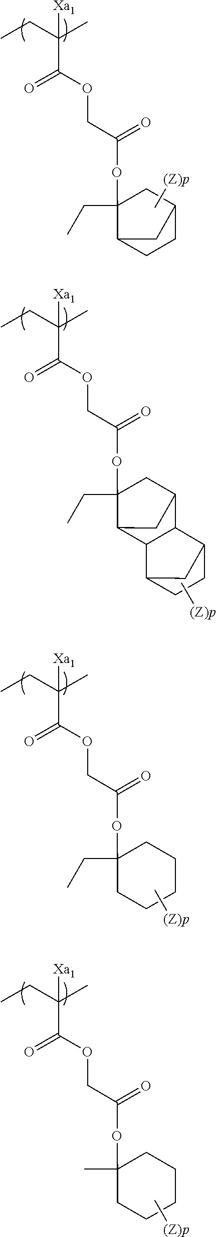

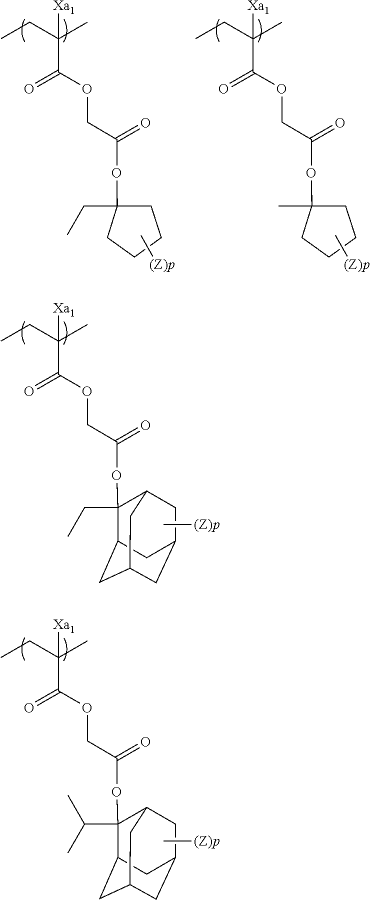

The resin (B1) is more preferably a resin having, as the repeating unit represented by formula (AI), at least either one of a repeating unit represented by formula (I) and a repeating unit represented by formula (II).

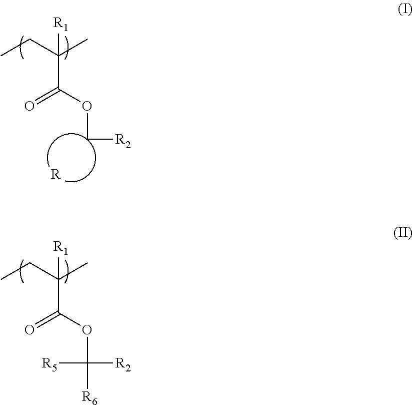

In formulae (I) and (II), each of R₁ and R₃ independently represents a hydrogen atom, a methyl group which may have a substituent, or a group represented by —CH₂—R₉. R₉ represents a monovalent organic group. Examples of the monovalent organic group include an alkyl group having a carbon number of 5 or less and an acyl group having a carbon number of 5 or less. Of these, an alkyl group having a carbon number of 3 or less is preferred, and a methyl group is more preferred.

Each of R₂, R₄, R₅ and R₆ independently represents an alkyl group or a cycloalkyl group.

R represents an atomic group necessary for forming an alicyclic structure together with the carbon atom.

R₁ is preferably a hydrogen atom, a methyl group, a trifluoromethyl group or a hydroxymethyl group.

The alkyl group in R₂ may be linear or branched and may have a substituent.

The cycloalkyl group in R₂ may be monocyclic or polycyclic and may have a substituent.

R₂ is preferably an alkyl group, more preferably an alkyl group having a carbon number of 1 to 10, still more preferably an alkyl group having a carbon number of 1 to 5, and examples thereof include a methyl group and an ethyl group.

The alicyclic structure formed by R together with the carbon atom is preferably a monocyclic alicyclic structure, and the carbon number thereof is preferably from 3 to 7, more preferably 5 or 6.

R₃ is preferably a hydrogen atom or a methyl group, more preferably a methyl group.

The alkyl group in R₄, R₅ and R₆ may be linear or branched and may have a substituent. The alkyl group is preferably an alkyl group having a carbon number of 1 to 4, such as methyl group, ethyl group, n-propyl group, isopropyl group, n-butyl group, isobutyl group and tert-butyl group.

The cycloalkyl group in R₄, R₅ and R₆ may be monocyclic or polycyclic and may have a substituent. The cycloalkyl group is preferably a monocyclic cycloalkyl group such as cyclopentyl group and cyclohexyl group, or a polycyclic cycloalkyl group such as norbornyl group, tetracyclodecanyl group, tetracyclododecanyl group and adamantyl group.

Examples of the substituent which the alkyl group of R₂, R₄, R₅ and R₆ may further have include an aryl group (e.g., phenyl, naphthyl), an aralkyl group, a hydroxyl group, an alkoxy group (e.g., methoxy, ethoxy, butoxy, octyloxy, dodecyloxy), an acyl group (e.g., acetyl, propanoyl, benzoyl), and an oxo group. The carbon number of the substituent is preferably 15 or less.

Examples of the substituent which the cycloalkyl group of R₂, R₄, R₅ and R₆ may further have include an alkyl group (e.g., methyl, ethyl, propyl, isopropyl, butyl, tert-butyl, hexyl), and the groups exemplified above as the substituent which the alkyl group of R₂ may further have. The carbon number of the substituent is preferably 15 or less.

The repeating unit represented by formula (I) is preferably a repeating unit represented by the following formula (V):

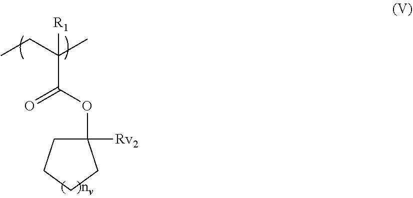

In formula (V), each of Rv₁ and Rv₂ independently represents an alkyl group having a carbon number of 1 to 10.

nᵥ represents an integer of 1 to 6.

nᵥ is preferably 1 or 2, more preferably 1.

The alkyl group having a carbon number of 1 to 10 in Rv₁ and Rv₂ may be linear or branched and may have a substituent. Examples of the substituent include a cycloalkyl group (preferably having a carbon number of 3 to 10), a halogen atom, a hydroxyl group, an alkoxy group (preferably having a carbon number of 1 to 4), a carboxyl group and an alkoxycarbonyl group (preferably having a carbon number of 2 to 6). The carbon number is preferably 8 or less.

The repeating unit represented by formula (II) is preferably a repeating unit represented by the following formula (II-1):

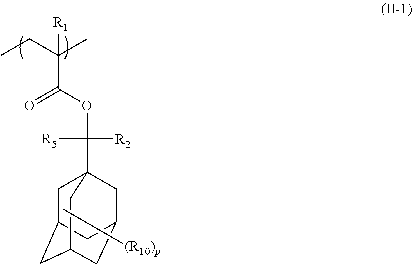

In formula (II-1), R₃ to R₅ have the same meanings as those in formula (II).

R₁₀ represents a polar group-containing substituent. In the case where a plurality of R₁₀'s are present, each R₁₀ may be the same as or different from every other R₁₀. Examples of the polar group-containing substituent include a hydroxyl group, a cyano group, an amino group, an alkylamide group, a sulfonamide group itself, and a linear or branched alkyl group or cycloalkyl group substituted with the group above. An alkyl group having a hydroxyl group is preferred. The branched alkyl group is preferably an isopropyl group.

p represents an integer of 0 to 15. p is preferably an integer of 0 to 2, more preferably 0 or 1.

The resin (B1) may contain acid-decomposable group-containing repeating units in combination. Also, the composition of the present invention may contain a plurality of kinds of resins (B1), and the resins may contain different acid-decomposable groups. In this case, it is preferred to contain at least two kinds of repeating units represented by formula (I), contain a repeating unit represented by formula (I) and a repeating unit represented by formula (II), or contain a repeating unit represented by formula (V) and a repeating unit represented by formula (II). In the case of containing at least two kinds of repeating units represented by formula (I), examples of the combination include a combination of a repeating unit where R₂ in formula (I) is an ethyl group and a repeating unit where the R₂ is a methyl group, and a combination of a repeating unit where R₂ in formula (I) is an ethyl group and a repeating unit where the R₂ is a cycloalkyl group; in the case of containing a repeating unit represented by formula (I) and a repeating unit represented by formula (II), examples of the combination include a combination of a repeating unit where R₂ in formula (I) is an ethyl group and a repeating unit where R₄ and R₅ in formula (II) are a methyl group and R₆ is an adamantyl group, and a combination of a repeating unit where R₂ in formula (I) is an ethyl group and a repeating unit where R₄ and R₅ in formula (II) are a methyl group and R₆ is a cyclohexyl group; in the case of containing a repeating unit represented by formula (V) and a repeating unit represented by formula (II), examples of the combination include a combination of a repeating unit where Rv₂ in formula (V) is an ethyl group and n is 1 and a repeating unit where R₄ and R₅ in formula (II) are a methyl group and R₆ is an adamantyl group, and a combination of a repeating unit where Rv₂ in formula (V) is an ethyl group and n is 2 and a repeating unit where R₄ and R₅ in formula (II) are a methyl group and R₆ is a cyclohexyl group.

In the resin (B1), when acid-decomposable repeating units are used in combination, preferred examples of the combination are set forth below. In the following formulae, each R independently represents a hydrogen atom or a methyl group.

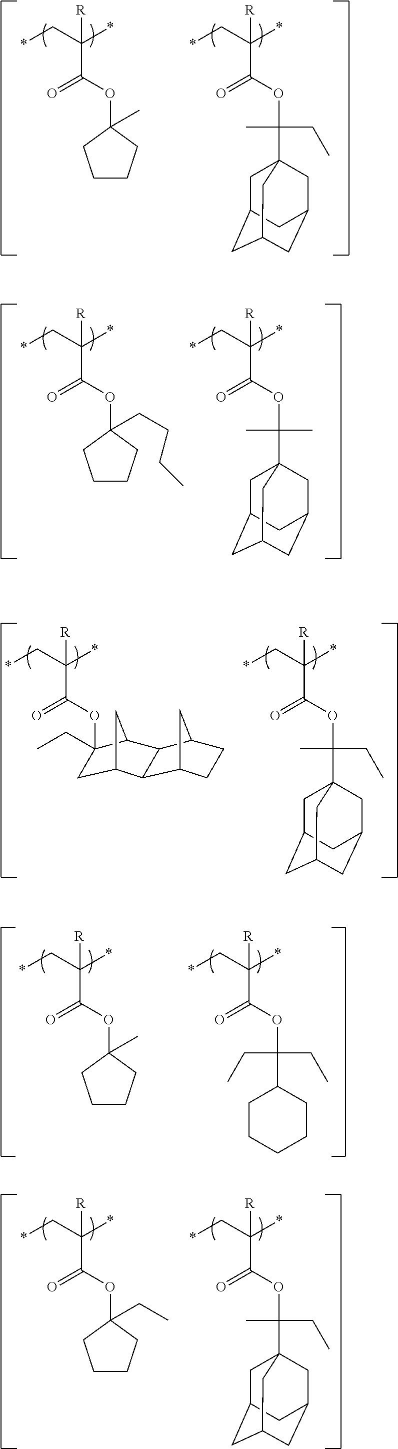

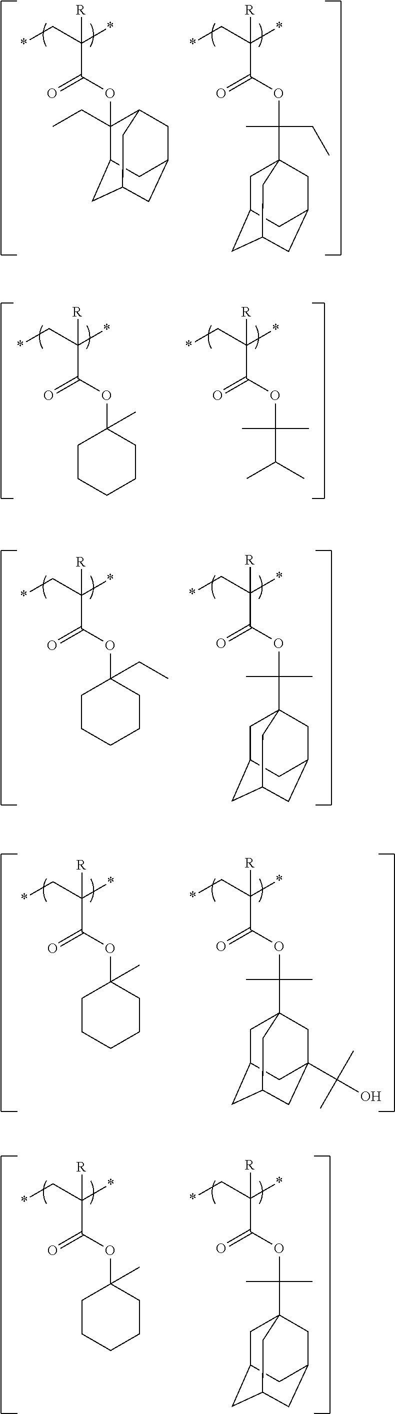

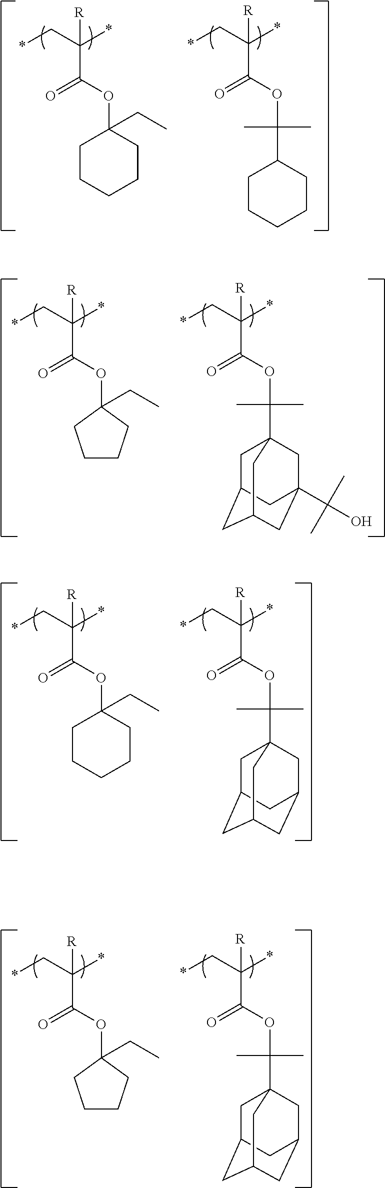

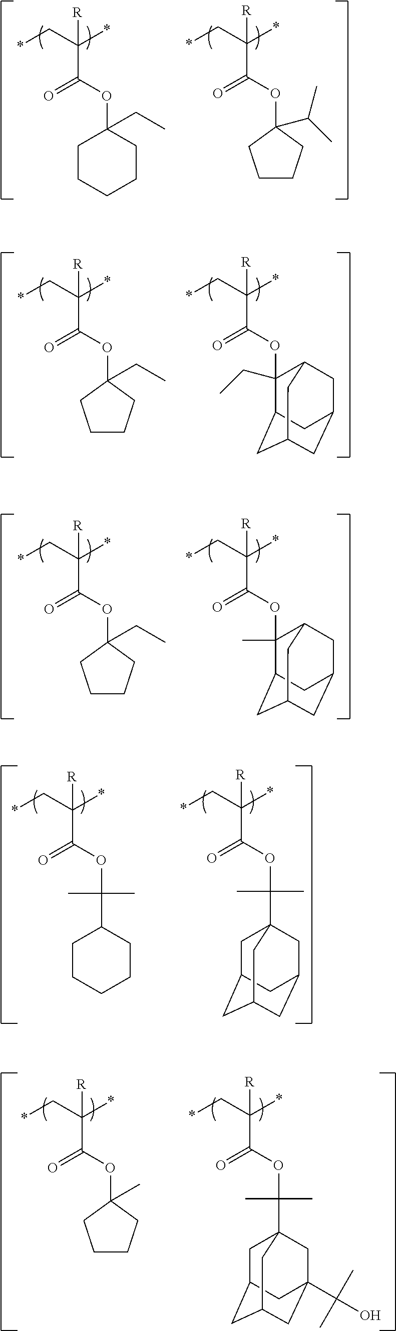

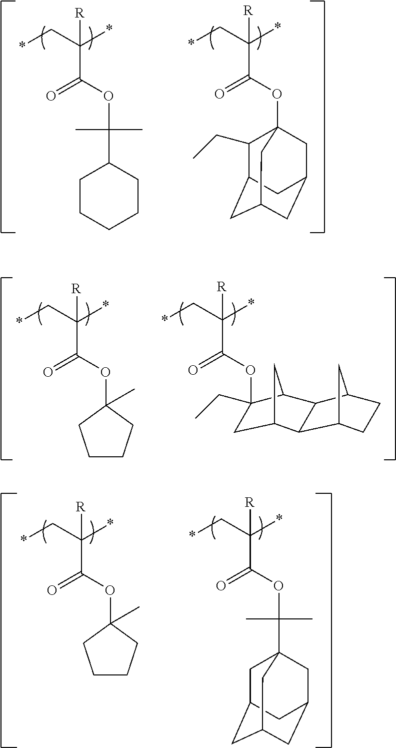

The resin (B1) preferably contains a repeating unit having a lactone structure, more preferably a repeating unit having a lactone structure substituted with a cyano group.

It is preferred to contain a lactone structure-containing repeating unit represented by the following formula (III):

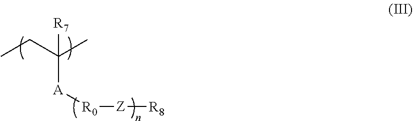

In formula (III), A represents an ester bond (a group represented by —COO—) or an amide bond (a group represented by —CONH—).

R₀ represents, when a plurality of R₀'s are present, each independently represents, an alkylene group, a cycloalkylene group or a combination thereof.

Z represents, when a plurality of Z's are present, each independently represents, an ether bond, an ester bond, an amide bond, a urethane bond (a group represented by (—O—C(═O)—N(R)—) or (—N(R)—C(═O)—O—)), or a urea bond (a group represented by —N(R)—C(═O)—N(R)—). Here, R represents a hydrogen atom, an alkyl group, a cycloalkyl group or an aryl group.

R₈ represents a monovalent organic group having a lactone structure.

n is a repetition number of the structure represented by —R₀—Z— and represents an integer of 1 to 5, preferably 0 or 1.

R₇ represents a hydrogen atom, a halogen atom or an alkyl group.

The alkylene group and cycloalkylene group of R₀ may have a substituent.

Z is preferably an ether bond or an ester bond, more preferably an ester bond.

The alkyl group of R₇ is preferably an alkyl group having a carbon number of 1 to 4, more preferably a methyl group or an ethyl group, still more preferably a methyl group. The alkyl group in R₇ may be substituted, and examples of the substituent include a halogen atom such as fluorine atom, chlorine atom and bromine atom, a mercapto group, a hydroxy group, an alkoxy group such as methoxy group, ethoxy group, isopropoxy group, tert-butoxy group and benzyloxy group, and an acyloxy group such as acetyloxy group and propionyloxy group. R₇ is preferably a hydrogen atom, a methyl group, a trifluoromethyl group or a hydroxymethyl group.

The chain alkylene group in R₀ is preferably a chain alkylene group having a carbon number of 1 to 10, more preferably a chain alkylene group having a carbon number of 1 to 5, and examples thereof include a methylene group, an ethylene group and a propylene group. The cycloalkylene is preferably a cycloalkylene having a carbon number of 3 to 20, and examples thereof include a cyclohexylene group, a cyclopentylene group, a norbornylene group and an adamantylene group. For bringing out the effects of the present invention, a chain alkylene group is more preferred, and a methylene group is still more preferred.

The lactone structure-containing monovalent organic group represented by R₈ is not limited as long as it has a lactone structure. Specific examples thereof include lactone structures represented by formulae (LC1-1) to (LC1-17) and of these, a structure represented by (LC1-4) is preferred. Structures where n2 in (LC1-1) to (LC1-17) is an integer of 2 or less are more preferred.

R₈ is preferably a monovalent organic group having an unsubstituted lactone structure or a monovalent organic group containing a lactone structure having a methyl group, a cyano group or an alkoxycarbonyl group as the substituent, more preferably a monovalent organic group containing a lactone structure having a cyano group as the substituent (cyanolactone).

Specific examples of the repeating unit having a lactone structure-containing group represented by formula (III) are illustrated below, but the present invention is not limited thereto.

In specific examples, R represents a hydrogen atom, an alkyl group which may have a substituent, or a halogen atom and preferably represents a hydrogen atom, a methyl group, a hydroxymethyl group or an acetoxymethyl group.

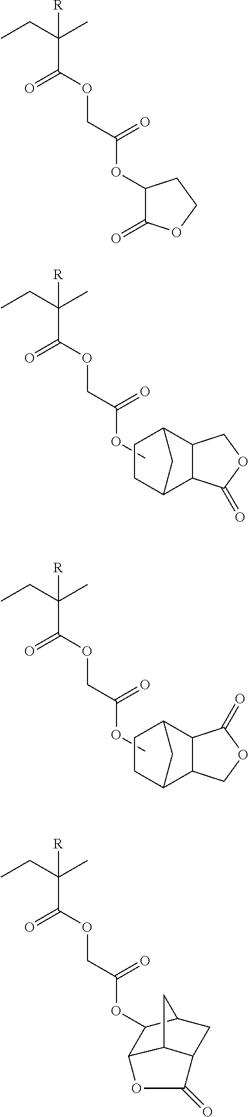

The lactone structure-containing repeating unit is more preferably a repeating unit represented by the following formula (III-1):

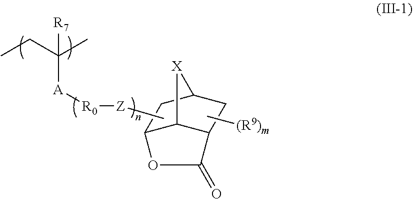

In formula (III-1), R₇, A, R₀, Z and n have the same meanings as in formula (III).

R₉ represents, when a plurality of R₉'s are present, each independently represents, an alkyl group, a cycloalkyl group, an alkoxycarbonyl group, a cyano group, a hydroxyl group or an alkoxy group, and when a plurality of R₉'s are present, two members thereof may combine to form a ring.

X represents an alkylene group, an oxygen atom or a sulfur atom.

m is the number of substituents and represents an integer of 0 to 5. m is preferably 0 or 1.

The alkyl group of R₉ is preferably an alkyl group having a carbon number of 1 to 4, more preferably a methyl group or an ethyl group, and most preferably a methyl group. Examples of the cycloalkyl group include a cyclopropyl group, a cyclobutyl group, a cyclopentyl group and a cyclohexyl group. The alkoxycarbonyl group is preferably an alkoxycarbonyl group having a carbon number of 2 to 5, and examples thereof include a methoxycarbonyl group, an ethoxycarbonyl group, an n-butoxycarbonyl group and a tert-butoxycarbonyl group. Examples of the alkoxy group include a methoxy group, an ethoxy group, a propoxy group, an isopropoxy group and a butoxy group. These groups may have a substituent, and the substituent includes a hydroxy group, an alkoxy group such as methoxy group and ethoxy group, a cyano group, and a halogen atom such as fluorine atom. R₉ is preferably a methyl group, a cyano group or an alkoxycarbonyl group, more preferably a cyano group.

Examples of the alkylene group of X include a methylene group and an ethylene group. X is preferably an oxygen atom or a methylene group, more preferably a methylene group.

When m is an integer of 1 or more, at least one R₉ is preferably substituted at the α-position or β-position, more preferably at the α-position, of the carbonyl group of lactone.

Specific examples of the repeating unit having a lactone structure-containing group represented by formula (III-1) are illustrated below, but the present invention is not limited thereto. In specific examples, R represents a hydrogen atom, an alkyl group which may have a substituent, or a halogen atom and preferably represents a hydrogen atom, a methyl group, a hydroxymethyl group or an acetoxymethyl group.

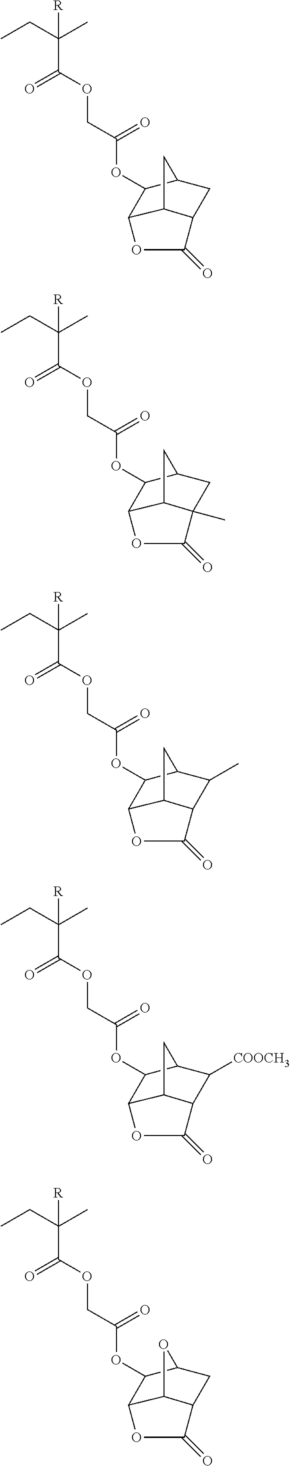

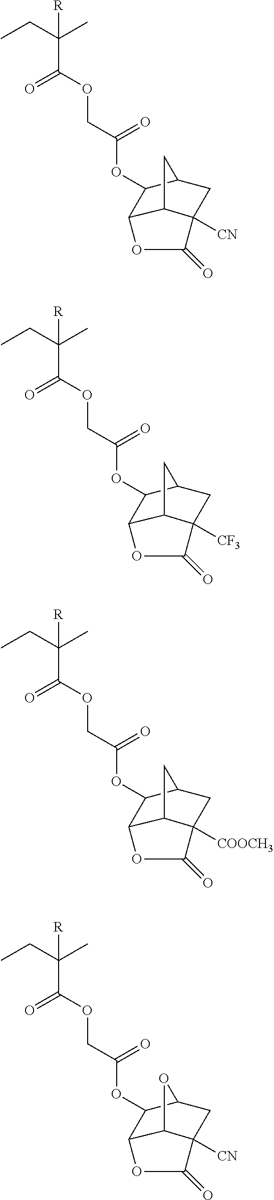

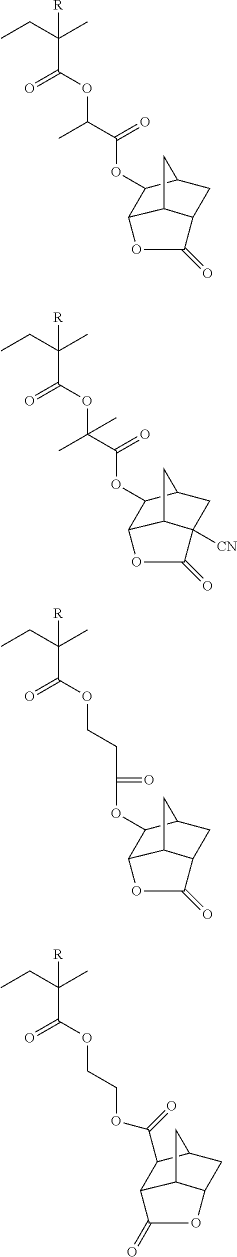

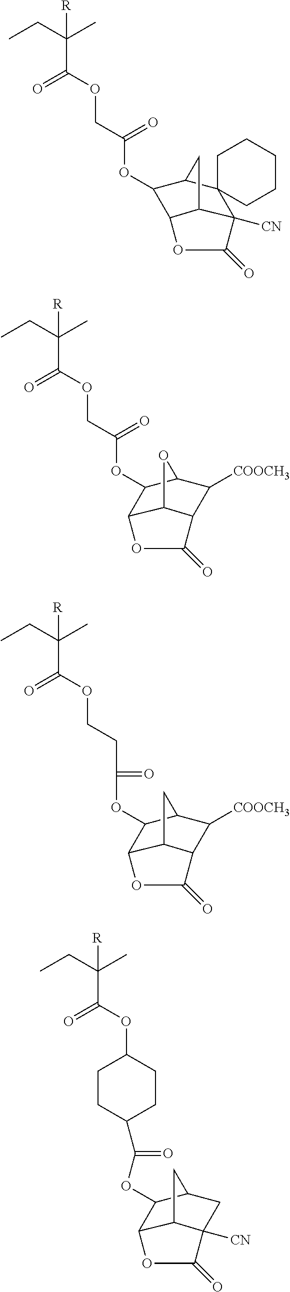

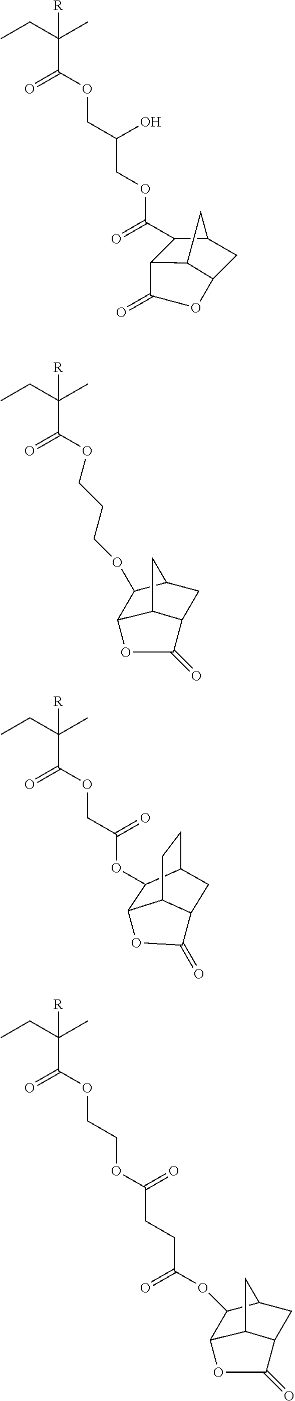

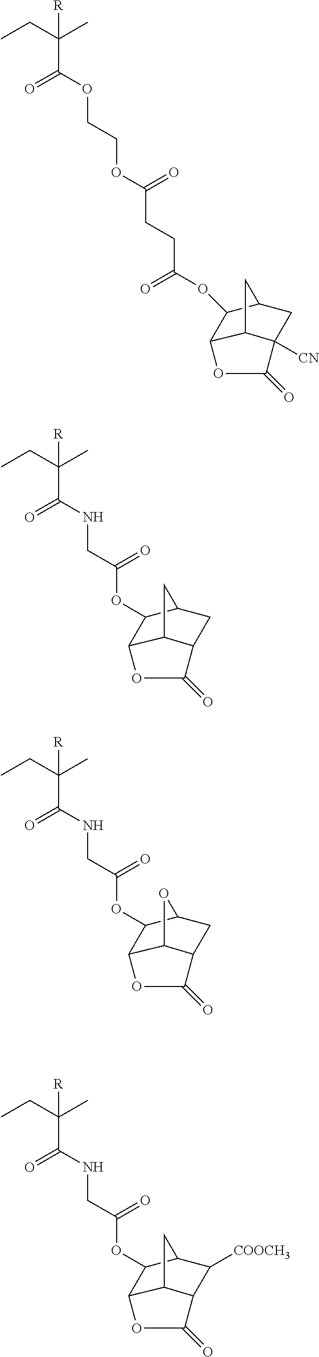

The content of the repeating unit represented by formula (III), in the case of containing a plurality of kinds of the repeating units, the content in total, is preferably from 15 to 60 mol %, more preferably from 20 to 60 mol %, still more preferably from 30 to 50 mol %, based on all repeating units in the resin (B1).

The resin (B1) may further contain a repeating unit having a lactone group, other than the repeating unit represented by formula (III).

As for the lactone group, any group may be used as long as it has a lactone structure, but the lactone structure is preferably a 5- to 7-membered ring lactone structure, and a structure where another ring structure is condensed to a 5- to 7-membered ring lactone structure in the form of forming a bicyclo or spiro structure is preferred. The resin more preferably contains a repeating unit having a lactone structure represented by any one of the following formulae (LC1-1) to (LC1-17). The lactone structure may be bonded directly to the main chain. Among these lactone structures, preferred are (LC1-1), (LC1-4), (LC1-5), (LC1-6), (LC1-13), (LC1-14) and (LC1-17). By using a specific lactone structure, LWR and development defect are improved.

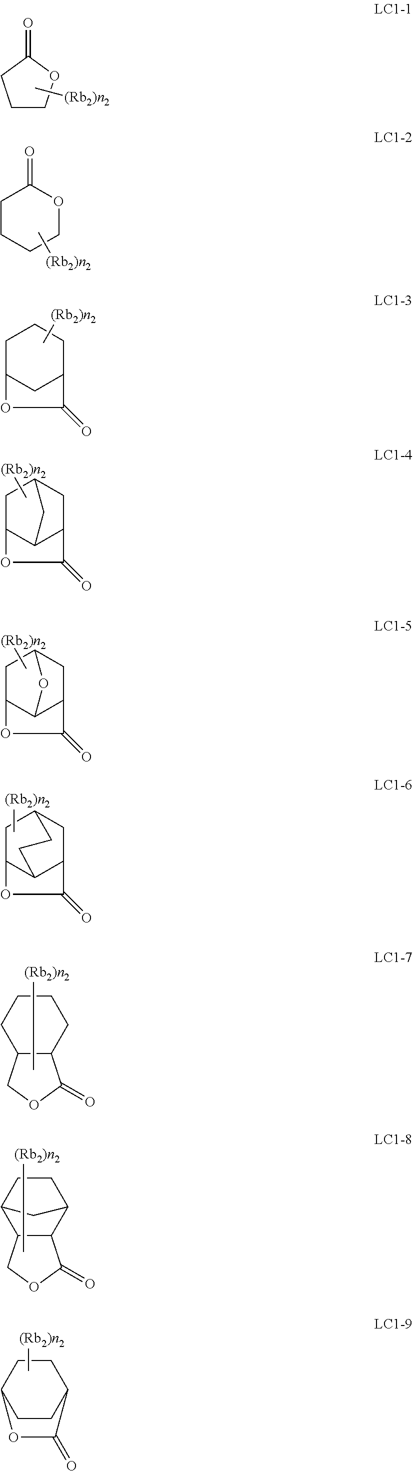

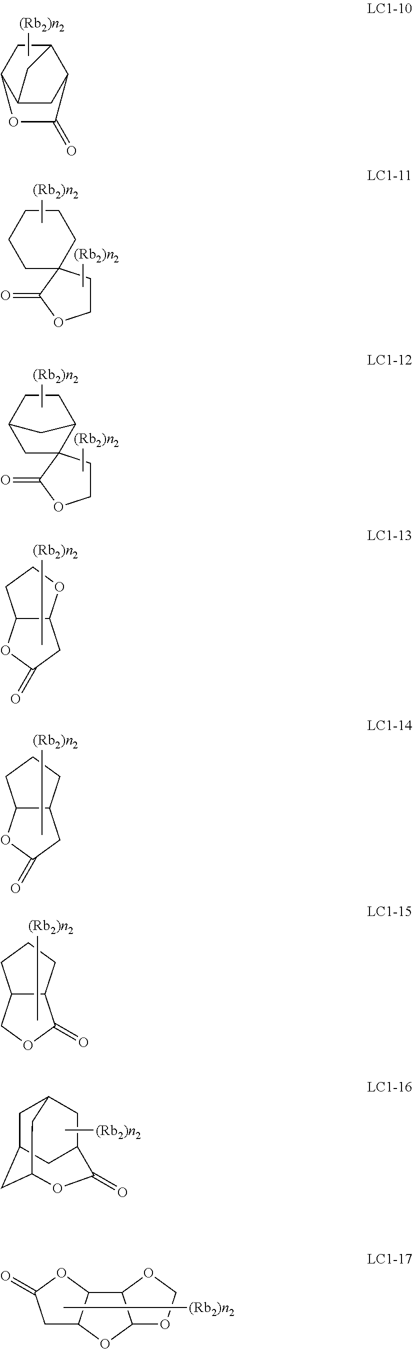

The lactone structure moiety may or may not have a substituent (Rb₂). Preferred examples of the substituent (Rb₂) include an alkyl group having a carbon number of 1 to 8, a cycloalkyl group having a carbon number of 4 to 7, an alkoxy group having a carbon number of 1 to 8, an alkoxycarbonyl group having a carbon number of 2 to 8, a carboxyl group, a halogen atom, a hydroxyl group, a cyano group and an acid-decomposable group. Among these, an alkyl group having a carbon number of 1 to 4, a cyano group and an acid-decomposable group are more preferred. n₂ represents an integer of 0 to 4. When n2 is an integer of 2 or more, each substituent (Rb₂) may be the same as or different from every other substituents (Rb₂) and also, the plurality of substituents (Rb₂) may combine with each other to form a ring.

As for the repeating unit having a lactone structure, other than the repeating unit represented by formula (III), a repeating unit represented by the following formula (AII′) is also preferred.

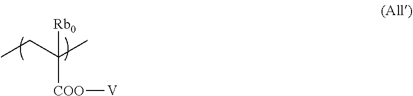

In formula (AII′), Rb₀ represents a hydrogen atom, a halogen atom or an alkyl group having a carbon number of 1 to 4. Preferred substituents which the alkyl group of Rb₀ may have include a hydroxyl group and a halogen atom. The halogen atom of Rb₀ includes a fluorine atom, a chlorine atom, a bromine atom and an iodine atom. Rb₀ is preferably a hydrogen atom, a methyl group, a hydroxymethyl group or a trifluoromethyl group, more preferably a hydrogen atom or a methyl group.

V represents a group having a structure represented by any one of formulae (LC1-1) to (LC1-17).

Specific examples of the repeating unit having a lactone structure, other than the repeating unit represented by formula (III), are illustrated below, but the present invention is not limited thereto.

(In the formulae, Rx represents H, CH₃, CH₂OH or CF₃.)

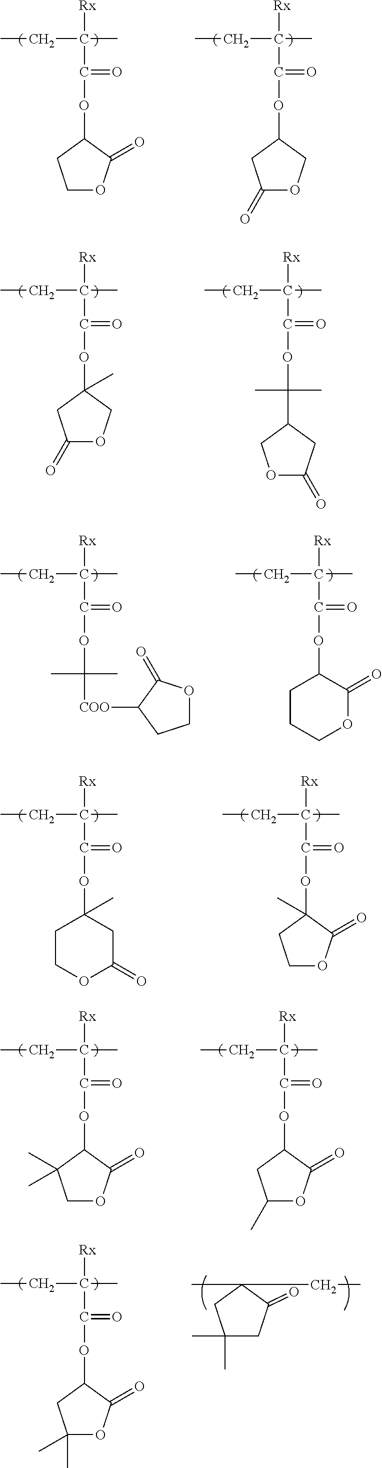

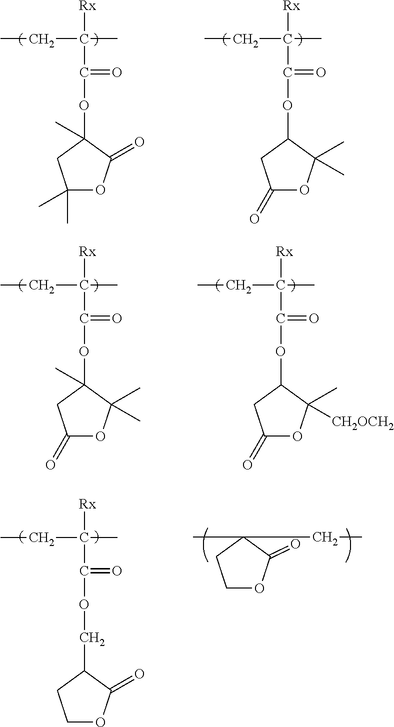

(In the formulae, Rx represents H, CH₃, CH₂OH or CF₃.)

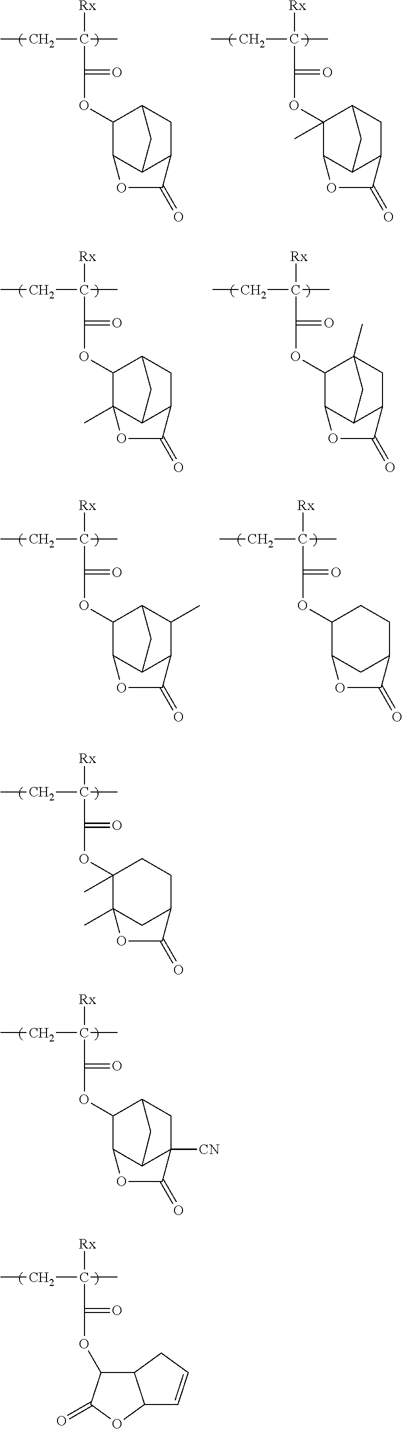

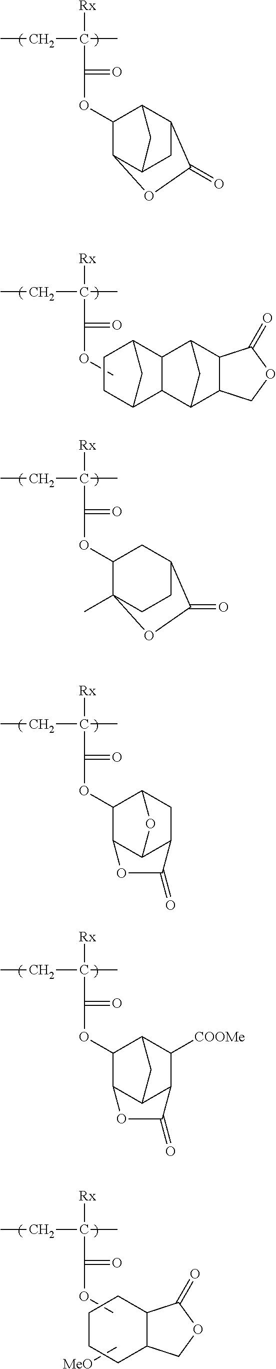

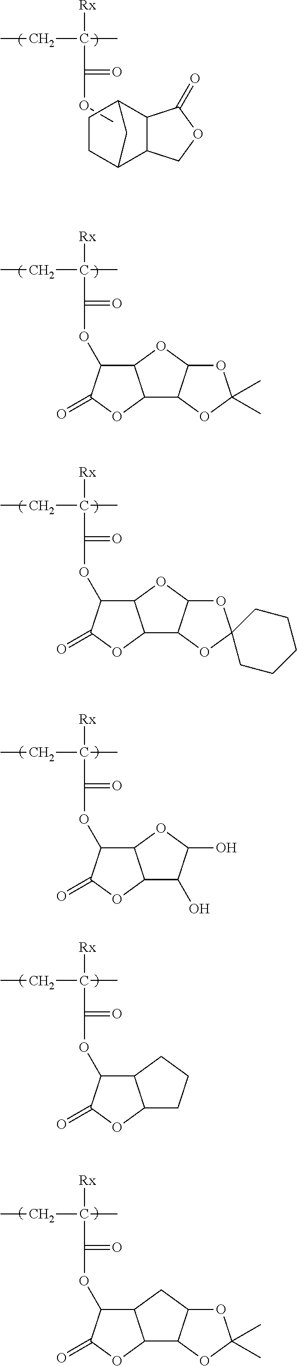

(In the formulae, Rx represents H, CH₃, CH₂OH or CF₃.)

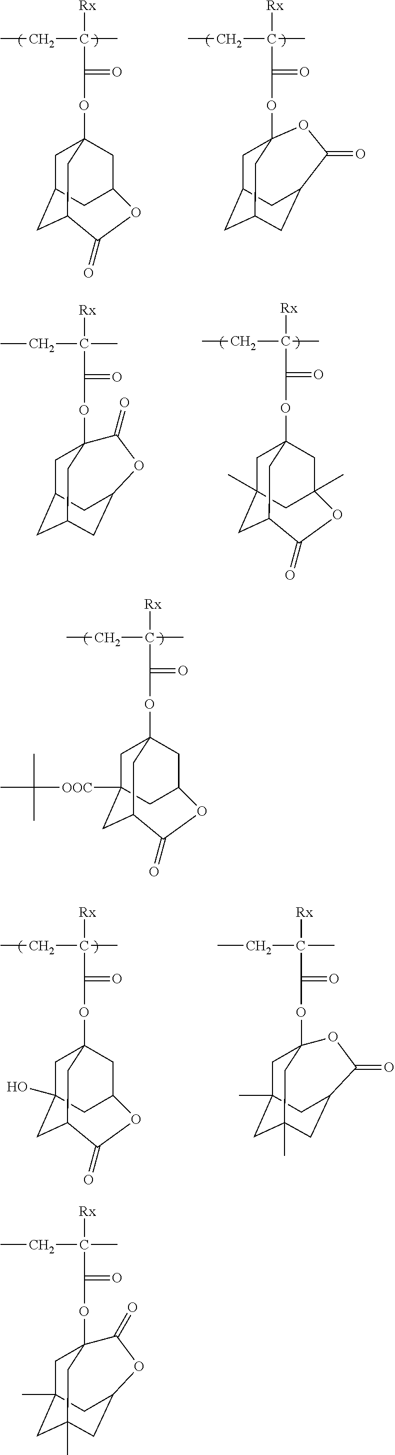

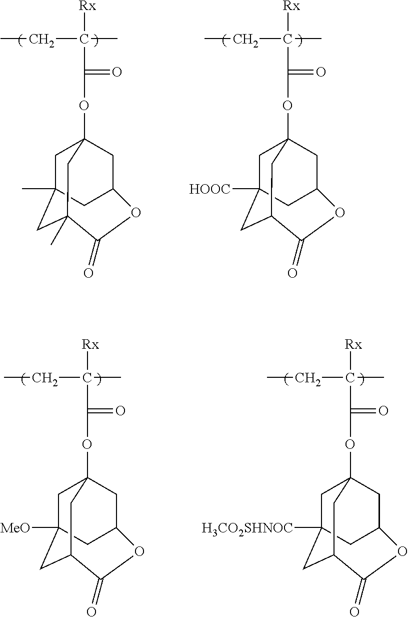

Particularly preferred repeating units having a lactone group, other than the repeating unit represented by formula (III), include the following repeating units. By selecting an optimal lactone group, the pattern profile and the iso/dense bias are improved.

(In formulae, Rx represents H, CH₃, CH₂OH or CF₃.)

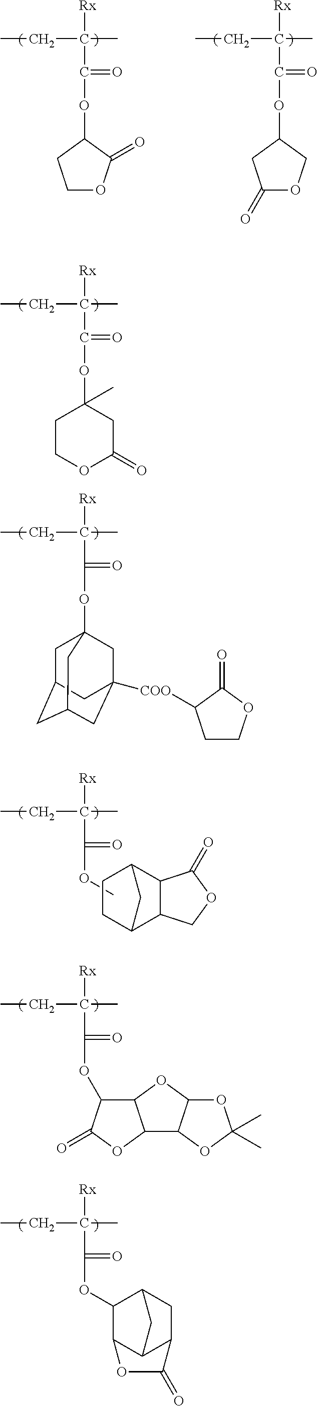

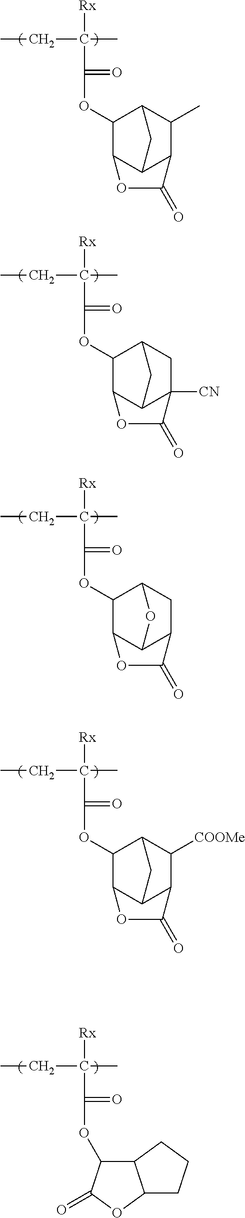

The repeating unit having a lactone group usually has an optical isomer, but any optical isomer may be used. One optical isomer may be used alone or a mixture of a plurality of optical isomers may be used. In the case of mainly using one optical isomer, the optical purity (ee) thereof is preferably 90% or more, more preferably 95% or more.

As the lactone group, a lactone group substituted with a cyano group is also preferred.

The content of the repeating unit having a lactone group, other than the repeating unit represented by formula (III), is preferably from 15 to 60 mol %, more preferably from 20 to 50 mol %, still more preferably from 30 to 50 mol %, based on all repeating units in the resin.

Two or more kinds of lactone repeating units selected from formula (III) may also be used in combination for raising the effects of the present invention. When used in combination, out of formula (III), two or more kinds of lactone repeating units where n is 1 are preferably selected and used in combination.

The resin (B1) preferably contains a repeating unit having a hydroxyl group or a cyano group, other than formulae (AI) and (III). Thanks to this repeating unit, the adherence to substrate and the affinity for developer are enhanced. The repeating unit having a hydroxyl group or a cyano group is preferably a repeating unit having an alicyclic hydrocarbon structure substituted with a hydroxyl group or a cyano group and preferably has no acid-decomposable group. The alicyclic hydrocarbon structure in the alicyclic hydrocarbon structure substituted with a hydroxyl group or a cyano group is preferably an adamantyl group, a diamantyl group or a norbornyl group. As for the preferred alicyclic hydrocarbon structure substituted with a hydroxyl group or a cyano group, partial structures represented by the following formulae (VIIa) to (VIId) are preferred.

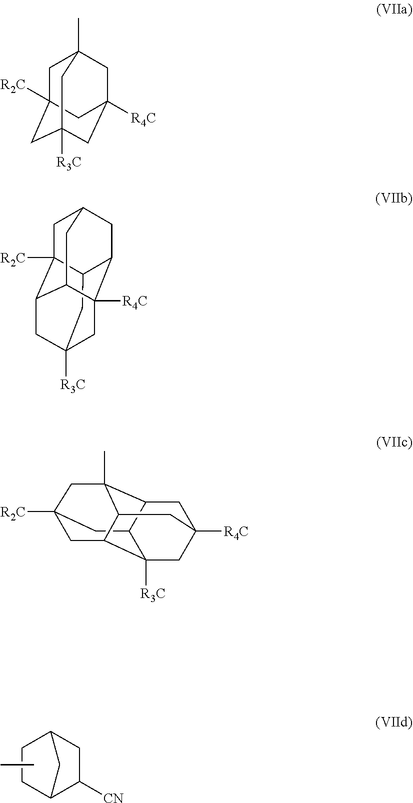

In formulae (VIIa) to (VIIc), each of R_{2c} to R_{4c} independently represents a hydrogen atom, a hydroxyl group or a cyano group, provided that at least one of R_{2c} to R_{4c} represents a hydroxyl group or a cyano group. A structure where one or two members out of R_{2c} to R_{4c} are a hydroxyl group with the remaining being a hydrogen atom is preferred. In formula (VIIa), it is more preferred that two members out of R_{2c} to R_{4c} are a hydroxyl group and the remaining is a hydrogen atom.

The repeating unit having a partial structure represented by formulae (VIIa) to (VIId) includes repeating units represented by the following formulae (AIIa) to (AIId):

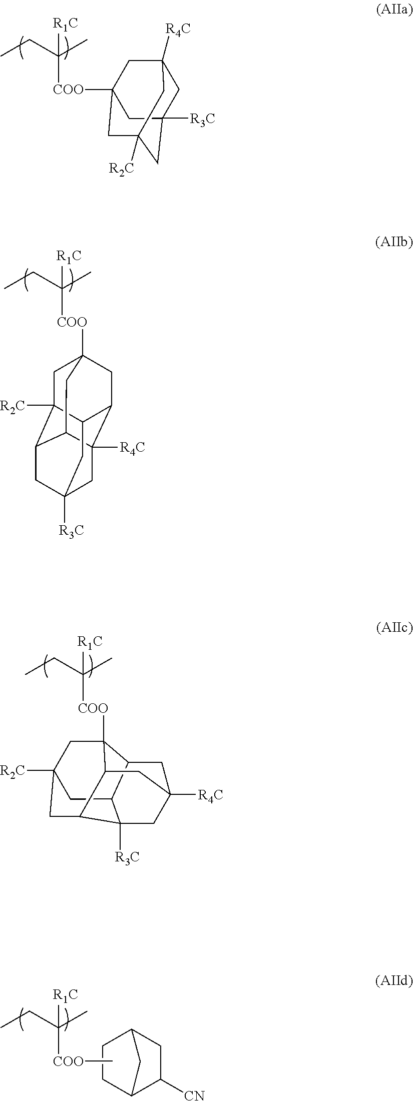

In formulae (AIIa) to (AIId), R_{1c} represents a hydrogen atom, a methyl group, a trifluoromethyl group or a hydroxymethyl group.

R_{2c} to R_{4c} have the same meanings as R_{2c} to R_{4c} in formulae (VIIa) to (VIIc).

The content of the repeating unit having a hydroxyl group or a cyano group is preferably from 5 to 40 mol %, more preferably from 5 to 30 mol %, still more preferably from 10 to 25 mol %, based on all repeating units in the resin (B1).

Specific examples of the repeating unit having a hydroxyl group or a cyano group are illustrated below, but the present invention is not limited thereto.

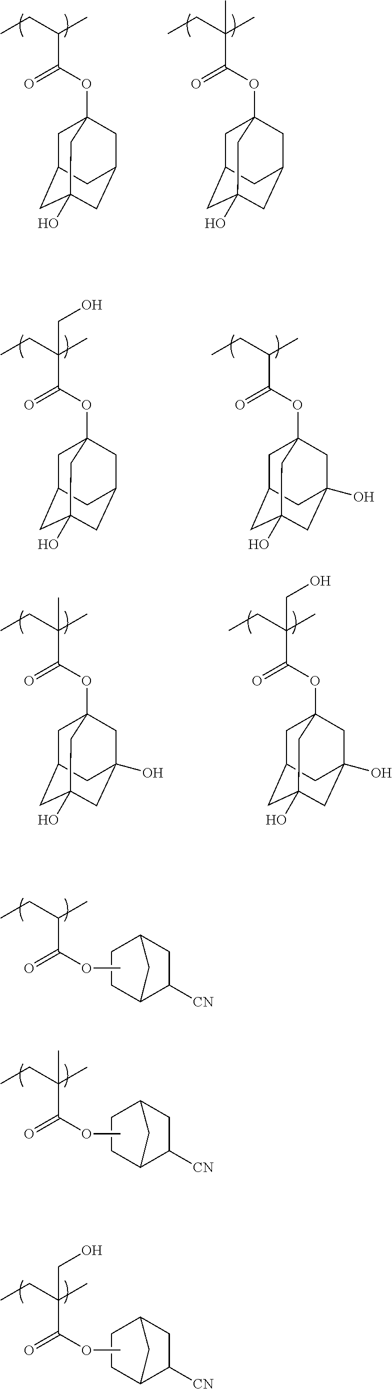

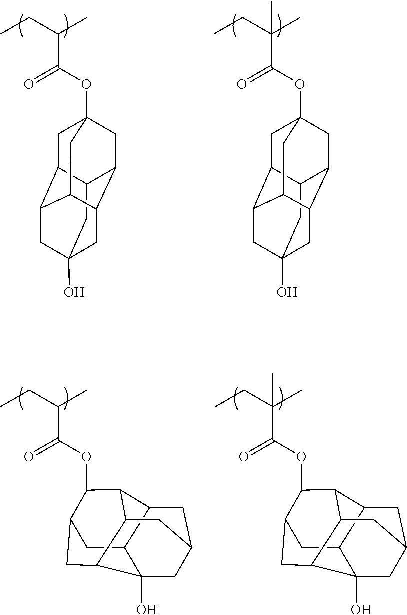

The resin used for the actinic ray-sensitive or radiation-sensitive resin composition of the present invention may contain a repeating unit having an alkali-soluble group. The alkali-soluble group includes a carboxyl group, a sulfonamide group, a sulfonylimide group, a bis-sulfonylimide group, and an aliphatic alcohol substituted with an electron-withdrawing group at the α-position (e.g., hexafluoroisopropanol). It is preferred to contain a repeating unit having a carboxyl group. By virtue of containing an alkali-soluble group-containing repeating unit, the resolution increases in the usage of forming contact holes. As for the repeating unit having an alkali-soluble group, all of a repeating unit where an alkali-soluble group is directly bonded to the main chain of the resin, such as repeating unit by an acrylic acid or a methacrylic acid, a repeating unit where an alkali-soluble group is bonded to the main chain of the resin through a linking group, and a repeating unit where an alkali-soluble group is introduced into the polymer chain terminal by using an alkali-soluble group-containing polymerization initiator or chain transfer agent at the polymerization, are preferred. The linking group may have a monocyclic or polycyclic hydrocarbon structure. Above all, a repeating unit by an acrylic acid or a methacrylic acid is preferred.

The content of the repeating unit having an alkali-soluble group is preferably from 0 to 20 mol %, more preferably from 3 to 15 mol %, still more preferably from 5 to 10 mol %, based on all repeating units in the resin (B1).

Specific examples of the repeating unit having an alkali-soluble group are illustrated below, but the present invention is not limited thereto.

In specific examples, Rx represents H, CH₃, CH₂OH or CF₃.

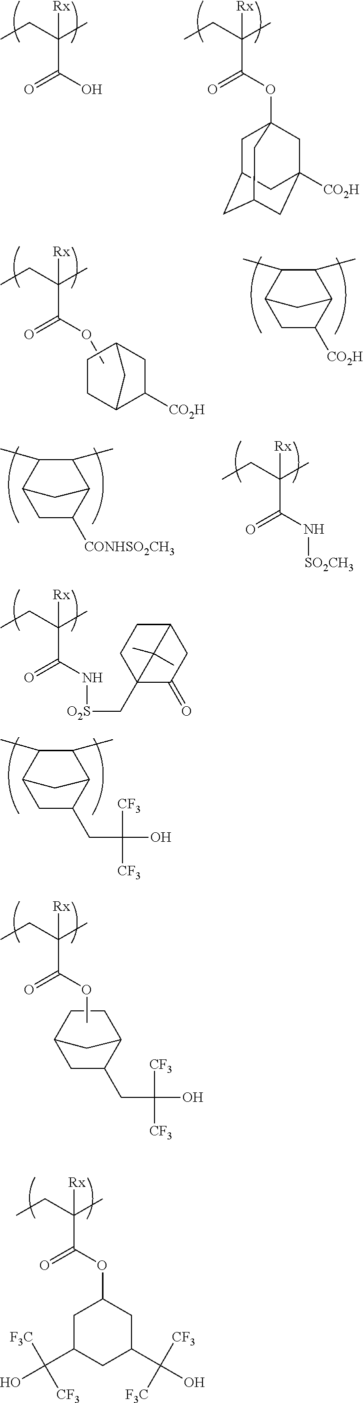

The resin (B1) for use in the present invention may further contain a repeating unit having a polar group-free alicyclic hydrocarbon structure and not exhibiting acid decomposability. Such a repeating unit includes a repeating unit represented by formula (IV):

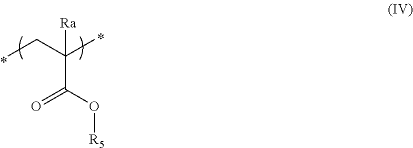

In formula (IV), R₅ represents a hydrocarbon group having at least one cyclic structure and having no polar group (e.g., hydroxyl group, cyano group).

Ra represents a hydrogen atom, an alkyl group or a —CH₂—O—Ra₂ group, wherein Ra₂ represents a hydrogen atom, an alkyl group or an acyl group. Ra is preferably a hydrogen atom, a methyl group, a hydroxymethyl group or a trifluoromethyl group, more preferably a hydrogen atom or a methyl group.

The cyclic structure contained in R₅ includes a monocyclic hydrocarbon group and a polycyclic hydrocarbon group. Examples of the monocyclic hydrocarbon group include a cycloalkyl group having a carbon number of 3 to 12, such as cyclopentyl group, cyclohexyl group, cycloheptyl group and cyclooctyl group, and a cycloalkenyl group having a carbon number of 3 to 12, such as cyclohexenyl group. The monocyclic hydrocarbon group is preferably a monocyclic hydrocarbon group having a carbon number of 3 to 7, more preferably a cyclopentyl group or a cyclohexyl group.

The polycyclic hydrocarbon group includes a ring gathered hydrocarbon group and a crosslinked cyclic hydrocarbon group. Examples of the ring gathered hydrocarbon group include a bicyclohexyl group and a perhydronaphthalenyl group. Examples of the crosslinked cyclic hydrocarbon ring include a bicyclic hydrocarbon ring such as pinane ring, bornane ring, norpinane ring, norbornane ring and bicyclooctane ring (e.g., bicyclo[2.2.2]octane ring, bicyclo[3.2.1]octane ring), a tricyclic hydrocarbon ring such as homobledane ring, adamantane ring, tricyclo[5.2.1.0^{2,6}]decane ring and tricyclo[4.3.1.1^{2,5}]undecane ring, and a tetracyclic hydrocarbon ring such as tetracyclo[4.4.0.1^{2,5}.1^{7,10}]dodecane ring and perhydro-1,4-methano-5,8-methanonaphthalene ring. The crosslinked cyclic hydrocarbon ring also includes a condensed cyclic hydrocarbon ring, for example, a condensed ring formed by condensing a plurality of 5- to 8-membered cycloalkane rings, such as perhydronaphthalene (decalin) ring, perhydroanthracene ring, perhydrophenathrene ring, perhydroacenaphthene ring, perhydrofluorene ring, perhydroindene ring and perhydrophenalene ring.

Preferred examples of the crosslinked cyclic hydrocarbon ring include a norbornyl group, an adamantyl group, a bicyclooctanyl group and a tricycle[5,2,1,0^{2,6}]decanyl group. Of these crosslinked cyclic hydrocarbon rings, a norbornyl group and an adamantyl group are more preferred.

These alicyclic hydrocarbon groups may have a substituent, and preferred examples of the substituent include a halogen atom, an alkyl group, a hydroxyl group protected by a protective group, and an amino group protected by a protective group. The halogen atom is preferably bromine atom, chlorine atom or fluorine atom, and the alkyl group is preferably a methyl group, an ethyl group, a butyl group or a tert-butyl group. This alkyl group may further have a substituent, and the substituent which the alkyl group may further have includes a halogen atom, an alkyl group, a hydroxyl group protected by a protective group, and an amino group protected by a protective group.

Examples of the protective group include an alkyl group, a cycloalkyl group, an aralkyl group, a substituted methyl group, a substituted ethyl group, an alkoxycarbonyl group and an aralkyloxycarbonyl group. The alkyl group is preferably an alkyl group having a carbon number of 1 to 4; the substituted methyl group is preferably a methoxymethyl group, a methoxythiomethyl group, a benzyloxymethyl group, a tert-butoxymethyl group or a 2-methoxyethoxymethyl group; the substituted ethyl group is preferably a 1-ethoxyethyl group or a 1-methyl-1-methoxyethyl group; the acyl group is preferably an aliphatic acyl group having a carbon number of 1 to 6, such as formyl group, acetyl group, propionyl group, butyryl group, isobutyryl group, valeryl group and pivaloyl group; and the alkoxycarbonyl group is preferably an alkoxycarbonyl group having a carbon number of 2 to 4.

The content of the repeating unit having a polar group-free alicyclic hydrocarbon structure and not exhibiting acid decomposability is preferably from 0 to 40 mol %, more preferably from 0 to 20 mol %, based on all repeating units in the resin (B1).

Specific examples of the repeating unit having a polar group-free alicyclic hydrocarbon structure and not exhibiting acid decomposability are illustrated below, but the present invention is not limited thereto. In the formulae, Ra represents H, CH₃, CH₂OH or CF₃.

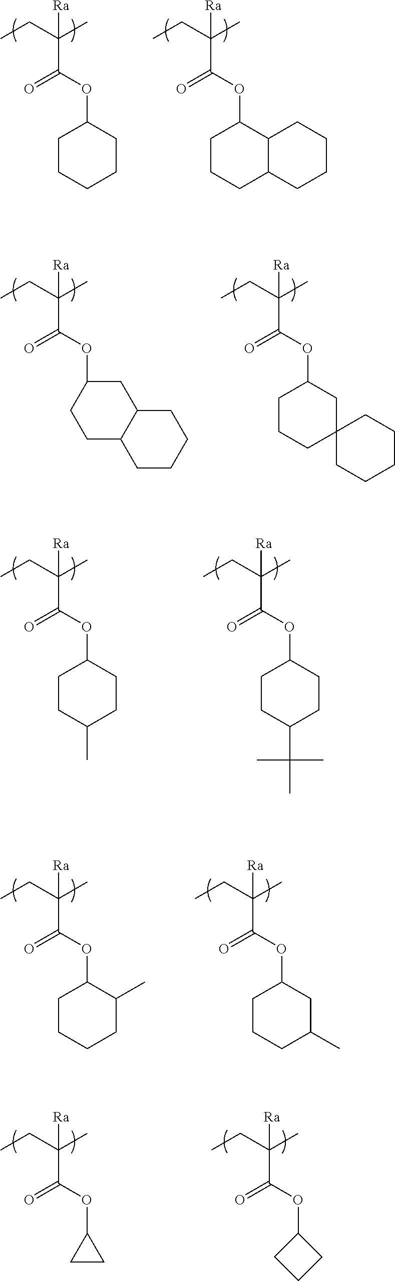

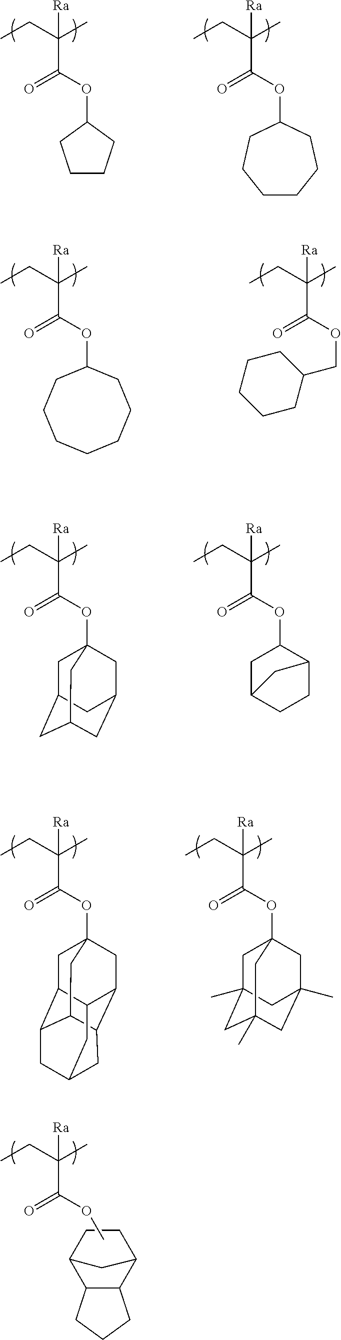

The resin (B1) for use in the composition of the present invention may contain, in addition to the above-described repeating structural units, various repeating structural units for the purpose of controlling the dry etching resistance, suitability for standard developer, adherence to substrate, resist profile and properties generally required of a resist, such as resolution, heat resistance and sensitivity.

Examples of such a repeating structural unit include, but are not limited to, repeating structural units corresponding to the monomers described below.

Thanks to such a repeating structural unit, the performance required of the resin for use in the composition of the present invention, particularly

(1) solubility in the coating solvent,

(2) film-forming property (glass transition point),

(3) alkali developability,

(4) film loss (selection of hydrophilic, hydrophobic or alkali-soluble group),

(5) adherence of unexposed area to substrate,

(6) dry etching resistance, and the like can be subtly controlled.

Examples of the monomer include a compound having one addition-polymerizable unsaturated bond selected from acrylic acid esters, methacrylic acid esters, acrylamides, methacrylamides, allyl compounds, vinyl ethers and vinyl esters.

Other than these, an addition-polymerizable unsaturated compound copolymerizable with the monomers corresponding to the above-described various repeating structural units may be copolymerized.

In the resin (B1) for use in the composition of the present invention, the molar ratio of respective repeating structural units contained is appropriately set to control the dry etching resistance of resist, suitability for standard developer, adherence to substrate, resist profile and performances generally required of a resist, such as resolution, heat resistance and sensitivity.

In the case where the composition of the present invention is used for ArF exposure, the resin (B1) for use in the composition of the present invention preferably has substantially no aromatic group (specifically, in the resin, the ratio of an aromatic group-containing repeating is preferably 5 mol % or less, more preferably 3 mol % or less, and ideally 0 mol %) in view of transparency to ArF light, and the resin (B1) preferably has a monocyclic or polycyclic alicyclic hydrocarbon structure.

Also, the resin (B1) preferably contains no fluorine atom and no silicon atom in view of compatibility with the later-described hydrophobic resin (C).

The resin (B1) for use in the composition of the present invention is preferably a resin where all repeating units are composed of a (meth)acrylate-based repeating unit. In this case, all repeating units may be a methacrylate-based repeating unit, all repeating units may be an acrylate-based repeating unit, or all repeating units may be composed of a methacrylate-based repeating unit and an acrylate-based repeating unit, but the content of the acrylate-based repeating unit is preferably 50 mol % or less based on all repeating units. It is also preferred that the resin is a copolymerized polymer containing from 20 to 50 mol % of an acid decomposable group-containing (meth)acrylate-based repeating unit, from 20 to 50 mol % of a lactone group-containing (meth)acrylate-based repeating unit, from 5 to 30 mol % of a (meth)acrylate-based repeating unit having an alicyclic hydrocarbon structure substituted with a hydroxyl group or a cyano group, and from 0 to 20 mol % of other (meth)acrylate-based repeating units.

In the case of irradiating the composition of the present invention with KrF excimer laser light, electron beam, X-ray or high-energy beam at a wavelength of 50 nm or less (e.g., EUV), the resin (B1) preferably further contains a hydroxystyrene-based repeating unit, more preferably a hydroxystyrene-based repeating unit, a hydroxystyrene-based repeating unit protected by an acid-decomposable group, and an acid-decomposable repeating unit such as tertiary alkyl(meth)acrylate.

Preferred examples of the hydroxystyrene-based repeating unit having an acid-decomposable group include repeating units composed of a tert-butoxycarbonyloxystyrene, a 1-alkoxyethoxystyrene or a tertiary alkyl(meth)acrylate. Repeating units composed of a 2-alkyl-2-adamantyl(meth)acrylate or a dialkyl(1-adamantyl)methyl(meth)acrylate are more preferred.

The resin (B1) for use in the present invention can be synthesized by an ordinary method (for example, radical polymerization).

Examples of the general synthesis method include a batch polymerization method of dissolving monomer species and an initiator in a solvent and heating the solution, thereby effecting the polymerization, and a dropping polymerization method of adding dropwise a solution containing monomer species and an initiator to a heated solvent over 1 to 10 hours. A dropping polymerization method is preferred. Examples of the reaction solvent include ethers such as tetrahydrofuran, 1,4-dioxane and diisopropyl ether, ketones such as methyl ethyl ketone and methyl isobutyl ketone, an ester solvent such as ethyl acetate, an amide solvent such as dimethylformamide and dimethylacetamide, and the later-described solvent capable of dissolving the composition of the present invention, such as propylene glycol monomethyl ether acetate, propylene glycol monomethyl ether and cyclohexanone. The polymerization is more preferably performed using the same solvent as the solvent used in the positive resist composition of the present invention. By the use of the same solvent, production of particles during storage can be suppressed.

The polymerization reaction is preferably performed in an inert gas atmosphere such as nitrogen or argon. As for the polymerization initiator, the polymerization is started using a commercially available radical initiator (e.g., azo-based initiator, peroxide). The radical initiator is preferably an azo-based initiator, and an azo-based initiator having an ester group, a cyano group or a carboxyl group is preferred. Preferred examples of the initiator include azobisisobutyronitrile, azobisdimethylvaleronitrile and dimethyl 2,2′-azobis(2-methylpropionate). The initiator is added additionally or in parts, if desired. After the completion of reaction, the reaction product is pored in a solvent, and the desired polymer is collected by a method such as powder or solid recovery. The concentration at the reaction is from 5 to 50 mass %, preferably from 30 to 50 mass %, and the reaction temperature is usually from 10 to 150° C., preferably from 30 to 120° C., more preferably from 60 to 100° C.

After the completion of reaction, the reaction product is left standing to cool to room temperature and purified. The purification may be performed by a normal method, for example, a liquid-liquid extraction method of combining water washing or an appropriate solvent to remove residual monomers or oligomer components; a purification method in a solution sate, such as ultrafiltration of extracting and removing only those having a molecular weight lower than a specific molecular weight; a reprecipitation method of adding dropwise the resin solution in a poor solvent to solidify the resin in the poor solvent and thereby remove residual monomers and the like; or a purification method in a solid state, such as washing of the resin slurry with a poor solvent after separation by filtration. For example, the resin is precipitated as a solid through contact with a solvent in which the resin is sparingly soluble or insoluble (poor solvent) and which is in a volumetric amount of 10 times or less, preferably from 10 to 5 times, the reaction solution.

The solvent used at the operation of precipitation or reprecipitation from the polymer solution (precipitation or reprecipitation solvent) may be sufficient if it is a poor solvent to the polymer, and the solvent which can be used may be appropriately selected from a hydrocarbon, a halogenated hydrocarbon, a nitro compound, an ether, a ketone, an ester, a carbonate, an alcohol, a carboxylic acid, water, a mixed solvent containing such a solvent, and the like, according to the kind of the polymer. Among these solvents, a solvent containing at least an alcohol (particularly, methanol or the like) or water is preferred as the precipitation or reprecipitation solvent.

The amount of the precipitation or reprecipitation solvent used may be appropriately selected by taking into consideration the efficiency, yield and the like, but in general, the amount used is from 100 to 10,000 parts by mass, preferably from 200 to 2,000 parts by mass, more preferably from 300 to 1,000 parts by mass, per 100 parts by mass of the polymer solution.

The temperature at the precipitation or reprecipitation may be appropriately selected by taking into consideration the efficiency or operability but is usually on the order of 0 to 50° C., preferably in the vicinity of room temperature (for example, approximately from 20 to 35° C.). The precipitation or reprecipitation operation may be performed using a commonly employed mixing vessel such as stirring tank by a known method such as batch system and continuous system.

The precipitated or reprecipitated polymer is usually subjected to commonly employed solid-liquid separation such as filtration and centrifugation, then dried and used. The filtration is performed using a solvent-resistant filter element preferably under pressure. The drying is performed under atmospheric pressure or reduced pressure (preferably under reduced pressure) at a temperature of approximately from 30 to 100° C., preferably on the order of 30 to 50° C.

Incidentally, after the resin is once precipitated and separated, the resin may be again dissolved in a solvent and then put into contact with a solvent in which the resin is sparingly soluble or insoluble. That is, there may be used a method comprising, after the completion of radical polymerization reaction, bringing the polymer into contact with a solvent in which the polymer is sparingly soluble or insoluble, to precipitate a resin (step a), separating the resin from the solution (step b), anew dissolving the resin in a solvent to prepare a resin solution A (step c), bringing the resin solution A into contact with a solvent in which the resin is sparingly soluble or insoluble and which is in a volumetric amount of less than 10 times (preferably 5 times or less) the resin solution A, to precipitate a resin solid (step d), and separating the precipitated resin (step e).

The weight average molecular weight of the resin (B1) for use in the present invention is preferably from 1,000 to 200,000, more preferably from 2,000 to 20,000, still more preferably from 3,000 to 15,000, yet still more preferably from 3,000 to 10,000, in terms of polystyrene by the GPC method. When the weight average molecular weight is from 1,000 to 200,000, decrease in the heat resistance, dry etching resistance and developability can be avoided and the film-forming property can be prevented from deteriorating due to increase in the viscosity.

The polydispersity (molecular weight distribution) is usually from 1 to 3, preferably from 1 to 2.6, more preferably from 1 to 2, still more preferably from 1.4 to 2.0. As the molecular weight distribution is smaller, the resolution and resist profile are more excellent, the side wall of the resist pattern is smoother, and the roughness is more improved.

In the present invention, the content of the resin (B1) in the entire composition (when the composition contains the later-described resin (B2), in terms of the total amount) is preferably from 30 to 99 mass %, more preferably from 60 to 90 mass %, based on the entire solid content. Also, as to the resin for use in the present invention, one kind may be used or a plurality of kinds may be used in combination.

[2′] (B2) Resin not having a group capable of decomposing by the action of an acid

The photosensitive composition of the present invention may contain (B2) a resin not having a group capable of decomposing by the action of an acid.

The expression “not having a group capable of decomposing by the action of an acid” means to lack of or be extremely low in the decomposability by the action of an acid in the image forming process where the photosensitive composition of the present invention is usually used, and have substantially no group contributing to the image formation by the decomposition of an acid (for example, the acid-decomposable group in the resin (B1)). Such a resin includes a resin having an alkali-soluble group and a resin having a group that decomposes by the action of an alkali to increase the solubility in an alkali developer.

The resin (B2) is preferably a resin containing at least one repeating unit derived from a (meth)acrylic acid derivative and/or an alicyclic olefin derivative.

Examples of the alkali-soluble group contained in the resin (B2) are the same as those described in the acid-decomposable group of the resin (B1). For example, a carboxyl group, a phenolic hydroxyl group, an aliphatic hydroxyl group substituted with an electron-withdrawing group at the 1- or 2-position, an electron withdrawing group-substituted amino group (such as sulfonamide group, sulfonimide group and bis-sulfonylimide group), and an electron withdrawing group-substituted methylene or methine group (such as methylene or methine group substituted with at least two members selected from ketone groups and ester groups) are preferred.

Examples of the electron-withdrawing group include a halogen atom (preferably fluorine atom), a cyano group, an oxy group, a carbonyl group, a carbonyloxy group, an oxycarbonyl group, a nitrile group, a nitro group, a sulfonyl group, a sulfinyl group, and a combination thereof.

The group (b) capable of decomposing by the action of an alkali to increase the solubility in an alkali developer, contained in the resin (B2), is preferably a lactone group or an acid anhydride group, more preferably a lactone group. Specific examples of the repeating unit containing (b) a group capable of decomposing by the action of an alkali developer to increase the solubility in an alkali developer are the same as those of the repeating unit having a lactone group in the resin (B1).

The resin (B2) may contain a repeating unit having a functional group other than the functional group above. As for the repeating unit having the other functional group, an appropriate functional group can be introduced by taking into consideration the dry etching resistance hydrophilicity/hydrophobicity, interaction and the like.

Examples of the other repeating unit include a repeating unit having a polar functional group such as hydroxyl group, cyano group, carbonyl group and ester group, a repeating unit having a monocyclic or polycyclic hydrocarbon structure, a repeating unit having a silicon atom, a halogen atom or a fluoroalkyl group, and a repeating unit having a plurality of these functional groups.

Specific examples and preferred compositional ratio of various repeating units in the resin (B2) are the same as those in the resin (B1).

Specific preferred examples of the resin (B2) are illustrated below.

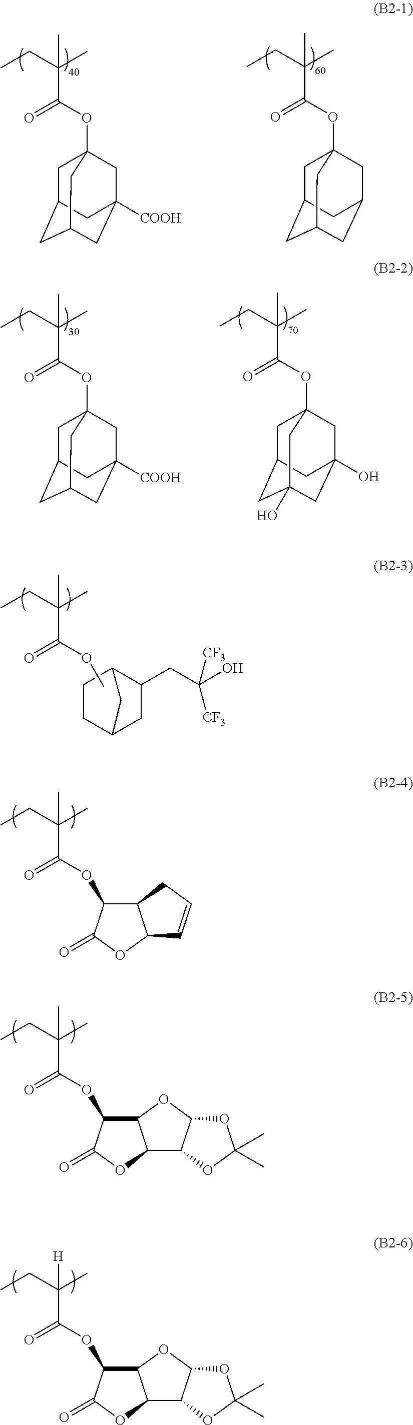

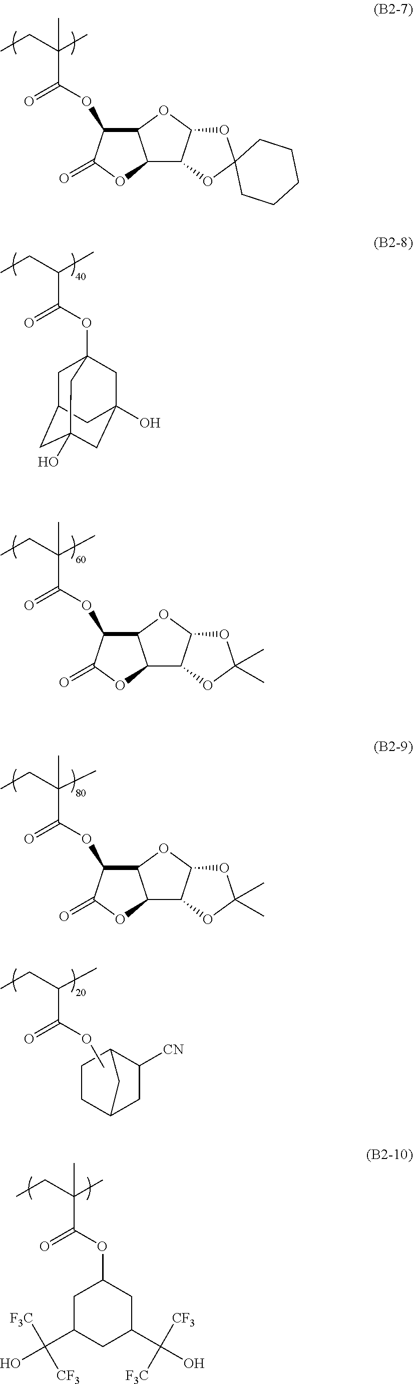

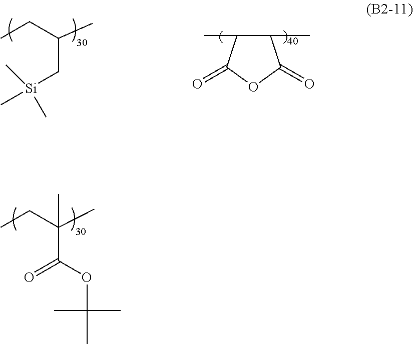

The amount of the resin (B2) added is from 0 to 30 mass % based on the resin (B1) and is preferably from 0 to 20 mass %, more preferably from 0 to 15 mass %, based on the resin (B1).

Preferred ranges of the molecular weight and polydispersity of the resin (B2) are the same as those of the resin (B1).

As for the resin (B2), a commercial product may be used, or a resin synthesized in the same manner as the resin (B1) may be used.

[3] (C) Compound capable of generating an acid upon irradiation with an actinic ray or radiation

The composition of the present invention preferably contains a compound capable of generating an acid upon irradiation with an actinic ray or radiation (hereinafter, sometimes referred to as an “acid generator”).

The acid generator is not particularly limited as long as it is a known acid generator, but preferred acid generators include the compounds represented by the following formulae (ZI), (ZII) and (ZIII):

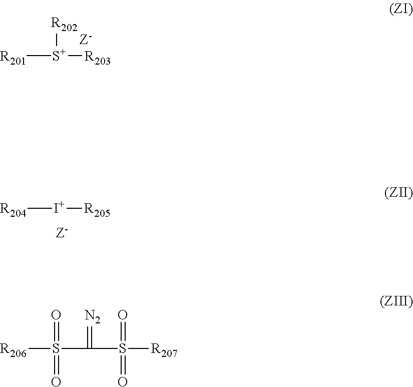

In formula (ZI), each of R₂₀₁, R₂₀₂ and R₂₀₃ independently represents an organic group.

The carbon number of the organic group as R₂₀₁, R₂₀₂ and R₂₀₃ is generally from 1 to 30, preferably from 1 to 20.

Two members out of R₂₀₁ to R₂₀₃ may combine to form a ring structure, and the ring may contain an oxygen atom, a sulfur atom, an ester bond, an amide bond or a carbonyl group. The group formed by combining two members out of R₂₀₁ to R₂₀₃ includes an alkylene group (e.g., butylene, pentylene).

Z⁻ represents a non-nucleophilic anion (an anion having an extremely low ability of causing a nucleophilic reaction).

Examples of Z⁻ include a sulfonate anion (e.g., aliphatic sulfonate anion, aromatic sulfonate anion, camphorsulfonate anion), a carboxylate anion (e.g., aliphatic carboxylate anion, aromatic carboxylate anion, aralkylcarboxylate anion), a sulfonylimide anion, a bis(alkylsulfonyl)imide anion and a tris(alkylsulfonyl)methide anion.

The aliphatic moiety in the aliphatic sulfonate anion and aliphatic carboxylate may be an alkyl group or a cycloalkyl group but is preferably a linear or branched alkyl group having a carbon number of 1 to 30 or a cycloalkyl group having a carbon number of 3 to 30.

The aromatic group in the aromatic sulfonate anion and aromatic carboxylate anion is preferably an aryl group having a carbon number of 6 to 14, and examples thereof include a phenyl group, a tolyl group and a naphthyl group.

The alkyl group, cycloalkyl group and aryl group above may have a substituent. Specific examples thereof include a nitro group, a halogen atom such as fluorine atom, a carboxyl group, a hydroxyl group, an amino group, a cyano group, an alkoxy group (preferably having a carbon number of 1 to 15), a cycloalkyl group (preferably having a carbon number of 3 to 15), an aryl group (preferably having a carbon number of 6 to 14), an alkoxycarbonyl group (preferably having a carbon number of 2 to 7), an acyl group (preferably having a carbon number of 2 to 12), an alkoxycarbonyloxy group (preferably having a carbon number of 2 to 7), an alkylthio group (preferably having a carbon number of 1 to 15), an alkylsulfonyl group (preferably having a carbon number of 1 to 15), an alkyliminosulfonyl group (preferably having a carbon number of 2 to 15), an aryloxysulfonyl group (preferably having a carbon number of 6 to 20), an alkylaryloxysulfonyl group (preferably having a carbon number of 7 to 20), a cycloalkylaryloxysulfonyl group (preferably having a carbon number of 10 to 20), an alkyloxyalkyloxy group (preferably having a carbon number of 5 to 20), and a cycloalkylalkyloxyalkyloxy group (preferably having a carbon number of 8 to 20). As for the aryl group and ring structure in each group, examples of the substituent further include an alkyl group (preferably having a carbon number of 1 to 15).

The aralkyl group in the aralkylcarboxylate anion is preferably an aralkyl group having a carbon number of 7 to 12, and examples thereof include a benzyl group, a phenethyl group, a naphthylmethyl group, a naphthylethyl group and a naphthylbutyl group.

Examples of the sulfonylimide anion include saccharin anion.

The alkyl group in the bis(alkylsulfonyl)imide anion and tris(alkylsulfonyl)methide anion is preferably an alkyl group having a carbon number of 1 to 5. Examples of the substituent of such an alkyl group include a halogen atom, a halogen atom-substituted alkyl group, an alkoxy group, an alkylthio group, an alkyloxysulfonyl group, an aryloxysulfonyl group and a cycloalkylaryloxysulfonyl group, with a fluorine atom and a fluorine atom-substituted alkyl group being preferred.

Other examples of Z⁻ include fluorinated phosphorus (e.g., PF₆⁻), fluorinated boron (e.g., BF₄⁻) and fluorinated antimony (e.g., SbF₆⁻).

Z⁻ is preferably an aliphatic sulfonate anion substituted with a fluorine atom at least on the α-position of the sulfonic acid, an aromatic sulfonate anion substituted with a fluorine atom or a fluorine atom-containing group, a bis(alkylsulfonyl)imide anion with the alkyl group being substituted with a fluorine atom, or a tris(alkylsulfonyl)methide anion with the alkyl group being substituted with a fluorine atom. The non-nucleophilic anion is more preferably a perfluoroaliphatic sulfonate anion (more preferably having a carbon number of 4 to 8) or a benzenesulfonate anion having a fluorine atom, still more preferably nonafluorobutanesulfonate anion, perfluorooctanesulfonate anion, pentafluorobenzenesulfonate anion or 3,5-bis(trifluoromethyl)benzenesulfonate anion.

As regards the acid strength, the pKa of the acid generated is preferably −1 or less in view of enhancing the sensitivity.

Examples of the organic group of R₂₀₁, R₂₀₂ and R₂₀₃ include an aryl group (preferably having a carbon number of 6 to 15), a linear or branched alkyl group (preferably having a carbon number of 1 to 10), and a cycloalkyl group (preferably having a carbon number of 3 to 15).

At least one of three members R₂₀₁, R₂₀₂ and R₂₀₃ is preferably an aryl group, and it is more preferred that all of these three members are an aryl group. The aryl group may be a heteroaryl group such as indole residue and pyrrole residue, other than a phenyl group or a naphthyl group. These aryl groups may further have a substituent, and examples of the substituent include, but are not limited to, a nitro group, a halogen atom such as fluorine atom, a carboxyl group, a hydroxyl group, an amino group, a cyano group, an alkoxy group (preferably having a carbon number of 1 to 15), a cycloalkyl group (preferably having a carbon number of 3 to 15), an aryl group (preferably having a carbon number of 6 to 14), an alkoxycarbonyl group (preferably having a carbon number of 2 to 7), an acyl group (preferably having a carbon number of 2 to 12), and an alkoxycarbonyloxy group (preferably having a carbon number of 2 to 7).

Also, two members selected from R₂₀₁, R₂₀₂ and R₂₀₃ may combine through a single bond or a linking group. Examples of the linking group include, but are not limited to, an alkylene group (preferably having a carbon number of 1 to 3), —O—, —S—, —CO— and —SO₂—.

Preferred structures when at least one of R₂₀₁, R₂₀₂ and R₂₀₃ is not an aryl group include cation structures such as compounds described in JP-A-2004-233661, paragraphs 0046 and 0047, and JP-A-2003-35948, paragraphs 0040 to 0046, Compounds (1-1) to (1-70) illustrated in U.S. Patent Application Publication 2003/0224288A1, and Compounds (IA-1) to (IA-54) and (IB-1) to (IB-24) illustrated in U.S. Patent Application Publication 2003/0077540A1.

In particular, when at least one of R₂₀₁, R₂₀₂ and R₂₀₃ is not an aryl group, the following embodiment (1) or (2) is more preferred.

(1) An embodiment where at least one of R₂₀₁, R₂₀₂ and R₂₀₃ is a group represented by Ar—CO—X— and the remaining groups are a linear or branched alkyl group or a cycloalkyl group; in this case, when the number of the remaining groups is 2, two linear or branched alkyl groups or cycloalkyl groups may combine with each other to form a ring.

Here, Ar represents an aryl group which may have a substituent. The aryl group is specifically the same as the aryl group of R₂₀₁, R₂₀₂ and R₂₀₃ and is preferably a phenyl group which may have a substituent.

X represents an alkylene group. The alkylene group is specifically an alkylene group having a carbon number of 1 to 6 and is preferably a linear or branched alkylene group having a carbon number of 1 to 3.

The remaining linear or branched alkyl group or cycloalkyl group preferably has a carbon number of 1 to 6. This atomic group may further have a substituent. Also, when the number of remaining groups is 2, these groups are preferably combined with each other to form a ring structure (preferably a 5- to 7-membered ring).

(2) An embodiment where one or two of R₂₀₁, R₂₀₂ and R₂₀₃ is(are) an aryl group which may have a substituent, and the remaining group(s) is(are) a linear or branched alkyl group or a cycloalkyl group.

At this time, the aryl group is specifically the same as the aryl group of R₂₀₁, R₂₀₂ and R₂₀₃ and is preferably a phenyl group or a naphthyl group. Also, the aryl group preferably has, as a substituent, any of a hydroxyl group, an alkoxy group and an alkyl group. The substituent is preferably an alkoxy group having a carbon number of 1 to 12, more preferably an alkoxy group having a carbon number of 1 to 6.

The remaining linear or branched alkyl group or cycloalkyl group preferably has a carbon number of 1 to 6. This atomic group may further have a substituent. Also, when the number of remaining groups is 2, these two groups may combine with each other to form a ring structure.

In formulae (ZII) and (ZIII), each of R₂₀₄ to R₂₀₇ independently represents an aryl group, an alkyl group or a cycloalkyl group.

The aryl group, alkyl group and cycloalkyl group of R₂₀₄ to R₂₀₇ are the same as the aryl group, alkyl group and cycloalkyl group of R₂₀₁ to R₂₀₃ in the compound (ZI).

The aryl group, alkyl group and cycloalkyl group of R₂₀₄ to R₂₀₇ may have a substituent. Examples of the substituent include those which the aryl group, alkyl group and cycloalkyl group of R₂₀₁ to R₂₀₃ in the compound (ZI) may have.

Z⁻ represents a non-nucleophilic anion, and examples thereof are the same as those of the non-nucleophilic anion of Z⁻ in formula (ZI).

Other examples of the acid generator include compounds represented by the following formulae (ZIV), (ZV) and (ZVI):

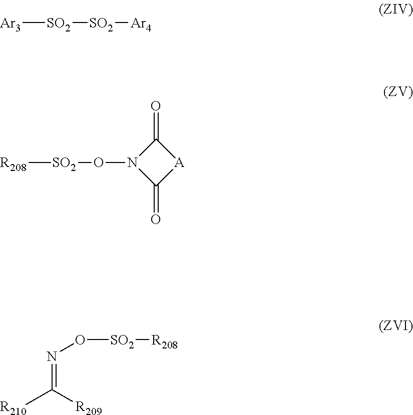

In formulae (ZIV) to (ZVI), each of Ar₃ and Ar₄ independently represents an aryl group.

Each of R₂₀₈, R₂₀₉ and R₂₁₀ independently represents an alkyl group, a cycloalkyl group or an aryl group.

A represents an alkylene group, an alkenylene group or an arylene group.

Specific examples of the aryl group of Ar₃, Ar₄, R₂₀₈, R₂₀₉ and R₂₁₀ are the same as those of the aryl group as R₂₀₁, to R₂₀₃ in formula (ZI).

Specific examples of the alkyl and cycloalkyl groups of R₂₀₈, R₂₀₉ and R₂₁₀ are the same as those of the alkyl and cycloalkyl groups of R₂₀₁ to R₂₀₃ in formula (ZI).

The alkylene group of A includes an alkylene group having a carbon number of 1 to 12 (e.g., methylene, ethylene, propylene, isopropylene, butylene, isobutylene), the alkenylene of A includes an alkenylene group having a carbon number of 2 to 12 (e.g., vinylene, propenylene, butenylene), and the arylene group of A includes an arylene group having a carbon number of 6 to 10 (e.g., phenylene, tolylene, naphthylene).

Out of the acid generators, particularly preferred examples are illustrated below.

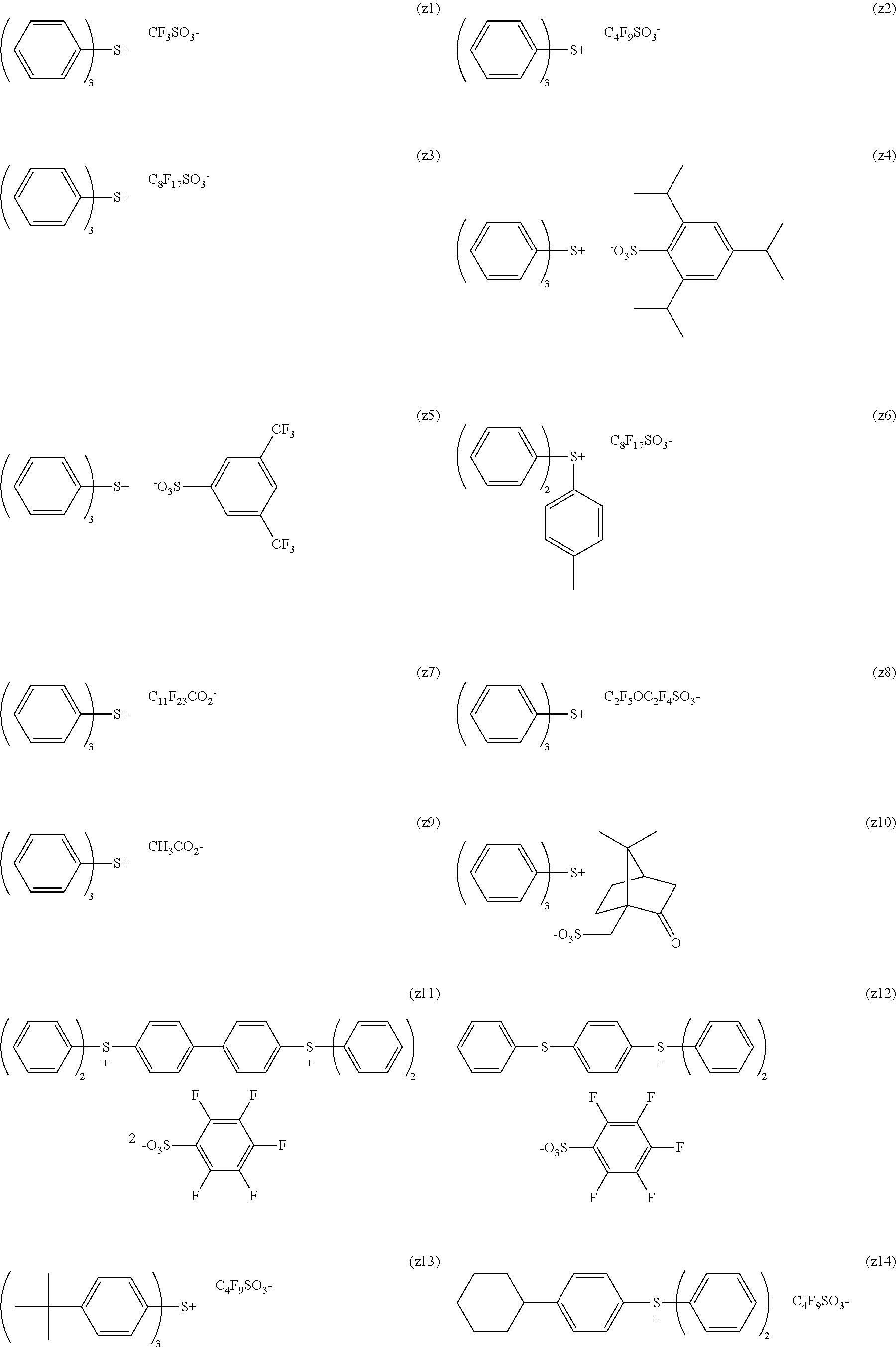

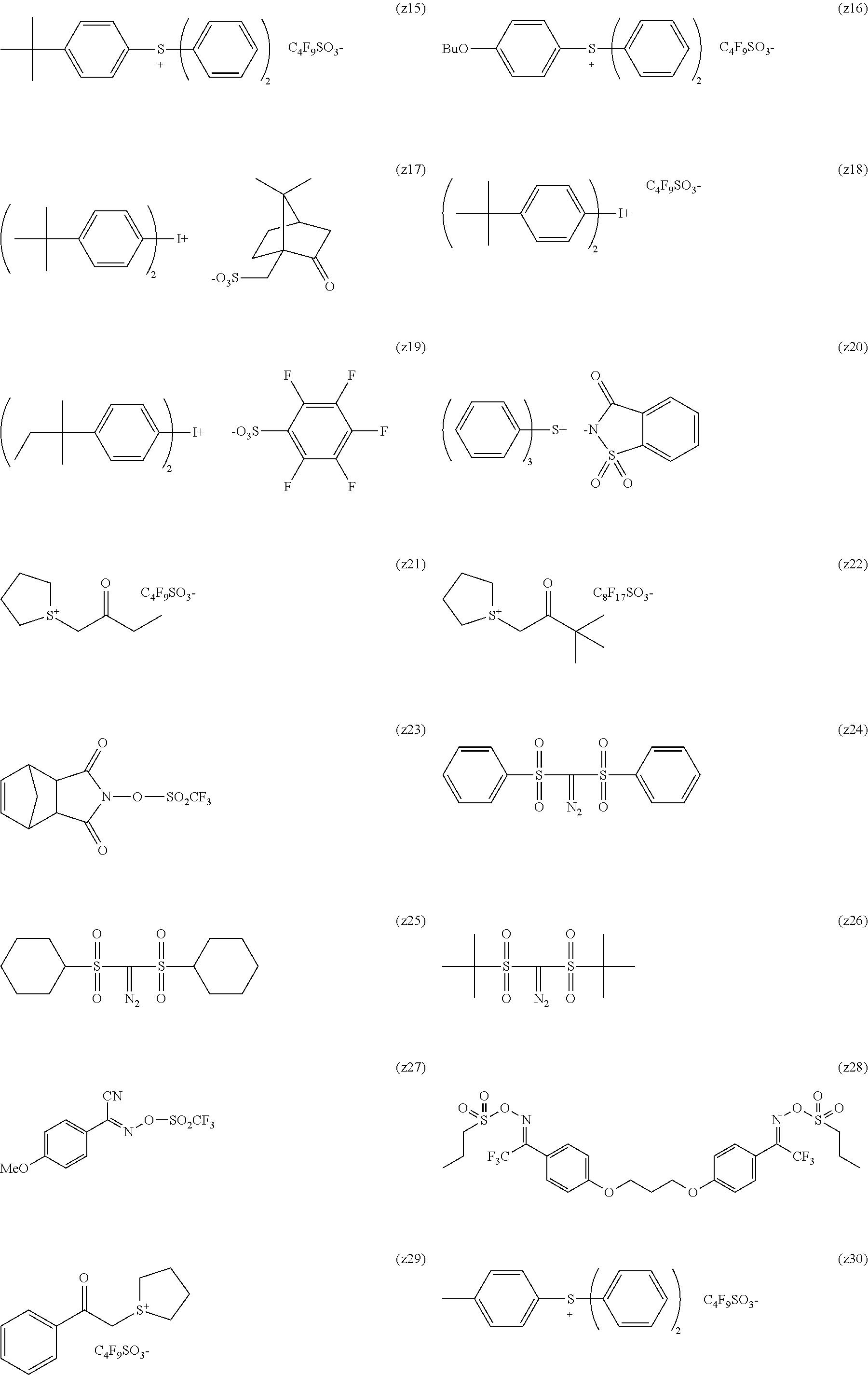

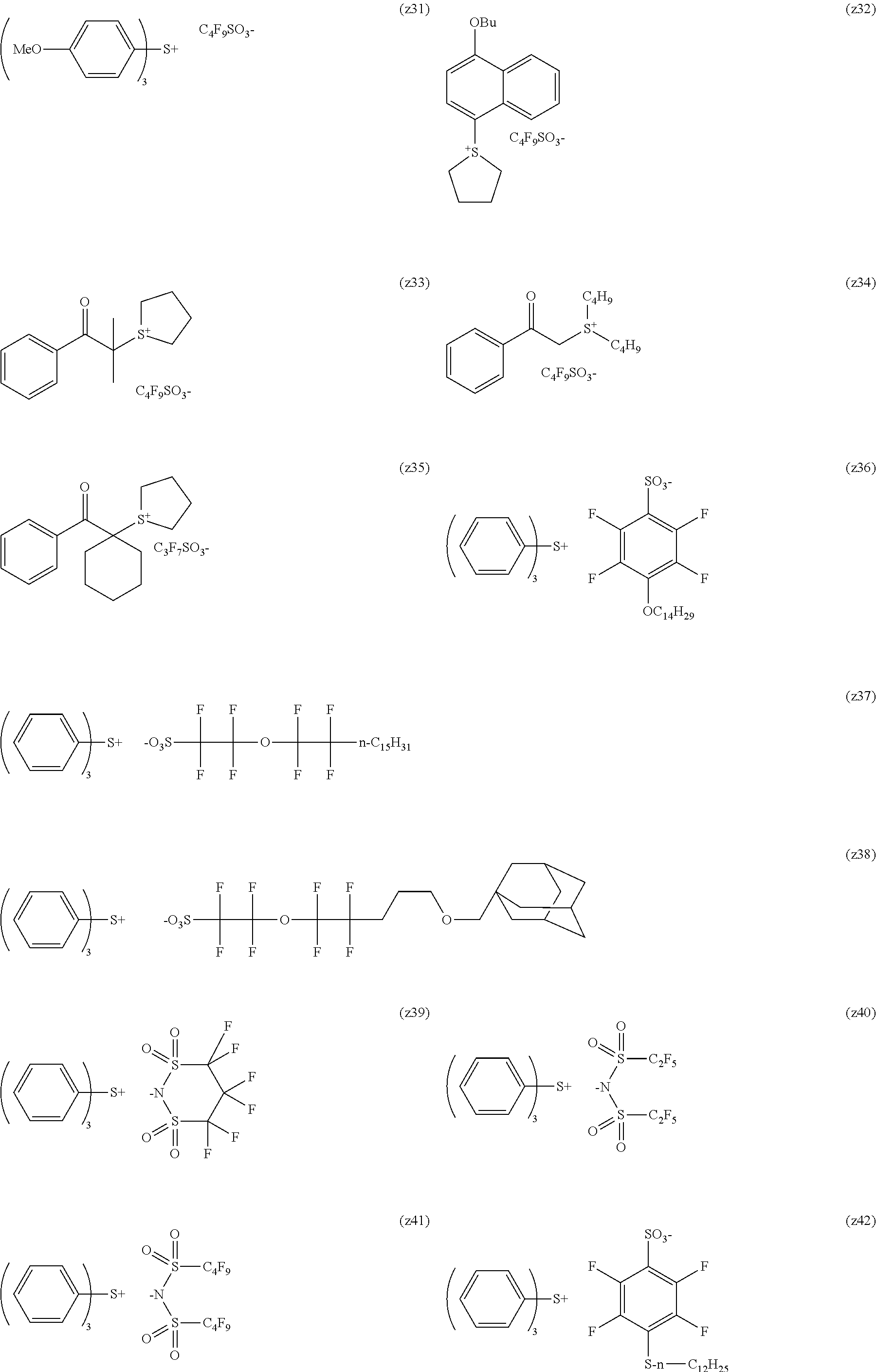

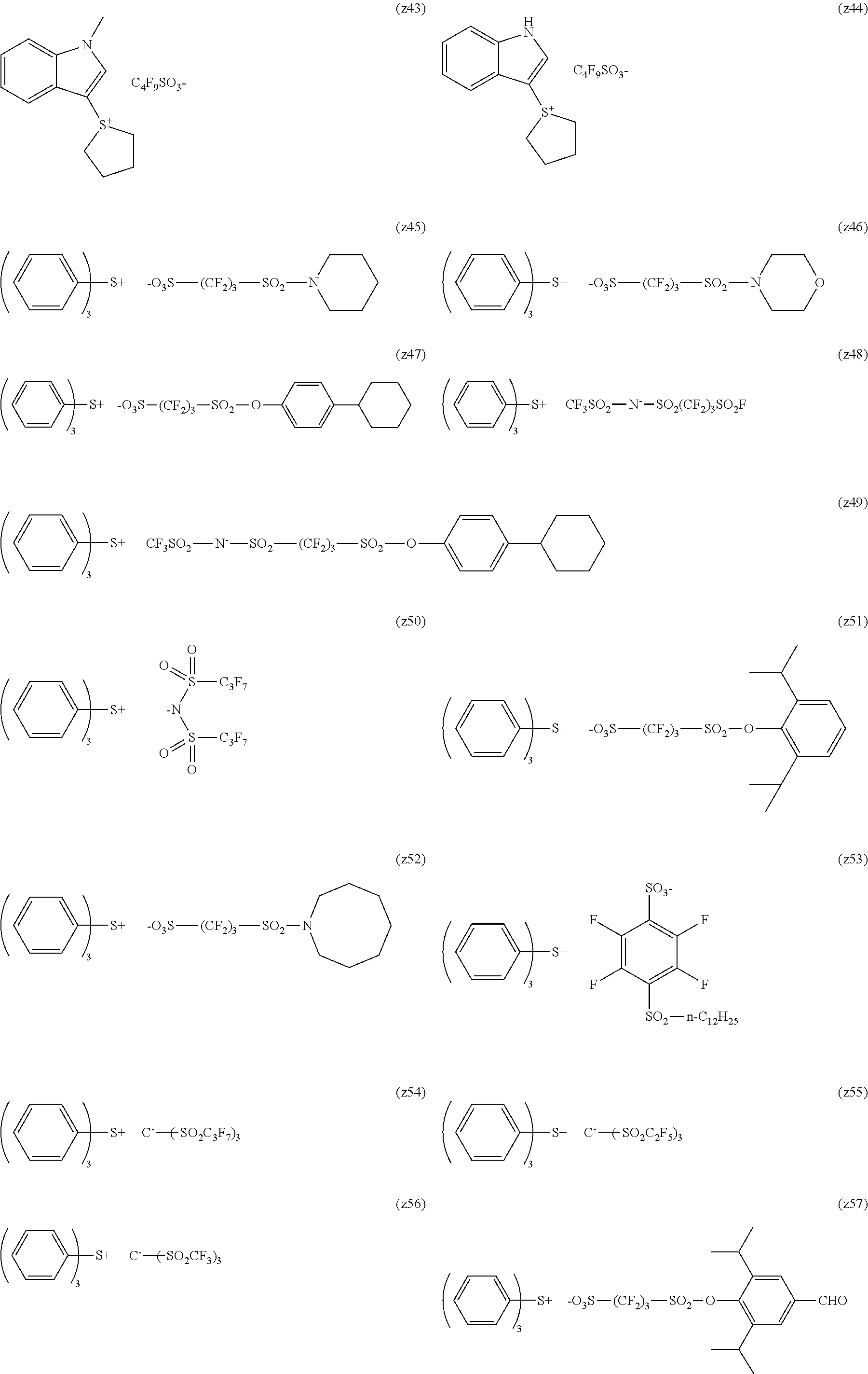

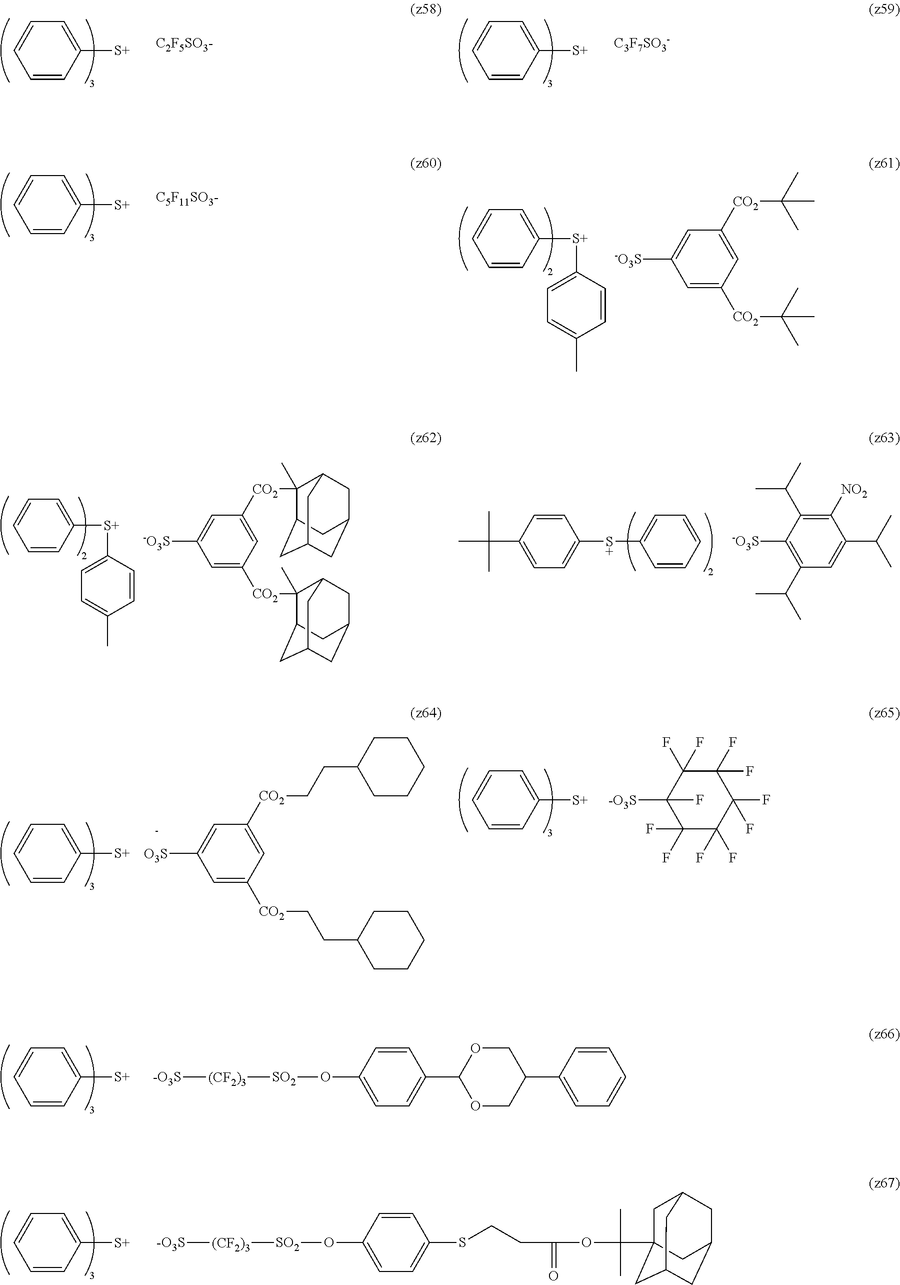

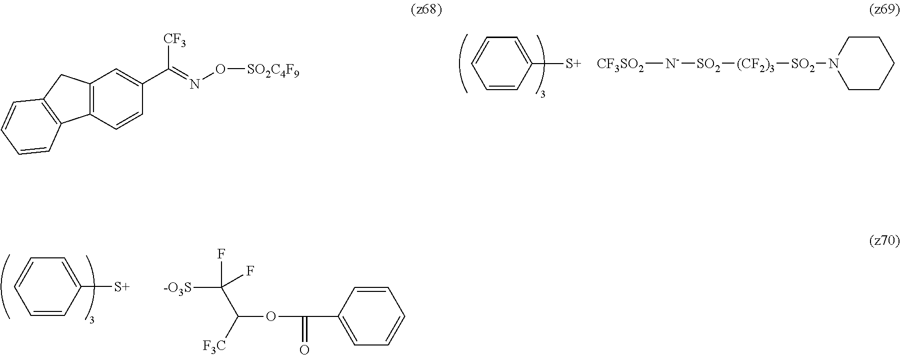

As for the acid generator, one kind may be used alone, or two or more kinds may be used in combination.

In the composition of the present invention, the cation structure of the acid generator is preferably the same as the cation structure of the compound (PA). Similarly to the compound (PA), cation structures represented by (ZI-1), (ZI-2) and (ZI-5) are preferred, and cation structures represented by (ZI-1) and (ZI-5) are more preferred.

The content of the acid generator in the composition is preferably from 0.1 to 30 mass %, more preferably from 3 to 20 mass %, still more preferably from 7 to 15 mass %, based on the entire solid content of the composition.

### [4] Hydrophobic Resin (HR)

In the composition of the present invention, a hydrophobic resin (HR) may be further added. The hydrophobic resin (HR) is unevenly distributed to the surface layer of the film and in the case of exposing a film composed of the composition of the present invention through an immersion medium, the film formed can be enhanced in the receding contact angle on the resist film surface for the liquid medium as well as in the followability of the immersion liquid. Thanks to the addition of a hydrophobic resin (HR), in particular, the receding contact angle on the film surface is enhanced. The receding contact angle of the film is preferably from 60 to 90°, more preferably 70° or more. The hydrophobic resin (HR) is, as described above, unevenly distributed to the interface but unlike a surfactant, need not have necessarily a hydrophilic group in the molecule and may not contribute to uniform mixing of polar/nonpolar substances.

The receding contact angle is a contact angle measured when a contact line recedes on the liquid droplet-substrate interface, and this is generally known to be useful in simulating the mobility of a liquid droplet in a dynamic state. In a simple manner, the receding contact angle can be defined as a contact angle upon receding of the liquid droplet interface when a liquid droplet ejected from a needle tip is landed on a substrate and then the liquid droplet is again suctioned into the needle. In general, the receding contact angle can be measured by a contact angle measuring method called an expansion-contraction method.

In the immersion exposure step, the immersion liquid must move on a wafer following the movement of an exposure head that is scanning the wafer at a high speed and forming an exposure pattern. Therefore, the contact angle of the immersion liquid with the resist film in a dynamic state is important, and the resist is required to have a performance of allowing a liquid droplet to follow the high-speed scanning of an exposure head with no remaining.

The hydrophobic resin (HR) preferably has at least either a fluorine atom or a silicon atom.

The fluorine atom or silicon atom in the hydrophobic resin (HR) may be present in the main chain of the resin or may be substituted on the side chain.

The hydrophobic resin (HR) is preferably a resin having, as the fluorine atom-containing partial structure, a fluorine atom-containing alkyl group, a fluorine atom-containing cycloalkyl group or a fluorine atom-containing aryl group.

The fluorine atom-containing alkyl group (preferably having a carbon number of 1 to 10, more preferably from 1 to 4) is a linear or branched alkyl group with at least one hydrogen atom being replaced by a fluorine atom and may further have other substituents.

The fluorine atom-containing cycloalkyl group is a monocyclic or polycyclic cycloalkyl group with at least one hydrogen atom being replaced by a fluorine atom and may further have other substituents.

The fluorine atom-containing aryl group includes an aryl group (e.g., phenyl, naphthyl) with at least one hydrogen atom being replaced by a fluorine atom and may further have other substituents.

Preferred examples of the fluorine atom-containing alkyl group, fluorine atom-containing cycloalkyl group and fluorine atom-containing aryl group include the groups represented by the following formulae (F2) to (F4), but the present invention is not limited thereto.

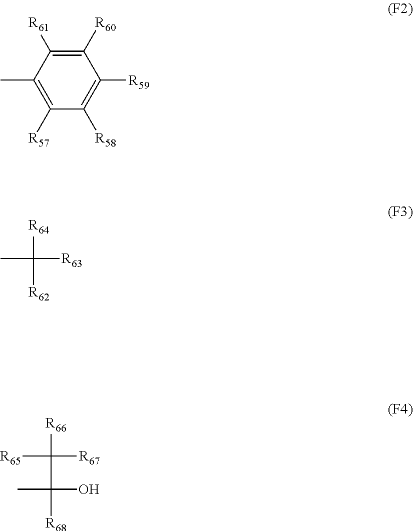

In formulae (F2) to (F4), each of R₅₇ to R₆₈ independently represents a hydrogen atom, a fluorine atom or an alkyl group, provided that at least one of R₅₇ to R₆₁, at least one of R₆₂ to R₆₄ and at least one of R₆₅ to R₆₈ are a fluorine atom or an alkyl group (preferably having a carbon number of 1 to 4) with at least one hydrogen atom being replaced by a fluorine atom. It is preferred that all of R₅₇ to R₆₁ and R₆₅ to R₆₇ are a fluorine atom. Each of R₆₂, R₆₃ and R₆₈ is preferably an alkyl group (preferably having a carbon number of 1 to 4) with at least one hydrogen atom being replaced by a fluorine atom, more preferably a perfluoroalkyl group having a carbon number of 1 to 4. R₆₂ and R₆₃ may combine with each other to form a ring.

Specific examples of the group represented by formula (F2) include p-fluorophenyl group, pentafluorophenyl group and 3,5-di(trifluoromethyl)phenyl group.

Specific examples of the group represented by formula (F3) include trifluoromethyl group, pentafluoropropyl group, pentafluoroethyl group, heptafluorobutyl group, hexafluoroisopropyl group, heptafluoroisopropyl group, hexafluoro(2-methyl)isopropyl group, nonafluorobutyl group, octafluoroisobutyl group, nonafluorohexyl group, nonafluoro-tert-butyl group, perfluoroisopentyl group, perfluorooctyl group, perfluoro(trimethyl)hexyl group, 2,2,3,3-tetrafluorocyclobutyl group and perfluorocyclohexyl group. Among these, hexafluoroisopropyl group, heptafluoroisopropyl group, hexafluoro(2-methyl)isopropyl group, octafluoroisobutyl group, nonafluoro-tert-butyl group and perfluoroisopentyl group are preferred, and hexafluoroisopropyl group and heptafluoroisopropyl group are more preferred.

Specific examples of the group represented by formula (F4) include —C(CF₃)₂OH, —C(C₂F₅)₂OH, —C(CF₃)(CH₃)OH and —CH(CF₃)OH, with —C(CF₃)₂OH being preferred.

Specific examples of the repeating unit having a fluorine atom are illustrated below, but the present invention is not limited thereto.

In specific examples, X₁ represents a hydrogen atom, —CH₃, —F or —CF₃, and X₂ represents —F or —CF₃. Incidentally, specific examples also include fluorine atom-containing repeating units contained in Resins (HR-1) to (HR-65) illustrated later.

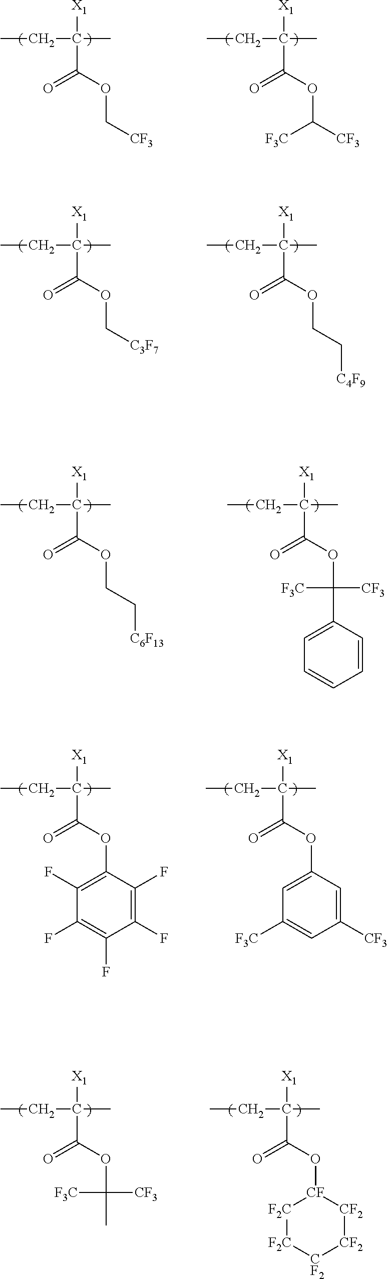

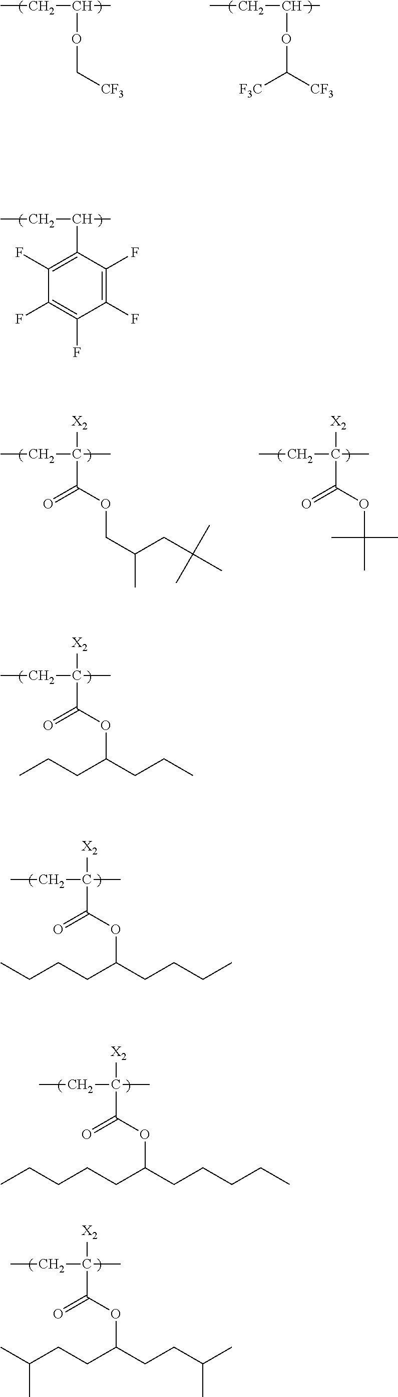

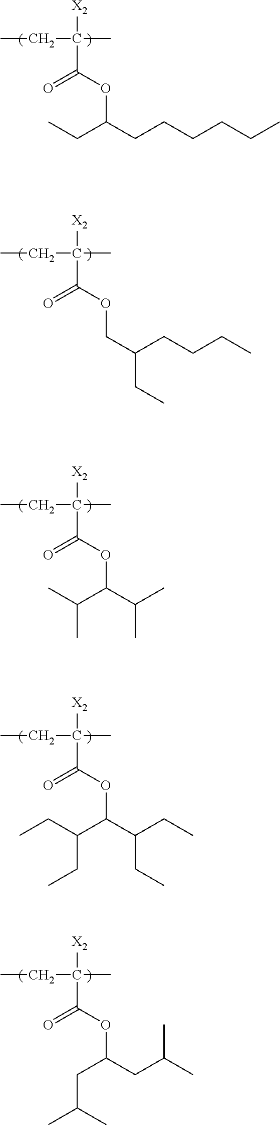

The hydrophobic resin (HR) may contain a silicon atom and is preferably a resin having an alkylsilyl structure (preferably a trialkylsilyl group) or a cyclic siloxane structure, as the silicon atom-containing partial structure.

Specific examples of the alkylsilyl structure and cyclic siloxane structure include the groups represented by the following formulae (CS-1) to (CS-3):

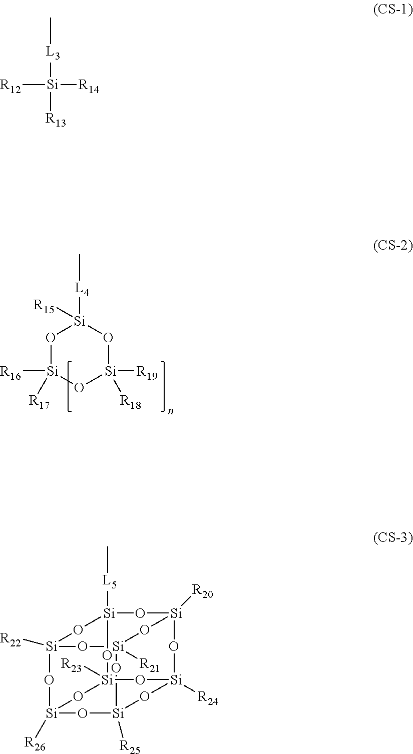

In formulae (CS-1) to (CS-3), each of R₁₂ to R₂₆ independently represents a linear or branched alkyl group (preferably having a carbon number of 1 to 20) or a cycloalkyl group (preferably having a carbon number of 3 to 20).

Each of L₃ to L₅ represents a single bond or a divalent linking group. The divalent linking group is a sole group or a combination of two or more groups selected from the group consisting of an alkylene group, a phenylene group, an ether group, a thioether group, a carbonyl group, an ester group, an amide group, a urethane group and a urea group.

n represents an integer of 1 to 5. n is preferably an integer of 1 to 3.

Specific examples of the repeating unit having a group represented by formulae (CS-1) to (CS-3) are illustrated below, but the present invention is not limited thereto. In this connection, specific examples also include silicon atom-containing repeating units contained in Resins (HR-1) to (HR-65) illustrated later. In specific examples, X₁ represents a hydrogen atom, —CH₃, —F or —CF₃.

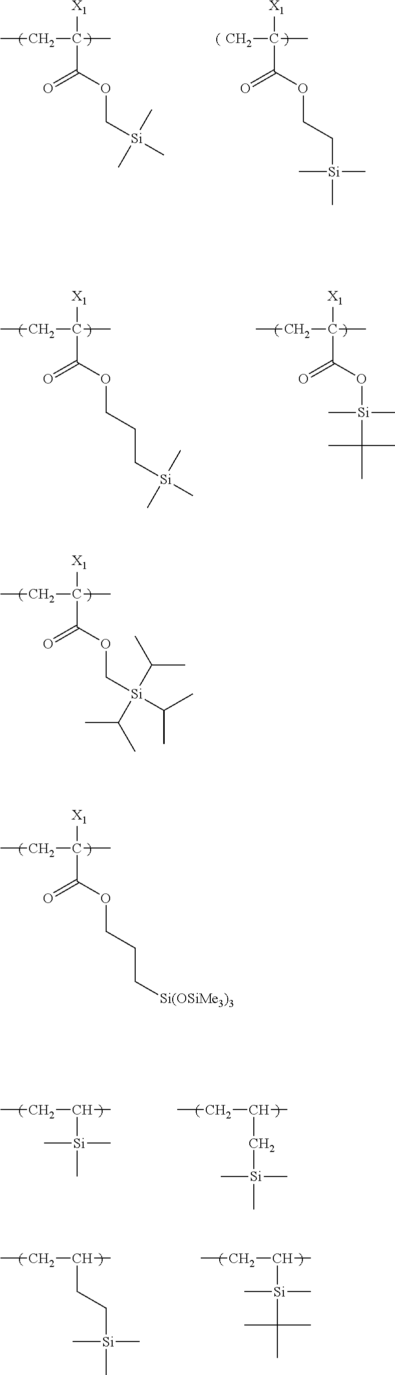

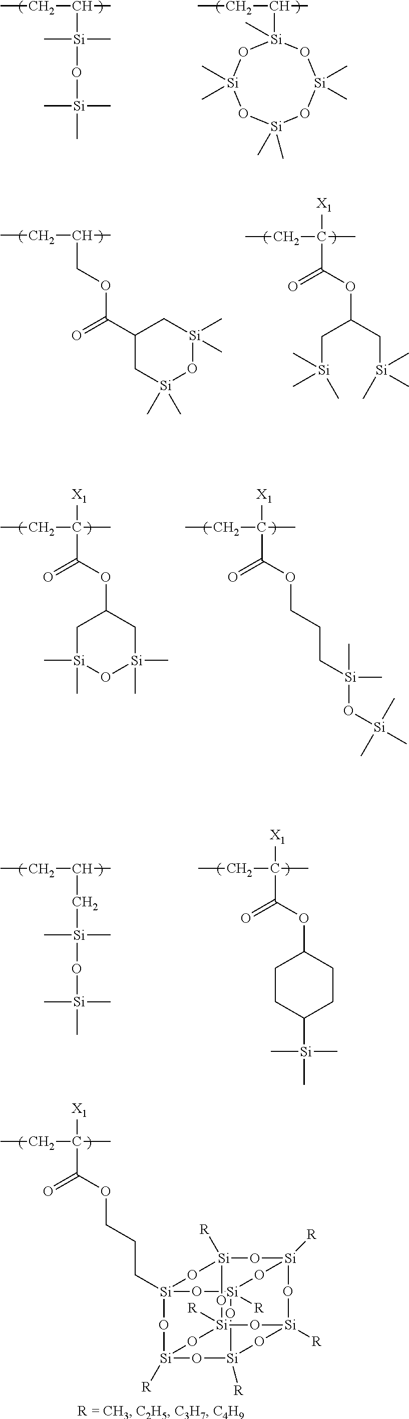

Furthermore, the hydrophobic resin (HR) may contain at least one group selected from the group consisting of the following (x) to (z):

(x) an alkali-soluble group,

(y) a group capable of decomposing by the action of an alkali developer to increase the solubility in an alkali developer, and

(z) a group capable of decomposing by the action of an acid.

Examples of the alkali-soluble group (x) include a phenolic hydroxyl group, a carboxylic acid group, a fluorinated alcohol group, a sulfonic acid group, a sulfonamide group, a sulfonylimide group, an (alkylsulfonyl)(alkylcarbonyl)methylene group, an (alkylsulfonyl)(alkylcarbonyl)imide group, a bis(alkylcarbonyl)methylene group, a bis(alkylcarbonyl)imide group, a bis(alkylsulfonyl)methylene group, a bis(alkylsulfonyl)imide group, a tris(alkylcarbonyl)methylene group and a tris(alkylsulfonyl)methylene group.

Preferred alkali-soluble groups are a fluorinated alcohol group (preferably hexafluoroisopropanol), a sulfonimide group and a bis(carbonyl)methylene group.

Examples of the repeating unit having (x) an alkali-soluble group include a repeating unit where an alkali-soluble group is directly bonded to the main chain of the resin, such as repeating unit by an acrylic acid or a methacrylic acid, and a repeating unit where an alkali-soluble group is bonded to the main chain of the resin through a linking group, and an alkali-soluble group may also be introduced into the polymer chain terminal by using an alkali-soluble group-containing polymerization initiator or chain transfer agent at the polymerization. All of these cases are preferred.

The content of the repeating unit having (x) an alkali-soluble group is preferably from 1 to 50 mol %, more preferably from 3 to 35 mol %, still more preferably from 5 to 20 mol %, based on all repeating units in the polymer.

Specific examples of the repeating unit having (x) an alkali-soluble group are illustrated below, but the present invention is not limited thereto. In specific examples, Rx represents a hydrogen atom, CH₃, CH₂OH or CF₃.

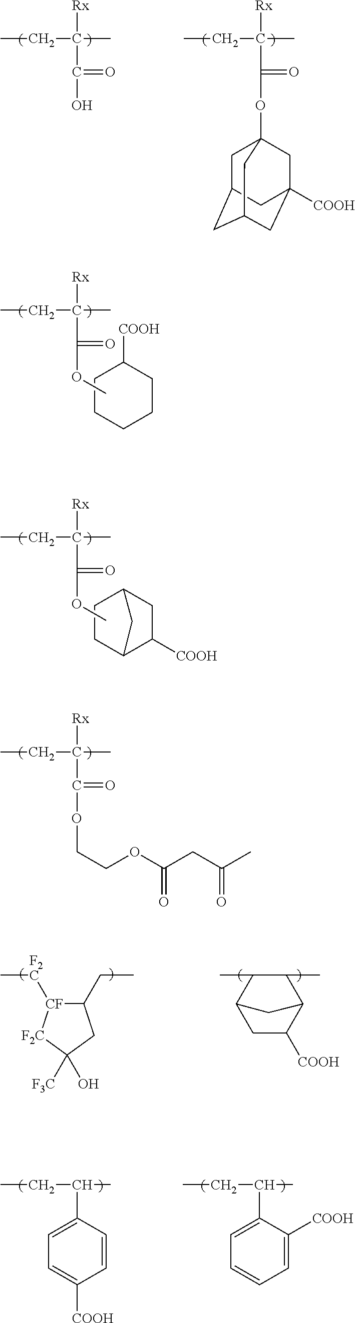

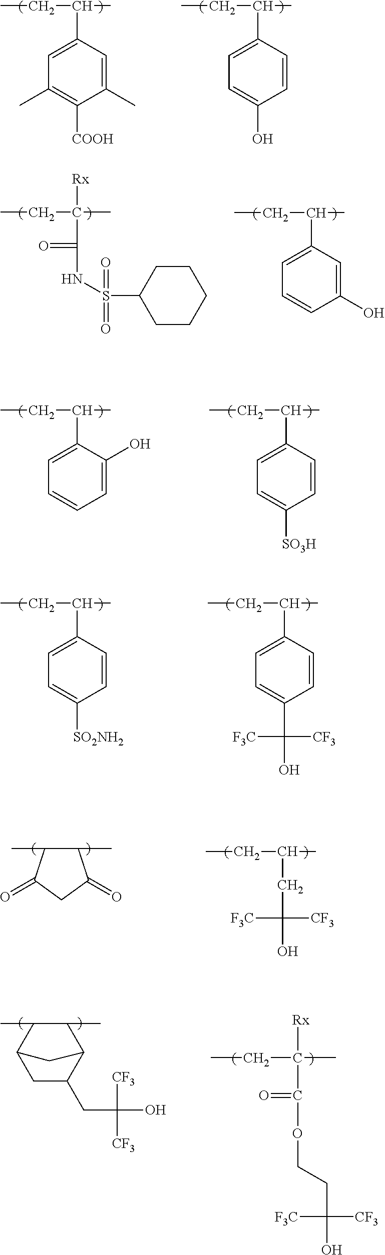

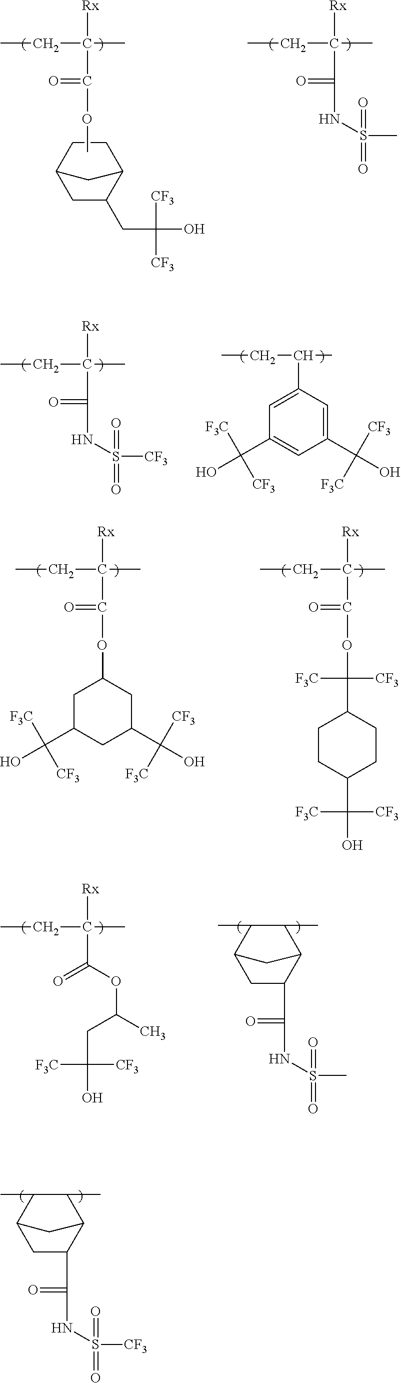

Examples of the (y) group capable of decomposing by the action of an alkali developer to increase the solubility in an alkali developer include a lactone structure-containing group, an acid anhydride group and an acid imide group, with a lactone structure-containing group being preferred.

As for the repeating unit having (y) a group capable of decomposing by the action of an alkali developer to increase the solubility in an alkali developer, both a repeating unit where (y) a group capable of decomposing by the action of an alkali developer to increase the solubility in an alkali developer is bonded to the main chain of the resin, such as repeating unit by an acrylic acid ester or a methacrylic acid ester, and an introduction into the polymer chain terminal by using, at the polymerization, a polymerization initiator or chain transfer agent containing (y) a group capable of increasing the solubility in an alkali developer, are preferred.

The content of the repeating unit having (y) a group capable of increasing the solubility in an alkali developer is preferably from 1 to 40 mol %, more preferably from 3 to 30 mol %, still more preferably from 5 to 15 mol %, based on all repeating units in the polymer.

Specific examples of the repeating unit having (y) a group capable of increasing the solubility in an alkali developer are the same as those of the repeating unit having a lactone structure described for the resin (B1).

Examples of the repeating unit having (z) a group capable of decomposing by the action of an acid, contained in the hydrophobic resin (HR), are the same as those of the repeating unit having an acid-decomposable group described for the resin (B1). In the hydrophobic resin (HR), the content of the repeating unit having (z) a group capable of decomposing by the action of an acid is preferably from 1 to 80 mol %, more preferably from 10 to 80 mol %, still more preferably from 20 to 60 mol %, based on all repeating units in the polymer.

The hydrophobic resin (HR) may further contain a repeating unit represented by the following formula (CIII):

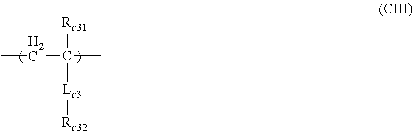

In formula (CIII), R_{c31} represents a hydrogen atom, an alkyl group (an alkyl group which may be substituted with a fluorine atom or the like), a cyano group or a —CH₂—O—R_{ac2} group, wherein R_{ac2} represents a hydrogen atom, an alkyl group or an acyl group. R_{c31} is preferably a hydrogen atom, a methyl group, a hydroxymethyl group or a trifluoromethyl group, more preferably a hydrogen atom or a methyl group.

R_{c32} represents a group having an alkyl group, a cycloalkyl group, an alkenyl group, a cycloalkenyl group or an aryl group. Each of these groups may be substituted, for example, with a group containing a fluorine atom or a silicon atom.

L_{c3} represents a single bond or a divalent linking group.

In formula (CIII), the alkyl group in R_{c32} may be linear or branched and is preferably an alkyl group having a carbon number of 3 to 20. The cycloalkyl group in may be monocyclic, polycyclic or spirocyclic and is preferably a cycloalkyl group having a carbon number of 3 to 20. The alkenyl group in R_{c32} is preferably an alkenyl group having a carbon number of 3 to 20. The cycloalkenyl group in R_{c32} is preferably a cycloalkenyl group having a carbon number of 3 to 20. The aryl group in R_{c32} is preferably an aryl group having a carbon number of 6 to 20.

R_{c32} is preferably an unsubstituted alkyl group or a fluorine atom-substituted alkyl group. The divalent linking group of L_{c3} is preferably an alkylene group (preferably having a carbon number of 1 to 5), an oxy group, a phenylene group or an ester bond (a group represented by —COO—). It is also preferred that the hydrophobic resin (HR) further contains a repeating unit represented by the following formula (CII-AB):

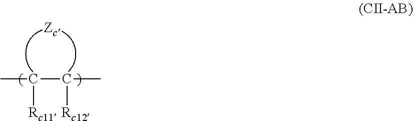

In formula (CII-AB), each of R_{c11}′ and R_{c12}′ independently represents a hydrogen atom, a cyano group, a halogen atom or an alkyl group.

Z_{c}′ represents an atomic group for forming an alicyclic structure containing two bonded carbon atoms (C—C).

Specific examples of the repeating units represented by formulae (CIII) and (CII-AB) are illustrated below, but the present invention is not limited thereto. In the formulae, Ra represents H, CH₃, CH₂OH, CF₃ or CN.

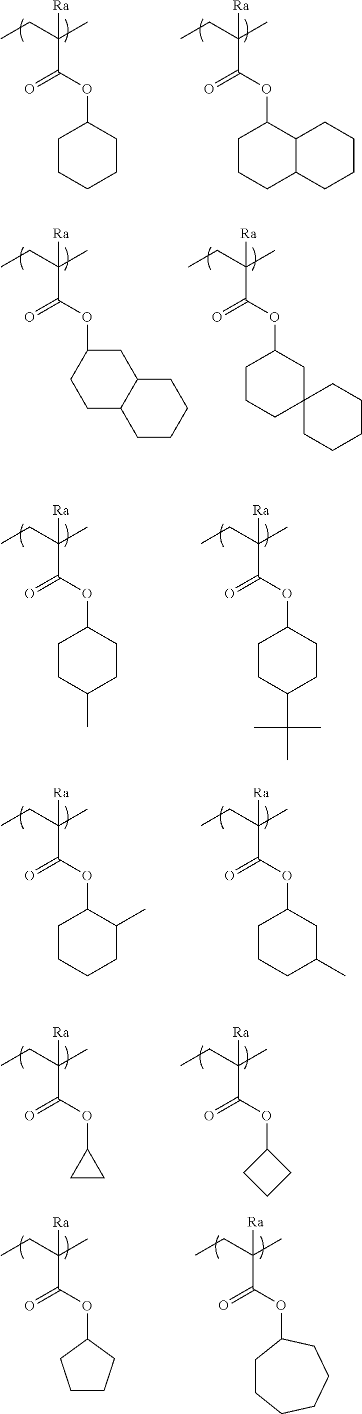

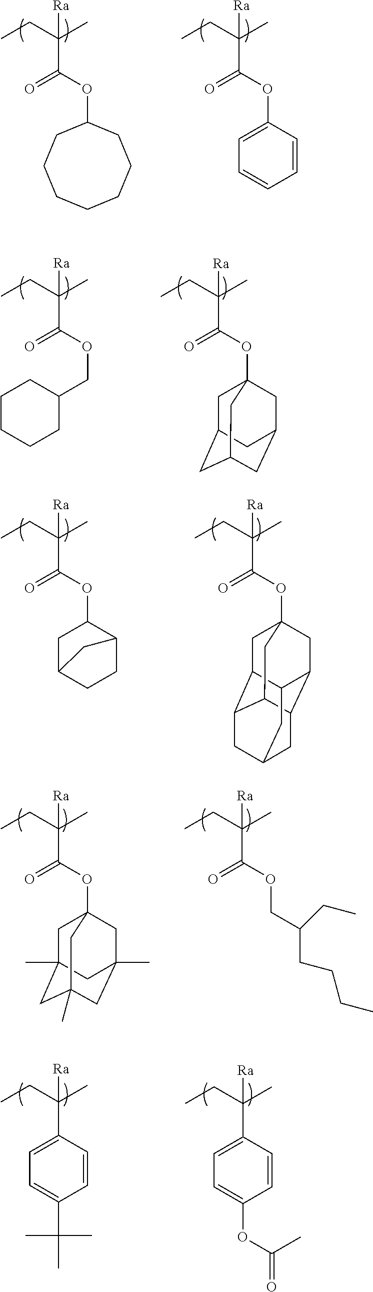

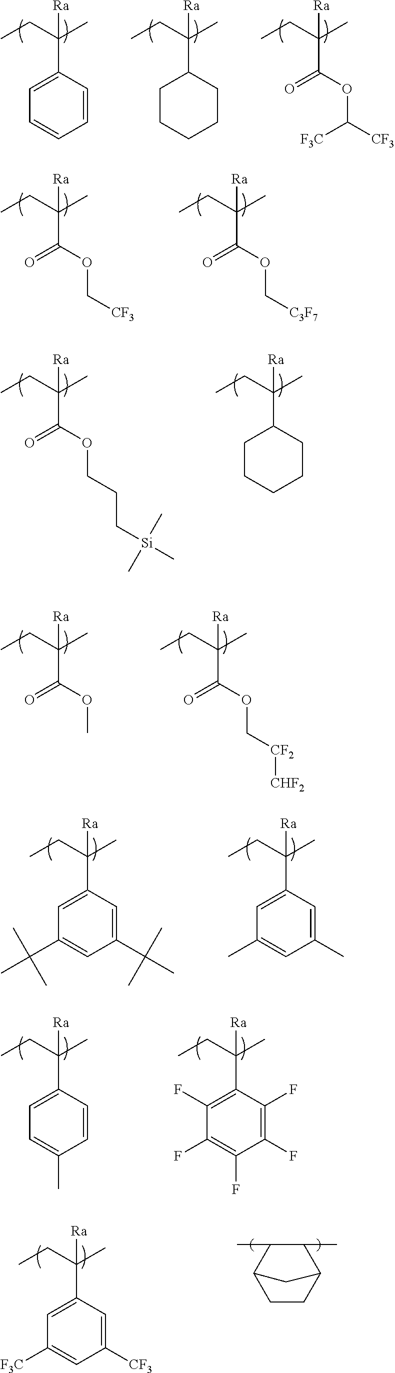

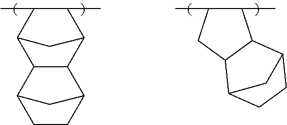

In the case where the hydrophobic resin (HR) contains a fluorine atom, the fluorine atom content is preferably from 5 to 80 mass %, more preferably from 10 to 80 mass %, based on the weight average molecular weight of the hydrophobic resin (HR). Also, the fluorine atom-containing repeating unit preferably occupies from 10 to 100 mol %, more preferably from 30 to 100 mol %, in the hydrophobic resin (HR).

In the case where the hydrophobic resin (HR) contains a silicon atom, the silicon atom content is preferably from 2 to 50 mass %, more preferably from 2 to 30 mass %, based on the weight average molecular weight of the hydrophobic resin (HR). Also, the silicon atom-containing repeating unit preferably occupies from 10 to 100 mol %, more preferably from 20 to 100 mol %, in the hydrophobic resin (HR).

The standard polystyrene-equivalent weight average molecular weight of the hydrophobic resin (HR) is preferably from 1,000 to 100,000, more preferably from 1,000 to 50,000, still more preferably from 2,000 to 15,000.

The content of the hydrophobic resin (HR) in the composition is preferably from 0.01 to 10 mass %, more preferably from 0.05 to 8 mass %, still more preferably from 0.1 to 5 mass %, based on the entire solid content of the composition of the present invention.

In the hydrophobic resin (HR), similarly to the resin (B1), it is of course preferred that the content of impurities such as metal is small, and also, the content of residual monomers or oligomer components is preferably from 0 to 10 mass %, more preferably from 0 to 5 mass %, still more preferably from 0 to 1 mass %. When these conditions are satisfied, a resist free of extraneous substances in liquid or change with aging of sensitivity or the like can be obtained. Furthermore, in view of resolution, resist profile, side wall of resist pattern, roughness and the like, the molecular weight distribution (Mw/Mn, sometimes referred to as “polydispersity”) is preferably from 1 to 5, more preferably from 1 to 3, still more preferably from 1 to 2.

As for the hydrophobic resin (HR), various commercial products may be used, or the resin may be synthesized by an ordinary method (for example, radical polymerization). Specifically, the resin can be obtained in the same manner as the resin (B1).

Specific examples of the hydrophobic resin (HR) are illustrated below. Also, the molar ratio of repeating units (corresponding to repeating units starting from the left), weight average molecular weight and polydispersity of each resin are shown in the Table later.

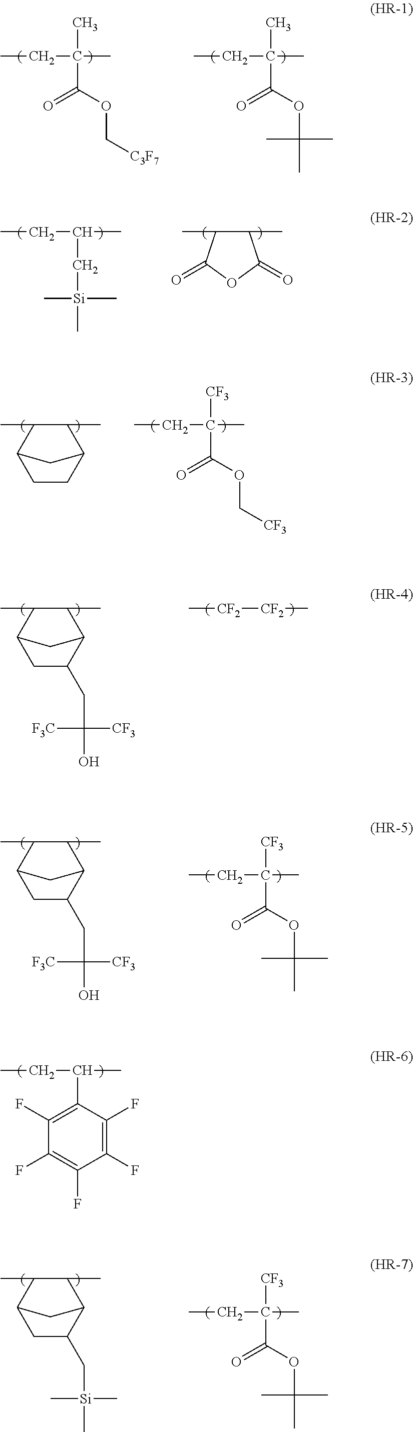

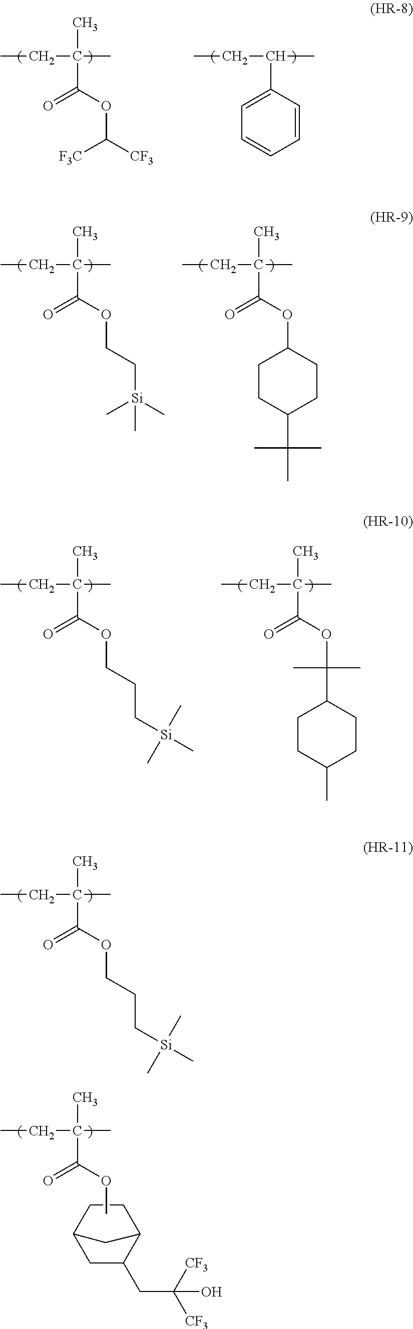

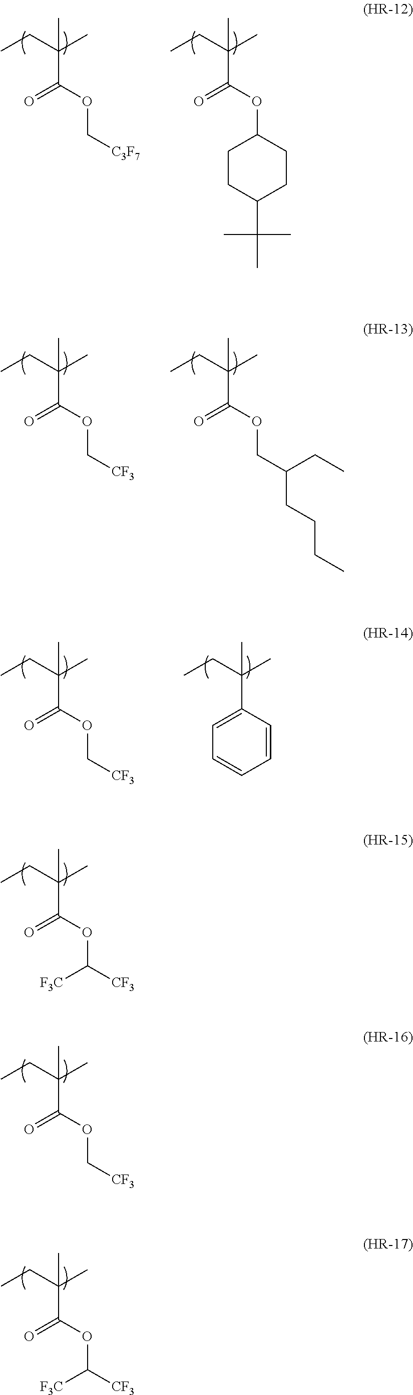

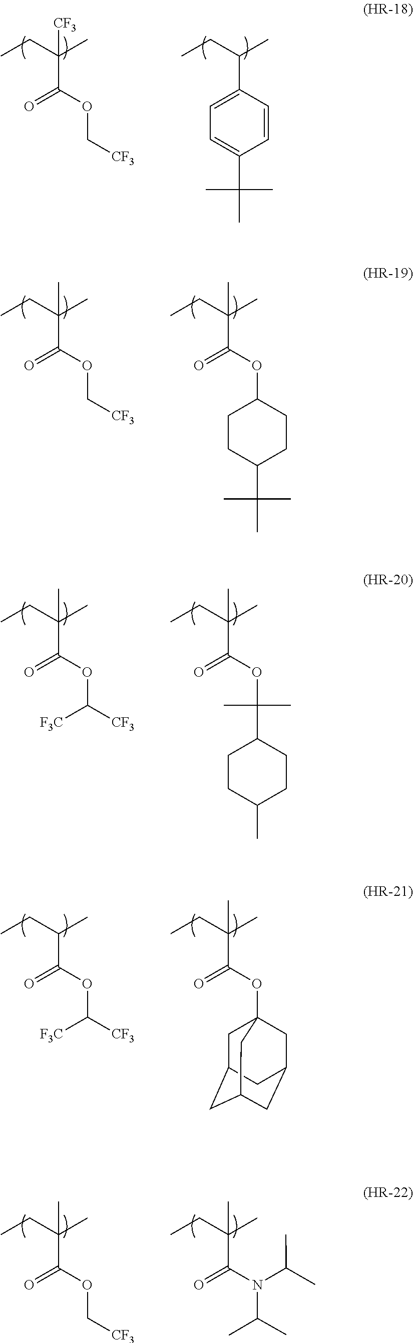

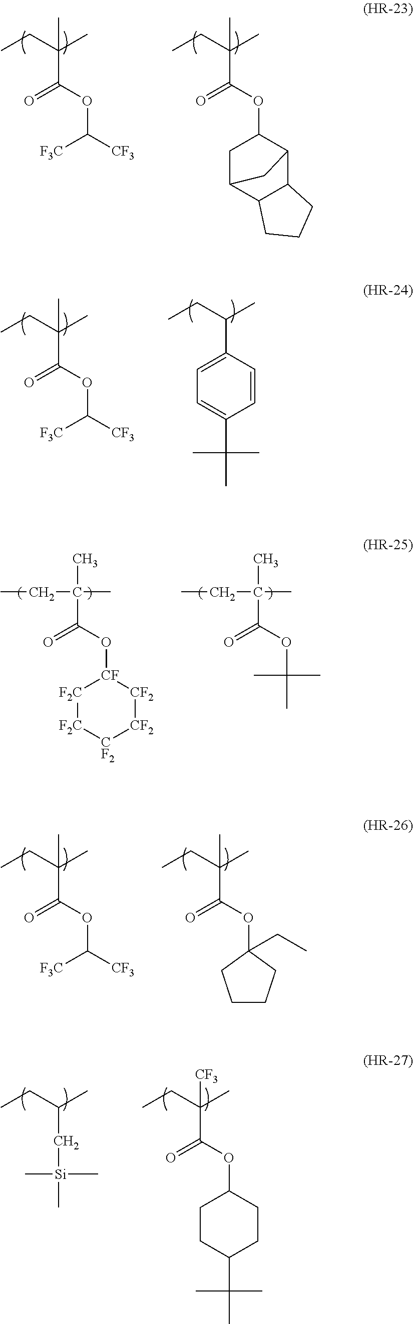

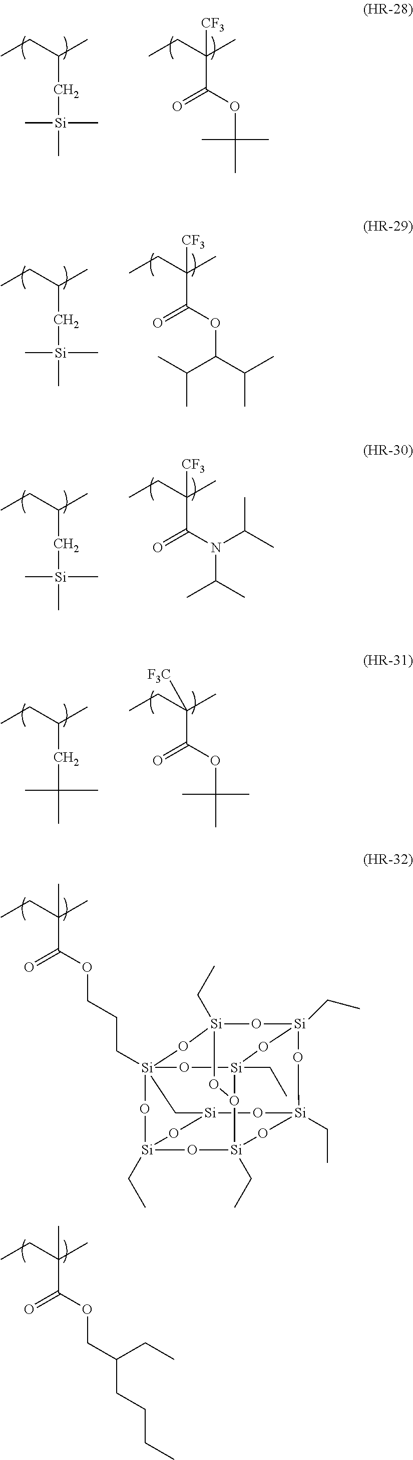

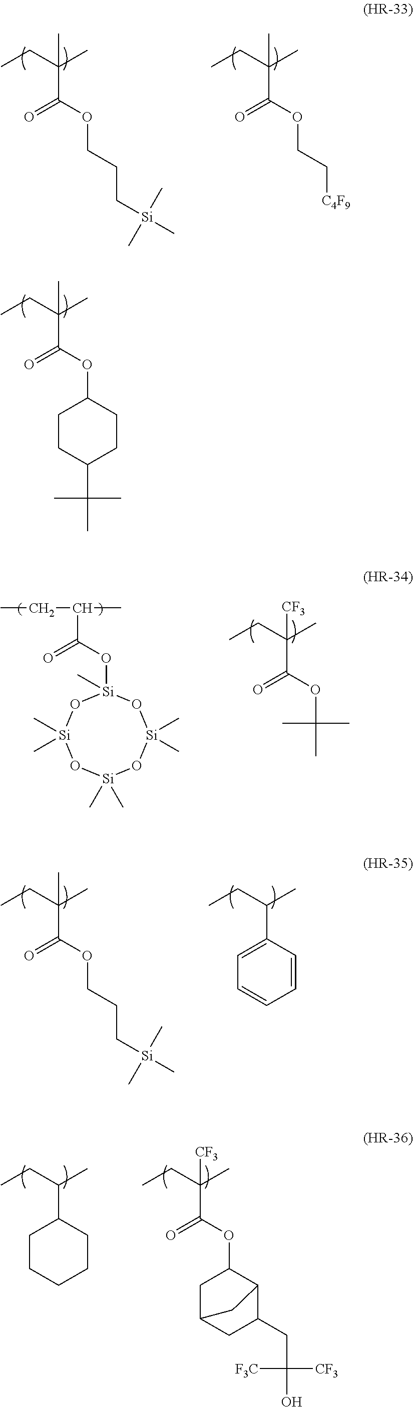

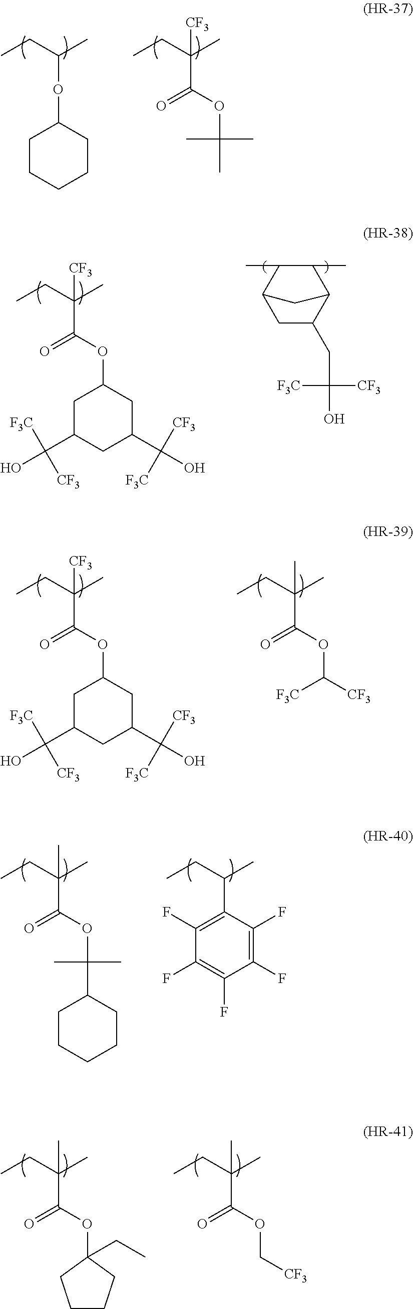

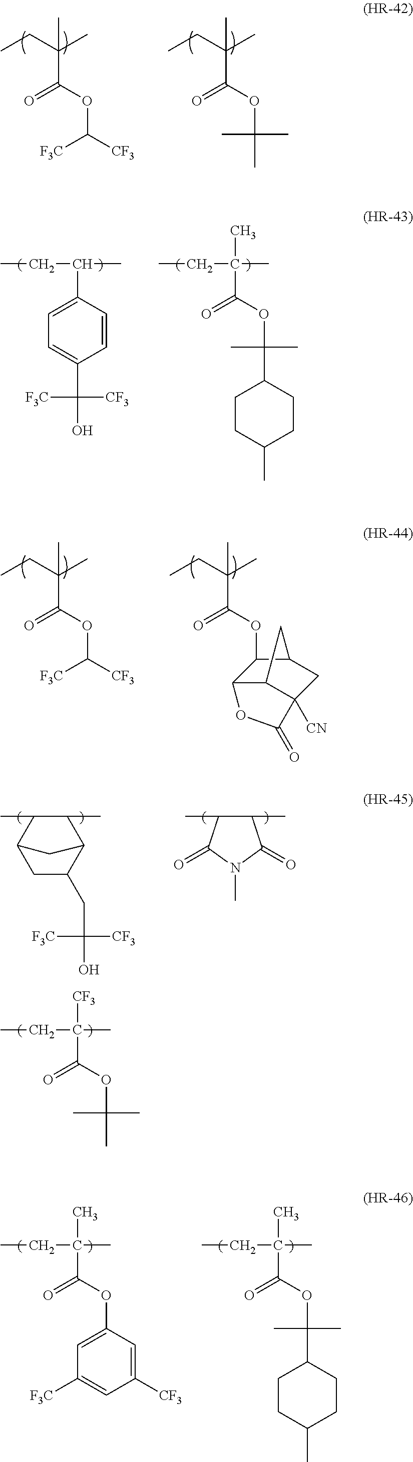

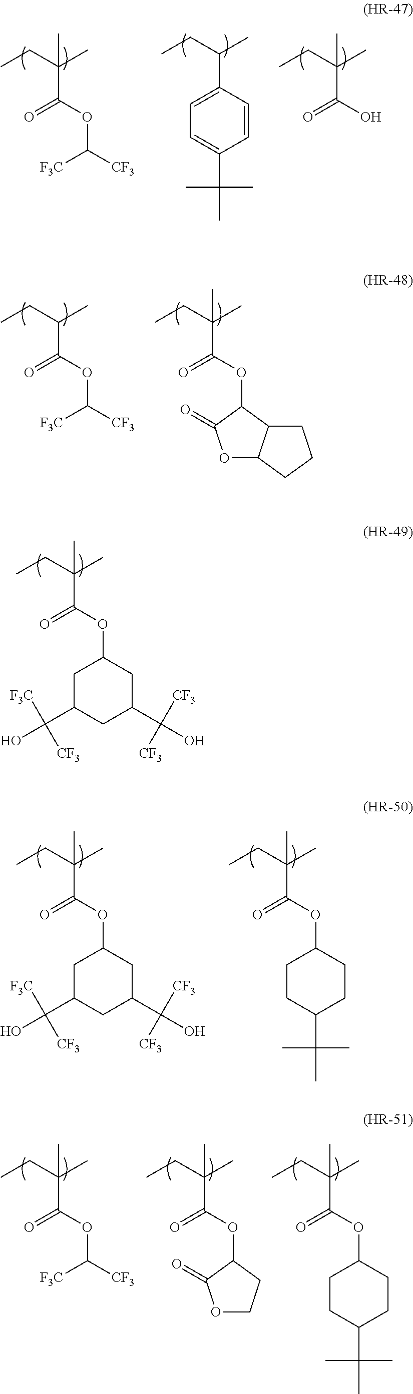

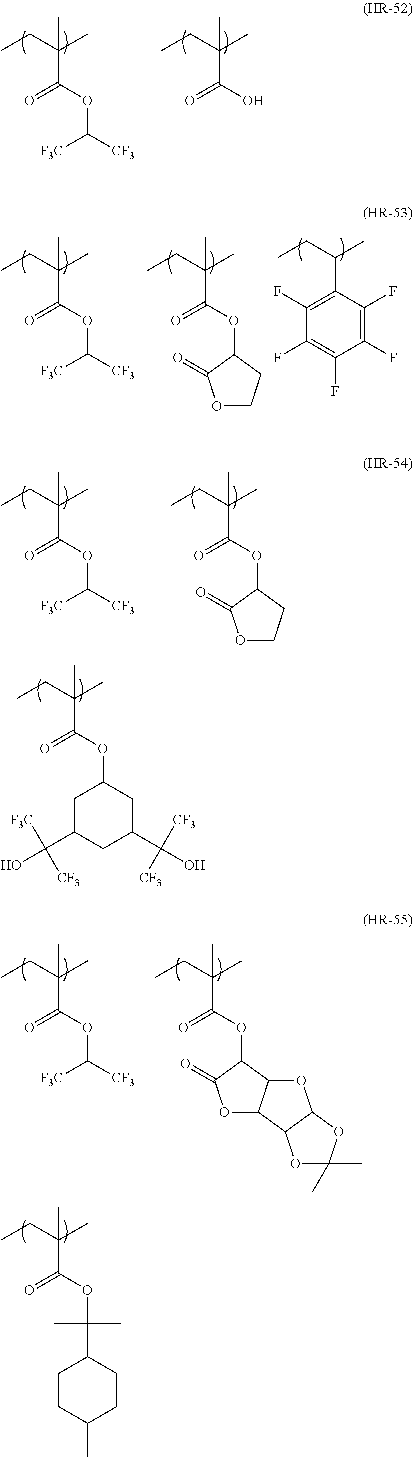

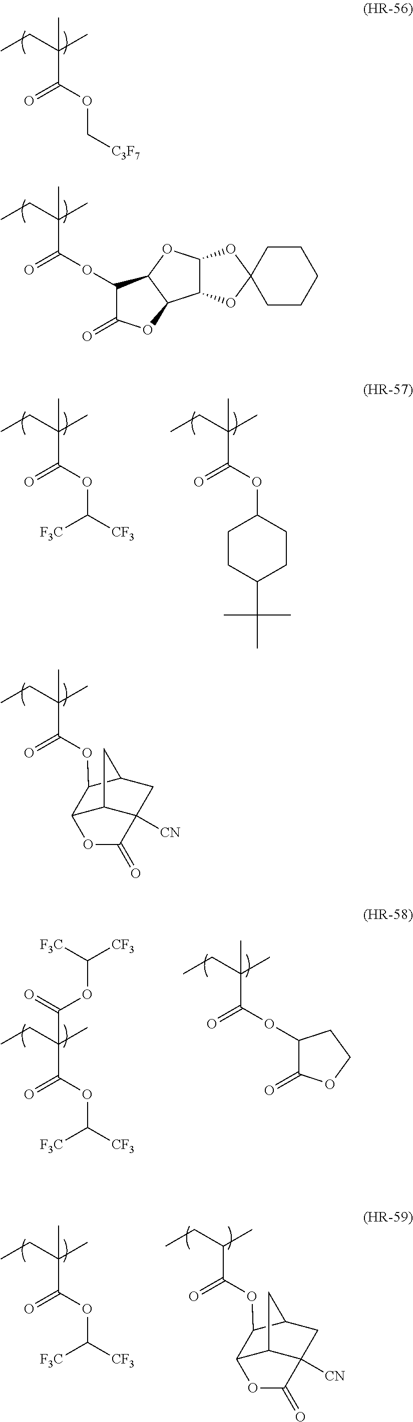

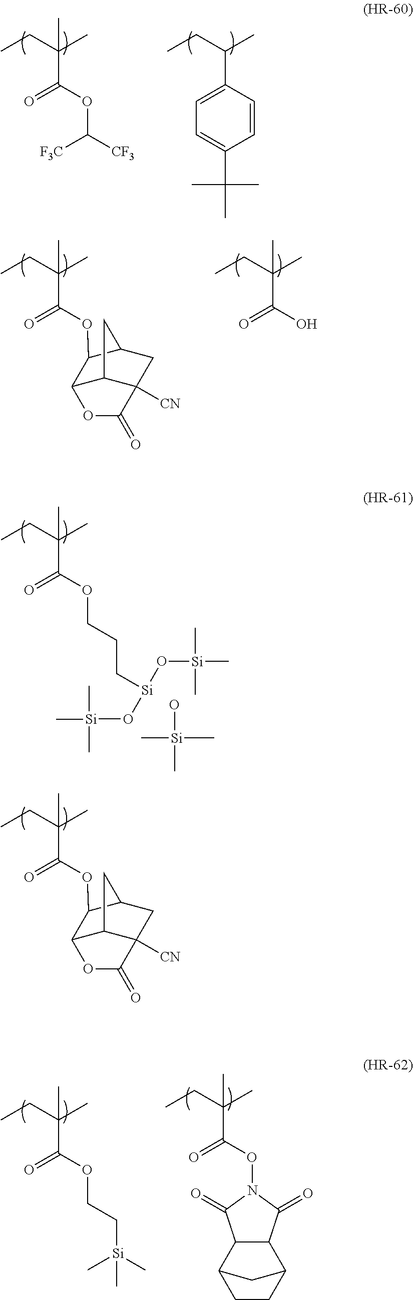

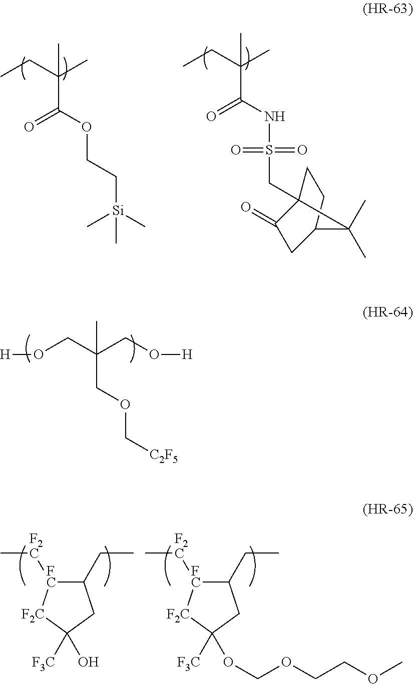

TABLE 2

| Resin | Composition | Mw | Mw/Mn |
|---|---|---|---|
| HR-1 | 50/50 | 4900 | 1.4 |
| HR-2 | 50/50 | 5100 | 1.6 |
| HR-3 | 50/50 | 4800 | 1.5 |
| HR-4 | 50/50 | 5300 | 1.6 |
| HR-5 | 50/50 | 4500 | 1.4 |
| HR-6 | 100 | 5500 | 1.6 |
| HR-7 | 50/50 | 5800 | 1.9 |
| HR-8 | 50/50 | 4200 | 1.3 |
| HR-9 | 50/50 | 5500 | 1.8 |
| HR-10 | 40/60 | 7500 | 1.6 |
| HR-11 | 70/30 | 6600 | 1.8 |
| HR-12 | 40/60 | 3900 | 1.3 |
| HR-13 | 50/50 | 9500 | 1.8 |
| HR-14 | 50/50 | 5300 | 1.6 |
| HR-15 | 100 | 6200 | 1.2 |
| HR-16 | 100 | 5600 | 1.6 |
| HR-17 | 100 | 4400 | 1.3 |
| HR-18 | 50/50 | 4300 | 1.3 |
| HR-19 | 50/50 | 6500 | 1.6 |
| HR-20 | 30/70 | 6500 | 1.5 |
| HR-21 | 50/50 | 6000 | 1.6 |
| HR-22 | 50/50 | 3000 | 1.2 |
| HR-23 | 50/50 | 5000 | 1.5 |
| HR-24 | 50/50 | 4500 | 1.4 |
| HR-25 | 30/70 | 5000 | 1.4 |
| HR-26 | 50/50 | 5500 | 1.6 |
| HR-27 | 50/50 | 3500 | 1.3 |
| HR-28 | 50/50 | 6200 | 1.4 |
| HR-29 | 50/50 | 6500 | 1.6 |
| HR-30 | 50/50 | 6500 | 1.6 |
| HR-31 | 50/50 | 4500 | 1.4 |
| HR-32 | 30/70 | 5000 | 1.6 |
| HR-33 | 30/30/40 | 6500 | 1.8 |
| HR-34 | 50/50 | 4000 | 1.3 |
| HR-35 | 50/50 | 6500 | 1.7 |
| HR-36 | 50/50 | 6000 | 1.5 |
| HR-37 | 50/50 | 5000 | 1.6 |
| HR-38 | 50/50 | 4000 | 1.4 |
| HR-39 | 20/80 | 6000 | 1.4 |
| HR-40 | 50/50 | 7000 | 1.4 |
| HR-41 | 50/50 | 6500 | 1.6 |
| HR-42 | 50/50 | 5200 | 1.6 |
| HR-43 | 50/50 | 6000 | 1.4 |
| HR-44 | 70/30 | 5500 | 1.6 |
| HR-45 | 50/20/30 | 4200 | 1.4 |
| HR-46 | 30/70 | 7500 | 1.6 |
| HR-47 | 40/58/2 | 4300 | 1.4 |
| HR-48 | 50/50 | 6800 | 1.6 |
| HR-49 | 100 | 6500 | 1.5 |
| HR-50 | 50/50 | 6600 | 1.6 |
| HR-51 | 30/20/50 | 6800 | 1.7 |
| HR-52 | 95/5 | 5900 | 1.6 |
| HR-53 | 40/30/30 | 4500 | 1.3 |
| HR-54 | 50/30/20 | 6500 | 1.8 |
| HR-55 | 30/40/30 | 7000 | 1.5 |
| HR-56 | 60/40 | 5500 | 1.7 |
| HR-57 | 40/40/20 | 4000 | 1.3 |
| HR-58 | 60/40 | 3800 | 1.4 |
| HR-59 | 80/20 | 7400 | 1.6 |
| HR-60 | 40/40/15/5 | 4800 | 1.5 |
| HR-61 | 60/40 | 5600 | 1.5 |
| HR-62 | 50/50 | 5900 | 2.1 |
| HR-63 | 80/20 | 7000 | 1.7 |
| HR-64 | 100 | 5500 | 1.8 |
| HR-65 | 50/50 | 9500 | 1.9 |

### [5] Basic Compound

The actinic ray-sensitive or radiation-sensitive composition of the present invention preferably contains a basic compound.

The basic compound is preferably a nitrogen-containing organic basic compound.

The usable compound is not particularly limited, but, for example, compounds classified into the following (1) to (4) are preferably used.

### (1) Compound Represented by the Following Formula (BS-1):

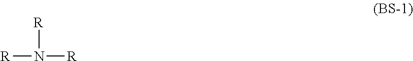

In formula (BS-1), each R independently represents any of a hydrogen atom, an alkyl group (linear or branched), a cycloalkyl group (monocyclic or polycyclic), an aryl group and an aralkyl group, but it is not allowed that three R's all are a hydrogen atom.

The carbon number of the alkyl group as R is not particularly limited but is usually from 1 to 20, preferably from 1 to 12.

The carbon number of the cycloalkyl group as R is not particularly limited but is usually from 3 to 20, preferably from 5 to 15.

The carbon number of the aryl group as R is not particularly limited but is usually from 6 to 20, preferably from 6 to 10. Specific examples thereof include a phenyl group and a naphthyl group.

The carbon number of the aralkyl group as R is not particularly limited but is usually from 7 to 20, preferably from 7 to 11. Specific examples thereof include a benzyl group.

In the alkyl group, cycloalkyl group, aryl group and aralkyl group as R, a hydrogen atom may be replaced by a substituent. Examples of the substituent include an alkyl group, a cycloalkyl group, an aryl group, an aralkyl group, a hydroxyl group, a carboxyl group, an alkoxy group, an aryloxy group, an alkylcarbonyloxy group and an alkyloxycarbonyl group.

In the compound represented by formula (BS-1), it is preferred that only one of three R's is a hydrogen atom or all R's are not a hydrogen atom.

Specific examples of the compound of formula (BS-1) include tri-n-butylamine, tri-n-pentylamine, tri-n-octylamine, tri-n-decylamine, triisodecylamine, dicyclohexylmethylamine, tetradecylamine, pentadecylamine, hexadecylamine, octadecylamine, didecylamine, methyloctadecylamine, dimethylundecylamine, N,N-dimethyldodecylamine, methyldioctadecylamine, N,N-dibutylaniline, N,N-dihexylaniline, 2,6-diisopropylaniline and 2,4,6-tri(tert-butyl)aniline.

Also, one preferred embodiment is a compound where in formula (BS-1), at least one R is an alkyl group substituted with a hydroxyl group. Specific examples of the compound include triethanolamine and N,N-dihydroxyethylaniline.

The alkyl group as R may contain an oxygen atom in the alkyl chain to form an oxyalkylene chain. The oxyalkylene chain is preferably —CH₂CH₂O—. Specific examples thereof include tris(methoxyethoxyethyl)amine and compounds illustrated in U.S. Pat. No. 6,040,112, column 3, line 60 et seq.

### (2) Compound Having a Nitrogen-Containing Heterocyclic Structure

The heterocyclic structure may or may not have aromaticity, may contain a plurality of nitrogen atoms, and may further contain a heteroatom other than nitrogen. Specific examples of the compound include a compound having an imidazole structure (e.g., 2-phenylbenzimidazole, 2,4,5-triphenylimidazole), a compound having a piperidine structure (e.g., N-hydroxyethylpiperidine, bis(1,2,2,6,6-pentamethyl-4-piperidyl)sebacate), a compound having a pyridine structure (e.g., 4-dimethylaminopyridine), and a compound having an antipyrine structure (e.g., antipyrine, hydroxyantipyrine).

A compound having two or more ring structures is also suitably used. Specific examples thereof include 1,5-diazabicyclo[4.3.0]non-5-ene and 1,8-diazabicyclo[5.4.0]undec-7-ene.

### (3) Phenoxy Group-Containing Amine Compound

The phenoxy group-containing amine compound is a compound where the alkyl group of an amine compound has a phenoxy group at the terminal opposite the nitrogen atom. The phenoxy group may have a substituent such as alkyl group, alkoxy group, halogen atom, cyano group, nitro group, carboxyl group, carboxylic acid ester group, sulfonic acid group, aryl group, aralkyl group, acyloxy group, aryloxy group.

A compound having at least one oxyalkylene chain between the phenoxy group and the nitrogen atom is preferred. The number of oxyalkylene chains in one molecule is preferably from 3 to 9, more preferably from 4 to 6. Among oxyalkylene chains, —CH₂CH₂O— is preferred.

Specific examples of the compound include 2-[2-{2-(2,2-dimethoxy-phenoxyethoxy)ethyl}-bis-(2-methoxyethyl)]-amine and Compounds (C1-1) to (C3-3) illustrated in paragraph [0066] of U.S. Patent Application Publication No. 2007/0224539A1.

### (4) Ammonium Salt

An ammonium salt is also appropriately used. The ammonium salt is preferably a hydroxide or a carboxylate. More specifically, a tetraalkylammonium hydroxide typified by tetrabutylammonium hydroxide is preferred.

Other examples of the compound usable in the composition of the present invention include compounds synthesized in Examples of JP-A-2002-363146 and compounds described in paragraph 0108 of JP-A-2007-298569.

As for the basic compound, one kind of a compound is used alone, or two or more kinds of compounds are used in combination.

The amount of the basic compound used is usually from 0.001 to 10 mass %, preferably from 0.01 to 5 mass %, based on the solid content of the composition.

The molar ratio of acid generator/basic compound is preferably from 2.5 to 300. That is, the molar ratio is preferably 2.5 or more in view of sensitivity and resolution and is preferably 300 or less from the standpoint of suppressing the reduction in resolution due to thickening of the resist pattern with aging after exposure until heat treatment. The molar ratio is more preferably from 5.0 to 200, still more preferably from 7.0 to 150.

### [6] (D) Low Molecular Compound Having a Group Capable of Leaving by the Action of an Acid

The composition of the present invention may contain (D) a low molecular compound having a group capable of leaving by the action of an acid (sometimes referred to as a “component (D)”). The group capable of leaving by the action of an acid is not particularly limited but is preferably an acetal group, a carbonate group, a carbamate group, a tertiary ester group, a tertiary hydroxyl group or a hemiaminal ether group, more preferably a carbamate group or a hemiaminal ether group.

The molecular weight of the low molecular compound having a group capable of leaving by the action of an acid is preferably from 100 to 1,000, more preferably from 100 to 700, still more preferably from 100 to 500.

In the case where the low molecular compound having a group capable of leaving by the action of an acid has a tertiary ester structure, the compound is preferably a carboxylic acid ester or unsaturated carboxylic acid ester represented by the following formula (1a):

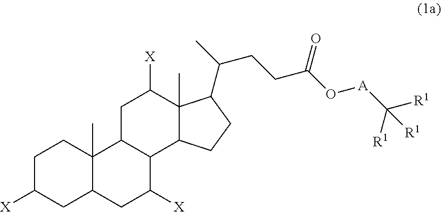

In formula (1a), each R¹ independently represents a monovalent alicyclic hydrocarbon group (preferably having a carbon number of 4 to 20), a derivative thereof or an alkyl group (preferably having a carbon number of 1 to 4) and at the same time, at least one R¹ is the alicyclic hydrocarbon group or a derivative thereof, or while any two R¹'s are combined with each other to form a divalent alicyclic hydrocarbon group (preferably having a carbon number of 4 to 20) or a derivative thereof together with the carbon atom to which they are bonded, the remaining R¹ represents an alkyl group (preferably having a carbon number of 1 to 4), a monovalent alicyclic hydrocarbon group (preferably having a carbon number of 4 to 20) or a derivative thereof.

Each X independently represents a hydrogen atom or a hydroxy group, and at least one X is a hydroxy group.

A represents a single bond or a divalent linking group and is preferably a single bond or a group represented by -D-COO—, wherein D represents an alkylene group (preferably having a carbon number of 1 to 4).

In formula (1a), A represents a single bond or a divalent linking group, and examples of the divalent linking group include a methylene group, a methylenecarbonyl group, a methylenecarbonyloxy group, an ethylene group, an ethylenecarbonyl group, an ethylenecarbonyloxy group, a propylene group, a propylenecarbonyl group and a propylenecarbonyloxy group, with a methylenecarbonyloxy group being preferred.

In formula (1a), examples of the monovalent alicyclic hydrocarbon group (preferably having a carbon number of 4 to 20) of R¹ and the divalent alicyclic hydrocarbon group (preferably having a carbon number of 4 to 20) formed by combining any two R¹'s with each other include a group composed of an alicyclic ring derived from norbornane, tricyclodecane, tetracyclododecane, adamantane or cycloalkanes such as cyclobutane, cyclopentane, cyclohexane, cycloheptane and cyclooctane; and a group where the group composed of an alicyclic ring is substituted with one or more in kind or number of an alkyl group having a carbon number of 1 to 4, such as methyl group, ethyl group, n-propyl group, i-propyl group, n-butyl group, 2-methylpropyl group, 1-methylpropyl group and tert-butyl group, and a cycloalkyl group. Among these alicyclic hydrocarbon groups, preferred are a group composed of an alicyclic ring derived from norbornane, tricyclodecane, tetracyclododecane, adamantane, cyclopentane or cyclohexane, and a group where the group composed of an alicyclic ring is substituted with the alkyl group above.

Examples of the derivative of the alicyclic hydrocarbon group include a group having one or more in kind or number of substituents such as a hydroxyl group; a carboxyl group; an oxo group (i.e., ═O); a hydroxyalkyl group having a carbon number of 1 to 4, e.g., hydroxymethyl group, 1-hydroxyethyl group, 2-hydroxyethyl group, 1-hydroxypropyl group, 2-hydroxypropyl group, 3-hydroxypropyl group, 1-hydroxybutyl group, 2-hydroxybutyl group, 3-hydroxybutyl group and 4-hydroxybutyl group; an alkoxyl group having a carbon number of 1 to 4, e.g., methoxy group, ethoxy group, n-propoxy group, i-propoxy group, n-butoxy group, 2-methylpropoxy group, 1-methylpropoxy group and tert-butoxy group; a cyano group; and a cyanoalkyl group having a carbon number of 2 to 5, e.g., cyanomethyl group, 2-cyanoethyl group, 3-cyanopropyl group and 4-cyanobutyl group. Among these substituents, a hydroxyl group, a carboxyl group, a hydroxymethyl group, a cyano group and a cyanomethyl group are preferred.

Examples of the alkyl group of R¹ include an alkyl group having a carbon number of 1 to 4, such as methyl group, ethyl group, n-propyl group, i-propyl group, n-butyl group, 2-methylpropyl group, 1-methylpropyl group and tert-butyl group. Among these alkyl groups, a methyl group, an ethyl group, an n-propyl group and an i-propyl group are preferred.

Specific preferred examples include the following compounds.

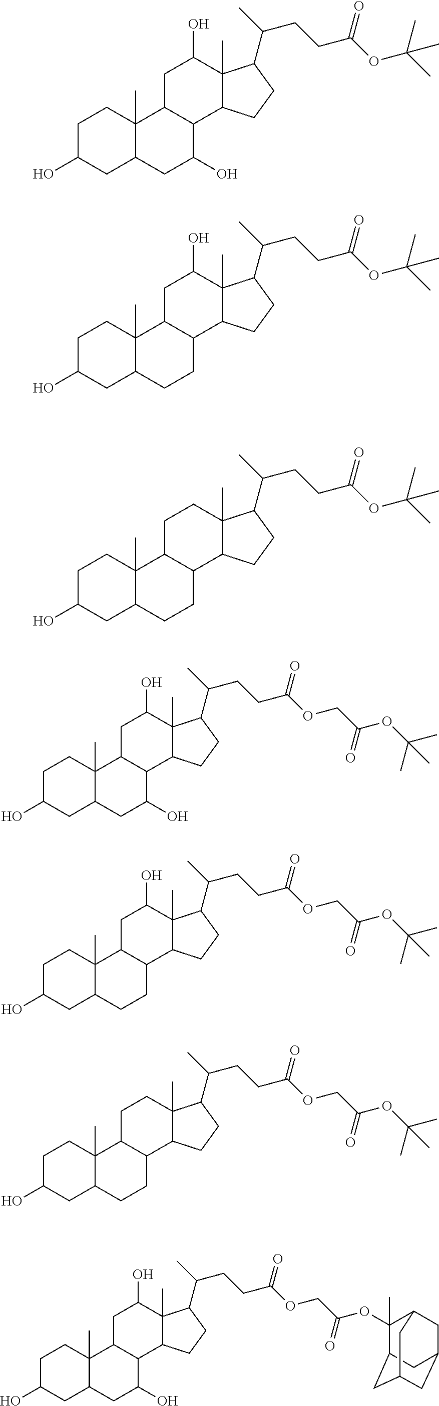

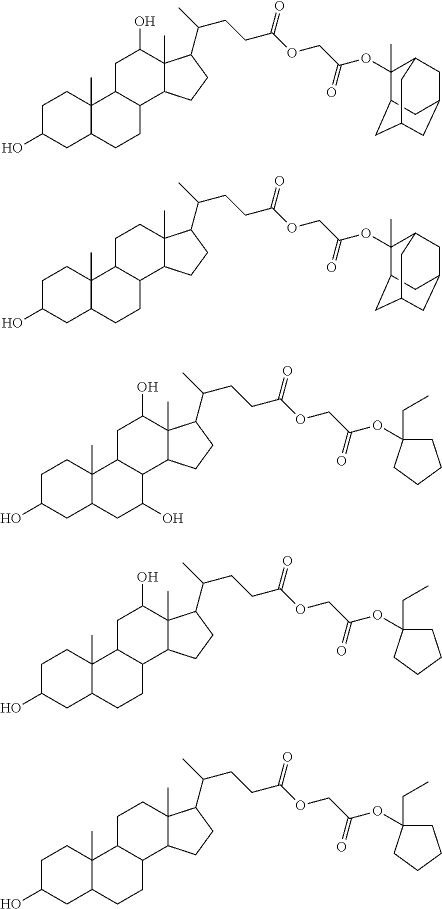

In the case where the low molecular compound is an unsaturated carboxylic acid ester, the compound is preferably a (meth)acrylic acid ester. Specific examples of the (meth)acrylic acid tertiary eater having a tertiary alkyl group as the group capable of leaving by the action of an acid are illustrated below, but the present invention is not limited thereto.

(In the formulae, Rx represents H, CH₃, CF₃ or CH₂OH, and each of Rxa and Rxb represents an alkyl group having a carbon number of 1 to 4.)

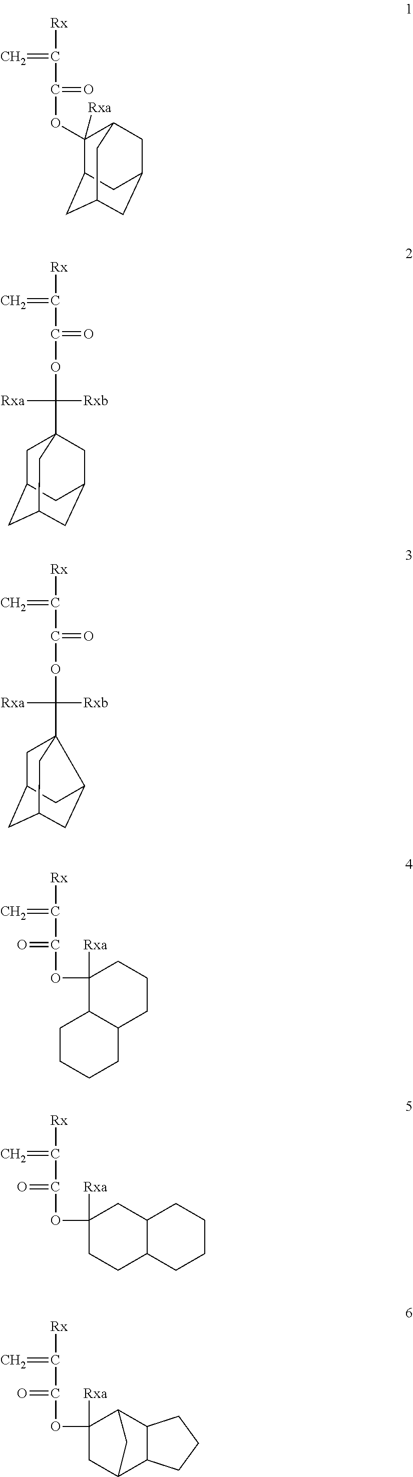

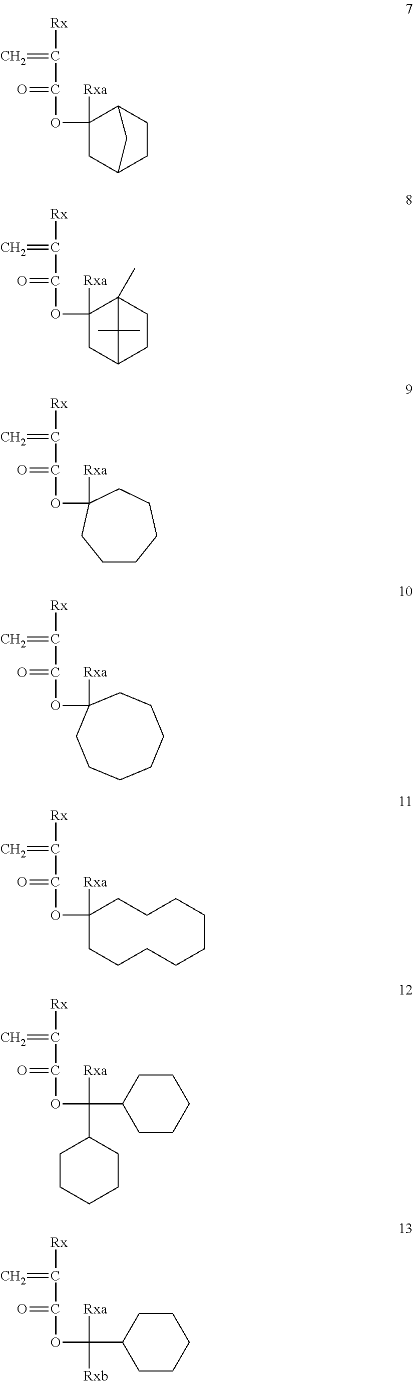

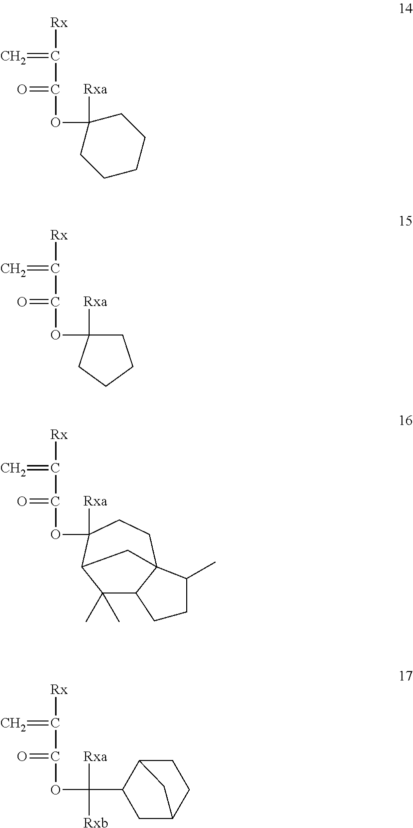

As for the (D) low molecular compound having a group capable of leaving by the action of an acid, a commercial product may be used or a compound synthesized by a known method may be used.

Also, an amine derivative having on the nitrogen atom a group capable of leaving by the action of an acid is preferred as the component D.

The component D may contain a carbamate group having a protective group on the nitrogen atom. The protective group constituting the carbamate group can be represented by the following formula (d-1):

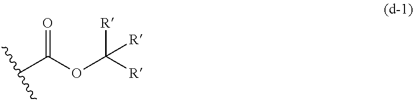

In formula (d-1), each R′ independently represents a hydrogen atom, a linear, branched or cyclic alkyl group, an aryl group, an aralkyl group or an alkoxyalkyl group. Each R′ may combine with every other R′ to form a ring.

R′ is preferably a linear or branched alkyl group, a cycloalkyl group or an aryl group, more preferably a linear or branched alkyl group or a cycloalkyl group.

The component D may also be composed by arbitrarily combining the above-described basic compound and the structure represented by formula (d-1).

The component D is more preferably a compound having a structure represented by the following formula (A).

Incidentally, the component D may be a compound corresponding to the above-described basic compound as long as it is a low molecular compound having a group capable of leaving by the action of an acid.

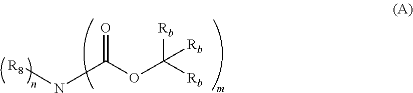

In formula (A), each Ra independently represents a hydrogen atom, an alkyl group, a cycloalkyl group, an aryl group or an aralkyl group. Also, when n=2, two Ra's may be the same or different, and two Ra's may combine with each other to form a divalent heterocyclic hydrocarbon group (preferably having a carbon number of 20 or less) or a derivative thereof.

Each Rb independently represents a hydrogen atom, an alkyl group, a cycloalkyl group, an aryl group or an aralkyl group, provided that in —C(Rb)(Rb)(Rb), when one or more Rb's are a hydrogen atom, at least one of remaining Rb's is a cyclopropyl group or a 1-alkoxyalkyl group.

At least two Rb's may combine to form an alicyclic hydrocarbon group, an aromatic hydrocarbon group, a heterocyclic hydrocarbon group or a derivative thereof.

n represents an integer of 0 to 2, m represents an integer of 1 to 3, and n+m=3.

In formula (A), each of the alkyl group, cycloalkyl group, aryl group and aralkyl group of Ra and Rb may be substitute with a functional group such as hydroxyl group, cyano group, amino group, pyrrolidino group, piperidino group, morpholino group and oxo group, an alkoxy group or a halogen atom.

Examples of the alkyl group, cycloalkyl group, aryl group and aralkyl group (each of these alkyl, cycloalkyl, aryl and aralkyl groups may be substituted with the above-described functional group, an alkoxy group or a halogen atom) of Ra and Rb include:

a group derived from a linear or branched alkane such as methane, ethane, propane, butane, pentane, hexane, heptane, octane, nonane, decane, undecane and dodecane, or a group where the group derived from an alkane is substituted with one or more in kind or number of cycloalkyl groups such as cyclobutyl group, cyclopentyl group and cyclohexyl group;

a group derived from a cycloalkane such as cyclobutane, cyclopentane, cyclohexane, cycloheptane, cyclooctane, norbornane, adamantane and noradamantane, or a group where the group derived from a cycloalkane is substituted with one or more in kind or number of linear or branched alkyl groups such as methyl group, ethyl group, n-propyl group, i-propyl group, n-butyl group, 2-methylpropyl group, 1-methylpropyl group and tert-butyl group;

a group derived from an aromatic compound such as benzene, naphthalene and anthracene, or a group where the group derived from an aromatic compound is substituted with one or more in kind or number of linear or branched alkyl groups such as methyl group, ethyl group, n-propyl group, i-propyl group, n-butyl group, 2-methylpropyl group, 1-methylpropyl group and tert-butyl group;

a group derived from a heterocyclic compound such as pyrrolidine, piperidine, morpholine, tetrahydrofuran, tetrahydropyran, indole, indoline, quinoline, perhydroquinoline, indazole and benzimidazole, or a group where the group derived from a heterocyclic compound is substituted with one or more in kind or number of linear or branched alkyl groups and aromatic compound-derived groups; a group where the group derived from a linear or branched alkane or the group derived from a cycloalkane is substituted with one or more in kind or number of aromatic compound-derived groups such as phenyl group, naphthyl group and anthracenyl group; and a group where the substituent above is substituted with a functional group such as hydroxyl group, cyano group, amino group, pyrrolidino group, piperidino group, morpholino group and oxo group.

Examples of the divalent heterocyclic hydrocarbon group (preferably having a carbon number of 1 to 20) formed by combining Ra's with each other or a derivative thereof include a group derived from a heterocyclic compound such as pyrrolidine, piperidine, morpholine, 1,4,5,6-tetrahydropyrimidine, 1,2,3,4-tetrahydroquinoline, 1,2,3,6-tetrahydropyridine, homopiperazine, 4-azabenzimidazole, benzotriazole, 5-azabenzotriazole, 1H-1,2,3-triazole, 1,4,7-triazacyclononane, tetrazole, 7-azaindole, indazole, benzimidazole, imidazo[1,2-a]pyridine, (1S,4S)-(+)-2,5-diazabicyclo[2.2.1]heptane, 1,5,7-triazabicyclo[4.4.0]dec-5-ene, indole, indoline, 1,2,3,4-tetrahydroquinoxaline, perhydroquinoline and 1,5,9-triazacyclododecane, and a group where the group derived from a heterocyclic compound is substituted with one or more in kind or number of linear or branched alkane-derived groups, cycloalkane-derived groups, aromatic compound-derived groups, heterocyclic compound-derived groups and functional groups such as hydroxyl group, cyano group, amino group, pyrrolidino group, piperidino group, morpholino group and oxo group.

Specific examples of the component D particularly preferred in the present invention are illustrated below, but the present invention is not limited thereto.

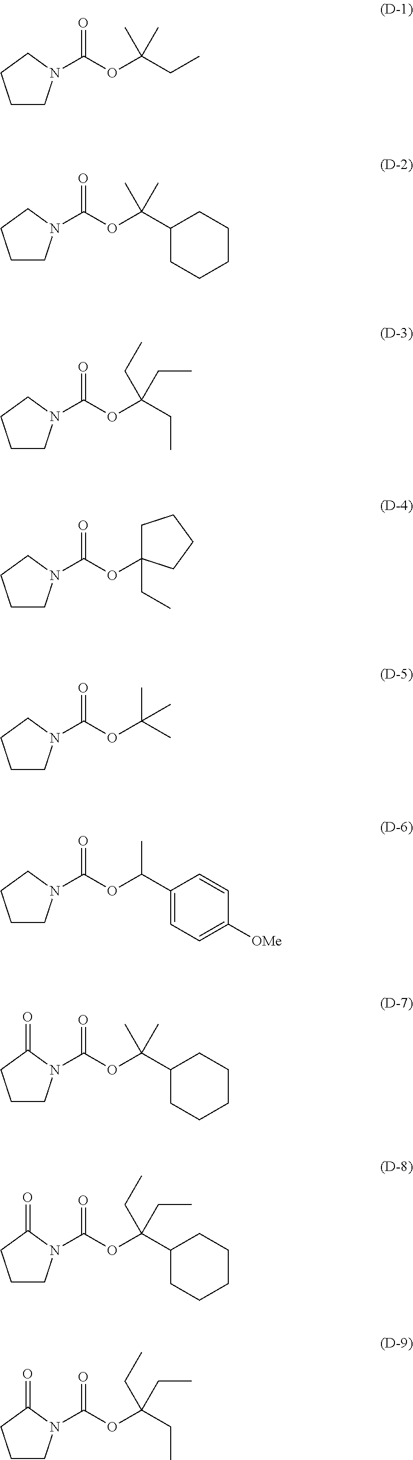

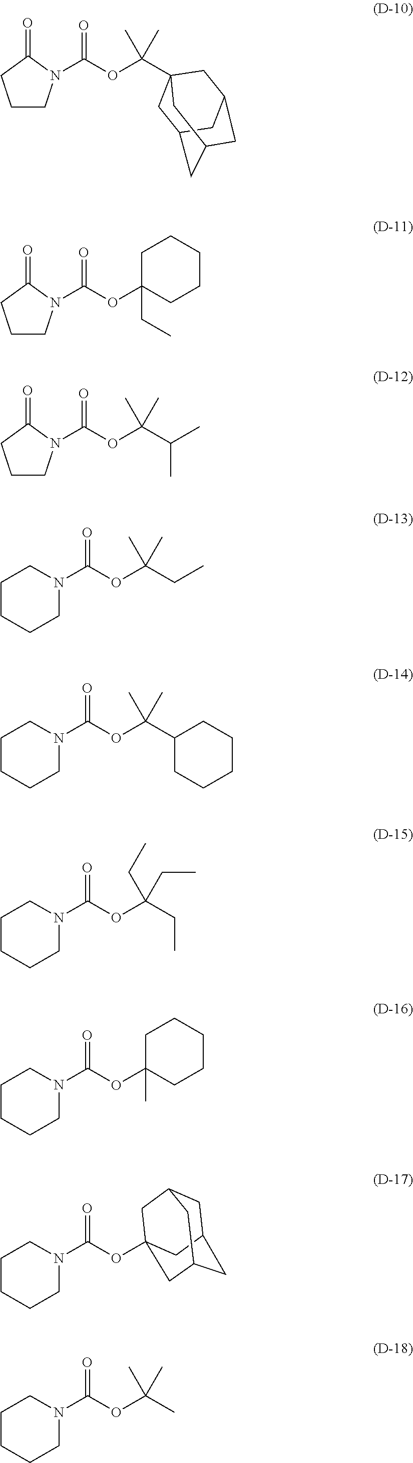

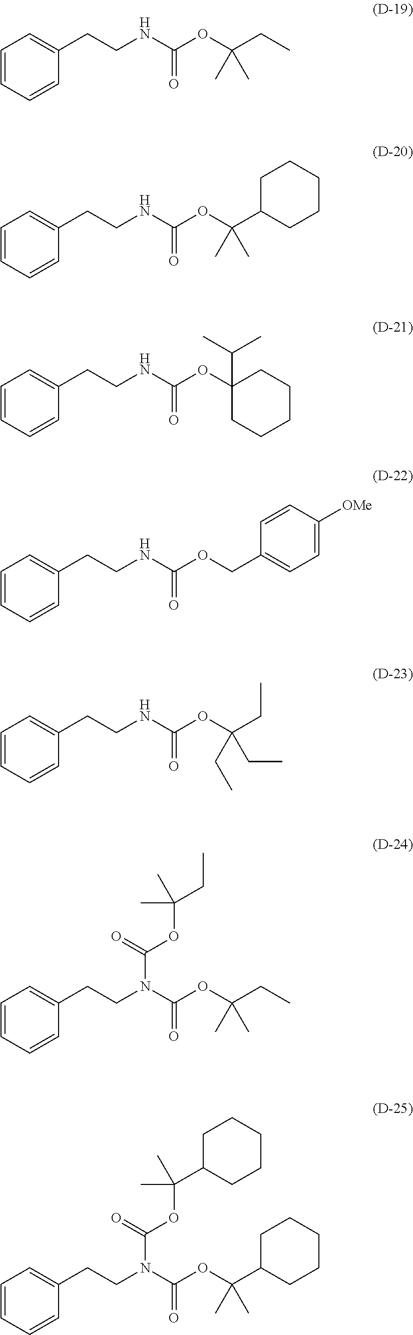

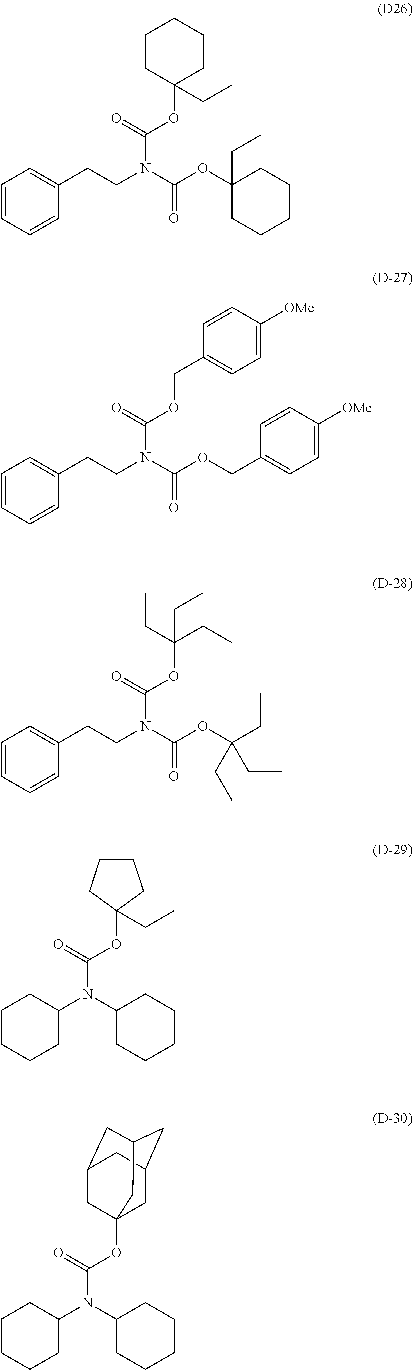

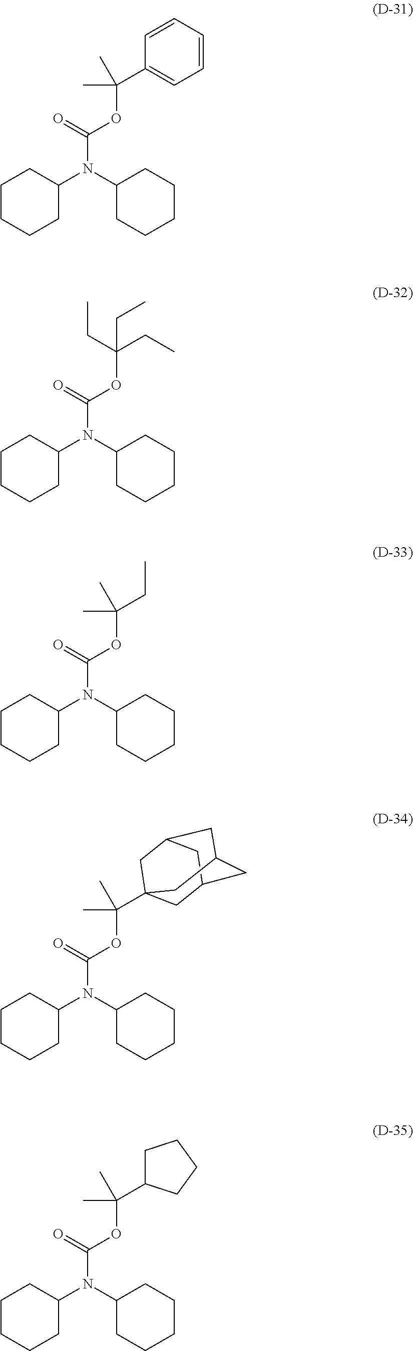

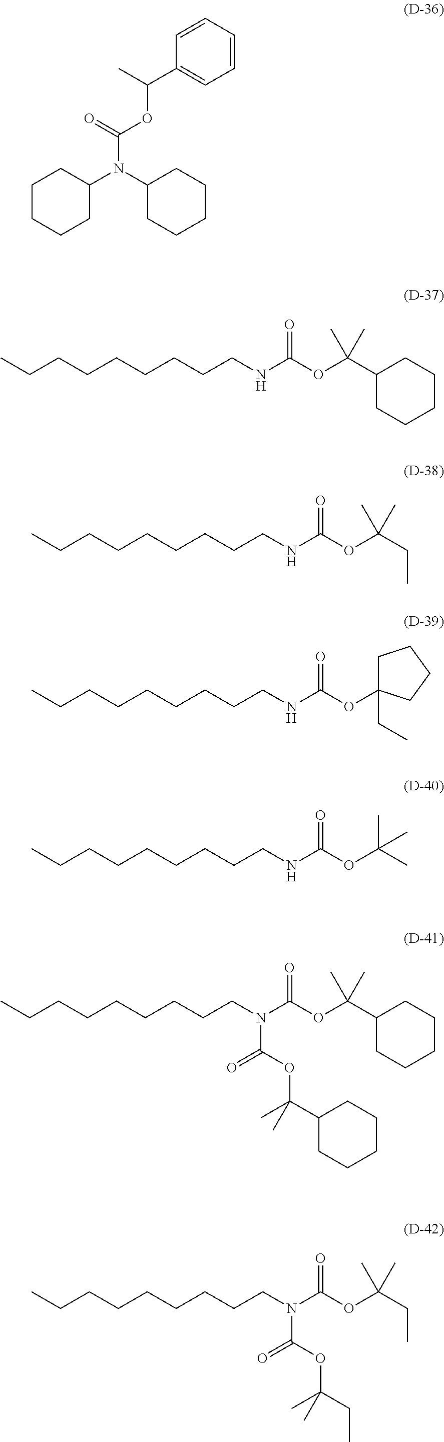

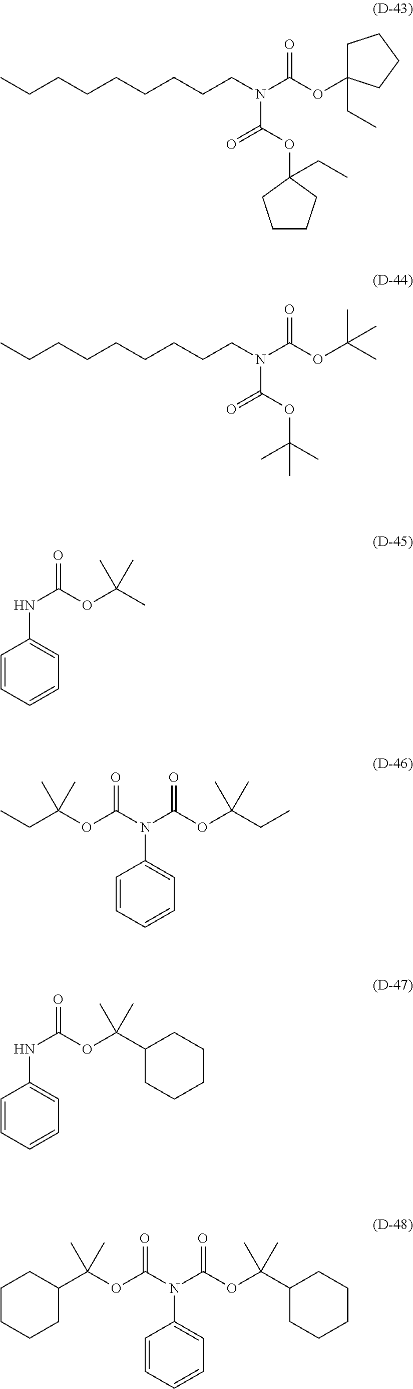

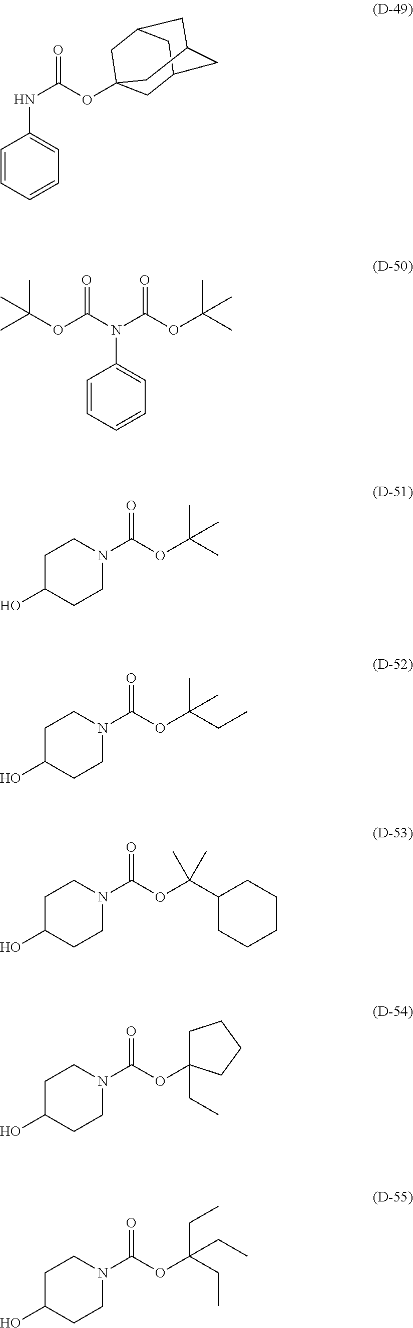

The compound represented by formula (A) can be easily synthesized from a commercially available amine by the method described, for example, in Protective Groups in Organic Synthesis, 4th edition. A most general method is a method of causing a dicarbonic acid ester or a haloformic acid ester to act on a commercially available amine to obtain the compound. In the formulae, X represents a halogen atom, and Ra and Rb have the same meanings as Ra and Rb, respectively, in formula (A).

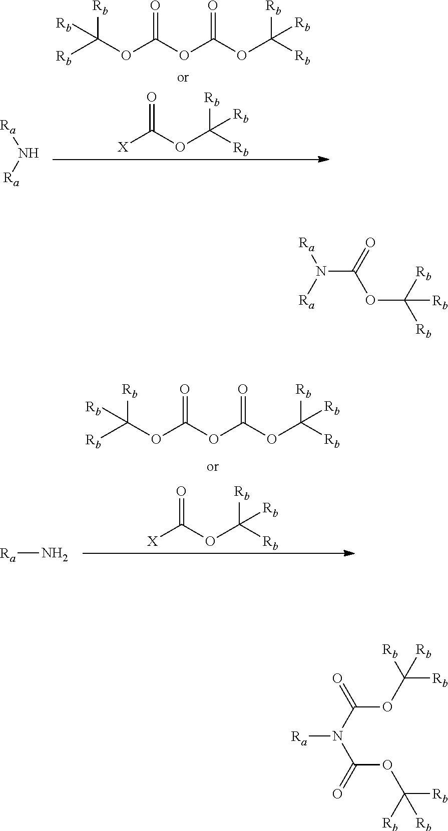

In the present invention, as for the (D) low molecular compound having a group capable of leaving by the action of an acid, one kind of a compound may be used alone, or two or more kinds of compounds may be mixed and used.

In the present invention, the amount used of the (D) low molecular compound having a group capable of leaving by the action of an acid is usually from 0.001 to 20 mass %, preferably from 0.001 to 10 mass %, more preferably from 0.01 to 5 mass %, based on the entire solid compound of the composition combined with the basic compound described above.

The ratio between the acid generator and the (D) low molecular compound having a group capable of leaving by the action of an acid, which are used in the composition, is preferably acid generator/[(D) low molecular compound having a group capable of leaving by the action of an acid+the above-described basic compound] (by mol)=from 2.5 to 300. That is, the molar ratio is preferably 2.5 or more in view of sensitivity and resolution and is preferably 300 or less from the standpoint of suppressing the reduction in resolution due to thickening of the resist pattern with aging after exposure until heat treatment. The acid generator/[(D) low molecular compound having a group capable of leaving by the action of an acid+the above-described basic compound] (by mol) is more preferably from 5.0 to 200, still more preferably from 7.0 to 150.

### [7] Surfactant

The composition of the present invention may further contain a surfactant. In the case of containing a surfactant, the surfactant is preferably a fluorine-containing and/or silicon-containing surfactant.

Examples of the surfactant above include Megaface F176 and Megaface R08 produced by Dainippon Ink & Chemicals, Inc.; PF656 and PF6320 produced by OMNOVA; Troysol S-366 produced by Troy Chemical; Florad FC430 produced by Sumitomo 3M Inc.; and polysiloxane polymer KP-341 produced by Shin-Etsu Chemical Co., Ltd.

A surfactant other than the fluorine-containing and/or silicon-containing surfactant may also be used. Specific examples thereof include polyoxyethylene alkyl ethers and polyoxyethylene alkylaryl ethers.

In addition, known surfactants may be appropriately used. Examples of the surfactant which can be used include surfactants described in paragraph [0273] et seq. of U.S. Patent Application Publication No. 2008/0248425A1.

One kind of a surfactant may be used alone, or two or more kinds of surfactants may be used in combination.

The amount of the surfactant used is preferably from 0 to 2 mass %, more preferably from 0.0001 to 2 mass %, still more preferably from 0.0005 to 1 mass %, based on the entire solid content (the entire amount excluding the solvent) of the actinic ray-sensitive or radiation-sensitive composition.

On the other hand, it is also preferred to set the amount added of the surfactant to 10 ppm or less or not to contain a surfactant. In this case, the hydrophobic resin is more unevenly distributed to the surface, whereby the resist film surface can be made more hydrophobic and the followability of water at the immersion exposure can be enhanced.

### [8] Solvent

The solvent that can be used at the time of preparing the composition is not particularly limited as long as it dissolves respective components, but examples thereof include an alkylene glycol monoalkyl ether carboxylate (e.g., propylene glycol monomethyl ether acetate), an alkylene glycol monoalkyl ether (e.g., propylene glycol monomethyl ether), an alkyl lactate (e.g., ethyl lactate, methyl lactate), a cyclic lactone (e.g., γ-butyrolactone; preferably having a carbon number of 4 to 10), a chain or cyclic ketone (e.g., 2-heptanone, cyclohexanone; preferably having a carbon number of 4 to 10), an alkylene carbonate (e.g., ethylene carbonate, propylene carbonate), an alkyl carboxylate (preferably an alkyl acetate such as butyl acetate), and an alkyl alkoxyacetate (e.g., ethyl ethoxypropionate). Other examples of the usable solvent include the solvents described in paragraph [0244] et seq. of U.S. Patent Application Publication No. 2008/0248425A1.

Among these, an alkylene glycol monoalkyl ether carboxylate and an alkylene glycol monoalkyl ether are preferred.

One of these solvents may be used alone, or two or more thereof may be mixed and used. In the case of mixing two or more kinds of solvent, a solvent having a hydroxyl group and a solvent having no hydroxyl group are preferably mixed. The mass ratio of the solvent containing a hydroxyl group to the solvent containing no hydroxyl group is from 1/99 to 99/1, preferably from 10/90 to 90/10, more preferably from 20/80 to 60/40.

The solvent having a hydroxyl group is preferably an alkylene glycol monoalkyl ether, and the solvent having no hydroxyl group is preferably an alkylene glycol monoalkyl ether carboxylate.

[9] (H) Substance Capable of Decomposing by the Action of an Acid to Produce an Acid Stronger than Carboxylic Acid

The composition of the present invention may contain (H) a substance capable of decomposing by the action of an acid to produce an acid stronger than carboxylic acid (hereinafter sometimes referred to as an “acid-increasing agent”).

The acid produced from the acid-increasing agent preferably has a large acid strength. Specifically, the dissolution constant (pKa) of the acid is preferably 3 or less, more preferably 2 or less. The acid generated from the acid-increasing agent is preferably a sulfonic acid.

Examples of the acid-increasing agent include acid-increasing agents described in International Publication Nos. 95/29968 and 98/24000, JP-A-8-305262, JP-A-9-34106, JP-A-8-248561, JP-T-8-503082 (the term “JP-T” as used herein means a published Japanese translation of a PCT patent application), U.S. Pat. No. 5,445,917, JP-T-8-503081, U.S. Pat. Nos. 5,534,393, 5,395,736, 5,741,630, 5,334,489, 5,582,956, 5,578,424, 5,453,345 and 5,445,917, European Patents 665,960, 757,628 and 665,961, U.S. Pat. No. 5,667,943, JP-A-10-1508, JP-A-10-282642, JP-A-9-512498, JP-A-2000-62337 and JP-A-2005-17730, and one of these acid-increasing agents may be used or two or more thereof may be used in combination.

Specifically, compounds represented by the following formulae (1) to (6) are preferred.

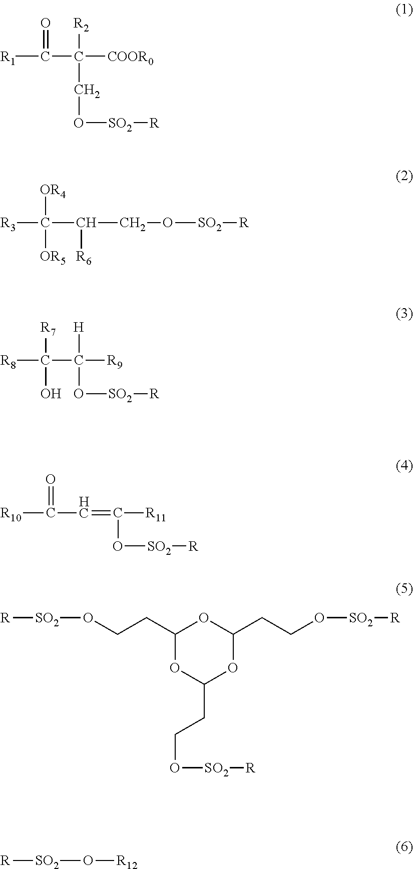

In formulae (1) to (6), R represents an alkyl group, a cycloalkyl group, an aryl group or an aralkyl group,

R₀ represents a group capable of leaving by the action of an acid,

R₁ represents an alkyl group, a cycloalkyl group, an aryl group, an aralkyl group, an alkoxy group or an aryloxy group,

R₂ represents an alkyl group or an aralkyl group,

R₃ represents an alkyl group, a cycloalkyl group, an aryl group or an aralkyl group,

each of R₄ and R₅ independently represents an alkyl group, R4 and R5 may combine with each other to form a ring,

R₆ represents a hydrogen atom or an alkyl group,

R₇ represents a hydrogen atom, an alkyl group, a cycloalkyl group, an aryl group or an aralkyl group,

R₈ represents an alkyl group, a cycloalkyl group, an aryl group or an aralkyl group,

R₉ represents a hydrogen atom, an alkyl group, a cycloalkyl group, an aryl group or an aralkyl group,

R₉ may combine with R₇ to form a ring,

R₁₀ represents an alkyl group, a cycloalkyl group, an alkoxy group, an aryl group, an aralkyl group, an aryloxy group or an alkenyloxy group,

R₁₁ represents an alkyl group, a cycloalkyl group, an alkoxy group, an aryl group, an aralkyl group, an aryloxy group or an alkenyl group,

R₁₀ and R₁₁ may combine with each other to form a ring, and

R₁₂ represents an alkyl group, a cycloalkyl group, an aryl group, an alkenyl group or a cyclic imide group.

In formulae (1) to (6), the alkyl group includes an alkyl group having a carbon number of 1 to 8, and specific examples thereof include a methyl group, an ethyl group, a propyl group, an isopropyl group, a butyl group and an octyl group.

The cycloalkyl group includes a cycloalkyl group having a carbon number of 4 to 10, and specific examples thereof include a cyclopropyl group, a cyclopentyl group, a cyclohexyl group, a cycloheptyl group, an adamantyl group, a boronyl group, an isoboronyl group, a tricyclodecanyl group, a dicyclopentenyl group, a norbornane epoxy group, a menthyl group, an isomenthyl group, a neomenthyl group and a tetracyclododecanyl group.

The aryl group includes an aryl group having a carbon number of 6 to 14, and specific examples thereof include a phenyl group, a naphthyl group and a tolyl group.

The aralkyl group includes an aralkyl group having a carbon number of 7 to 20, and specific examples thereof include a benzyl group, a phenethyl group and a naphthylethyl group.

The alkoxy group includes an alkoxy group having a carbon number of 1 to 8, and specific examples thereof include a methoxy group, an ethoxy group, a propoxy group and a butoxy group.

The alkenyl group includes an alkenyl group having a carbon number of 2 to 6, and specific examples thereof include a vinyl group, a propenyl group, an allyl group, a butenyl group, a pentenyl group, a hexenyl group and a cyclohexenyl group.

The aryloxy group includes an aryloxy group having a carbon number of 6 to 14, and specific examples thereof include a phenoxy group and a naphthoxy group.

The alkenyloxy group includes an alkenyloxy group having a carbon number of 2 to 8, and specific examples thereof include a vinyloxy group and an allyloxy group.

Each of the above-described substituents may further have a substituent, and examples of the substituent include a halogen atom such as Cl, Br and F, a —CN group, an —OH group, an alkyl group having a carbon number of 1 to 4, a cycloalkyl group having a carbon number of 3 to 8, an alkoxy group having a carbon number of 1 to 4, an acylamino group such as acetylamino group, an aralkyl group such as benzyl group and phenethyl group, an aryloxyalkyl group such as phenoxyethyl group, an alkoxycarbonyl group having a carbon number of 2 to 5, and an acyloxy group having a carbon number of 2 to 5, but the range of the substituent is not limited thereto.

Examples of the ring formed by combining R4 and R5 with each other include a 1,3-dioxolane ring and a 1,3-dioxane ring.

Examples of the ring formed by combining R7 and R9 with each other include a cyclopentyl ring and a cyclohexyl ring.

Examples of the ring formed by combining R10 and R₁₁ with each other include a 3-oxocyclohexenyl ring and a 3-oxoindenyl ring, which each may contain an oxygen atom in the ring.

Examples of the group capable of leaving by the action of an acid of R₀ include a tertiary alkyl group such as tert-butyl group and tert-amyl group, an isoboronyl group, a 1-alkoxyethyl group such as 1-ethoxyethyl group, 1-butoxyethyl group, 1-isobutoxyethyl group and 1-cyclohexyl oxyethyl group, an alkoxymethyl group such as 1-methoxymethyl group and 1-ethoxymethyl group, a tetrahydropyranyl group, a tetrahydrofuranyl group, a trialkylsilyl group, and a 3-oxocyclohexyl group.

When R₁₂ represents a cyclic imide group, the cyclic imide may be a cyclic imide having a carbon number of 4 to 20, such as succinic acid imide, phthalic acid imide, cyclohexanedicarboxylic acid imide and norbornenedicarboxylic acid imide.

Specific examples of the compounds represented by formulae (1) to (6) include the compounds illustrated in paragraph [0215] et seq. of JP-A-2008-209889.

### [10] Other Components

The composition of the present invention may appropriately contain, in addition to the above-described components, an onium carboxylate, a dissolution inhibiting compound having a molecular weight of 3,000 or less described, for example, in Proceeding of SPIE, 2724, 355 (1996), an acid-increasing agent, a dye, a plasticizer, a photosensitizer, a light absorber, and the like.

### [Pattern Forming Method]

The composition of the present invention is used by dissolving the components above in a solvent, filtering the solution, and applying it on a support. The filter is preferably a polytetrafluoroethylene-, polyethylene- or nylon-made filter having a pore size of 0.1 μm or less, more preferably 0.05 μm or less, still more preferably 0.03 μm or less.

The composition of the present invention can be applied on such a substrate (e.g., silicon/silicon dioxide-coated substrate) as used in the production of an integrated circuit device, by an appropriate coating method such as spinner. The coating is then dried, whereby a photosensitive resist film can be formed.

The resist film is irradiated with an actinic ray or radiation through a predetermined mask, preferably baked (heated) and then subjected to development and rinsing, whereby a good pattern can be obtained. Incidentally, in the case of irradiation with an electron beam, lithography without a mask (direct lithography) is generally performed.

After the film formation, the pattern forming method preferably contains a pre-baking step (PB) before the exposure step.

Also, the pattern forming method preferably contains a post-exposure baking step (PEB) after the exposure step but before the development step.

As for the heating temperature, both PB and PEB are preferably performed at 70 to 140° C., more preferably at 80 to 135° C.

The heating time is preferably from 30 to 300 seconds, more preferably from 30 to 180 seconds, still more preferably from 30 to 90 seconds.

The heating can be performed using a device attached to an ordinary exposure/developing machine or may be performed using a hot plate or the like.

Thanks to baking, the reaction in the exposed area is accelerated, and the sensitivity and pattern profile are improved.

The actinic ray or radiation is not particularly limited but is, for example, KrF excimer laser (248 nm), ArF excimer laser (193 nm), EUV light (13 nm) or electron beam, and ArF excimer laser, EUV light and electron beam are preferred.

As for the alkali developer used in the development step, a quaternary ammonium salt typified by tetramethylammonium hydroxide (TMAH) is usually used, but other than this compound, an aqueous alkali solution of inorganic alkali, primary to tertiary amine, alcohol amine, cyclic amine or the like can also be used.

Furthermore, this alkali developer may be used after adding thereto alcohols and a surfactant each in an appropriate amount.

The alkali concentration of the alkali developer is usually from 0.1 to 20 mass %.

The pH of the alkali developer is usually from 10.0 to 15.0.

As for the rinsing solution, pure water is used, and an appropriate amount of a surfactant may be added thereto before use.

Before forming the photosensitive resist film, an antireflection film may be previously provided by coating on the substrate.

The antireflection film used may be either an inorganic film type such as titanium, titanium dioxide, titanium nitride, chromium oxide, carbon and amorphous silicon, or an organic film type composed of a light absorber and a polymer material. As for the organic antireflection film, there may also be used a commercially available organic antireflection film such as DUV30 Series and DUV-40 Series produced by Brewer Science, Inc. and AR-2, AR-3 and AR-5 produced by Shipley Co., Ltd.

With respect to the resist film formed from the actinic ray-sensitive or radiation-sensitive resin composition of the present invention, the exposure may also be performed by filling a liquid (immersion medium) having a refractive index higher than that of air between the film and a lens at the irradiation with an actinic ray or radiation (immersion exposure). By this exposure, the resolution can be enhanced. The immersion medium used may be any liquid as long as it has a refractive index higher than that of air, but pure water is preferred.

The immersion liquid used in the immersion exposure is described below.

The immersion liquid is preferably a liquid being transparent to light at the exposure wavelength and having as small a temperature coefficient of refractive index as possible so as to minimize the distortion of an optical image projected on the resist film. Particularly, when the exposure light source is an ArF excimer laser (wavelength: 193 nm), water is preferably used in view of easy availability and easy handleability in addition to the above-described aspects.

Furthermore, a medium having a refractive index of 1.5 or more can also be used from the standpoint that the refractive index can be more enhanced. This medium may be either an aqueous solution or an organic solvent.

In the case of using water as the immersion liquid, for the purpose of decreasing the surface tension of water and increasing the surface activity, an additive (liquid) which does not dissolve the resist film on a wafer and at the same time, gives only a negligible effect on the optical coat at the undersurface of the lens element, may be added in a small ratio. The additive is preferably an aliphatic alcohol having a refractive index nearly equal to that of water, and specific examples thereof include methyl alcohol, ethyl alcohol and isopropyl alcohol. By virtue of adding an alcohol having a refractive index nearly equal to that of water, even when the alcohol component in water is evaporated and its content concentration is changed, the change in the refractive index of the entire liquid can be advantageously made very small. On the other hand, if a substance opaque to light at 193 nm or an impurity greatly differing in the refractive index from water is mixed, this incurs distortion of the optical image projected on the resist film. Therefore, the water used is preferably distilled water. Pure water obtained by further filtering the distilled water through an ion exchange filter or the like may also be used.

The electrical resistance of water is preferably 18.3 MQcm or more, and TOC (total organic carbon) is preferably 20 ppb or less. Also, the water is preferably subjected to a deaeration treatment.

The lithography performance can be enhanced by elevating the refractive index of the immersion liquid. From such a standpoint, an additive for elevating the refractive index may be added to water, or deuterium water (D₂O) may be used in place of water.

In the case of exposing the film formed of the composition of the present invention through an immersion medium, as described above, a hydrophobic resin (HR) can be further added, if desired.

In order to prevent the film from directly contacting with the immersion liquid, a film (hereinafter, sometimes referred to as a “topcoat”) sparingly soluble in the immersion liquid may be provided between the film formed of the composition of the present invention and the immersion liquid. The functions required of the topcoat are suitability for coating as an overlayer of the resist, transparency to radiation particularly at 193 nm, and sparing solubility in the immersion liquid. The topcoat is preferably unmixable with the resist and capable of being uniformly applied as an overlayer of the resist.

In view of transparency to light at 193 nm, the topcoat is preferably a polymer not abundantly containing an aromatic, and specific examples thereof include a hydrocarbon polymer, an acrylic acid ester polymer, a polymethacrylic acid, a polyacrylic acid, a polyvinyl ether, a silicon-containing polymer and a fluorine-containing polymer. The above-described hydrophobic resins (HR) is suitable also as the topcoat. If impurities are dissolved out into the immersion liquid from the topcoat, the optical lens is contaminated. In this viewpoint, the amount of residual monomer components of the polymer contained in the topcoat is preferably smaller.

On peeling off the topcoat, a developer may be used or a releasing agent may be separately used. The releasing agent is preferably a solvent less permeating the film. From the standpoint that the peeling step can be performed simultaneously with the development step of the film, the topcoat is preferably peelable with an alkali developer and for enabling the peeling with an alkali developer, the topcoat is preferably acidic, but in view of non-intermixing with the film, the topcoat may be neutral or alkaline.

With no difference in the refractive index between the topcoat and the immersion liquid, the resolution is enhanced. In the case of using water as the immersion liquid at the exposure to ArF excimer laser (wavelength: 193 nm), the topcoat for ArF immersion exposure preferably has a refractive index close to the refractive index of the immersion liquid. From the standpoint of having a refractive index close to that of the immersion liquid, the topcoat preferably contains a fluorine atom. Also, in view of transparency and refractive index, the topcoat is preferably a thin film.

The topcoat is preferably unmixable with the film and further unmixable with the immersion liquid. From this standpoint, when the immersion liquid is water, the solvent used for the topcoat is preferably a medium that is sparingly soluble in the solvent used for the composition of the present invention and insoluble in water. In the case where the immersion liquid is an organic solvent, the topcoat may be either water-soluble or water-insoluble.

### EXAMPLES

The present invention is described in greater detail below by referring to Examples, but the present invention should not be construed as being limited thereto.

### Synthesis Example 1

### Synthesis of Compound (PA-1)

Under nitrogen flow, a mixture of 8.35 g (26.4 mmol) of 1,1,2,2,3,3-hexafluoropropane-1,3-disulfonyl fluoride and 15 ml of THF was cooled on ice and thereto, a mixed solution of 2.77 g (27.7 mmol) of 1-methylpiperazine and 30 ml of triethylamine was added dropwise over 60 minutes. The resulting solution was stirred under ice cooling for 1 hour and further stirred at room temperature for 1 hour, and 3.94 g (26.4 mmol) of trifluoromethanesulfonamide was added thereto. This mixture was stirred at 80° C. for 12 hours, and 100 ml of chloroform was added. The organic layer was washed with water and dried over sodium sulfate, and 20 ml of methanol and 50 ml of aqueous 1.5 N hydrochloric acid were added. The precipitated white solid was filtered to obtain 16.5 g of Compound (A) shown below and after adding 12.5 g of Compound (A) to 200 ml of water, sodium hydrogencarbonate was added until the pH became 7, thereby preparing a mixed solution (Solution A).

Subsequently, in a three-neck flask, 20 g of bromobutane and 12.5 g of 1-naphthol were dissolved in 300 g of NMP (N-methylpyrrolidone), and 12 g of potassium carbonate and 14 g of potassium iodide were added thereto. The mixture was heated at 120° C. for 8 hours, and 300 g of water was added to the reaction solution. The resulting solution was extracted with 100 g of hexane three times, and the obtained organic layers were combined. The combined organic layer was washed with 100 g of an aqueous 1N sodium hydroxide solution once, with 100 g of water once and with 100 g of brine once, and then concentrated to obtain 13.1 g of Compound (B).

In a three-neck flask, 6.5 g of Compound (B) was dissolved in 32 g of Eaton's reagent, and 2.9 g of tetramethylene sulfoxide was added dropwise with stirring. The resulting solution was further stirred for 3 hours, and after pouring the obtained reaction solution in 120 g of water, sodium hydrogencarbonate was added thereto until the pH became 7. To this mixed solution, Solution A and 25 g of chloroform were added, and the organic layer was separated. The aqueous layer was further extracted twice by using 25 g of chloroform, and the obtained organic layers were combined. The combined organic layer was washed with water twice and concentrated, and the obtained crude product was recrystallized using 10 g of ethyl acetate to obtain 11 g of the objective Compound (PA-1).

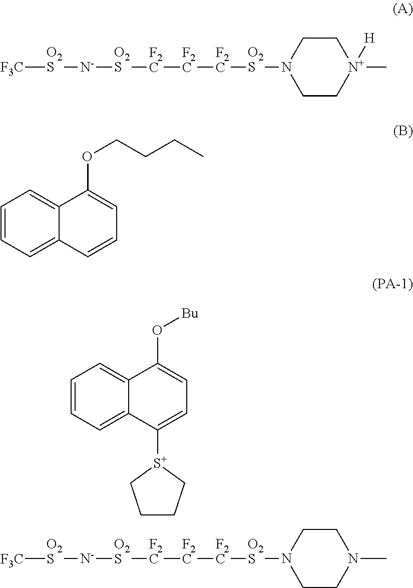

### (Method for Measuring Molar Extinction Coefficient)

Compound (PA-1) (25 mg) was precisely weighed into a 100 ml-volume measuring flask, and acetonitrile was added to the marked line (Solution X). Furthermore, 2 ml of Solution X was transferred to a 50 ml-volume measuring flask by using a whole pipette, and acetonitrile was added to the marked line. The resulting solution was measured by taking UV measurement using CARY-5G manufactured by VARIAN, and the absorbance at 193 nm was determined. The molar extinction coefficient ε was calculated in accordance with the Lambert-Beer law and calculated from the absorbance (A) at 193 nm and the concentration (c) of the measurement solvent.

Other compounds (PA) described later were synthesized in the same manner and measured for the molar extinction coefficient.

### Synthesis Example 2

### Synthesis of Resin D

Under nitrogen flow, 25.5 g of cyclohexanone was charged into a three-neck flask, and the flask was heated to 80° C. Thereto, a solution obtained by dissolving the compounds (monomers) shown below in amounts of 6.78 g, 1.25 g, 2.71 g and 1.95 g starting from the left and a polymerization initiator V-601 (produced by Wako Pure Chemical Industries, Ltd., 0.798 g) in 46 g of cyclohexane was added dropwise over 6 hours. After the completion of dropwise addition, the reaction was further allowed to proceed at 80° C. for 2 hours. The reaction solution was left standing to cool and then added dropwise to a mixed solution of 420 g of hexane/180 g of ethyl acetate over 20 minutes. The precipitated powder was collected by filtration and dried, as a result, 9.8 g of Resin D was obtained. The weight average molecular weight of Resin D was 8,500 in terms of standard polystyrene, and the polydispersity (Mw/Mn) was 1.55.

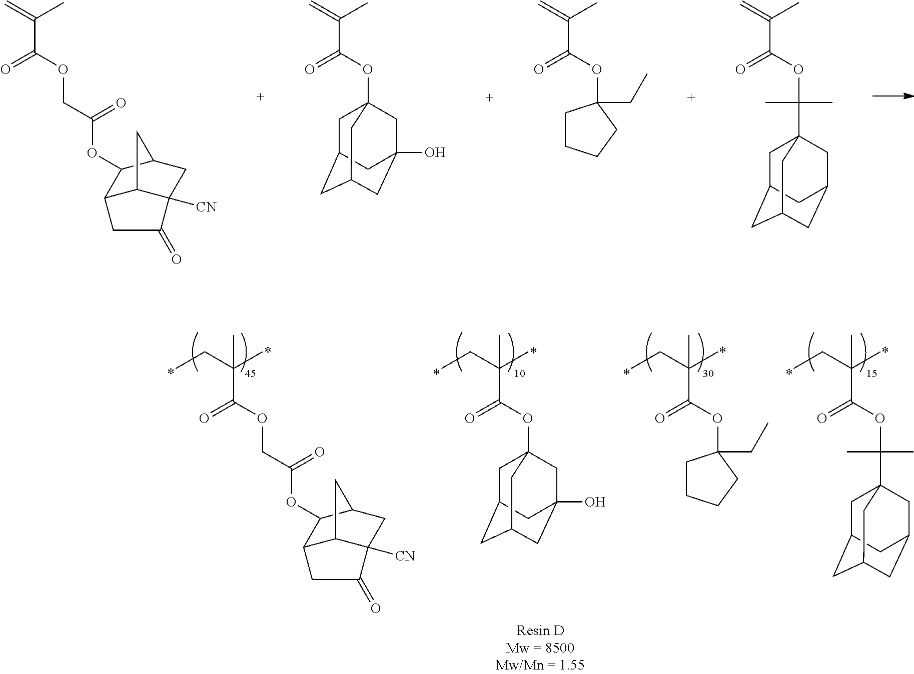

### <Preparation of Resist>

The components shown in Table 3 below were dissolved in a solvent to prepare a solution having a solid content concentration of 4 mass %, and the obtained solution was filtered through a polyethylene filter having a pore size of 0.05 μm to prepare an actinic ray-sensitive or radiation-sensitive resin composition. The actinic ray-sensitive or radiation-sensitive resin compositions prepared were evaluated by the following methods, and the results are shown in Table 3.

As for each component in the Table, the ratio when using a plurality of kinds is a ratio by mass.

Incidentally, in Table 3, when the actinic ray-sensitive or radiation-sensitive resin composition contains a hydrophobic resin (HR), the mode of addition is denoted by “added”, and when the actinic ray-sensitive or radiation-sensitive resin composition does not contain a hydrophobic resin (HR) and after the formation of film, a topcoat protective film containing a hydrophobic resin (HR) is formed as an overlayer thereof, the mode of addition is denoted by “TC”.

### <Evaluation of Resist>

### Exposure Condition 1: ArF Immersion Exposure

### Examples 1 to 44, Comparative Examples 1 to 3, and Examples 54 to 56 and 58 to 65

An organic antireflection film, ARC29A (produced by Nissan Chemical Industries, Ltd.), was applied on a silicon wafer and baked at 205° C. for 60 seconds to form an antireflection film having a film thickness of 98 nm, and the actinic ray-sensitive or radiation-sensitive resin composition prepared above was applied thereon and baked at 130° C. for 60 seconds to form a film having a film thickness of 120 nm. In the case of using a topcoat, a solution with a concentration of 3 mass % prepared by dissolving a topcoat resin in decane/octanol (mass ratio: 9/1) was applied on the film obtained above and then baked at 85° C. for 60 seconds to form a topcoat layer having a film thickness of 50 nm. The resulting wafer was exposed through a 6% halftone mask having a 1:1 line-and-space pattern of 45 nm in line width by using an ArF excimer laser immersion scanner (XT1700i, manufactured by ASML, NA: 1.2). As for the immersion liquid, ultrapure water was used. Thereafter, the wafer was heated at 130° C. for 60 seconds, developed with an aqueous tetramethylammonium hydroxide solution (2.38 mass %) for 30 seconds, rinsed with pure water and spin-dried to form a pattern.

### Exposure Condition 2: ArF Dry Exposure

### Examples 45 to 53, 57 and Comparative Example 4

An organic antireflection film, ARC29A (produced by Nissan Chemical Industries, Ltd.), was applied on a silicon wafer and baked at 205° C. for 60 seconds to form an antireflection film having a film thickness of 78 nm, and the positive resist composition prepared was applied thereon and baked at 130° C. for 60 seconds to form a resist film having a film thickness of 120 nm. The obtained wafer was exposed through a 6% halftone mask having a 1:1 line-and-space pattern of 75 nm in line width by using an ArF excimer laser scanner (PAS5500/1100, manufactured by ASML, NA: 0.75), then heated at 130° C. for 60 seconds, developed with an aqueous tetramethylammonium hydroxide solution (2.38 mass %) for 30 seconds, rinsed with pure water and spin-dried to obtain a resist pattern.

### (Evaluation of LWR):

The obtained line pattern with line/space of 1/1 (dry: line width of 75 nm, immersion: line width of 45 nm) was observed by a scanning electron microscope (S9380, manufactured by Hitachi, Ltd.) and with respect to the range in 2 μm of the longitudinal edge of the line pattern, the line width was measured at 50 points. The standard deviation of the variation in measurement was determined, and 3σ was computed. A smaller value indicates higher performance.

### (Evaluation of DOF)

In Exposure Condition 1, the amplitude of depth-of-focus for reproducing a line width of 45 nm±10% was measured, and in Exposure Condition 2, the amplitude of depth-of-focus for reproducing a line width of 75 nm±10% was measured as DOF (μm). A larger value indicates a wider defocus latitude and is better.

TABLE 3

| | Composition | | | | | | | | |
|---|---|---|---|---|---|---|---|---|---|
| | | Acid | | Basic Compound | Resin (HR) | | | Results | |
| | Compound | Generator | Resin (B1) | or Compound | (35 mg), | Surfactant | Solvent | LWR | DOF |
| | (PA) (g) | (g) | (10 g) | (D) (g) | mode of use | (g) | (mass ratio) | (nm) | (μm) |
| Example 1 | PA-10 | PAG-C | D | DIA | HR-47 | W-4 | S1 | 4.0 | 0.12 |
| | (0.31) | (1.55) | | (0.05) | added | (0.01) | (100) | | |
| Example 2 | PA-10 | PAG-C | C | PEA | HR-28 | W-2 | S1/S4/S6 | 4.1 | 0.12 |
| | (0.28) | (1.53) | | (0.05) | added | (0.02) | (80/5/15) | | |
| Example 3 | PA-2 | PAG-B | B | TEA | HR-53 | W-1 | S1/S6 | 5.7 | 0.12 |
| | (0.32) | (1.43) | | (0.03) | added | (0.01) | (95/5) | | |
| Example 4 | PA-10 | PAG-C | B | DIA | HR-26 | W-4 | S1/S3 | 4.2 | 0.12 |
| | (0.15) | (1.23) | | (0.03) | added | (0.01) | (90/10) | | |
| Example 5 | PA-4 | PAG-C | D | PEA | HR-28 | W-4 | S1/S5 | 6.0 | 0.11 |
| | (0.20) | (1.32) | | (0.02) | added | (0.01) | (80/20) | | |
| Example 6 | PA-12 | PAG-C | D | DIA | HR-47 | W-4 | S1/S4/S6 | 4.2 | 0.12 |
| | (0.35) | (1.44) | | (0.02) | added | (0.01) | (80/5/15) | | |
| Example 7 | PA-1 | PAG-C | D | PBI | HR-26 | W-3 | S1 | 4.7 | 0.12 |
| | (0.25) | (1.23) | | (0.04) | added | (0.02) | (100) | | |
| Example 8 | PA-13 | PAG-C | D | DBA | HR-53 | none | S1/S3 | 4.4 | 0.12 |
| | (0.31) | (1.48) | | (0.03) | added | | (90/10) | | |
| Example 9 | PA-1 | PAG-B | C | TPSA | HR-47 | W-4 | S1 | 4.6 | 0.12 |
| | (0.10) | (1.32) | | (0.05) | added | (0.01) | (100) | | |
| Example 10 | PA-8 | PAG-C | D | DIA | HR-47 | W-4 | S1 | 5.2 | 0.09 |
| | (0.36) | (1.57) | | (0.05) | added | (0.01) | (100) | | |
| Example 11 | PA-14 | PAG-G | D | PEA | HR-47 | W-4 | S1/S5 | 5.3 | 0.11 |
| | (0.20) | (1.62) | | (0.05) | added | (0.01) | (60/40) | | |
| Example 12 | PA-1 | PAG-B | B | TEA | HR-47 | W-4 | S1 | 4.7 | 0.12 |
| | (0.31) | (1.52) | | (0.04) | added | (0.01) | (100) | | |
| Example 13 | PA-5 | PAG-C | D | PEA | HR-47 | W-4 | S1 | 6.2 | 0.11 |
| | (0.29) | (1.42) | | (0.04) | added | (0.01) | (100) | | |
| Example 14 | PA-9 | PAG-C | D | PEA | HR-49 | W-4 | S1/S3 | 6.3 | 0.10 |
| | (0.28) | (1.39) | | (0.04) | TC | (0.01) | (90/10) | | |
| Example 15 | PA-11 | PAG-G | D | PEA | HR-28 | W-4 | S1 | 4.8 | 0.11 |
| | (0.33) | (1.35) | | (0.04) | added | (0.01) | (100) | | |
| Example 16 | PA-11 | PAG-C | D | PEA | HR-53 | W-4 | S1/S3 | 4.9 | 0.11 |
| | (0.35) | (1.46) | | (0.04) | added | (0.01) | (90/10) | | |
| Example 17 | PA-6 | PAG-E | D | PEA | HR-47 | W-4 | S1/S5 | 6.5 | 0.08 |
| | (0.40) | (1.57) | | (0.04) | added | (0.01) | (60/40) | | |
| Example 18 | PA-6 | PAG-C | D | DIA | HR-49 | W-3 | S1/S3 | 6.6 | 0.08 |
| | (0.21) | (1.18) | | (0.05) | TC | (0.02) | (90/10) | | |
| Example 19 | PA-3 | PAG-F | D | DBA | HR-47 | W-3 | S1/S3 | 5.0 | 0.11 |
| | (0.25) | (1.15) | | (0.03) | added | (0.01) | (90/10) | | |
| Example 20 | PA-3 | PAG-C | D | DIA | HR-47 | W-4 | S1/S2/S3 | 5.1 | 0.11 |
| | (0.23) | (1.29) | | (0.05) | added | (0.01) | (85/5/10) | | |
| Example 21 | PA-14 | PAG-C | D | DBA | HR-47 | W-4 | S1 | 5.4 | 0.11 |
| | (0.30) | (1.39) | | (0.03) | added | (0.01) | (100) | | |
| Example 22 | PA-6 | PAG-A | D | DIA | HR-47 | none | S1 | 6.8 | 0.08 |
| | (0.38) | (1.50) | | (0.05) | added | | (100) | | |
| Example 23 | PA-6 | PAG-A | C | DBA | HR-53 | W-4 | S1/S3 | 6.9 | 0.08 |
| | (0.29) | (1.44) | | (0.03) | added | (0.01) | (90/10) | | |
| Example 24 | PA-2 | PAG-B | D | DBA | HR-26 | W-2 | S1/S2/S3 | 5.5 | 0.12 |
| | (0.32) | (1.52) | | (0.03) | added | (0.02) | (85/5/10) | | |
| Example 25 | PA-2 | PAG-B | C | DIA | HR-26 | W-4 | S1/S3 | 5.6 | 0.12 |
| | (0.30) | (1.40) | | (0.05) | added | (0.01) | (90/10) | | |
| Example 26 | PA-2 | PAG-C | D | DIA | HR-26 | W-3 | S1/S6 | 5.8 | 0.12 |
| | (0.19) | (1.28) | | (0.05) | added | (0.01) | (80/20) | | |
| Example 27 | PA-4 | PAG-D | D | DBA | HR-47 | W-2 | S1 | 5.9 | 0.11 |
| | (0.19) | (1.54) | | (0.03) | added | (0.02) | (100) | | |
| Example 28 | PA-6 | PAG-A | B | DIA | HR-53 | W-1 | S1/S3 | 7.0 | 0.08 |
| | (0.35) | (1.50) | | (0.05) | added | (0.005) | (90/10) | | |
| Example 29 | PA-5 | PAG-D | D | DIA | HR-47 | none | S1/S3 | 6.1 | 0.11 |
| | (0.27) | (1.19) | | (0.05) | added | | (90/10) | | |
| Example 30 | PA-7 | PAG-C | D | DIA | HR-47 | W-4 | S1 | 6.4 | 0.08 |
| | (0.31) | (1.46) | | (0.05) | added | (0.01) | (100) | | |
| Example 31 | PA-14 | PAG-A | D | DBA | HR-47 | W-4 | S1 | 6.7 | 0.11 |
| | (0.30) | (1.50) | | (0.03) | added | (0.01) | (100) | | |
| Example 32 | PA-10 | PAG-C | D | APCA | HR-47 | W-4 | S1 | 4.0 | 0.12 |
| | (0.31) | (1.55) | | (0.05) | added | (0.01) | (100) | | |
| Example 33 | PA-14 | PAG-A | C | DIA | HR-53 | W-4 | S1 | 6.8 | 0.11 |
| | (0.27) | (1.50) | | (0.05) | added | (0.01) | (100) | | |
| Example 34 | PA-1 | PAG-B | D | DBA | HR-26 | W-4 | S1 | 4.5 | 0.12 |
| | (0.19) | (1.39) | | (0.03) | added | (0.01) | (100) | | |
| Example 35 | PA-14 | PAG-A | B | DIA | HR-28 | W-4 | S1/S5 | 6.9 | 0.11 |
| | (0.30) | (1.50) | | (0.05) | added | (0.01) | (60/40) | | |
| Example 36 | PA-10 | PAG-C | E | DIA | HR-47 | W-4 | S1 | 4.2 | 0.12 |
| | (0.35) | (1.50) | | (0.05) | added | (0.01) | (100) | | |
| Example 37 | PA-10 | PAG-C | F | DIA | HR-47 | W-4 | S1 | 4.1 | 0.12 |
| | (0.35) | (1.50) | | (0.05) | added | (0.01) | (100) | | |
| Example 38 | PA-10 | PAG-C | G | DIA | HR-47 | W-4 | S1 | 4.1 | 0.12 |
| | (0.35) | (1.50) | | (0.05) | added | (0.01) | (100) | | |
| Example 39 | PA-10 | PAG-C | C | APCA | HR-28 | W-2 | S1/S4/S6 | 4.1 | 0.12 |
| | (0.28) | (1.53) | | (0.05) | added | (0.02) | (80/5/15) | | |
| Example 40 | PA-10 | PAG-C | H | DIA | HR-47 | W-4 | S1 | 4.1 | 0.11 |
| | (0-35) | (1.50) | | (0.05) | added | (0.01) | (100) | | |
| Example 41 | PA-10 | PAG-C/PAG-H | D | DIA | HR-47 | W-4 | S1 | 4.1 | 0.11 |
| | (0.35) | (0.70/0.75) | | (0.05) | added | (0.01) | (100) | | |
| Example 42 | PA-10 | PAG-C | D | DIA/PEA | HR-47 | W-4 | S1 | 4.0 | 0.11 |
| | (0.35) | (1.50) | | (0.02/0.02) | added | (0.01) | (100) | | |
| Example 43 | PA-10 | PAG-C | C/D | DIA | HR-47 | W-4 | S1 | 4.1 | 0.12 |
| | (0.35) | (1.50) | (5 g/5 g) | (0.05) | added | (0.01) | (100) | | |
| Example 44 | PA-10 | PAG-C | B | APCA | HR-26 | W-4 | S1/S3 | 4.2 | 0.12 |
| | (0.15) | (1.23) | | (0.05) | added | (0.01) | (90/10) | | |
| Example 45 | PA-10 | PAG-C | D | DIA | none | W-4 | S1 | 4.0 | 0.12 |
| | (0.31) | (1.55) | | (0.05) | | (0.01) | (100) | | |
| Example 46 | PA-10 | PAG-C | C | PEA | none | W-2 | S1/S4/S6 | 4.1 | 0.12 |
| | (0.28) | (1.53) | | (0.05) | | (0.02) | (80/5/15) | | |
| Example 47 | PA-2 | PAG-B | B | TEA | none | W-1 | S1/S6 | 5.7 | 0.12 |
| | (0.32) | (1.43) | | (0.03) | | (0.01) | (95/5) | | |
| Example 48 | PA-4 | PAG-C | D | PEA | none | W-4 | S1/S5 | 6.0 | 0.11 |
| | (0.20) | (1.32) | | (0.02) | | (0.01) | (80/20) | | |
| Example 49 | PA-6 | PAG-A | B | DIA | none | W-1 | S1/S3 | 7.0 | 0.08 |
| | (0.35) | (1.50) | | (0.05) | | (0.005) | (90/10) | | |
| Example 50 | PA-7 | PAG-C | D | DIA | none | W-4 | S1(100) | 6.4 | 0.08 |
| | (0.31) | (1.46) | | (0.05) | | (0.01) | | | |
| Comparative | PA-Ref | PAG-A | D | DIA | HR-47 | W-4 | S1 | 8.7 | 0.05 |
| Example 1 | (0.30) | (1.56) | | (0.05) | added | (0.01) | (100) | | |
| Comparative | PA-Ref | PAG-B | D | DIA | HR-47 | W-4 | S1 | 8.5 | 0.06 |
| Example 2 | (0.30) | (1.56) | | (0.05) | added | (0.01) | (100) | | |
| Example 51 | PA-15 | PAG-I | I | PEA | none | W-2 | S1/S6 | 8.3 | 0.06 |
| | (0.30) | (1.56) | | (0.02) | | (0.02) | (60/40) | | |
| Example 52 | PA-16 | PAG-D | J | PEA | none | W-4 | S1/S5 | 7.9 | 0.06 |
| | (0.30) | (1.56) | | (0.02) | | (0.01) | (80/20) | | |
| Example 53 | PA-17 | PAG-J | K | PEA/DIA | none | W-4 | S1/S5 | 8.3 | 0.06 |
| | (0.30) | (1.56) | | (0.01/0.01) | | (0.01) | (80/20) | | |
| Example 54 | PA-14 | PAG-A | A | DIA | HR-53 | W-4 | S1/S5 | 7.6 | 0.11 |
| | (0.29) | (1.56) | | (0.05) | added | (0.01) | (60/40) | | |
| Example 55 | PA-18 | PAG-C | D | PBI | HR-26 | W-3 | S1 | 4.4 | 0.12 |
| | (0.25) | (1.23) | | (0.04) | added | (0.02) | (100) | | |
| Example 56 | PA-19 | PAG-D | D | DIA | HR-47 | none | S1/S3 | 6.0 | 0.12 |
| | (0.27) | (1.19) | | (0.05) | added | | (90/10) | | |
| Example 57 | PA-20 | PAG-A | B | DIA | none | W-1 | S1/S3 | 6.8 | 0.09 |
| | (0.35) | (1.50) | | (0.05) | | (0.005) | (90/10) | | |
| Example 58 | PA-21 | PAG-C | D | DIA | HR-47 | W-4 | S1 | 4.9 | 0.09 |
| | (0.36) | (1.57) | | (0.05) | added | (0.01) | (100) | | |
| Example 59 | PA-22 | PAG-C | D | PEA | HR-49 | W-4 | S1/S3 | 6.1 | 0.11 |
| | (0.28) | (1.39) | | (0.04) | TC | (0.01) | (90/10) | | |
| Example 60 | PA-23 | PAG-C | D | DIA | HR-47 | W-4 | S1 | 3.8 | 0.12 |
| | (0.31) | (1.55) | | (0.05) | added | (0.01) | (100) | | |
| Example 61 | PA-24 | PAG-C | D | DIA | HR-47 | W-4 | S1/S2/S3 | 4.8 | 0.11 |
| | (0.23) | (1.29) | | (0.05) | added | (0.01) | (85/5/10) | | |
| Example 62 | PA-25 | PAG-C | D | PEA | HR-49 | W-4 | S1/S3 | 4.6 | 0.12 |
| | (0.28) | (1.39) | | (0.04) | TC | (0.01) | (90/10) | | |
| Comparative | PA-REF2 | PAG-C | D | PEA | HR-28 | W-4 | S1/S5 | 9.0 | 0.03 |
| Example 3 | (0.20) | (1.32) | | (0.02) | added | (0.01) | (80/20) | | |
| Comparative | PA-Ref | PAG-A | B | DIA | none | W-1 | S1/S3 | 8.5 | 0.03 |
| Example 4 | (0.30) | (1.50) | | (0.05) | | (0.005) | (90/10) | | |
| Example 63 | PA-10 (0.16) | PAG-C | D | DIA | HR-47 | W-4 | S1 | 4.1 | 0.12 |
| | PA-11 (0.14) | (1.55) | | (0.05) | added | (0.01) | (100) | | |
| Example 64 | PA-25 | PAG-K | D | PEA | HR-49 | W-4 | S1/S3 | 4.5 | 0.12 |
| | (0.28) | (1.39) | | (0.04) | TC | (0.01) | (90/10) | | |
| Example 65 | PA-25 | PAG-L | B | PEA | HR-49 | W-4 | S1/S3 | 4.6 | 0.12 |
| | (0.28) | (1.45) | | (0.04) | TC | (0-01) | (90/10) | | |

Abbreviations in the Tables stand for those illustrated in specific examples or the followings.

[Compound (PA)] ε: Molar Extinction Coefficient (l/mol/cm)

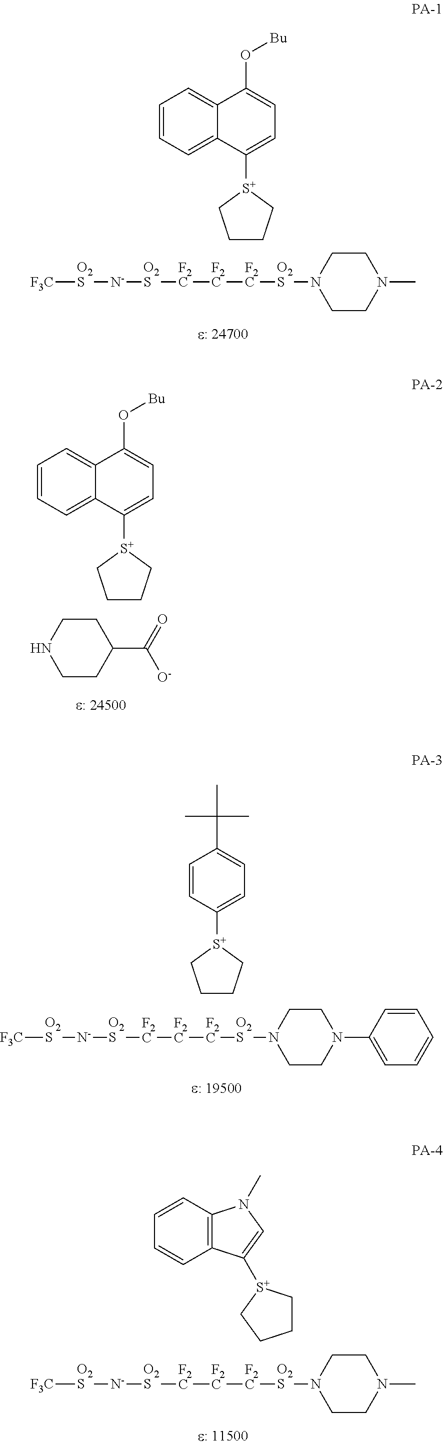

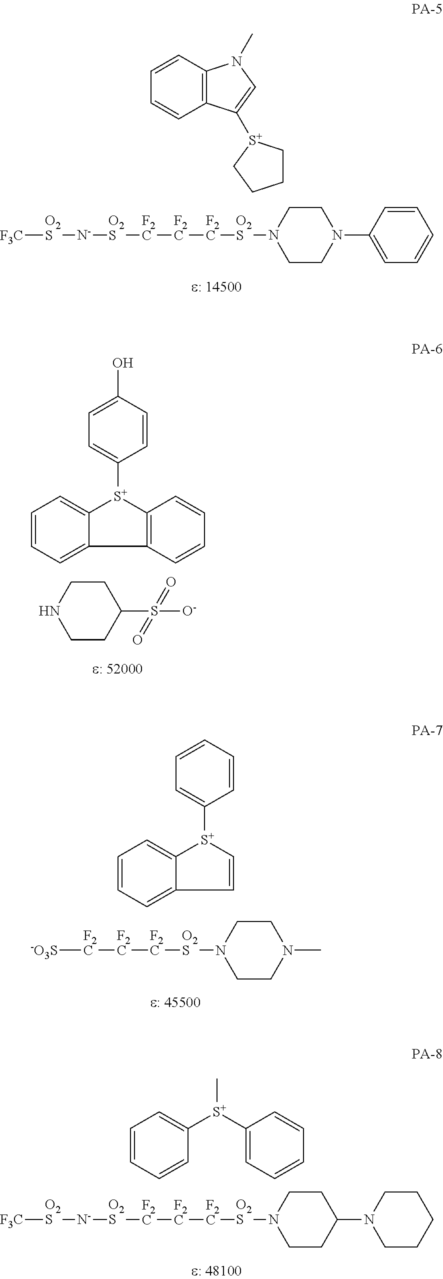

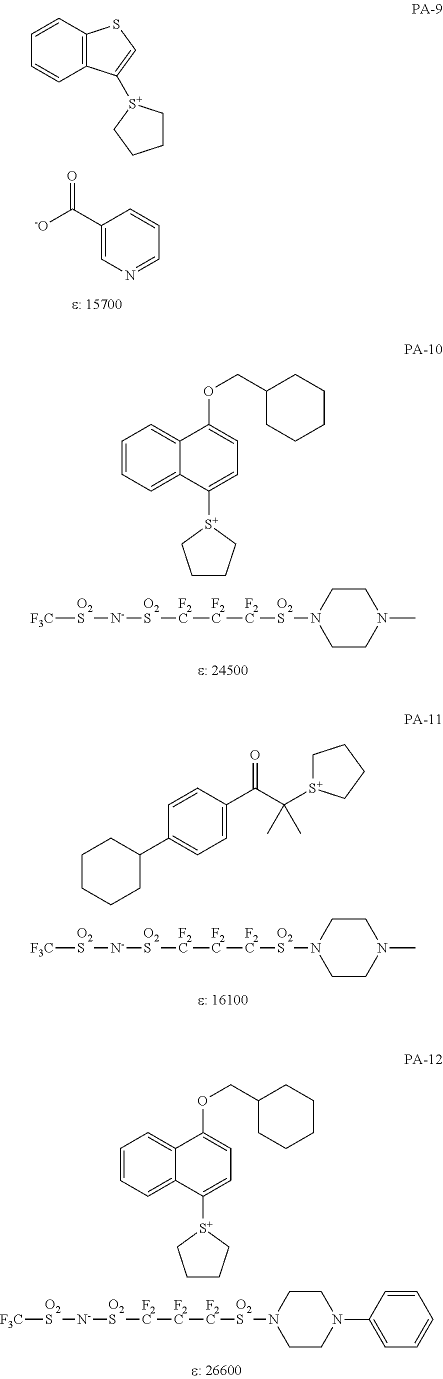

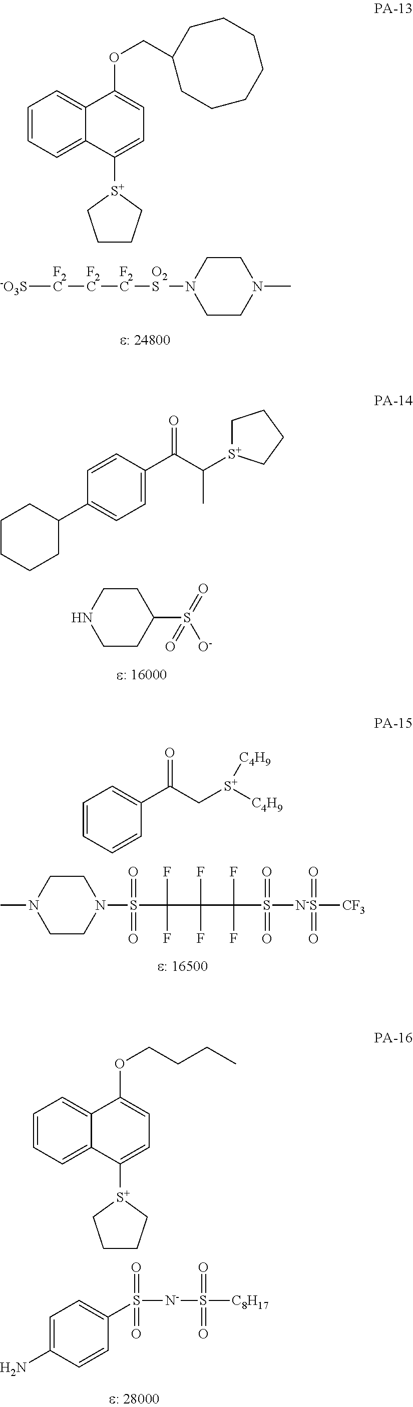

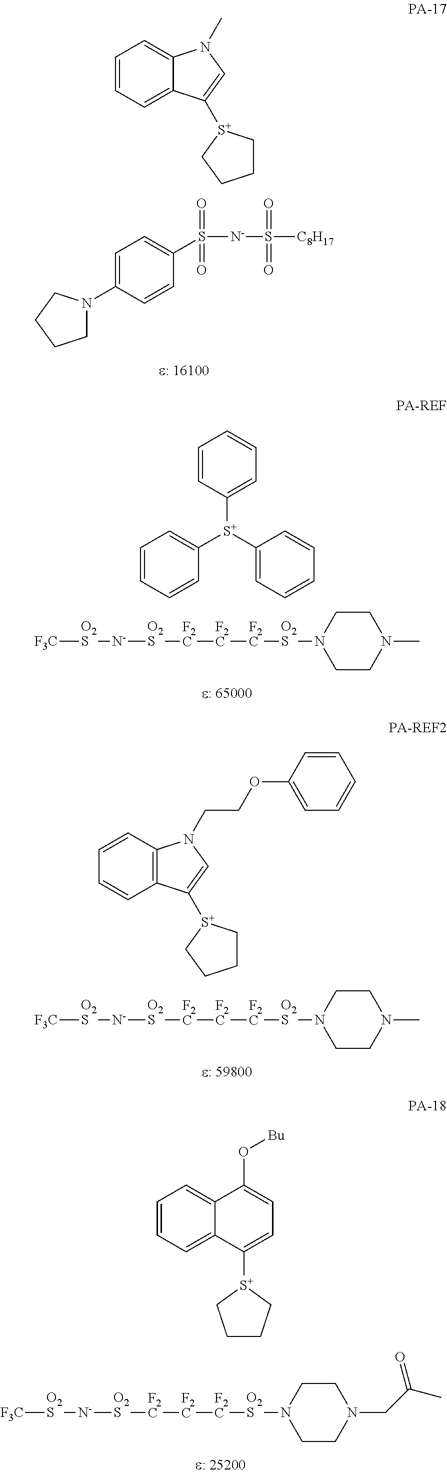

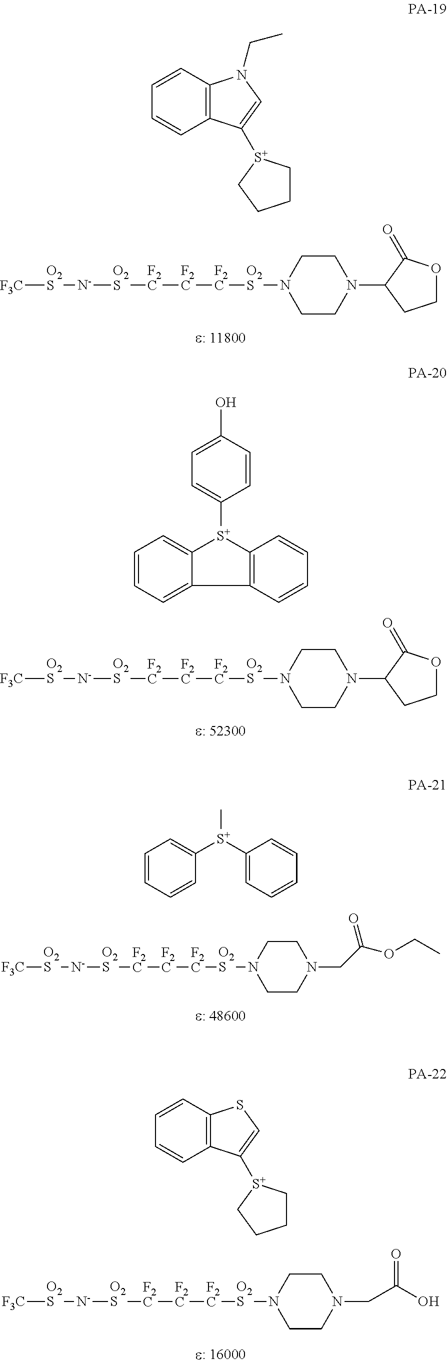

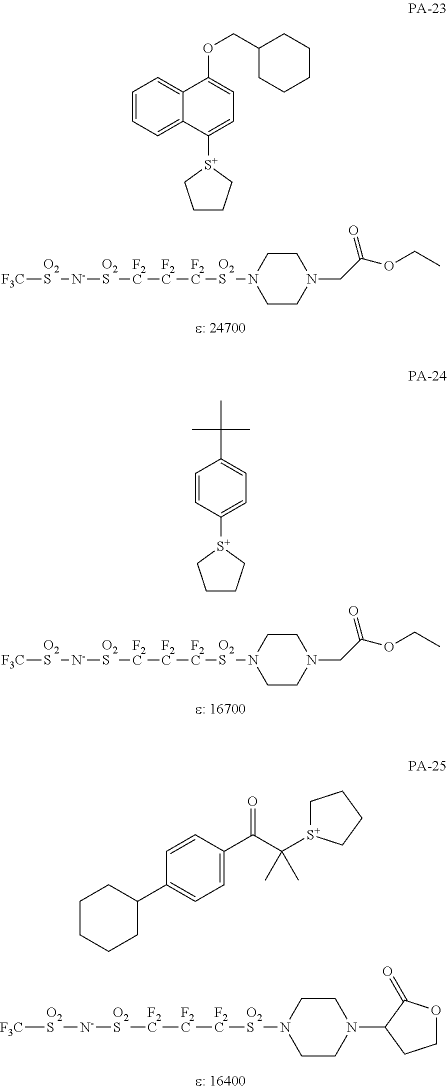

### [Acid Generator]

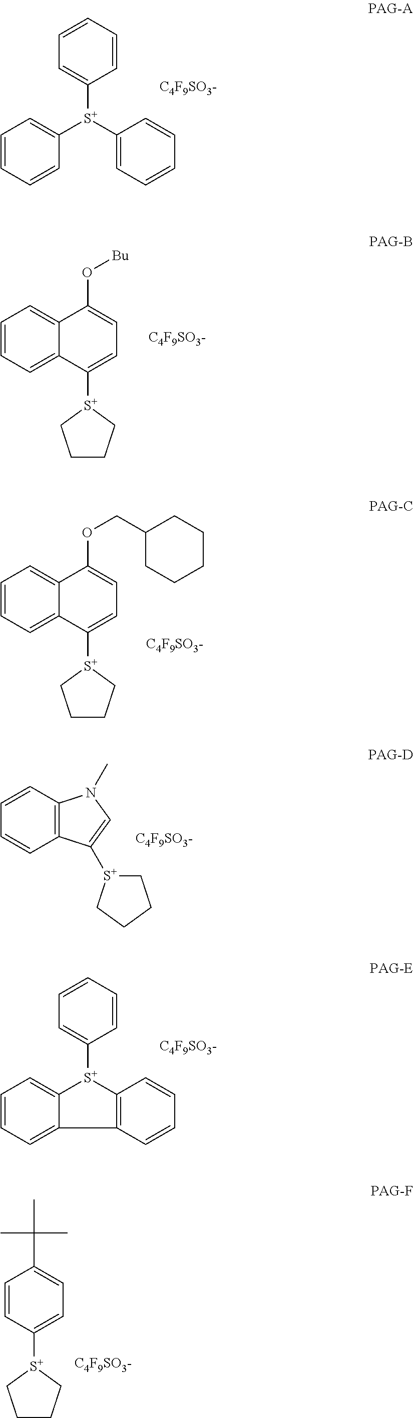

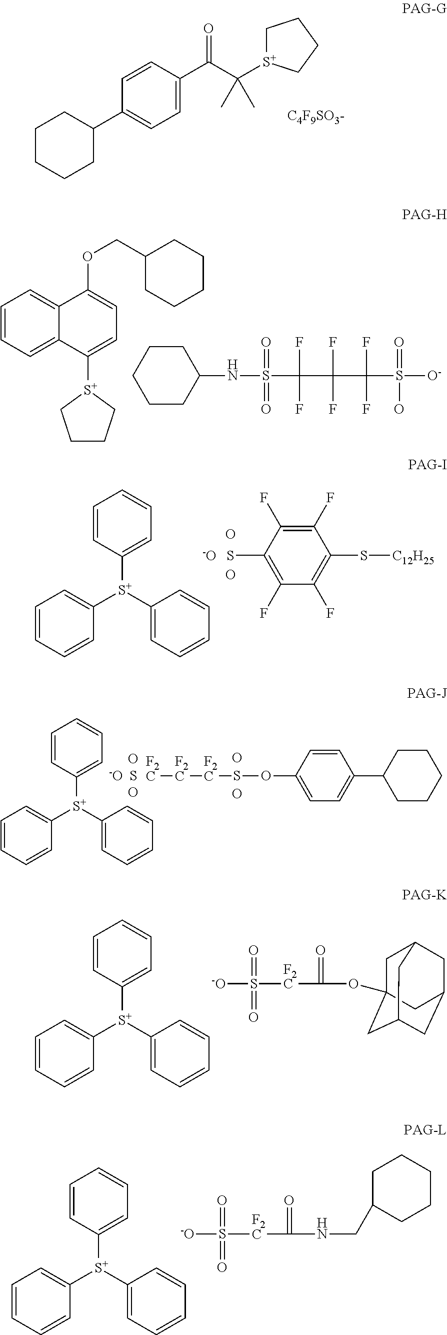

### [Resin (B1)]

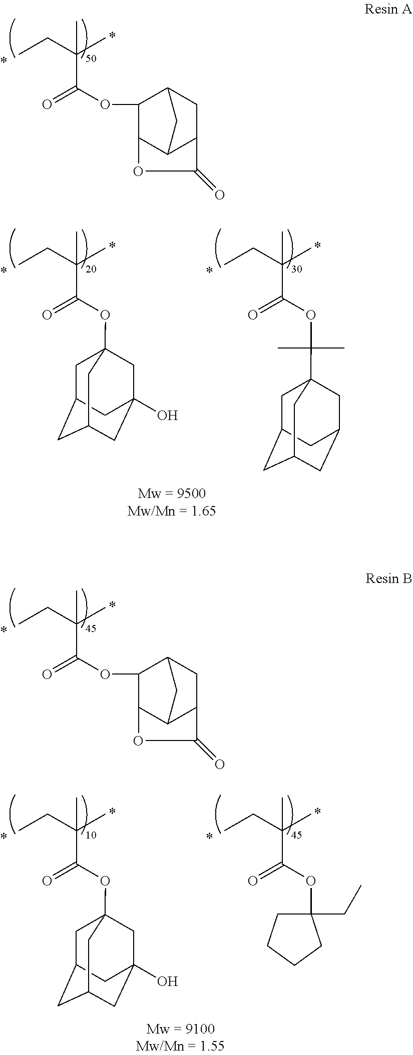

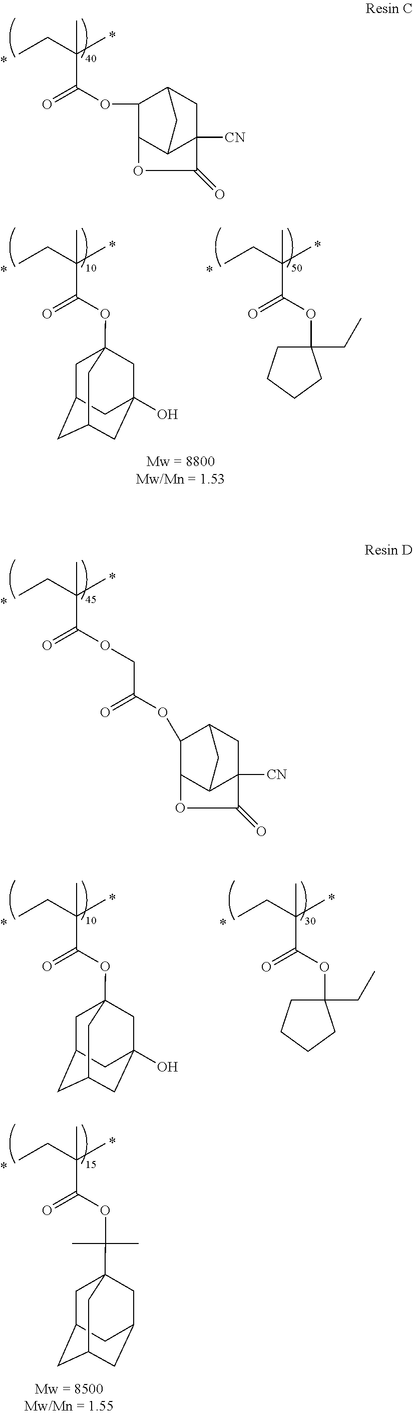

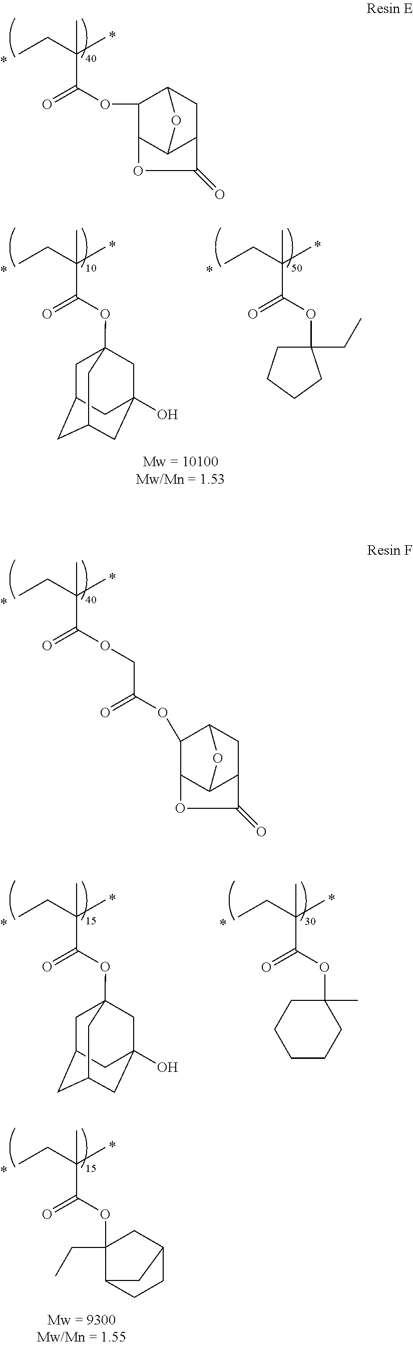

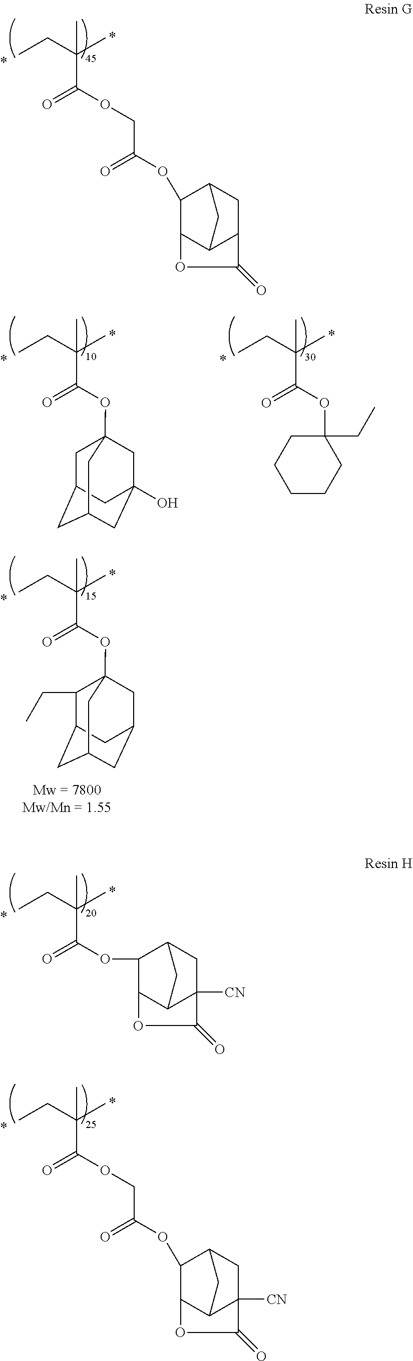

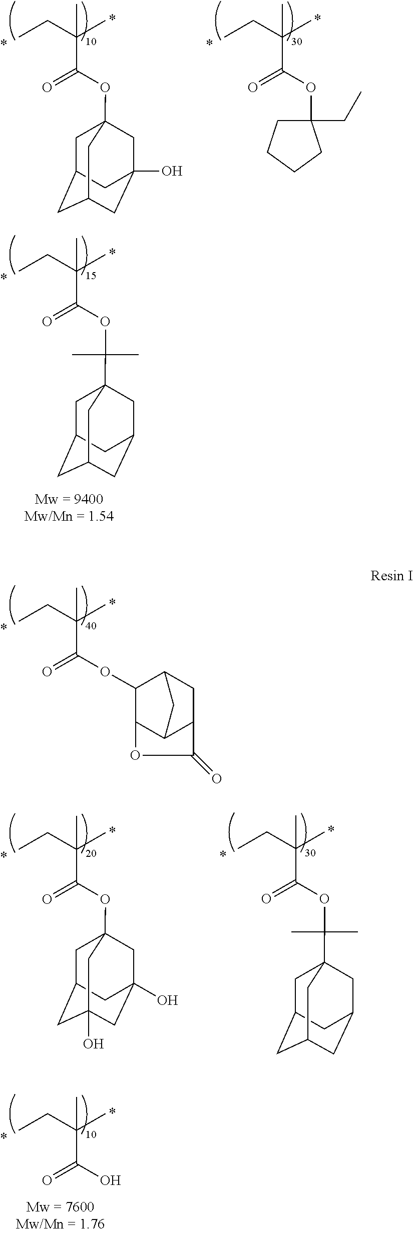

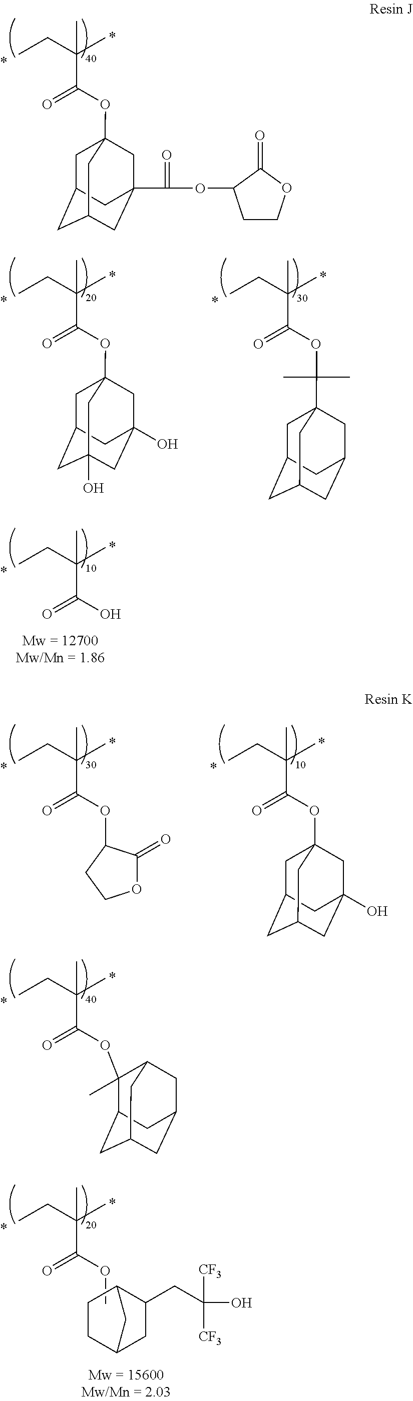

### [Basic Compound]

TPSA: Triphenylsulfonium acetate

### DIA: 2,6-Diisopropylaniline

### TEA: Triethanolamine

### DBA: N,N-Dibutylaniline

### PBI: 2-Phenylbenzimidazole

### PEA: N-Phenyldiethanolamine

### [(D) Low Molecular Compound Having a Group Capable of Leaving by the Action of an Acid (Compound (D))]

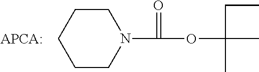

### [Surfactant]

W-1: Megaface F176 (produced by Dainippon Ink & Chemicals, Inc.) (fluorine-containing)
W-2: Megaface R08 (produced by Dainippon Ink & Chemicals, Inc.) (fluorine- and silicon-containing)
W-3: polysiloxane polymer KP-341 (produced by Shin-Etsu Chemical Co., Ltd.) (silicon-containing)
W-4: Troysol S-366 (produced by Troy Chemical)

### [Solvent]

S1: Propylene glycol monomethyl ether acetate (PGMEA; 1-methoxy-2-acetoxypropane)

### S2: 2-Heptanone

### S3: Cyclohexanone

### S4: γ-Butyrolactone

S5: Propylene glycol monomethyl ether (PGME; 1-methoxy-2-propanol)
S6: Ethyl lactate
S7: Propylene carbonate

As apparent from Table 3, in Comparative Examples 1 to 4 where the compound (PA) is out of the scope of the present invention, LWR and DOF performances are poor.

On the other hand, all of the compositions of the present invention are verified to be excellent in LWR and DOF performances.

Also, enhancement of LWR and DOF performances in the immersion exposure of Examples 1 to 44, 54 to 56 and 58 to 65 contrasted with Comparative Examples 1 to 3 tends to be larger than the enhancement of LWR and DOF performances in the dry exposure of Examples 45 to 53 and 57 contrasted with Comparative Example 4.

### INDUSTRIAL APPLICABILITY

According to the present invention, an actinic ray-sensitive or radiation-sensitive resin composition improved in LWR and DOF and suitable also for an immersion process with a line width of 45 nm or less, and a resist film and a pattern forming method each using the composition, are provided.

This application is based on Japanese patent applications No. 2009-199037 filed on Aug. 28, 2009 and No. 2010-142061 filed on Jun. 22, 2010, the entire content of which is hereby incorporated by reference, the same as if set forth at length.

1. An actinic ray-sensitive or radiation-sensitive resin composition, comprising:
(PA) a compound having a proton acceptor functional group and undergoing decomposition upon irradiation with an actinic ray or radiation to generate a compound reduced in or deprived of proton acceptor property or changed to be acidic from being proton acceptor-functioning,
wherein a molar extinction coefficient ε of the compound (PA) at a wavelength of 193 nm as measured in acetonitrile solvent is 55,000 or less.

2. The actinic ray-sensitive or radiation-sensitive resin composition according to claim 1, further comprising:
(B1) a resin capable of increasing a solubility of the resin (B1) in an alkali developer by an action of an acid,
wherein the resin (B1) contains a resin having a repeating unit represented by the following formula (V), and
the compound (PA) is a compound capable of decomposing upon irradiation with an actinic ray or radiation to generate a compound represented by the following formula (PA-1):
Q-A_{PA1}-(X)ₙ—R (PA-1)
wherein Q represents —SO₃H, —CO₂H or —W₁—NH—W₂—Rf;
each of X, W₁ and W₂ independently represents —SO₂— or —CO—;
Rf represents an alkyl group which may be substituted with a halogen atom, a cycloalkyl group which may be substituted with a halogen atom, or an aryl group which may be substituted with a halogen atom;
A_{PA1} represents a single bond or a divalent linking group;
n represents 0 or 1; and
R represents a monovalent organic group having a proton acceptor functional group: 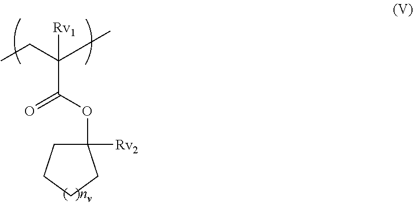
wherein each of Rv₁ and Rv₂ independently represents an alkyl group having a carbon number of 1 to 10; and
nᵥ represents an integer of 1 to 6.

3. The actinic ray-sensitive or radiation-sensitive resin composition according to claim 2, wherein the compound (PA) is represented by the following formula (II) or (III): 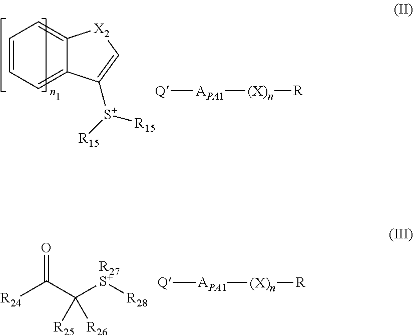
wherein each R₁₅ independently represents an alkyl group or a cycloalkyl group, two R₁₅'s may combine with each other to form a ring;
X₂ represents any one of —CR₂₁═CR₂₂—, —NR₂₃—, —S— and —O—;
each of R₂₁ to R₂₃ independently represents a hydrogen atom, an alkyl group, a cycloalkyl group, an alkoxy group or an aryl group;
R₂₄ represents an aryl group;
each of R₂₅ and R₂₆ independently represents a hydrogen atom, an alkyl group or a cycloalkyl group, R₂₅ and R₂₆ may combine with each other to form a ring;
each of R₂₇ and R₂₈ independently represents a hydrogen atom, an alkyl group, a cycloalkyl group, an allyl group or a vinyl group, R₂₇ and R₂₈ may combine with each other to form a ring;
n₁ represents an integer of 0 to 3;
Q′ represents —SO₃—, —CO₂— or —W₁—N⁻—W₂—Rf; and
X, W₁, W₂, Rf, A_{PA1}, R and n have the same meanings as X, W₁, W₂, Rf, A_{PA1}, R and n in formula (PA-1).

4. The actinic ray-sensitive or radiation-sensitive resin composition according to claim 3,
wherein Q′ in formula (II) or (III) is —W₁—N⁻—W₂—Rf,
wherein each of W₁ and W₂ independently represents —SO₂— or —CO—; and
Rf represents an alkyl group which may be substituted with a halogen atom, a cycloalkyl group which may be substituted with a halogen atom, or an aryl group which may be substituted with a halogen atom.

5. The actinic ray-sensitive or radiation-sensitive resin composition according to claim 1, further comprising:
(C) a compound capable of generating an acid upon irradiation with an actinic ray or radiation.

6. The actinic ray-sensitive or radiation-sensitive resin composition according to claim 1,
wherein the resin (B1) has a lactone group substituted with a cyano group.

7. The actinic ray-sensitive or radiation-sensitive resin composition according to claim 1.
wherein the resin (B1) contains a repeating unit having a lactone structure represented by the following formula (III): 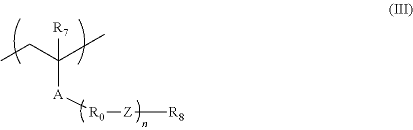
wherein A represents an ester bond or an amide bond;
R₀ represents an alkylene group, a cycloalkylene group or a combination thereof, and when a plurality of R₀'s are present, the plurality of R₀'s are the same or different;
Z represents an ether bond, an ester bond, an amide bond, a group represented by —O—C(═O)—N(R)— or —N(R)—C(═O)—O—, or a group represented by —N(R)—C(═O)—N(R)—, and when a plurality of Z's are present, the plurality of Z's are the same or different, in which R represents a hydrogen atom, an alkyl group, a cycloalkyl group or an aryl group;
R₈ represents a monovalent organic group having a lactone structure;
n represents an integer of 1 to 5; and
R₇ represents a hydrogen atom, a halogen atom or an alkyl group.

8. The actinic ray-sensitive or radiation-sensitive resin composition according to claim 1, further comprising:
a hydrophobic resin.

9. A resist film, which is formed from the actinic ray-sensitive or radiation-sensitive resin composition according to claim 1.

10. A pattern forming method, comprising:
forming a resist film by using the actinic ray-sensitive or radiation-sensitive resin composition according to claim 1; and
exposing and developing the resist film.

11. The pattern forming method according to claim 10,
wherein exposure in the exposing is immersion exposure.
